(19) United States
(12) Patent Application Publication
(10) Pub. No.: US 2023/0067378 A1
(43) Pub. Date: Mar. 2, 2023

(54) COMPOSITIONS AND METHODS FOR THE TREATMENT OF ESTROGEN-DEPENDENT DISORDERS

(71) Applicant: ObsEva S.A., Plan-les-Ouates (CH)

(72) Inventors: Jean-Pierre GOTTELAND, Geneva (CH); Ernest LOUMAYE, Cologny (CH); Oliver POHL, Plan-les-Ouates (CH)

(21) Appl. No.: 17/291,192

(22) PCT Filed: Nov. 6, 2019
(86) PCT No.: PCT/EP2019/080362

§ 371 Date: May 4, 2021

Related U.S. Application Data
(60) Provisional application No. 62/756,946, filed on Nov. 7, 2018.

(51) Int. Cl. A61K 31/519; A61K 31/513; A61K 31/567; A61K 31/57; A61K 31/585; A61P 5/30

(52) U.S. Cl. CPC A61K 31/519; A61K 31/513; A61P 5/30; A61K 31/57; A61K 31/585; A61K 31/567

(57) ABSTRACT

The present disclosure provides compositions and methods for treating estrogen-dependent disorders, such as disorders of the female reproductive system, including uterine fibroids and endometriosis, among others. Compounds described herein that may be used to treat such indications include gonadotropin-releasing hormone (GnRH) antagonists. Suitable GnRH antagonists useful in conjunction with the compositions and methods described herein include thieno[3,4d]pyrimidine derivatives, such as 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid and the choline salt thereof, among others. Using the compositions and methods described herein, GnRH antagonists may be periodically administered to a patient, such as one or more times per day, week, or month, in combination with periodic add-back therapy, thereby preventing bone mineral density loss that may otherwise accompany estrogen depletion effectuated by GnRH antagonist activity. Advantageously, using the dosing schedules of the present disclosure, the periodic administration the GnRH antagonist and add-back therapy may commence together, such as on the same day.

### FIELD OF THE INVENTION

The invention relates to the therapeutic treatment of disorders of the female reproductive system, including uterine fibroids and heavy menstrual blood loss associated therewith.

### BACKGROUND OF THE INVENTION

Estrogen-dependent disorders represent a challenging class of diseases that have a high incidence in the general population and are often associated with particularly severe symptomology.

Uterine fibroids, for example, also referred to as leiomyomata, are among the most common benign tumors in women. Symptoms associated with uterine fibroids commonly include heavy or prolonged menstrual bleeding, pelvic pressure and pelvic organ compression, back pain, and adverse reproductive outcomes. Heavy menstrual bleeding may lead to iron deficiency anemia, a key symptom of uterine fibroids and the leading cause of surgical interventions that may include hysterectomy. Endometriosis is another estrogen-dependent gynecological condition, characterized by the presence of endometrial-like tissue outside the uterus. A chronic inflammatory reaction induced by the ectopic endometrial cells, endometriosis may result in infertility and a variety of pain symptoms including dysmenorrhea, dyspareunia, chronic pelvic pain, dysura, and dyschezia, among others.

Additional examples of estrogen-dependent diseases include adenomyosis and rectovaginal endometriosis, which are particularly severe endometrial growth disorders characterized by the invasion of endometrial tissue into the uterine myometrium and rectovaginal zones, respectively. The term adenomyosis or uterine adenomyosis is used to describe the presence of both endometrial glands and stroma deep within the myometrium. This condition is associated with hypertrophy and hyperplasia of the subjacent muscle cells, which may ultimately result in an altered size and globulous morphology of the uterus. Due to the severity of this disorder, one of the key symptoms is strong menstrual and even non-menstrual pelvic pain with abnormal uterine bleeding. Like adenomyosis, rectovaginal endometriosis patients present with a variety of pain symptoms including dysmenorrhea, dyspareunia, chronic pelvic pain, dysuria, and dyschezia. Treatment options for rectovaginal endometriosis are limited. Since medical therapies are either ineffective or have considerable side effects, rectovaginal endometriosis patients often undergo surgical procedures to reduce the endometrial node, and may even be subject to resection of the bowel if the node infiltrates the rectal or sigmoidal wall.

There exists a need for new, effective therapeutics for alleviating the symptoms associated with these and other estrogen-dependent disorders, as well as for treating their underlying pathology.

### SUMMARY OF THE INVENTION

The present disclosure relates to compositions and methods for the treatment of estrogen-dependent disorders, such as uterine fibroids and endometriosis, among others. Using the compositions and methods of the disclosure, a patient, such as a female human patient, may be administered a gonadotropin-releasing hormone (GnRH) antagonist so as to treat the underlying biochemical etiology of one or more of these diseases and/or to alleviate one or more symptoms associated with such conditions. For example, in the case of a patient having uterine fibroids, the patient may be administered a GnRH antagonist so as to reduce the serum concentration of follicle-stimulating hormone (FSH) and/or luteinizing hormone (LH) in the patient, thereby suppressing endogenous levels of β17-estradiol (E2). Without being limited by mechanism, the diminished E2 concentration induced by the GnRH antagonist can result in a beneficial effect on symptomology, manifesting in the patient, for example, as a reduction in uterine fibroid volume and/or uterine blood loss. Similarly, in the context of a patient having endometriosis, using the compositions and methods described herein, the patient may be administered a GnRH antagonist so as to reduce endogenous E2 levels, thereby diminishing the volume of endometrial tissue extending outside of the uterus and/or alleviating such symptoms as global pelvic pain, dysmenorrhea, dyspareunia, and dyschezia. The compositions and methods of the disclosure can also be used to treat particularly severe endometrial growth disorders, including adenomyosis and rectovaginal endometriosis, among other pathologies mediated by excessive E2 production.

GnRH antagonists that may be used in conjunction with the compositions and methods described herein include optionally substituted thieno[3,4d]pyrimidine derivatives, such as 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid or the choline salt thereof. In some embodiments, the GnRH antagonist is an optionally substituted 3-aminoalkyl pyrimidine-2,4(1H,3H)-dione derivative, such as sodium 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoate, also referred to as elagolix, or the carboxylic acid conjugate thereof. The GnRH antagonist may be, for example, an optionally substituted thieno[2,3d]pyrimidine derivative, such as N-(4-(1-(2,6-difluorobenzyl)-5-((dimethylamino)methyl)-3-(6-methoxy-3-pyridazinyl)-2,4-dioxo-1,2,3,4-tetrahydrothieno[2,3-d]pyrimidin-6-yl)phenyl)-N-methoxyurea, also referred to as relugolix, or a pharmaceutically acceptable salt thereof. In some embodiments, the GnRH antagonist is an optionally substituted propane-1,3-dione derivative, such as (2R)—N-{5-[3-(2,5-difluorophenyl)-2-(1,3-dihydro-2H-benzimidazol-2-ylidene)-3-oxopropanoyl]-2-fluorobenzene-1-sulfonyl}-2-hydroxypropanimidamide, also referred to as opigolix or ASP-1707. Additional GnRH antagonists that may be used in conjunction with the compositions and methods described herein include SK12670 and BAY-784, as well as derivatives and variants thereof, among others.

Using the compositions and methods described herein, the GnRH antagonist may be administered to the patient in combination with add-back therapy, which provides the patient with a supply of estrogen in order to counteract potentially harmful side effects that could otherwise accompany excessive depletion of endogenous E2. Particularly, suppression of a patient's circulating E2 concentration to levels substantially less than 20 pg/ml may lead to partial reductions in bone mineral density. The dual administration of a GnRH antagonist and add-back therapy may have the effect of partially compensating for the reduction in endogenous E2 engendered by the GnRH antagonist, thereby suppressing E2 to a level sufficient to treat the disease of interest without permitting bone mineral density loss. The add-back therapy may contain an estrogen, such as β17-estradiol, ethinyl estradiol, or a conjugated estrogen (e.g., a conjugated equine estrogen), and/or a progestin, such as norethindrone or a compound that is metabolized in vivo to produce norethindrone. For example, the progestin may be an ester of norethindrone that is de-esterified in vivo to produce norethindrone, such as norethindrone acetate. Additional progestins that may be incorporated into an add-back therapy described herein include progesterone, norgestimate, medroxyprogesterone, and drospirenone. In some embodiments, the add-back therapy contains a combination of an estrogen and a progestin, such as about 1.0 mg of β17-estradiol and about 0.5 mg of norethindrone or a compound that is metabolized in vivo to produce norethindrone, such as norethindrone acetate. Additional examples of add-back therapy include those that contain about 0.5 mg of β17-estradiol and about 0.1 mg of norethindrone or a compound that is metabolized in vivo to produce norethindrone, such as norethindrone acetate, among other forms of add-back therapy described herein.

The present disclosure is based, in part, on the surprising discovery that concurrent commencement of GnRH antagonist administration and add-back therapy administration effectuates a reduction in endogenous E2 levels that is superior in magnitude and longer lasting relative to dosing regimens in which the patient is pre-treated with a GnRH antagonist alone before receiving the GnRH antagonist in combination with add-back therapy. This finding is unexpected given the mechanism by which add-back therapy exerts its biological effects. Add-back therapy partially neutralizes the effects of a GnRH antagonist by providing the patient with a supply of estrogen. Although add-back therapy is provided in order to prevent undesirable side effects, such as bone mineral density loss, the add-back therapy nonetheless partially counteracts the E2-reducing activity of the GnRH antagonist. It is this E2-reducing effect that is primarily responsible for the efficacy of a GnRH antagonist in treating an estrogen-dependent disorder, such as uterine fibroids and endometriosis, among others described herein. The discovery that simultaneous onset of the administration of a GnRH antagonist and add-back therapy to a patient engenders a greater, more sustained reduction in circulating E2 relative to instances in which a patient is first treated with the GnRH antagonist alone before receiving add-back therapy is, thus, entirely counter-intuitive.

In uterine fibroids patients, the superior reduction in E2 achieved by concurrent onset of GnRH antagonist and add-back therapy administration manifests as a marked improvement in uterine bleeding pattern. It has presently been discovered that patients administered a GnRH antagonist for the treatment of uterine fibroids with a simultaneous onset of the antagonist and add-back therapy exhibit substantially diminished uterine bleeding relative to patients that are pre-treated with the GnRH antagonist alone before receiving add-back therapy. This finding is particularly surprising for at least the following reasons: It has been observed that patients pre-treated with a GnRH antagonist for several weeks have endogenous E2 concentrations resembling those of post-menopausal subjects. When post-menopausal subjects are administered add-back therapy, the uterine blood loss exhibited by these subjects remains low. Given this result, one of skill in the art might conclude that subjects pre-treated with a GnRH antagonist before receiving add-back therapy would similarly exhibit minimal uterine blood loss. In contrast, the present disclosure is based, in part, on the unexpected finding that subjects that are provided a simultaneous onset of GnRH antagonist administration and add-back therapy administration exhibit significantly less uterine bleeding relative to subjects that are pre-treated with a GnRH antagonist alone before initiating add-back therapy. This unexpected and advantageous property is consistent with the discovery, detailed above, that concurrent onset of GnRH antagonist administration and add-back therapy administration engenders a superior, more sustained reduction in a patient's endogenous E2 concentration relative to dosing regimens in which a patient is pre-treated with a GnRH antagonist alone before initiating add-back therapy.

The compositions and methods described herein, thus, provide a series of important clinical benefits. Using the compositions and methods of the disclosure, a patient, such as a female human patient, may be concurrently administered a GnRH antagonist in combination with add-back therapy so as to treat the underlying cause of, and/or alleviate one or more symptoms associated with, an estrogen-dependent disease with a greater efficacy relative to patients that are pre-treated with the GnRH antagonist alone before receiving the add-back therapy. Significantly, the compositions and methods of the disclosure may provide this beneficial treatment outcome while still preventing the bone mineral density loss that could otherwise accompany hypoestrogenemia. Thus, using the compositions and methods described herein, a patient suffering from an estrogen-dependent disease (e.g., uterine fibroids and endometriosis, among others described herein) may be treated with a more effective E2-reducing dosing regimen while still safeguarding the patient from harmful side effects of E2 depletion.

To achieve these beneficial treatment effects, a patient, such as a female human patient, may be periodically administered a GnRH antagonist in combination with add-back therapy. The administration of each of these agents may commence at substantially the same time, such that administration of the later-provided agent begins while there is still a detectable concentration of the earlier-provided agent in the patient's circulating blood. For example, in accordance with the compositions and methods of the disclosure, administration of periodic GnRH antagonist therapy and add-back therapy for the treatment of an estrogen-dependent disease (e.g., uterine fibroids and/or endometriosis, among others described herein) may commence within from about 1 minute to about 7 days of one another, such as within about 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, 30 minutes, 31 minutes, 32 minutes, 33 minutes, 34 minutes, 35 minutes, 36 minutes, 37 minutes, 38 minutes, 39 minutes, 40 minutes, 41 minutes, 42 minutes, 43 minutes, 44 minutes, 45 minutes, 46 minutes, 47 minutes, 48 minutes, 49 minutes, 50 minutes, 51 minutes, 52 minutes, 53 minutes, 54 minutes, 55 minutes, 56 minutes, 57 minutes, 58 minutes, 59 minutes, 60 minutes, 61 minutes, 62 minutes, 63 minutes, 64 minutes, 65 minutes, 66 minutes, 67 minutes, 68 minutes, 69 minutes, 70 minutes, 71 minutes, 72 minutes, 73 minutes, 74 minutes, 75 minutes, 76 minutes, 77 minutes, 78 minutes, 79 minutes, 80 minutes, 81 minutes, 82 minutes, 83 minutes, 84 minutes, 85 minutes, 86 minutes, 87 minutes, 88 minutes, 89 minutes, 90 minutes, 91 minutes, 92 minutes, 93 minutes, 94 minutes, 95 minutes, 96 minutes, 97 minutes, 98 minutes, 99 minutes, 100 minutes, 101 minutes, 102 minutes, 103 minutes, 104 minutes, 105 minutes, 106 minutes, 107 minutes, 108 minutes, 109 minutes, 110 minutes, 111 minutes, 112 minutes, 113 minutes, 114 minutes, 115 minutes, 116 minutes, 117 minutes, 118 minutes, 119 minutes, 120 minutes, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 49 hours, 50 hours, 51 hours, 52 hours, 53 hours, 54 hours, 55 hours, 56 hours, 57 hours, 58 hours, 59 hours, 60 hours, 61 hours, 62 hours, 63 hours, 64 hours, 65 hours, 66 hours, 67 hours, 68 hours, 69 hours, 70 hours, 71 hours, 72 hours, 84 hours, 96 hours, 108 hours, 120 hours, 132 hours, 144 hours, 156 hours, or 168 hours of one another. In some embodiments, administration of the GnRH antagonist and administration of the add-back therapy commence on the same day. Following the first administration of each agent, the GnRH antagonist and add-back therapy may each independently be administered to the patient one or more times per day, week, or month. In some embodiments, after the initial administration, the GnRH antagonist and add-back therapy may be administered to the patient concurrently, such that each time the patient receives a dose of one agent, the patient also receives a dose of the other agent. However, after the initial administration, the patient need not be administered the GnRH antagonist and add-back therapy at the same time during each subsequent dose.

In some embodiments, the GnRH antagonist is a substituted thieno[3,4d]pyrimidine compound, such as 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid or the choline salt thereof, and, for example, the add-back therapy and the GnRH antagonist therapy are initiated within from about 12 hours to about 16 hours of one another (e.g., within 14 hours or 15 hours of one another). In some embodiments, the GnRH antagonist is a substituted 3-aminoalkyl pyrimidine-2,4(1H,3H)-dione compound, such as sodium 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethy}amino)butanoate or the carboxylic acid conjugate thereof, and, for example, the add-back therapy and the GnRH antagonist therapy are initiated within from about 2 hours to about 6 hours of one another (e.g., within from about 3 hours to about 5 hours of one another). In some embodiments, the GnRH antagonist is a substituted thieno[2,3d]pyrimidine compound, such as N-(4-(1-(2,6-difluorobenzyl)-5-((dimethylamino)methyl)-3-(6-methoxy-3-pyridazinyl)-2,4-dioxo-1,2,3,4-tetrahydrothieno[2,3-d]pyrimidin-6-yl)phenyl)-N′-methoxyurea or a pharmaceutically acceptable salt thereof, and, for example, the add-back therapy and the GnRH antagonist therapy are initiated within from about 37 hours to about 42 hours of one another (e.g., within from about 39 hours to about 41 hours of one another).

In a first aspect, the disclosure features a method of treating an estrogen-dependent disease in a patient (e.g., a mammalian patient, such as a human patient, and particularly a female human patient) in need thereof by periodically administering to the patient therapeutically effective amounts of a GnRH antagonist and add-back therapy. In an additional aspect, the disclosure features a method of treating an estrogen-dependent disease in a patient (e.g., a mammalian patient, such as a human patient, and particularly a female human patient) by:
a) diagnosing the patient as having an estrogen-dependent disease; and
b) periodically administering to the patient therapeutically effective amounts of a GnRH antagonist and add-back therapy.

In some embodiments, the estrogen-dependent disease is uterine fibroids. In some embodiments, the estrogen-dependent disease is endometriosis, such as rectovaginal endometriosis. In some embodiments, the estrogen-dependent disease is adenomyosis. In some embodiments, the estrogen-dependent disease is benign prostatic hypertrophy, precocious puberty, premenstrual syndrome, polycystic ovary syndrome, hirsutism, short stature, a sleep disorder, acne, baldness, or irritable bowel syndrome.

In another aspect, the disclosure features a method of reducing the volume of menstrual blood loss a human patient diagnosed as having uterine fibroids by periodically administering to the patient therapeutically effective amounts of a GnRH antagonist and add-back therapy. In an additional aspect, the disclosure features a method of reducing the volume of menstrual blood loss a human patient by:
a) diagnosing the patient as having uterine fibroids; and
b) periodically administering to the patient therapeutically effective amounts of a GnRH antagonist and add-back therapy.

In another aspect, the disclosure features a method of inducing amenorrhea in a human patient diagnosed as having uterine fibroids by periodically administering to the patient therapeutically effective amounts of a GnRH antagonist and add-back therapy. In an additional aspect, the disclosure features a method of inducing amenorrhea in a human patient by:
a) diagnosing the patient as having uterine fibroids; and
b) periodically administering to the patient therapeutically effective amounts of a GnRH antagonist and add-back therapy.

In another aspect, the disclosure features a method of reducing the volume of one or more uterine fibroids in a human patient diagnosed as having uterine fibroids by periodically administering to the patient therapeutically effective amounts of a GnRH antagonist and add-back therapy. In an additional aspect, the disclosure features a method of reducing the volume of one or more uterine fibroids in a human patient by:
a) diagnosing the patient as having uterine fibroids; and
b) periodically administering to the patient therapeutically effective amounts of a GnRH antagonist and add-back therapy.

In another aspect, the disclosure features a method of reducing the volume of one or more endometriosis nodes in a human patient diagnosed as having endometriosis by periodically administering to the patient therapeutically effective amounts of a GnRH antagonist and add-back therapy. In an additional aspect, the disclosure features a method of reducing the volume of one or more endometriosis nodes in a human patient by:
a) diagnosing the patient as having endometriosis; and
b) periodically administering to the patient therapeutically effective amounts of a GnRH antagonist and add-back therapy.

In another aspect, the disclosure features a method of reducing pelvic pain in a human patient diagnosed as having endometriosis (e.g., rectovaginal endometriosis) by periodically administering to the patient therapeutically effective amounts of a GnRH antagonist and add-back therapy. In an additional aspect, the disclosure features a method of reducing pelvic pain in a human patient by:
a) diagnosing the patient as having endometriosis (e.g., rectovaginal endometriosis); and
b) periodically administering to the patient therapeutically effective amounts of a GnRH antagonist and add-back therapy.

In another aspect, the disclosure features a method of reducing dysmenorrhea in a human patient diagnosed as having endometriosis (e.g., rectovaginal endometriosis) by periodically administering to the patient therapeutically effective amounts of a GnRH antagonist and add-back therapy. In an additional aspect, the disclosure features a method of reducing dysmenorrhea in a human patient by:
a) diagnosing the patient as having endometriosis (e.g., rectovaginal endometriosis); and
b) periodically administering to the patient therapeutically effective amounts of a GnRH antagonist and add-back therapy.

In another aspect, the disclosure features a method of reducing dyspareunia in a human patient diagnosed as having endometriosis (e.g., rectovaginal endometriosis) by periodically administering to the patient therapeutically effective amounts of a GnRH antagonist and add-back therapy. In an additional aspect, the disclosure features a method of reducing dyspareunia in a human patient by:
a) diagnosing the patient as having endometriosis (e.g., rectovaginal endometriosis); and
b) periodically administering to the patient therapeutically effective amounts of a GnRH antagonist and add-back therapy.

In another aspect, the disclosure features a method of reducing dyschezia in a human patient diagnosed as having endometriosis (e.g., rectovaginal endometriosis) by periodically administering to the patient therapeutically effective amounts of a GnRH antagonist and add-back therapy. In an additional aspect, the disclosure features a method of reducing dyschezia in a human patient by:
a) diagnosing the patient as having endometriosis (e.g., rectovaginal endometriosis); and
b) periodically administering to the patient therapeutically effective amounts of a GnRH antagonist and add-back therapy.

In another aspect, the disclosure features a method of reducing uterine bleeding in a human patient diagnosed as having endometriosis (e.g., rectovaginal endometriosis) by periodically administering to the patient therapeutically effective amounts of a GnRH antagonist and add-back therapy. In an additional aspect, the disclosure features a method of reducing uterine bleeding in a human patient by:
a) diagnosing the patient as having endometriosis (e.g., rectovaginal endometriosis); and
b) periodically administering to the patient therapeutically effective amounts of a GnRH antagonist and add-back therapy.

In another aspect, the disclosure features a method of inducing amenorrhea in a human patient diagnosed as having endometriosis (e.g., rectovaginal endometriosis) by periodically administering to the patient therapeutically effective amounts of a GnRH antagonist and add-back therapy. In an additional aspect, the disclosure features a method of inducing amenorrhea in a human patient by:
a) diagnosing the patient as having endometriosis (e.g., rectovaginal endometriosis); and
b) periodically administering to the patient therapeutically effective amounts of a GnRH antagonist and add-back therapy.

In another aspect, the disclosure features a method of reducing the volume of one or more rectovaginal endometriosis nodes (e.g., one or more type II or type IlIl rectovaginal endometriosis nodes) in a human patient diagnosed as having rectovaginal endometriosis by periodically administering to the patient therapeutically effective amounts of a GnRH antagonist and add-back therapy. In an additional aspect, the disclosure features a method of reducing the volume of one or more rectovaginal endometriosis nodes (e.g., one or more type II or type IlIl rectovaginal endometriosis nodes) in a human patient by:
a) diagnosing the patient as having rectovaginal endometriosis; and
b) periodically administering to the patient therapeutically effective amounts of a GnRH antagonist and add-back therapy.

In another aspect, the disclosure features a method of reducing uterine volume in a human patient diagnosed as having adenomyosis by periodically administering to the patient therapeutically effective amounts of a GnRH antagonist and add-back therapy. In an additional aspect, the disclosure features a method of reducing uterine volume in a human patient by:
a) diagnosing the patient as having adenomyosis; and
b) periodically administering to the patient therapeutically effective amounts of a GnRH antagonist and add-back therapy.

In another aspect, the disclosure features a method of reducing the thickness of the anterior and/or posterior region of the uterine myometrium in a human patient diagnosed as having adenomyosis by periodically administering to the patient therapeutically effective amounts of a GnRH antagonist and add-back therapy. In an additional aspect, the disclosure features a method of reducing the thickness of the anterior and/or posterior region of the uterine myometrium in a human patient by:
a) diagnosing the patient as having adenomyosis; and
b) periodically administering to the patient therapeutically effective amounts of a GnRH antagonist and add-back therapy.

In another aspect, the disclosure features a method of reducing pelvic pain in a human patient diagnosed as having adenomyosis by periodically administering to the patient therapeutically effective amounts of a GnRH antagonist and add-back therapy. In an additional aspect, the disclosure features a method of reducing pelvic pain in a human patient by:
a) diagnosing the patient as having adenomyosis; and
b) periodically administering to the patient therapeutically effective amounts of a GnRH antagonist and add-back therapy.

In another aspect, the disclosure features a method of reducing dysmenorrhea in a human patient diagnosed as having adenomyosis by periodically administering to the patient therapeutically effective amounts of a GnRH antagonist and add-back therapy. In an additional aspect, the disclosure features a method of reducing dysmenorrhea in a human patient by:
a) diagnosing the patient as having adenomyosis; and
b) periodically administering to the patient therapeutically effective amounts of a GnRH antagonist and add-back therapy.

In another aspect, the disclosure features a method of reducing dyspareunia in a human patient diagnosed as having adenomyosis by periodically administering to the patient therapeutically effective amounts of a GnRH antagonist and add-back therapy. In an additional aspect, the disclosure features a method of reducing dyspareunia in a human patient by:
a) diagnosing the patient as having adenomyosis; and
b) periodically administering to the patient therapeutically effective amounts of a GnRH antagonist and add-back therapy.

In another aspect, the disclosure features a method of reducing dyschezia in a human patient diagnosed as having adenomyosis by periodically administering to the patient therapeutically effective amounts of a GnRH antagonist and add-back therapy. In an additional aspect, the disclosure features a method of reducing dyschezia in a human patient by:
a) diagnosing the patient as having adenomyosis; and
b) periodically administering to the patient therapeutically effective amounts of a GnRH antagonist and add-back therapy.

In another aspect, the disclosure features a method of reducing uterine tenderness in a human patient diagnosed as having adenomyosis by periodically administering to the patient therapeutically effective amounts of a GnRH antagonist and add-back therapy. In an additional aspect, the disclosure features a method of reducing uterine tenderness in a human patient by:
a) diagnosing the patient as having adenomyosis; and
b) periodically administering to the patient therapeutically effective amounts of a GnRH antagonist and add-back therapy.

In another aspect, the disclosure features a method of reducing uterine bleeding in a human patient diagnosed as having adenomyosis by periodically administering to the patient therapeutically effective amounts of a GnRH antagonist and add-back therapy. In an additional aspect, the disclosure features a method of reducing uterine bleeding in a human patient by:
a) diagnosing the patient as having adenomyosis; and
b) periodically administering to the patient therapeutically effective amounts of a GnRH antagonist and add-back therapy.

In another aspect, the disclosure features a method of inducing amenorrhea in a human patient diagnosed as having adenomyosis by periodically administering to the patient therapeutically effective amounts of a GnRH antagonist and add-back therapy. In an additional aspect, the disclosure features a method of inducing amenorrhea in a human patient by:
a) diagnosing the patient as having adenomyosis; and
b) periodically administering to the patient therapeutically effective amounts of a GnRH antagonist and add-back therapy.

In any of the foregoing aspects or embodiments of the disclosure, administration of the GnRH antagonist and administration of the add-back therapy may commence at substantially the same time, for example, on the same day. In some embodiments, the first administration of the GnRH antagonist and the first administration of the add-back therapy coincide with one another. In some embodiments, administration of the GnRH antagonist and administration of the add-back therapy begin within from about 1 minute to about 7 days of one another, such as within about 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, 30 minutes, 31 minutes, 32 minutes, 33 minutes, 34 minutes, 35 minutes, 36 minutes, 37 minutes, 38 minutes, 39 minutes, 40 minutes, 41 minutes, 42 minutes, 43 minutes, 44 minutes, 45 minutes, 46 minutes, 47 minutes, 48 minutes, 49 minutes, 50 minutes, 51 minutes, 52 minutes, 53 minutes, 54 minutes, 55 minutes, 56 minutes, 57 minutes, 58 minutes, 59 minutes, 60 minutes, 61 minutes, 62 minutes, 63 minutes, 64 minutes, 65 minutes, 66 minutes, 67 minutes, 68 minutes, 69 minutes, 70 minutes, 71 minutes, 72 minutes, 73 minutes, 74 minutes, 75 minutes, 76 minutes, 77 minutes, 78 minutes, 79 minutes, 80 minutes, 81 minutes, 82 minutes, 83 minutes, 84 minutes, 85 minutes, 86 minutes, 87 minutes, 88 minutes, 89 minutes, 90 minutes, 91 minutes, 92 minutes, 93 minutes, 94 minutes, 95 minutes, 96 minutes, 97 minutes, 98 minutes, 99 minutes, 100 minutes, 101 minutes, 102 minutes, 103 minutes, 104 minutes, 105 minutes, 106 minutes, 107 minutes, 108 minutes, 109 minutes, 110 minutes, 111 minutes, 112 minutes, 113 minutes, 114 minutes, 115 minutes, 116 minutes, 117 minutes, 118 minutes, 119 minutes, 120 minutes, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 49 hours, 50 hours, 51 hours, 52 hours, 53 hours, 54 hours, 55 hours, 56 hours, 57 hours, 58 hours, 59 hours, 60 hours, 61 hours, 62 hours, 63 hours, 64 hours, 65 hours, 66 hours, 67 hours, 68 hours, 69 hours, 70 hours, 71 hours, 72 hours, 84 hours, 96 hours, 108 hours, 120 hours, 132 hours, 144 hours, 156 hours, or 168 hours of one another.

In some embodiments, administration of the GnRH antagonist and administration of the add-back therapy begin within from about 1 minute to about 48 hours of on another, such as within about 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, 30 minutes, 31 minutes, 32 minutes, 33 minutes, 34 minutes, 35 minutes, 36 minutes, 37 minutes, 38 minutes, 39 minutes, 40 minutes, 41 minutes, 42 minutes, 43 minutes, 44 minutes, 45 minutes, 46 minutes, 47 minutes, 48 minutes, 49 minutes, 50 minutes, 51 minutes, 52 minutes, 53 minutes, 54 minutes, 55 minutes, 56 minutes, 57 minutes, 58 minutes, 59 minutes, 60 minutes, 61 minutes, 62 minutes, 63 minutes, 64 minutes, 65 minutes, 66 minutes, 67 minutes, 68 minutes, 69 minutes, 70 minutes, 71 minutes, 72 minutes, 73 minutes, 74 minutes, 75 minutes, 76 minutes, 77 minutes, 78 minutes, 79 minutes, 80 minutes, 81 minutes, 82 minutes, 83 minutes, 84 minutes, 85 minutes, 86 minutes, 87 minutes, 88 minutes, 89 minutes, 90 minutes, 91 minutes, 92 minutes, 93 minutes, 94 minutes, 95 minutes, 96 minutes, 97 minutes, 98 minutes, 99 minutes, 100 minutes, 101 minutes, 102 minutes, 103 minutes, 104 minutes, 105 minutes, 106 minutes, 107 minutes, 108 minutes, 109 minutes, 110 minutes, 111 minutes, 112 minutes, 113 minutes, 114 minutes, 115 minutes, 116 minutes, 117 minutes, 118 minutes, 119 minutes, 120 minutes, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours.

In some embodiments, administration of the GnRH antagonist and administration of the add-back therapy begin within from about 1 minute to about 24 hours of one another, such as within from about 1 minute to about 21 hours of one another, from about 1 minute to about 18 hours of one another, within from about 1 minute to about 15 hours of one another, within from about 1 minute to about 12 hours of one another, within from about 1 minute to about 9 hours of one another, within from about 1 minute to about 6 hours of one another, within from about 1 minute to about 3 hours of one another, within from about 1 minute to about 60 minutes of one another, within from about 1 minute to about 30 minutes of one another, within from about 1 minute to about 29 minutes of one another, within from about 1 minute to about 28 minutes of one another, within from about 1 minute to about 27 minutes of one another, within from about 1 minute to about 26 minutes of one another, within from about 1 minute to about 25 minutes of one another, within from about 1 minute to about 24 minutes of one another, within from about 1 minute to about 23 minutes of one another, within from about 1 minute to about 22 minutes of one another, within from about 1 minute to about 21 minutes of one another, within from about 1 minute to about 20 minutes of one another, within from about 1 minute to about 19 minutes of one another, within from about 1 minute to about 18 minutes of one another, within from about 1 minute to about 17 minutes of one another, within from about 1 minute to about 16 minutes of one another, within from about 1 minute to about 15 minutes of one another, within from about 1 minute to about 14 minutes of one another, within from about 1 minute to about 13 minutes of one another, within from about 1 minute to about 12 minutes of one another, within from about 1 minute to about 11 minutes of one another, within from about 1 minute to about 10 minutes of one another, within from about 1 minute to about 9 minutes of one another, within from about 1 minute to about 8 minutes of one another, within from about 1 minute to about 7 minutes of one another, within from about 1 minute to about 6 minutes of one another, within from about 1 minute to about 5 minutes of one another, within from about 1 minute to about 4 minutes of one another, or within from about 1 minute to about 3 minutes of one another.

In some embodiments of any of the foregoing aspects of the disclosure, the GnRH antagonist is a compound represented by formula (I)

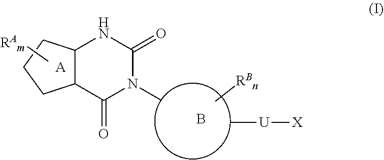

wherein ring A is a thiophene ring;

each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²a³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;

n is an integer from 0 to 2;

U is a single bond;

X is a group represented by —S-L-Y, —O-L-Y, —CO-L-Y, or —SO₂-L-Y, wherein L is an optionally substituted lower alkylene group;

Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and

Z is an optionally fused and optionally substituted cycloalkyl group, an optionally fused and optionally substituted heterocycloalkyl group, an optionally fused and optionally substituted aryl group, or an optionally fused and optionally substituted heteroaryl group;

or a pharmaceutically acceptable salt thereof.

In some embodiments of formula (I), the ring A is a thiophene ring represented by formula (IIa)

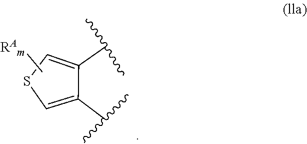

In some embodiments of formula (I) or (IIa), m is 1.

In some embodiments of formula (I) or (IIa), the ring A is an optionally substituted thiophene ring represented by formula (IIb)

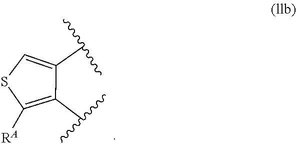

In some embodiments of formula (I), (IIa), or (IIb), each R^{A} is independently a halogen atom, an optionally substituted lower alkyl group, COOW¹, or CONW²W³, wherein W¹ to W³ independently are a hydrogen atom or an optionally substituted lower alkyl group, or W² and W³ may bind together with the neighboring nitrogen atom to form an optionally substituted cyclic amino group.

In some embodiments of formula (I), (IIa), or (IIb), each R^{A} is COOH or pharmaceutically acceptable salt thereof.

In some embodiments of formula (I), (IIa), or (IIb), the ring B is an optionally substituted benzene ring, pyridine ring, or thiophene ring.

In some embodiments of formula (I), (IIa), or (IIb), the ring B is represented by a formula selected from the group consisting of:

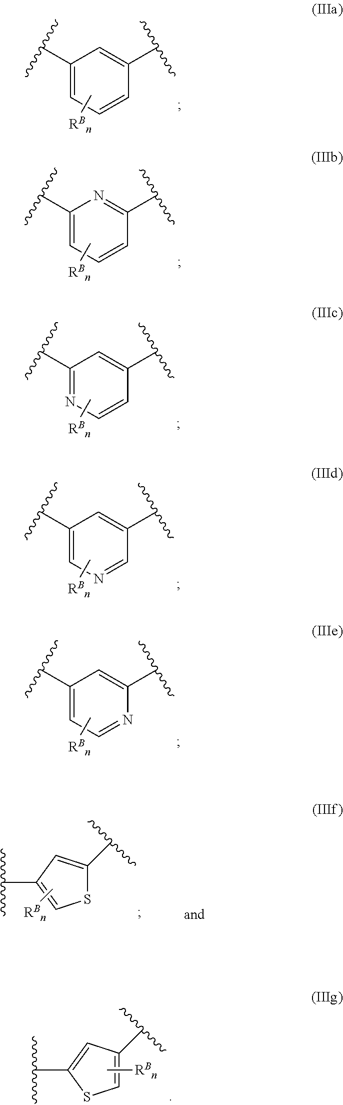

In some embodiments of formula (I), (IIa), (IIb), or any one of (IIIa)-(IIIg), n is 2.

In some embodiments of formula (I), (IIa), (IIb), or any one of (IIIa)-(IIIg), the ring B is represented by a formula selected from the group consisting of:

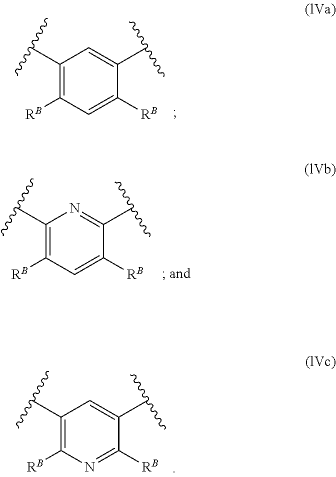

In some embodiments of formula (I), (IIa), (IIb), any one of (IIIa)-(IIIg), or any one of (IVa)-(IVc), each R^{B} is independently a halogen atom, an optionally substituted lower alkyl group, or OW⁴, wherein each W⁴ is independently a hydrogen atom or an optionally substituted lower alkyl group.

In some embodiments of formula (I), (IIa), (Ib), any one of (IIIa)-(IIIg), or any one of (IVa)-(IVc), each R^{B} is independently a fluorine atom, chlorine atom, bromine atom, methyl group, or methoxy group.

In some embodiments of formula (H), (IIa), (Ib), any one of (IIIa)-(IIIg), or any one of (IVa)-(IVc), U is a single bond.

In some embodiments of formula (H), (IIa), (Ib), any one of (IIIa)-(IIIg), or any one of (IVa)-(IVc), X is a group represented by —O-L-Y.

In some embodiments of formula (I), (IIa), (IIb), any one of (IIIa)-(IIIg), or any one of (IVa)-(IVc), L is a methylene group.

In some embodiments of formula (I), (IIa), (IIb), any one of (IIa)-(IIIg), or any one of (IVa)-(IVc), Y is an optionally substituted benzene ring represented by formula (V)

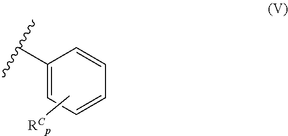

wherein each R^{C} is independently a halogen atom, an optionally substituted lower alkyl group, or OW⁹, wherein each W⁹ is independently a hydrogen atom or an optionally substituted lower alkyl group; and

p is an integer from 0 to 3.

In some embodiments of formula (I), (IIa), (IIb), any one of (IIa)-(IIIg), any one of (IVa)-(IVc), or (V), Y is a substituted benzene ring represented by formula (Va)

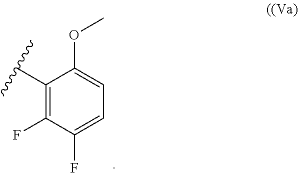 [SMILES: *c1c(OC)ccc(F)c1F]

In some embodiments, the compound is represented by formula (Ia)

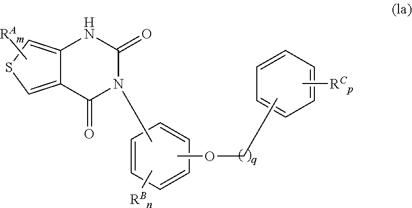

wherein each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;

m is an integer from 0 to 3;

each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;

n is an integer from 0 to 2;

q is an integer from 0 to 3;

each R^{C} is independently a halogen atom, an optionally substituted lower alkyl group, or OW⁹, wherein each W⁹ is independently a hydrogen atom or an optionally substituted lower alkyl group; and

p is an integer from 0 to 3;

or a pharmaceutically acceptable salt thereof.

In some embodiments, the compound is represented by formula (Ib)

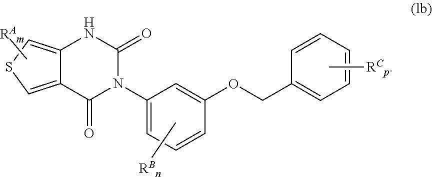

In some embodiments, the compound is represented by formula (Ic)

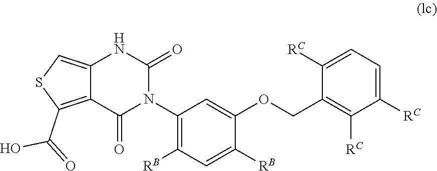

or a pharmaceutically acceptable salt thereof.

In some embodiments, the compound is 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid, represented by formula (VI)

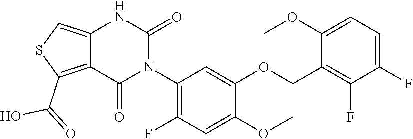 [SMILES: COc1cc(F)c(-n2c(=O)[nH]c3csc(C(=O)O)c3c2=O)cc1OCc1c(OC)ccc(F)c1F]

or a pharmaceutically acceptable salt thereof. In some embodiments, the compound 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid is administered to the patient in the form of an electrostatically neutral carboxylic acid. In some embodiments, the compound 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid is administered to the patient in the form of a pharmaceutically acceptable salt. In some embodiments, the compound 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid is administered to the patient in the form of the choline salt, choline 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate.

In some embodiments, the compound is the choline salt of the compound represented by formula (VI), choline 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate. It is to be understood that references herein to a compound represented by formula (VI) or a pharmaceutically acceptable salt thereof specifically include the choline salt of compound (VI), which is represented by formula (VIa), below.

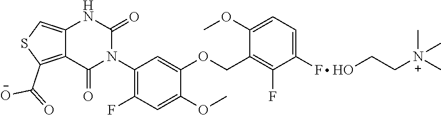

In some embodiments, the choline 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate is in a crystalline state.

In some embodiments, the choline 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate exhibits characteristic X-ray powder diffraction (XRPD) peaks at about 7.1° 2θ, about 11.5° 2θ, about 19.4° 2θ, about 21.5° 2θ, about 22.0° 2θ, about 22.6° 2θ, about 23.5° 2θ, and about 26.2° 2θ.

In some embodiments, the choline 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate exhibits ¹³C solid-state nuclear magnetic resonance (NMR) peaks centered at about 55.5 ppm, about 57.1 ppm, about 58.7 ppm, about 69.8 ppm, about 98.1 ppm, about 110.3 ppm, about 111.6 ppm, about 113.7 ppm, about 118.0 ppm, about 145.3 ppm, about 149.8 ppm, and about 155.8 ppm.

In some embodiments, the choline 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate exhibits ¹⁹F solid-state NMR peaks centered at about −151.8 ppm, −145.2 ppm, and −131.6 ppm.

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is orally administered to the patient.

In some embodiments, the compound of any one of formulas (I), (Ia)-(Ic), (Ia), (IIb), (IIa)-(IIIg), (IV)-(IVc), (V), (Va), or (VI) (e.g., (VIa)) is administered to the patient in an amount of from about 25 mg to about 400 mg per dose (e.g., in the recited amount or in an equivalent amount of a pharmaceutically acceptable salt, such as a choline salt), such as an amount of about 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, or 400 mg of the compound of any one of formulas (I), (Ia)-(Ic), (IIa), (IIb), (IIIa)-(IIIg), (IVa)-(IVc), (V), (Va), or (VI) (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of from about 35 mg to about 65 mg per dose (e.g., in the recited amount or in an equivalent amount of a pharmaceutically acceptable salt, such as a choline salt), such as an amount of about 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, or 65 mg per dose (e.g., in the recited amount or in an equivalent amount of a pharmaceutically acceptable salt, such as a choline salt). In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of about 50 mg per dose (e.g., in the recited amount or in an equivalent amount of a pharmaceutically acceptable salt, such as a choline salt).

In some embodiments, the compound of any one of formulas (I), (Ia)-(Ic), (Ia), (IIb), (IIIa)-(IIIg), (IVa)-(IVc), (V), (Va), or (VI) (e.g., (VIa)) is administered to the patient in an amount of from about 60 mg to about 90 mg per dose (e.g., in the recited amount or in an equivalent amount of a pharmaceutically acceptable salt, such as a choline salt), such as an amount of about 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, or 90 mg per dose (e.g., in the recited amount or in an equivalent amount of a pharmaceutically acceptable salt, such as a choline salt). In some embodiments, the compound of any one of formulas (I), (Ia)-(IC), (IIa), (IIb), (IIIa)-(IIIg), (IVa)-(IVc), (V), (Va), or (VI) (e.g., (VIa)) is administered to the patient in an amount of about 75 mg per dose (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of from about 50 mg to about 150 mg per dose (e.g., in the recited amount or in an equivalent amount of a pharmaceutically acceptable salt, such as a choline salt), such as an amount of about 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, or 150 mg per dose (e.g., in the recited amount or in an equivalent amount of a pharmaceutically acceptable salt, such as a choline salt). In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of about 100 mg per dose (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in an amount of from about 150 mg to about 250 mg per dose (e.g., in the recited amount or in an equivalent amount of a pharmaceutically acceptable salt, such as a choline salt), such as an amount of about 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, or 250 mg per dose (e.g., in the recited amount or in an equivalent amount of a pharmaceutically acceptable salt, such as a choline salt). In some embodiments, the compound of any one of formulas (I), (Ia)-(Ic), (IIa), (IIb), (IIa)-(IIIg), (IVa)-(IVc), (V), (Va), or (VI) (e.g., (VIa)) is administered to the patient in an amount of about 200 mg per dose (e.g., in the recited amount or in an equivalent amount of a pharmaceutically acceptable salt, such as a choline salt).

In some embodiments, the compound of any one of formulas (I), (Ia)-(Ic), (Ia), (IIb), (IIIa)-(IIIg), (IVa)-(IVc), (V), (Va), or (VI) (e.g., (VIa)) is administered to the patient in one or more doses (i.e., one or more times) per day, week, or month, such as from 1 to 10 times per day (e.g., 1 time, 2 times, 3 times, 4 times, 5 times, 6 times, 7 times, 8 times, 9 times, or 10 times per day, such as 1 time or 2 times per day), 1 to 100 times per week (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 25 times, 30 times, 35 times 40 times, 45 times, 50 times, 55 times, 60 times, 65 times, 70 times, 75 times, 80 times, 85 times, 90 times, 95 times, or 100 times per week, such as 7 times, 8 times, 9 times, 10 times, 11 times, 12 times, 13 times, 14 times per week), or 1 to 500 times per month (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 21 times, 22 times, 23 times, 24 times, 25 times, 26 times, 27 times, 28 times, 29 times, 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, 60 times, 61 times, 62 times, 63 times, 64 times, 65 times, 66 times, 67 times, 68 times, 69 times, 70 times, 71 times, 72 times, 73 times, 74 times, 75 times, 76 times, 77 times, 78 times, 79 times, 80 times, 81 times, 82 times, 83 times, 84 times, 85 times, 86 times, 87 times, 88 times, 89 times, 90 times, 91 times, 92 times, 93 times, 94 times, 95 times, 96 times, 97 times, 98 times, 99 times, 100 times, 200 times, 300 times, 400 times, or 500 times per month, such as 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, or 60 times per month), or more.

For example, the compound of any one of formulas (I), (Ia)-(Ic), (IIa), (IIb), (IIIa)-(IIIg), (IVa)-(IVc), (V), (Va), or (VI) (e.g., (VIa)) may be administered to the patient in one or more doses every 4 hours, 6 hours, 8 hours, 10 hours, 12 hours, 14 hours, 16 hours, 18 hours, 20 hours, 22 hours, 24 hours, 28 hours, 30 hours, 32 hours, 34 hours, 36 hours, 38 hours, 40 hours, 42 hours, 44 hours, 46 hours, 48 hours, 50 hours, 52 hours, 54 hours, 56 hours, 58 hours, 60 hours, 62 hours, 64 hours, 66 hours, 68 hours, 70 hours, or 72 hours, 74 hours, 76 hours, 78 hours, 80 hours, 82 hours, 84 hours, 86 hours, 88 hours, 90 hours, 92 hours, 94 hours, 96 hours, 98 hours, 100 hour, 102 hours, 104 hours, 105 hours, 106 hours, 108 hours, 110 hours, 112 hours, 114 hours, 116 hours, 118 hours, 120 hours, 122 hours, 124 hours, 126 hours, 128 hours, 130 hours, 132 hours, 134 hours, 136 hours, 138 hours, 140 hours, 142 hours, 144 hours, 146 hours, 148 hours, 150 hours, 152 hours, 154 hours, 156 hours, 158 hours, 160 hours, 162 hours, 164 hours, 166 hours, 168 hours, or more. In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in one or more doses per day, such as from 1 to 10 doses per 12 hours (e.g., 1 dose, 2 doses, 3 doses, 4 doses, 5 doses, 6 doses, 7 doses, 8 doses, 9 doses, 10 doses per 12 hours), from 1 to 10 doses per 24 hours (e.g., 1 dose, 2 doses, 3 doses, 4 doses, 5 doses, 6 doses, 7 doses, 8 doses, 9 doses, 10 doses per 24 hours), or from 1 to 10 doses per 48 hours (e.g., 1 dose, 2 doses, 3 doses, 4 doses, 5 doses, 6 doses, 7 doses, 8 doses, 9 doses, 10 doses per 48 hours), among others.

The compound of any one of formulas (I), (Ia)-(Ic), (IIa), (IIb), (IIIa)-(IIIg), (IVa)-(IVc), (V), (Va), or (VI) (e.g., (VIa)) may be administered to the patient in one or more unit dosage forms that collectively constitute a single dose. For example, a patient may be administered a single dose of the compound of a specified amount, such as a single dose of 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, or more (e.g., in the recited amount or in an equivalent amount of a pharmaceutically acceptable salt, such as a choline salt), by administration of one or more unit dosage forms of the compound to the patient. As a non-limiting example, a single dose of 200 mg of the compound may be administered to the subject by way of two individual 100-mg unit dosage forms of the compound. The two 100-mg unit dosage forms collectively constitute a single 200-mg dose of the compound if administered to the patient at substantially the same time.

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in one or more daily doses (e.g., from 1 to 10 doses, such as 1, 2, 3, 4, 5, 6, 7, 8, 9, or 10 doses, or more) totaling from about 35 mg to about 65 mg per day (e.g., in the recited amount or in an equivalent amount of a pharmaceutically acceptable salt, such as a choline salt), such as an amount of about 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, or 65 mg per day (e.g., in the recited amount or in an equivalent amount of a pharmaceutically acceptable salt, such as a choline salt). In some embodiments, the compound is administered to the patient in one or more daily doses (e.g., from 1 to 10 doses, such as 1, 2, 3, 4, 5, 6, 7, 8, 9, or 10 doses, or more) totaling an amount of about 50 mg per day (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in one or more daily doses (e.g., from 1 to 10 doses, such as 1, 2, 3, 4, 5, 6, 7, 8, 9, or 10 doses, or more) totaling from about 60 mg to about 90 mg per day (e.g., in the recited amount or in an equivalent amount of a pharmaceutically acceptable salt, such as a choline salt), such as an amount of about 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, or 90 mg per day (e.g., in the recited amount or in an equivalent amount of a pharmaceutically acceptable salt, such as a choline salt). In some embodiments, the compound is administered to the patient in one or more daily doses (e.g., from 1 to 10 doses, such as 1, 2, 3, 4, 5, 6, 7, 8, 9, or 10 doses, or more) totaling an amount of about 75 mg per day (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in one or more daily doses (e.g., from 1 to 10 doses, such as 1, 2, 3, 4, 5, 6, 7, 8, 9, or 10 doses, or more) totaling from about 50 mg to about 150 mg per day (e.g., in the recited amount or in an equivalent amount of a pharmaceutically acceptable salt, such as a choline salt), such as an amount of about 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 11 mg, g 11 mg, g 112 mg, g 113 mg, 4 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, or 150 mg per day (e.g., in the recited amount or in an equivalent amount of a pharmaceutically acceptable salt, such as a choline salt). In some embodiments, the compound is administered to the patient in one or more daily doses (e.g., from 1 to 10 doses, such as 1, 2, 3, 4, 5, 6, 7, 8, 9, or 10 doses, or more) totaling an amount of about 100 mg per day (e.g., in the recited amount or in an equivalent amount of a pharmaceutically acceptable salt, such as a choline salt).

In some embodiments, the compound of any one of formulas (I), (Ia)-(Ic), (Ia), (IIb), (IIIa)-(IIIg), (IVa)-(IVc), (V), (Va), or (VI) (e.g., (VIa)) is administered to the patient in one or more daily doses (e.g., from 1 to 10 doses, such as 1, 2, 3, 4, 5, 6, 7, 8, 9, or 10 doses, or more) totaling from about 150 mg to about 250 mg per day (e.g., in the recited amount or in an equivalent amount of a pharmaceutically acceptable salt, such as a choline salt), such as an amount of about 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, or 250 mg per day (e.g., in the recited amount or in an equivalent amount of a pharmaceutically acceptable salt, such as a choline salt). In some embodiments, the compound is administered to the patient in one or more daily doses (e.g., from 1 to 10 doses, such as 1, 2, 3, 4, 5, 6, 7, 8, 9, or 10 doses, or more) totaling an amount of about 200 mg per day (e.g., in the recited amount or in an equivalent amount of a pharmaceutically acceptable salt, such as a choline salt).

In some embodiments, the compound of any one of formulas (I), (Ia)-(Ic), (Ia), (IIb), (IIIa)-(IIIg), (IVa)-(IVc), (V), (Va), or (VI) (e.g., (VIa)) is administered to the patient in a single dose per day. For example, the compound may be administered to the patient in an amount (e.g., a single dose) of from about 35 mg to about 65 mg per day (e.g., in the recited amount or in an equivalent amount of a pharmaceutically acceptable salt, such as a choline salt), such as an amount of about 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, or 65 mg per day (e.g., in the recited amount or in an equivalent amount of a pharmaceutically acceptable salt, such as a choline salt). In some embodiments, the compound is administered to the patient in an amount (e.g., a single dose) of about 50 mg per day (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in a single dose per day. For example, the compound may be administered to the patient in an amount (e.g., a single dose) of from about 60 mg to about 90 mg per day (e.g., in the recited amount or in an equivalent amount of a pharmaceutically acceptable salt, such as a choline salt), such as an amount of about 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, or 90 mg per day (e.g., in the recited amount or in an equivalent amount of a pharmaceutically acceptable salt, such as a choline salt). In some embodiments, the compound is administered to the patient in an amount (e.g., a single dose) of about 75 mg per day (e.g., in the recited amount or in an equivalent amount of a pharmaceutically acceptable salt, such as a choline salt).

In some embodiments, the compound of any one of formulas (I), (Ia)-(Ic), (Ia), (IIb), (IIIa)-(IIIg), (IVa)-(IVc), (V), (Va), or (VI) (e.g., (VIa)) is administered to the patient in a single dose per day. For example, the compound may be administered to the patient in an amount (e.g., a single dose) of from about 50 mg to about 150 mg per day (e.g., in the recited amount or in an equivalent amount of a pharmaceutically acceptable salt, such as a choline salt), such as an amount of about 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, or 150 mg per day (e.g., in the recited amount or in an equivalent amount of a pharmaceutically acceptable salt, such as a choline salt). In some embodiments, the compound is administered to the patient in an amount (e.g., a single dose) of about 100 mg per day (e.g., in the recited amount or in an equivalent amount of a pharmaceutically acceptable salt, such as a choline salt).

In some embodiments, the compound of any one of formulas (I), (Ia)-(Ic), (IIa), (IIb), (IIa)-(IIIg), (IV)-(IVc), (V), (Va), or (VI) (e.g., (VIa)) is administered to the patient in a single dose per day, and is administered to the patient in an amount (e.g., a single dose) of from about 150 mg to about 250 mg per day (e.g., in the recited amount or in an equivalent amount of a pharmaceutically acceptable salt, such as a choline salt), such as an amount of about 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, or 250 mg per day (e.g., in the recited amount or in an equivalent amount of a pharmaceutically acceptable salt, such as a choline salt). In some embodiments, the compound is administered to the patient in an amount (e.g., a single dose) of about 200 mg per day (e.g., in the recited amount or in an equivalent amount of a pharmaceutically acceptable salt, such as a choline salt).

In some embodiments, the compound of any one of formulas (I), (Ia)-(Ic), (Ba), (IIb), (IIIa)-(IIIg), (IVa)-(IVc), (V), (Va), or (VI) (e.g., (VIa)) is periodically administered to the patient (e.g., using a dosing schedule defined above, such as an in amount of from about 35 mg per day to about 65 mg per day (e.g., 50 mg per day), an amount of from about 60 mg per day to about 90 mg per day (e.g., 75 mg per day), an amount of from about 50 mg per day to about 150 mg per day (e.g., 100 mg per day), or in an amount of from about 150 mg per day to about 250 mg per day (e.g., 200 mg per day)) over the course of a treatment period, such as a treatment period of one or more weeks, months, or years, for example, a treatment period of from about 1 week to about 48 months, or more (e.g., a treatment period of about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, 56 weeks, 57 weeks, 58 weeks, 59 weeks, 60 weeks, 61 weeks, 62 weeks, 63 weeks, 64 weeks, 65 weeks, 66 weeks, 67 weeks, 68 weeks, 69 weeks, 70 weeks, 71 weeks, 72 weeks, 73 weeks, 74 weeks, 75 weeks, 76 weeks, 77 weeks, 78 weeks, 79 weeks, 80 weeks, 81 weeks, 82 weeks, 83 weeks, 84 weeks, 85 weeks, 86 weeks, 87 weeks, 88 weeks, 89 weeks, 90 weeks, 91 weeks, 92 weeks, 93 weeks, 94 weeks, 95 weeks, 96 weeks, 97 weeks, 98 weeks, 99 weeks, 100 weeks, 101 weeks, 102 weeks, 103 weeks, 104 weeks, 105 weeks, 106 weeks, 107 weeks, 108 weeks, 109 weeks, 110 weeks, 111 weeks, 112 weeks, 113 weeks, 114 weeks, 115 weeks, 116 weeks, 117 weeks, 118 weeks, 119 weeks, 120 weeks, 121 weeks, 122 weeks, 123 weeks, 124 weeks, 125 weeks, 126 weeks, 127 weeks, 128 weeks, 129 weeks, 130 weeks, 131 weeks, 132 weeks, 133 weeks, 134 weeks, 135 weeks, 136 weeks, 137 weeks, 138 weeks, 139 weeks, 140 weeks, 141 weeks, 142 weeks, 143 weeks, 144 weeks, 145 weeks, 146 weeks, 147 weeks, 148 weeks, 149 weeks, 150 weeks, 151 weeks, 152 weeks, 153 weeks, 154 weeks, 155 weeks, 156 weeks, 157 weeks, 158 weeks, 159 weeks, 160 weeks, 161 weeks, 162 weeks, 163 weeks, 164 weeks, 165 weeks, 166 weeks, 167 weeks, 168 weeks, 169 weeks, 170 weeks, 171 weeks, 172 weeks, 173 weeks, 174 weeks, 175 weeks, 176 weeks, 177 weeks, 178 weeks, 179 weeks, 180 weeks, 181 weeks, 182 weeks, 183 weeks, 184 weeks, 185 weeks, 186 weeks, 187 weeks, 188 weeks, 189 weeks, 190 weeks, 191 weeks, 192 weeks, 193 weeks, 194 weeks, 195 weeks, 196 weeks, 197 weeks, 198 weeks, 199 weeks, or 200 weeks, or more). The compound may be administered to the patient in the recited amount or in an equivalent amount of a pharmaceutically acceptable salt, such as a choline salt. In some embodiments, the compound is periodically administered to the patient (e.g., using a dosing schedule defined above, such as an in amount of from about 35 mg per day to about 65 mg per day (e.g., 50 mg per day), an amount of from about 60 mg per day to about 90 mg per day (e.g., 75 mg per day), an amount of from about 50 mg per day to about 150 mg per day (e.g., 100 mg per day), or in an amount of from about 150 mg per day to about 250 mg per day (e.g., 200 mg per day)) over the course of a treatment period of from about 1 month to about 48 months, or more, such as a treatment period of about 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 28 months, 29 months, 30 months, 31 months, 32 months, 33 months, 34 months, 35 months, 36 months, 37 months, 38 months, 39 months, 40 months, 41 months, 42 months, 43 months, 44 months, 45 months, 46 months, 47 months, 48 months, or more.

In some embodiments, the compound of any one of formulas (I), (Ia)-(Ic), (Ia), (IIb), (IIIa)-(IIIg), (IVa)-(IVc), (V), (Va), or (VI) (e.g., (VIa)) is periodically administered to the patient (e.g., using a dosing schedule defined above, such as an in amount of from about 35 mg per day to about 65 mg per day (e.g., 50 mg per day), an amount of from about 60 mg per day to about 90 mg per day (e.g., 75 mg per day), an amount of from about 50 mg per day to about 150 mg per day (e.g., 100 mg per day), or in an amount of from about 150 mg per day to about 250 mg per day (e.g., 200 mg per day)) over the course of a treatment period of at least 4 weeks, such as a treatment period of from about 4 weeks to about 12 months, or more. The compound may be administered to the patient in the recited amount or in an equivalent amount of a pharmaceutically acceptable salt, such as a choline salt. For example, the compound may be periodically administered to the patient (e.g., using a dosing schedule defined above, such as in an amount of from about 35 mg per day to about 65 mg per day (e.g., 50 mg per day), an amount of from about 60 mg per day to about 90 mg per day (e.g., 75 mg per day), an amount of from about 50 mg per day to about 150 mg per day (e.g., 100 mg per day), or in an amount of from about 150 mg per day to about 250 mg per day (e.g., 200 mg per day)) over the course of a treatment period of about 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, or 48 weeks, or more. In some embodiments, the compound is periodically administered to the patient (e.g., using a dosing schedule defined above, such as in an amount of from about 35 mg per day to about 65 mg per day (e.g., 50 mg per day), an amount of from about 60 mg per day to about 90 mg per day (e.g., 75 mg per day), an amount of from about 50 mg per day to about 150 mg per day (e.g., 100 mg per day), or in an amount of from about 150 mg per day to about 250 mg per day (e.g., 200 mg per day) over the course of a treatment period of from about 1 month to about 12 months, or more, such as a treatment period of about 1 month, 2 months, 3 months, 4 months, 5 months, 6 months, 7 months, 8 months, 9 months, 10 months, 11 months, or 12 months, or more.

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is periodically administered to the patient (e.g., using a dosing schedule defined above, such as an in amount of from about 35 mg per day to about 65 mg per day (e.g., 50 mg per day), an amount of from about 60 mg per day to about 90 mg per day (e.g., 75 mg per day), an amount of from about 50 mg per day to about 150 mg per day (e.g., 100 mg per day), or in an amount of from about 150 mg per day to about 250 mg per day (e.g., 200 mg per day)) over the course of a treatment period of at least 8 weeks, such as a treatment period of from about 8 weeks to about 10 months, or more. The compound may be administered to the patient in the recited amount or in an equivalent amount of a pharmaceutically acceptable salt, such as a choline salt. For example, the compound may be periodically administered to the patient (e.g., using a dosing schedule defined above, such as an in amount of from about 35 mg per day to about 65 mg per day (e.g., 50 mg per day), an amount of from about 60 mg per day to about 90 mg per day (e.g., 75 mg per day), an amount of from about 50 mg per day to about 150 mg per day (e.g., 100 mg per day), or in an amount of from about 150 mg per day to about 250 mg per day (e.g., 200 mg per day)) over the course of a treatment period of about 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, or 40 weeks, or more. In some embodiments, the compound is periodically administered to the patient (e.g., using a dosing schedule defined above, such as an in amount of from about 35 mg per day to about 65 mg per day (e.g., 50 mg per day), an amount of from about 60 mg per day to about 90 mg per day (e.g., 75 mg per day), an amount of from about 50 mg per day to about 150 mg per day (e.g., 100 mg per day), or in an amount of from about 150 mg per day to about 250 mg per day (e.g., 200 mg per day)) over the course of a treatment period of from about 2 months to about 10 months, or more, such as a treatment period of about 2 months, 3 months, 4 months, 5 months, 6 months, 7 months, 8 months, 9 months, or 10 months, or more.

In some embodiments, the compound of any one of formulas (I), (Ia)-(IC), (IIa), (IIb), (IIIa)-(IIg), (IVa)-(IVc), (V), (Va), or (VI) (e.g., (VIa)) is periodically administered to the patient (e.g., using a dosing schedule defined above, such as an in amount of from about 35 mg per day to about 65 mg per day (e.g., 50 mg per day), an amount of from about 60 mg per day to about 90 mg per day (e.g., 75 mg per day), an amount of from about 50 mg per day to about 150 mg per day (e.g., 100 mg per day), or in an amount of from about 150 mg per day to about 250 mg per day (e.g., 200 mg per day)) over the course of a treatment period of at least 12 weeks, such as a treatment period of from about 12 weeks to about 48 weeks (e.g., a treatment period of from about 16 weeks to about 48 weeks), or more. The compound may be administered to the patient in the recited amount or in an equivalent amount of a pharmaceutically acceptable salt, such as a choline salt. For example, the compound may be periodically administered to the patient (e.g., using a dosing schedule defined above, such as an in amount of from about 35 mg per day to about 65 mg per day (e.g., 50 mg per day), an amount of from about 60 mg per day to about 90 mg per day (e.g., 75 mg per day), an amount of from about 50 mg per day to about 150 mg per day (e.g., 100 mg per day), or in an amount of from about 150 mg per day to about 250 mg per day (e.g., 200 mg per day)) over the course of a treatment period of about 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, or 48 weeks, or more. In some embodiments, the compound is periodically administered to the patient (e.g., using a dosing schedule defined above, such as an in amount of from about 35 mg per day to about 65 mg per day (e.g., 50 mg per day), an amount of from about 60 mg per day to about 90 mg per day (e.g., 75 mg per day), an amount of from about 50 mg per day to about 150 mg per day (e.g., 100 mg per day), or in an amount of from about 150 mg per day to about 250 mg per day (e.g., 200 mg per day)) over the course of a treatment period of from about 3 months to about 12 months, or more, such as a treatment period of about 3 months, 4 months, 5 months, 6 months, 7 months, 8 months, 9 months, 10 months, 11 months, or 12 months, or more.

In some embodiments, the compound of any one of formulas (I), (Ia)-(Ic), (Ia), (IIb), (IIIa)-(IIIg), (IVa)-(IVc), (V), (Va), or (VI) (e.g., (VIa)) is periodically administered to the patient (e.g., using a dosing schedule defined above, such as an in amount of from about 35 mg per day to about 65 mg per day (e.g., 50 mg per day), an amount of from about 60 mg per day to about 90 mg per day (e.g., 75 mg per day), an amount of from about 50 mg per day to about 150 mg per day (e.g., 100 mg per day), or in an amount of from about 150 mg per day to about 250 mg per day (e.g., 200 mg per day)) over the course of a treatment period of at least 24 weeks, such as a treatment period of from about 24 weeks to about 72 weeks, or more. The compound may be administered to the patient in the recited amount or in an equivalent amount of a pharmaceutically acceptable salt, such as a choline salt. For example, the compound may be periodically administered to the patient (e.g., using a dosing schedule defined above, such as an in amount of from about 35 mg per day to about 65 mg per day (e.g., 50 mg per day), an amount of from about 60 mg per day to about 90 mg per day (e.g., 75 mg per day), an amount of from about 50 mg per day to about 150 mg per day (e.g., 100 mg per day), or in an amount of from about 150 mg per day to about 250 mg per day (e.g., 200 mg per day)) over the course of a treatment period of about 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, 56 weeks, 57 weeks, 58 weeks, 59 weeks, 60 weeks, 61 weeks, 62 weeks, 63 weeks, 64 weeks, 65 weeks, 66 weeks, 67 weeks, 68 weeks, 69 weeks, 70 weeks, 71 weeks, 72 weeks, or more. In some embodiments, the compound is periodically administered to the patient (e.g., using a dosing schedule defined above, such as an in amount of from about 35 mg per day to about 65 mg per day (e.g., 50 mg per day), an amount of from about 60 mg per day to about 90 mg per day (e.g., 75 mg per day), an amount of from about 50 mg per day to about 150 mg per day (e.g., 100 mg per day), or in an amount of from about 150 mg per day to about 250 mg per day (e.g., 200 mg per day)) over the course of a treatment period of from about 6 months to about 18 months, or more, such as a treatment period of about 6 months, 7 months, 8 months, 9 months, 10 months, 11 months, 12 months, 13 months, 14 months, 15 months, 16 months, 17 months, or 18 months, or more.

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is periodically administered to the patient at a particular dose (e.g., a particular daily dose) over the course of a first treatment period, and is subsequently periodically administered to the patient at a higher or lower dose (e.g., a higher or lower daily dose) over the course of a second treatment period. For example, in some embodiments, the compound is administered to the patient in one or more daily doses (e.g., from 1 to 10 doses, such as 1, 2, 3, 4, 5, 6, 7, 8, 9, or 10 doses, or more, such as a single daily dose) totaling from about 150 mg to about 250 mg per day over the course of a first treatment period (e.g., in the recited amount or in an equivalent amount of a pharmaceutically acceptable salt, such as a choline salt), such as in one or more daily doses (e.g., from 1 to 10 doses, such as 1, 2, 3, 4, 5, 6, 7, 8, 9, or 10 doses, or more, such as a single daily dose) totaling an amount of about 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, or 250 mg per day over the course of a first treatment period (e.g., in the recited amount or in an equivalent amount of a pharmaceutically acceptable salt, such as a choline salt). In some embodiments, the compound is subsequently administered to the patient in one or more daily doses (e.g., from 1 to 10 doses, such as 1, 2, 3, 4, 5, 6, 7, 8, 9, or 10 doses, or more, such as a single daily dose) totaling from about 50 mg to about 150 mg per day over the course of a second treatment period (e.g., in the recited amount or in an equivalent amount of a pharmaceutically acceptable salt, such as a choline salt), such as in one or more daily doses (e.g., from 1 to 10 doses, such as 1, 2, 3, 4, 5, 6, 7, 8, 9, or 10 doses, or more, such as a single daily dose) totaling an amount of about 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, or 150 mg per day over the course of a second treatment period (e.g., in the recited amount or in an equivalent amount of a pharmaceutically acceptable salt, such as a choline salt).

In some embodiments, the compound of any one of formulas (I), (Ia)-(Ic), (Ia), (IIb), (IIIa)-(IIIg), (IVa)-(IVc), (V), (Va), or (VI) (e.g., (VIa)) is administered to the patient in one or more daily doses (e.g., from 1 to 10 doses, such as 1, 2, 3, 4, 5, 6, 7, 8, 9, or 10 doses, or more, such as a single daily dose) totaling from about 175 mg to about 225 mg per day over the course of a first treatment period (e.g., in the recited amount or in an equivalent amount of a pharmaceutically acceptable salt, such as a choline salt), such as in one or more daily doses (e.g., from 1 to 10 doses, such as 1, 2, 3, 4, 5, 6, 7, 8, 9, or 10 doses, or more, such as a single daily dose) totaling an amount of about 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, or 225 mg per day over the course of a first treatment period (e.g., in the recited amount or in an equivalent amount of a pharmaceutically acceptable salt, such as a choline salt). In some embodiments, the compound is subsequently administered to the patient in one or more daily doses (e.g., from 1 to 10 doses, such as 1, 2, 3, 4, 5, 6, 7, 8, 9, or 10 doses, or more, such as a single daily dose) totaling from about 75 mg to about 125 mg per day over the course of a second treatment period (e.g., in the recited amount or in an equivalent amount of a pharmaceutically acceptable salt, such as a choline salt), such as in one or more daily doses (e.g., from 1 to 10 doses, such as 1, 2, 3, 4, 5, 6, 7, 8, 9, or 10 doses, or more, such as a single daily dose) totaling an amount of about 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, or 125 mg per day over the course of a second treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIa)-(IIIg), (IVa)-(IVc), (V), (Va), or (VI) (e.g., (VIa)) is administered to the patient in one or more daily doses (e.g., from 1 to 10 doses, such as 1, 2, 3, 4, 5, 6, 7, 8, 9, or 10 doses, or more, such as a single daily dose) totaling from about 185 mg to about 215 mg per day over the course of a first treatment period (e.g., in the recited amount or in an equivalent amount of a pharmaceutically acceptable salt, such as a choline salt), such as in one or more daily doses (e.g., from 1 to 10 doses, such as 1, 2, 3, 4, 5, 6, 7, 8, 9, or 10 doses, or more, such as a single daily dose) totaling an amount of about 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, or 215 mg per day over the course of a first treatment period (e.g., in the recited amount or in an equivalent amount of a pharmaceutically acceptable salt, such as a choline salt).

In some embodiments, the compound is subsequently administered to the patient in one or more daily doses (e.g., from 1 to 10 doses, such as 1, 2, 3, 4, 5, 6, 7, 8, 9, or 10 doses, or more, such as a single daily dose) totaling from about 85 mg to about 115 mg per day over the course of a second treatment period (e.g., in the recited amount or in an equivalent amount of a pharmaceutically acceptable salt, such as a choline salt), such as in one or more daily doses (e.g., from 1 to 10 doses, such as 1, 2, 3, 4, 5, 6, 7, 8, 9, or 10 doses, or more, such as a single daily dose) totaling an amount of about 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, or 115 mg per day over the course of a second treatment period (e.g., in the recited amount or in an equivalent amount of a pharmaceutically acceptable salt, such as a choline salt).

In some embodiments, the compound of any one of formulas (I), (Ia)-(Ic), (IIa), (IIb), (IIIa)-(IIIg), (IVa)-(IVc), (V), (Va), or (VI) (e.g., (VIa)) is administered to the patient in one or more daily doses (e.g., from 1 to 10 doses, such as 1, 2, 3, 4, 5, 6, 7, 8, 9, or 10 doses, or more, such as a single daily dose) totaling about 200 mg per day over the course of a first treatment period (e.g., in the recited amount or in an equivalent amount of a pharmaceutically acceptable salt, such as a choline salt). In some embodiments, the compound is subsequently administered to the patient in one or more daily doses (e.g., from 1 to 10 doses, such as 1, 2, 3, 4, 5, 6, 7, 8, 9, or 10 doses, or more, such as a single daily dose) totaling about 100 mg per day over the course of a second treatment period (e.g., in the recited amount or in an equivalent amount of a pharmaceutically acceptable salt, such as a choline salt).

In some embodiments, the first and second treatment periods collectively have a duration of one or more weeks, months, or years, for example, a combined treatment period of from about 1 week to about 48 months, or more (e.g., a combined treatment period of about 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks, 47 weeks, 48 weeks, 49 weeks, 50 weeks, 51 weeks, 52 weeks, 53 weeks, 54 weeks, 55 weeks, 56 weeks, 57 weeks, 58 weeks, 59 weeks, 60 weeks, 61 weeks, 62 weeks, 63 weeks, 64 weeks, 65 weeks, 66 weeks, 67 weeks, 68 weeks, 69 weeks, 70 weeks, 71 weeks, 72 weeks, 73 weeks, 74 weeks, 75 weeks, 76 weeks, 77 weeks, 78 weeks, 79 weeks, 80 weeks, 81 weeks, 82 weeks, 83 weeks, 84 weeks, 85 weeks, 86 weeks, 87 weeks, 88 weeks, 89 weeks, 90 weeks, 91 weeks, 92 weeks, 93 weeks, 94 weeks, 95 weeks, 96 weeks, 97 weeks, 98 weeks, 99 weeks, 100 weeks, 101 weeks, 102 weeks, 103 weeks, 104 weeks, 105 weeks, 106 weeks, 107 weeks, 108 weeks, 109 weeks, 110 weeks, 111 weeks, 112 weeks, 113 weeks, 114 weeks, 115 weeks, 116 weeks, 117 weeks, 118 weeks, 119 weeks, 120 weeks, 121 weeks, 122 weeks, 123 weeks, 124 weeks, 125 weeks, 126 weeks, 127 weeks, 128 weeks, 129 weeks, 130 weeks, 131 weeks, 132 weeks, 133 weeks, 134 weeks, 135 weeks, 136 weeks, 137 weeks, 138 weeks, 139 weeks, 140 weeks, 141 weeks, 142 weeks, 143 weeks, 144 weeks, 145 weeks, 146 weeks, 147 weeks, 148 weeks, 149 weeks, 150 weeks, 151 weeks, 152 weeks, 153 weeks, 154 weeks, 155 weeks, 156 weeks, 157 weeks, 158 weeks, 159 weeks, 160 weeks, 161 weeks, 162 weeks, 163 weeks, 164 weeks, 165 weeks, 166 weeks, 167 weeks, 168 weeks, 169 weeks, 170 weeks, 171 weeks, 172 weeks, 173 weeks, 174 weeks, 175 weeks, 176 weeks, 177 weeks, 178 weeks, 179 weeks, 180 weeks, 181 weeks, 182 weeks, 183 weeks, 184 weeks, 185 weeks, 186 weeks, 187 weeks, 188 weeks, 189 weeks, 190 weeks, 191 weeks, 192 weeks, 193 weeks, 194 weeks, 195 weeks, 196 weeks, 197 weeks, 198 weeks, 199 weeks, or 200 weeks, or more).

In some embodiments, the first and second treatment periods collectively have a duration of from about 1 month to about 48 months, or more, such as a combined treatment period of about 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 28 months, 29 months, 30 months, 31 months, 32 months, 33 months, 34 months, 35 months, 36 months, 37 months, 38 months, 39 months, 40 months, 41 months, 42 months, 43 months, 44 months, 45 months, 46 months, 47 months, 48 months, or more.

In some embodiments, the first treatment period has a duration of at least 2 weeks, such as a duration of from about 2 weeks to about 6 months, or more. For example, the first treatment period may have a duration of about 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks, or more. In some embodiments, the first treatment period has a duration of from about 0.5 months to about 6 months, or more, such as a duration of about 0.5 months, 1 month, 2 months, 3 months, 4 months, 5 months, or 6 months, or more.

In some embodiments, the first treatment period has a duration of at least 4 weeks, such as a duration of from about 4 weeks to about 5 months, or more. For example, the first treatment period may have a duration of about 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, or 20 weeks, or more. In some embodiments, the first treatment period has a duration of from about 1 month to about 5 months, or more, such as a treatment period of about 1 month, 2 months, 3 months, 4 months, or 5 months, or more.

In some embodiments, the first treatment period has a duration of at least 6 weeks, such as a duration of from about 6 weeks to about 24 weeks (e.g., a duration of from about 8 weeks to about 24 weeks), or more. For example, the first treatment period may have a duration of about 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks, or more. In some embodiments, the first treatment period has a duration of from about 1.5 months to about 6 months, or more, such as a duration of about 1.5 months, 2 months, 3 months, 4 months, 5 months, or 6 months, or more.

In some embodiments, the first treatment period has a duration of about 12 weeks.

In some embodiments, the second treatment period has a duration of at least 2 weeks, such as a duration of from about 2 weeks to about 6 months, or more. For example, the second treatment period may have a duration of about 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks, or more. In some embodiments, the second treatment period has a duration of from about 0.5 months to about 6 months, or more, such as a duration of about 0.5 months, 1 month, 2 months, 3 months, 4 months, 5 months, or 6 months, or more.

In some embodiments, the second treatment period has a duration of at least 4 weeks, such as a duration of from about 4 weeks to about 5 months, or more. For example, the second treatment period may have a duration of about 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, or 20 weeks, or more. In some embodiments, the second treatment period has a duration of from about 1 month to about 5 months, or more, such as a treatment period of about 1 month, 2 months, 3 months, 4 months, or 5 months, or more.

In some embodiments, the second treatment period has a duration of at least 6 weeks, such as a duration of from about 6 weeks to about 24 weeks (e.g., a duration of from about 8 weeks to about 24 weeks), or more. For example, the second treatment period may have a duration of about 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks, or more. In some embodiments, the second treatment period has a duration of from about 1.5 months to about 6 months, or more, such as a duration of about 1.5 months, 2 months, 3 months, 4 months, 5 months, or 6 months, or more.

In some embodiments, the second treatment period has a duration of about 12 weeks.

In some embodiments of any of the foregoing aspects of the disclosure, the GnRH antagonist is a compound represented by any one of formulas (VII)-(XIV), below, such as elagolix, relugolix, or opigolix (ASP1707). In some embodiments, the GnRH antagonist is BAY-784 or SK-2706.

For example, in some embodiments, the GnRH antagonist is a compound represented by formula (VII)

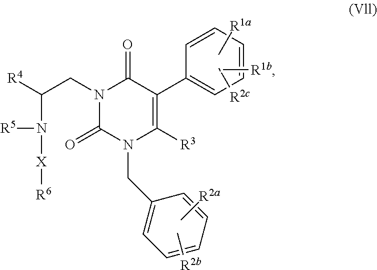

wherein R₁ₐ, R_{1b} and R_{1c} are the same or different and are each independently hydrogen, halogen, C₁₋₄alkyl, hydroxy or alkoxy, or R₁ₐ and R_{1b} taken together form —OCH₂O— or —OCH₂CH₂—;

R₂ₐ and R_{2b} are the same or different and are each independently hydrogen, halogen, trifluoromethyl, cyano or —SO₂CH₃;

R₃ is hydrogen or methyl;

R₄ is phenyl or C₃₋₇alkyl;

R₅ is hydrogen or C₁₋₄alkyl;

R₆ is —COOH or an acid isostere; and

X is C₁₋₆alkanediyl optionally substituted with from 1 to 3 C₁₋₆alkyl groups; or a pharmaceutically acceptable salt thereof.

In some embodiments, the GnRH antagonist is a compound represented by formula (VIII)

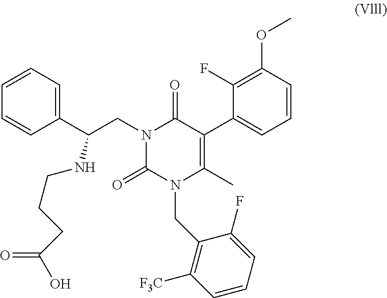 [SMILES: COc1cccc(-c2c(C)n(Cc3c(F)cccc3C(F)(F)F)c(=O)n(C[C@H](NCCCC(=O)O)c3ccccc3)c2=O)c1F]

or a pharmaceutically acceptable salt thereof. In some embodiments, the GnRH antagonist is the sodium salt of the compound represented by formula (VIII), which is represented by formula (IX), below.

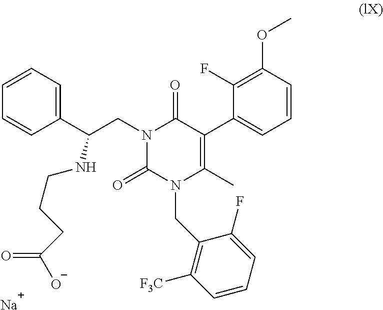 [SMILES: COc1cccc(-c2c(C)n(Cc3c(F)cccc3C(F)(F)F)c(=O)n(C[C@H](NCCCC(=O)[O-])c3ccccc3)c2=O)c1F ; [Na+]]

In some embodiments, the compound of any one of formulas (VII)-(IX) is administered to the patient in an amount of from about 50 mg to about 650 mg per dose (e.g., in the recited amount or in an equivalent amount of a pharmaceutically acceptable salt, such as a sodium salt), such as an amount of about 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, 400 mg, 401 mg, 402 mg, 403 mg, 404 mg, 405 mg, 406 mg, 407 mg, 408 mg, 409 mg, 410 mg, 411 mg, 412 mg, 413 mg, 414 mg, 415 mg, 416 mg, 417 mg, 418 mg, 419 mg, 420 mg, 421 mg, 422 mg, 423 mg, 424 mg, 425 mg, 426 mg, 427 mg, 428 mg, 429 mg, 430 mg, 431 mg, 432 mg, 433 mg, 434 mg, 435 mg, 436 mg, 437 mg, 438 mg, 439 mg, 440 mg, 441 mg, 442 mg, 443 mg, 444 mg, 445 mg, 446 mg, 447 mg, 448 mg, 449 mg, 450 mg, 451 mg, 452 mg, 453 mg, 454 mg, 455 mg, 456 mg, 457 mg, 458 mg, 459 mg, 460 mg, 461 mg, 462 mg, 463 mg, 464 mg, 465 mg, 466 mg, 467 mg, 468 mg, 469 mg, 470 mg, 471 mg, 472 mg, 473 mg, 474 mg, 475 mg, 476 mg, 477 mg, 478 mg, 479 mg, 480 mg, 481 mg, 482 mg, 483 mg, 484 mg, 485 mg, 486 mg, 487 mg, 488 mg, 489 mg, 490 mg, 491 mg, 492 mg, 493 mg, 494 mg, 495 mg, 496 mg, 497 mg, 498 mg, 499 mg, 500 mg, 501 mg, 502 mg, 503 mg, 504 mg, 505 mg, 506 mg, 507 mg, 508 mg, 509 mg, 510 mg, 511 mg, 512 mg, 513 mg, 514 mg, 515 mg, 516 mg, 517 mg, 518 mg, 519 mg, 520 mg, 521 mg, 522 mg, 523 mg, 524 mg, 525 mg, 526 mg, 527 mg, 528 mg, 529 mg, 530 mg, 531 mg, 532 mg, 533 mg, 534 mg, 535 mg, 536 mg, 537 mg, 538 mg, 539 mg, 540 mg, 541 mg, 542 mg, 543 mg, 544 mg, 545 mg, 546 mg, 547 mg, 548 mg, 549 mg, 550 mg, 551 mg, 552 mg, 553 mg, 554 mg, 555 mg, 556 mg, 557 mg, 558 mg, 559 mg, 560 mg, 561 mg, 562 mg, 563 mg, 564 mg, 565 mg, 566 mg, 567 mg, 568 mg, 569 mg, 570 mg, 571 mg, 572 mg, 573 mg, 574 mg, 575 mg, 576 mg, 577 mg, 578 mg, 579 mg, 580 mg, 581 mg, 582 mg, 583 mg, 584 mg, 585 mg, 586 mg, 587 mg, 588 mg, 589 mg, 590 mg, 591 mg, 592 mg, 593 mg, 594 mg, 595 mg, 596 mg, 597 mg, 598 mg, 599 mg, 600 mg, 601 mg, 602 mg, 603 mg, 604 mg, 605 mg, 606 mg, 607 mg, 608 mg, 609 mg, 610 mg, 611 mg, 612 mg, 613 mg, 614 mg, 615 mg, 616 mg, 617 mg, 618 mg, 619 mg, 620 mg, 621 mg, 622 mg, 623 mg, 624 mg, 625 mg, 626 mg, 627 mg, 628 mg, 629 mg, 630 mg, 631 mg, 632 mg, 633 mg, 634 mg, 635 mg, 636 mg, 637 mg, 638 mg, 639 mg, 640 mg, 641 mg, 642 mg, 643 mg, 644 mg, 645 mg, 646 mg, 647 mg, 648 mg, 649 mg, or 650 mg per dose (e.g., in the recited amount or in an equivalent amount of a pharmaceutically acceptable salt, such as a sodium salt). In some embodiments, the compound of any one of formulas (VII)-(IX) is administered to the patient in an amount of about 150 mg per dose, 300 mg per dose, 400 mg per dose, or 600 mg per dose (e.g., in the recited amount or in an equivalent amount of a pharmaceutically acceptable salt, such as a sodium salt).

In some embodiments, the compound of any one of formulas (VII)-(IX) is administered to the patient in one or more doses (i.e., one or more times) per day, week, or month, such as from 1 to 10 times per day (e.g., 1 time, 2 times, 3 times, 4 times, 5 times, 6 times, 7 times, 8 times, 9 times, or 10 times per day, such as 1 time or 2 times per day), 1 to 100 times per week (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 25 times, 30 times, 35 times 40 times, 45 times, 50 times, 55 times, 60 times, 65 times, 70 times, 75 times, 80 times, 85 times, 90 times, 95 times, or 100 times per week, such as 7 times, 8 times, 9 times, 10 times, 11 times, 12 times, 13 times, 14 times per week), or 1 to 500 times per month (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 21 times, 22 times, 23 times, 24 times, 25 times, 26 times, 27 times, 28 times, 29 times, 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, 60 times, 61 times, 62 times, 63 times, 64 times, 65 times, 66 times, 67 times, 68 times, 69 times, 70 times, 71 times, 72 times, 73 times, 74 times, 75 times, 76 times, 77 times, 78 times, 79 times, 80 times, 81 times, 82 times, 83 times, 84 times, 85 times, 86 times, 87 times, 88 times, 89 times, 90 times, 91 times, 92 times, 93 times, 94 times, 95 times, 96 times, 97 times, 98 times, 99 times, 100 times, 200 times, 300 times, 400 times, or 500 times per month, such as 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, or 60 times per month), or more.

For example, the compound of any one of formulas (VII)-(IX) may be administered to the patient in one or more doses every 4 hours, 6 hours, 8 hours, 10 hours, 12 hours, 14 hours, 16 hours, 18 hours, 20 hours, 22 hours, 24 hours, 28 hours, 30 hours, 32 hours, 34 hours, 36 hours, 38 hours, 40 hours, 42 hours, 44 hours, 46 hours, 48 hours, 50 hours, 52 hours, 54 hours, 56 hours, 58 hours, 60 hours, 62 hours, 64 hours, 66 hours, 68 hours, 70 hours, or 72 hours, 74 hours, 76 hours, 78 hours, 80 hours, 82 hours, 84 hours, 86 hours, 88 hours, 90 hours, 92 hours, 94 hours, 96 hours, 98 hours, 100 hour, 102 hours, 104 hours, 105 hours, 106 hours, 108 hours, 110 hours, 112 hours, 114 hours, 116 hours, 118 hours, 120 hours, 122 hours, 124 hours, 126 hours, 128 hours, 130 hours, 132 hours, 134 hours, 136 hours, 138 hours, 140 hours, 142 hours, 144 hours, 146 hours, 148 hours, 150 hours, 152 hours, 154 hours, 156 hours, 158 hours, 160 hours, 162 hours, 164 hours, 166 hours, 168 hours, or more. In some embodiments, the compound of any one of formulas (VII)-(IX) is administered to the patient in one or more doses per day, such as from 1 to 10 doses per 12 hours (e.g., 1 dose, 2 doses, 3 doses, 4 doses, 5 doses, 6 doses, 7 doses, 8 doses, 9 doses, 10 doses per 12 hours), from 1 to 10 doses per 24 hours (e.g., 1 dose, 2 doses, 3 doses, 4 doses, 5 doses, 6 doses, 7 doses, 8 doses, 9 doses, 10 doses per 24 hours), or from 1 to 10 doses per 48 hours (e.g., 1 dose, 2 doses, 3 doses, 4 doses, 5 doses, 6 doses, 7 doses, 8 doses, 9 doses, 10 doses per 48 hours), among others.

The compound of any one of formulas (VII)-(IX) may be administered to the patient in one or more unit dosage forms that collectively constitute a single dose. For example, a patient may be administered a single dose of the compound of a specified amount, such as a single dose of 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, or more (e.g., in the recited amount or in an equivalent amount of a pharmaceutically acceptable salt, such as a sodium salt), by administration of one or more unit dosage forms of the compound to the patient. As a non-limiting example, a single dose of 300 mg of the compound may be administered to the subject by way of two individual 150-mg unit dosage forms of the compound. The two 150-mg unit dosage forms collectively constitute a single 300-mg dose of the compound if administered to the patient at substantially the same time.

In some embodiments, the compound of any one of formulas (VII)-(IX) is administered to the patient in one or more daily doses (e.g., from 1 to 10 doses, such as 1, 2, 3, 4, 5, 6, 7, 8, 9, or 10 doses, or more) totaling from about 50 mg to about 650 mg per day (e.g., in the recited amount or in an equivalent amount of a pharmaceutically acceptable salt, such as a sodium salt), such as an amount of about 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, 400 mg, 401 mg, 402 mg, 403 mg, 404 mg, 405 mg, 406 mg, 407 mg, 408 mg, 409 mg, 410 mg, 411 mg, 412 mg, 413 mg, 414 mg, 415 mg, 416 mg, 417 mg, 418 mg, 419 mg, 420 mg, 421 mg, 422 mg, 423 mg, 424 mg, 425 mg, 426 mg, 427 mg, 428 mg, 429 mg, 430 mg, 431 mg, 432 mg, 433 mg, 434 mg, 435 mg, 436 mg, 437 mg, 438 mg, 439 mg, 440 mg, 441 mg, 442 mg, 443 mg, 444 mg, 445 mg, 446 mg, 447 mg, 448 mg, 449 mg, 450 mg, 451 mg, 452 mg, 453 mg, 454 mg, 455 mg, 456 mg, 457 mg, 458 mg, 459 mg, 460 mg, 461 mg, 462 mg, 463 mg, 464 mg, 465 mg, 466 mg, 467 mg, 468 mg, 469 mg, 470 mg, 471 mg, 472 mg, 473 mg, 474 mg, 475 mg, 476 mg, 477 mg, 478 mg, 479 mg, 480 mg, 481 mg, 482 mg, 483 mg, 484 mg, 485 mg, 486 mg, 487 mg, 488 mg, 489 mg, 490 mg, 491 mg, 492 mg, 493 mg, 494 mg, 495 mg, 496 mg, 497 mg, 498 mg, 499 mg, 500 mg, 501 mg, 502 mg, 503 mg, 504 mg, 505 mg, 506 mg, 507 mg, 508 mg, 509 mg, 510 mg, 511 mg, 512 mg, 513 mg, 514 mg, 515 mg, 516 mg, 517 mg, 518 mg, 519 mg, 520 mg, 521 mg, 522 mg, 523 mg, 524 mg, 525 mg, 526 mg, 527 mg, 528 mg, 529 mg, 530 mg, 531 mg, 532 mg, 533 mg, 534 mg, 535 mg, 536 mg, 537 mg, 538 mg, 539 mg, 540 mg, 541 mg, 542 mg, 543 mg, 544 mg, 545 mg, 546 mg, 547 mg, 548 mg, 549 mg, 550 mg, 551 mg, 552 mg, 553 mg, 554 mg, 555 mg, 556 mg, 557 mg, 558 mg, 559 mg, 560 mg, 561 mg, 562 mg, 563 mg, 564 mg, 565 mg, 566 mg, 567 mg, 568 mg, 569 mg, 570 mg, 571 mg, 572 mg, 573 mg, 574 mg, 575 mg, 576 mg, 577 mg, 578 mg, 579 mg, 580 mg, 581 mg, 582 mg, 583 mg, 584 mg, 585 mg, 586 mg, 587 mg, 588 mg, 589 mg, 590 mg, 591 mg, 592 mg, 593 mg, 594 mg, 595 mg, 596 mg, 597 mg, 598 mg, 599 mg, 600 mg, 601 mg, 602 mg, 603 mg, 604 mg, 605 mg, 606 mg, 607 mg, 608 mg, 609 mg, 610 mg, 611 mg, 612 mg, 613 mg, 614 mg, 615 mg, 616 mg, 617 mg, 618 mg, 619 mg, 620 mg, 621 mg, 622 mg, 623 mg, 624 mg, 625 mg, 626 mg, 627 mg, 628 mg, 629 mg, 630 mg, 631 mg, 632 mg, 633 mg, 634 mg, 635 mg, 636 mg, 637 mg, 638 mg, 639 mg, 640 mg, 641 mg, 642 mg, 643 mg, 644 mg, 645 mg, 646 mg, 647 mg, 648 mg, 649 mg, or 650 mg per day (e.g., in the recited amount or in an equivalent amount of a pharmaceutically acceptable salt, such as a sodium salt). In some embodiments, the compound is administered to the patient in one or more daily doses (e.g., from 1 to 10 doses, such as 1, 2, 3, 4, 5, 6, 7, 8, 9, or 10 doses, or more) totaling an amount of about 150 mg per day, 300 mg per day, 400 mg per day (e.g., 200 mg administered twice daily, or 600 mg per day (e.g., 300 mg administered twice daily) (e.g., in the recited amount or in an equivalent amount of a pharmaceutically acceptable salt, such as a sodium salt).

In some embodiments, the GnRH antagonist is a compound represented by formula (X)

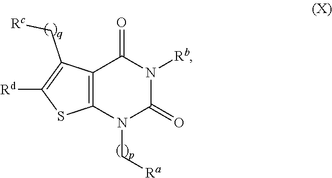

wherein R^{a} is a hydrogen atom, an optionally substituted aryl group (such as an aryl group that may have 1 to 5 substituents selected from halogen, nitro, cyano, amino, a carboxyl group that may be esterified or amidated, an alkylenedioxy, alkyl, alkoxy, alkylthio, alkylsulfinyl, and alkylsulfonyl), an optionally substituted cycloalkyl group, or an optionally substituted heterocyclic group;

R^{b} is an optionally substituted nitrogen-containing heterocyclic group;

R^{c} is an optionally substituted amino group;

R^{d} is an optionally substituted aryl group;

p is an integer from 0 to 3; and

q is an integer from 0 to 3;

or a pharmaceutically acceptable salt thereof.

In some embodiments, the GnRH antagonist is a compound represented by formula (XI)

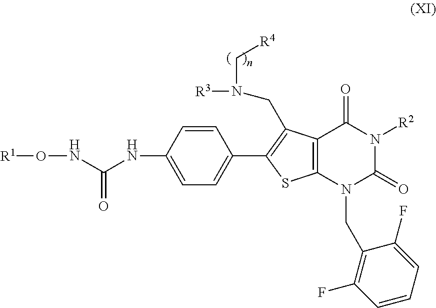

wherein R¹ is C₁₋₄alkyl;

R² is (1) a C₁₋₆alkyl which may have a substituent selected from the group consisting of (1′) a hydroxy group, (2) a C₁₋₄alkoxy, (3′) a C₁₋₄alkoxy-carbonyl, (4′) a di-C₁₋₄alkyl-carbamoyl, (5′) a 5- to 7-membered nitrogen-containing heterocyclic group, (6′) a C₁₋₄alkyl-carbonyl and (7′) a halogen, (2) a C₃₋₈ cycloalkyl which may have (1′) a hydroxy group or (2′) a mono-C₁₋₄alkyl-carbonylamino, (3) a 5- to 7-membered nitrogen-containing heterocyclic group which may have a substituent selected from the group consisting of (1′) a halogen, (2′) a hydroxy group, (3′) a C₁₋₄alkyl and (4′) a C₁-4alkoxy, (4) a phenyl which may have a substituent selected from the group consisting of (1′) a halogen, (2′) a C₁₋₄alkoxy-C₁₋₄alkyl, (3′) a mono-C₁₋₄alkyl-carbamoyl-C₁₋₄alkyl, (4′) a C₁₋₄alkoxy and (5′) a mono-C₁₋₄alkylcarbamoyl-C₁₋₄alkoxy, or (5) a C₁₋₄alkoxy;

R³ is C₁₋₄alkyl;

R⁴ is (1) hydrogen, (2) C₁₋₄alkoxy, (3) C₆₋₁₀aryl, (4)N—C₁₋₄alkyl-N—C₁₋₄alkylsulfonylamino, (5) hydroxyl, or (6) a 5- to 7-membered nitrogen-containing heterocyclic group which may have a substituent selected from the group consisting of (1′) oxo, (2′) a C₁₋₄alkyl, (3′) a hydroxy-C₁₋₄alkyl, (4′) a C₁₋₄alkoxy-carbonyl, (5′) a mono-C₁₋₄alkyl-carbamoyl and (6′) a C₁₋₄alkylsulfonyl; and

n is an integer from 1 to 4;

optionally provided that when R² is a phenyl which may have a substituent, R⁴ is a 5- to 7-membered nitrogen-containing heterocyclic group which may have a substituent selected from the group consisting of (1) oxo, (2) hydroxy-C₁₋₄alkyl, (3) C₁₋₄alkoxy-carbonyl, (4) mono-C₁₋₄alkyl-carbamoyl and (5) C₁₋₄alkylsulfonyl;

or a pharmaceutically acceptable salt thereof. In some embodiments, the GnRH antagonist is a compound represented by formula (XII), below, or a pharmaceutically acceptable salt thereof, such as a chloride salt thereof.

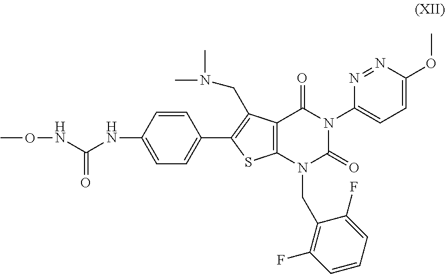

In some embodiments, the compound of any one of formulas (X)-(XII) is administered to the patient in an amount of from about 10 mg to about 60 mg per dose (e.g., in the recited amount or in an equivalent amount of a pharmaceutically acceptable salt, such as a chloride salt), such as an amount of about 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, or 60 mg per dose (e.g., in the recited amount or in an equivalent amount of a pharmaceutically acceptable salt, such as a chloride salt). In some embodiments, the compound of any one of formulas (X)-(XII) is administered to the patient in an amount of about 40 mg per dose (e.g., in the recited amount or in an equivalent amount of a pharmaceutically acceptable salt, such as a chloride salt).

In some embodiments, the compound of any one of formulas (X)-(XI) is administered to the patient in one or more doses (i.e., one or more times) per day, week, or month, such as from 1 to 10 times per day (e.g., 1 time, 2 times, 3 times, 4 times, 5 times, 6 times, 7 times, 8 times, 9 times, or 10 times per day, such as 1 time or 2 times per day), 1 to 100 times per week (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 25 times, 30 times, 35 times 40 times, 45 times, 50 times, 55 times, 60 times, 65 times, 70 times, 75 times, 80 times, 85 times, 90 times, 95 times, or 100 times per week, such as 7 times, 8 times, 9 times, 10 times, 11 times, 12 times, 13 times, 14 times per week), or 1 to 500 times per month (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 21 times, 22 times, 23 times, 24 times, 25 times, 26 times, 27 times, 28 times, 29 times, 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, 60 times, 61 times, 62 times, 63 times, 64 times, 65 times, 66 times, 67 times, 68 times, 69 times, 70 times, 71 times, 72 times, 73 times, 74 times, 75 times, 76 times, 77 times, 78 times, 79 times, 80 times, 81 times, 82 times, 83 times, 84 times, 85 times, 86 times, 87 times, 88 times, 89 times, 90 times, 91 times, 92 times, 93 times, 94 times, 95 times, 96 times, 97 times, 98 times, 99 times, 100 times, 200 times, 300 times, 400 times, or 500 times per month, such as 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, or 60 times per month), or more.

For example, the compound of any one of formulas (X)-(XII) may be administered to the patient in one or more doses every 4 hours, 6 hours, 8 hours, 10 hours, 12 hours, 14 hours, 16 hours, 18 hours, 20 hours, 22 hours, 24 hours, 28 hours, 30 hours, 32 hours, 34 hours, 36 hours, 38 hours, 40 hours, 42 hours, 44 hours, 46 hours, 48 hours, 50 hours, 52 hours, 54 hours, 56 hours, 58 hours, 60 hours, 62 hours, 64 hours, 66 hours, 68 hours, 70 hours, or 72 hours, 74 hours, 76 hours, 78 hours, 80 hours, 82 hours, 84 hours, 86 hours, 88 hours, 90 hours, 92 hours, 94 hours, 96 hours, 98 hours, 100 hour, 102 hours, 104 hours, 105 hours, 106 hours, 108 hours, 110 hours, 112 hours, 114 hours, 116 hours, 118 hours, 120 hours, 122 hours, 124 hours, 126 hours, 128 hours, 130 hours, 132 hours, 134 hours, 136 hours, 138 hours, 140 hours, 142 hours, 144 hours, 146 hours, 148 hours, 150 hours, 152 hours, 154 hours, 156 hours, 158 hours, 160 hours, 162 hours, 164 hours, 166 hours, 168 hours, or more. In some embodiments, the compound of any one of formulas (X)-(XII) is administered to the patient in one or more doses per day, such as from 1 to 10 doses per 12 hours (e.g., 1 dose, 2 doses, 3 doses, 4 doses, 5 doses, 6 doses, 7 doses, 8 doses, 9 doses, 10 doses per 12 hours), from 1 to 10 doses per 24 hours (e.g., 1 dose, 2 doses, 3 doses, 4 doses, 5 doses, 6 doses, 7 doses, 8 doses, 9 doses, 10 doses per 24 hours), or from 1 to 10 doses per 48 hours (e.g., 1 dose, 2 doses, 3 doses, 4 doses, 5 doses, 6 doses, 7 doses, 8 doses, 9 doses, 10 doses per 48 hours), among others.

The compound of any one of formulas (X)-(XII) may be administered to the patient in one or more unit dosage forms that collectively constitute a single dose. For example, a patient may be administered a single dose of the compound of a specified amount, such as a single dose of 25 mg, 50 mg, 75 mg, 100 mg, or more (e.g., in the recited amount or in an equivalent amount of a pharmaceutically acceptable salt, such as a chloride salt), by administration of one or more unit dosage forms of the compound to the patient. As a non-limiting example, a single dose of 40 mg of the compound may be administered to the subject by way of two individual 20-mg unit dosage forms of the compound. The two 20-mg unit dosage forms collectively constitute a single 40-mg dose of the compound if administered to the patient at substantially the same time.

In some embodiments, the compound of any one of formulas (X)-(XI) is administered to the patient in one or more daily doses (e.g., from 1 to 10 doses, such as 1, 2, 3, 4, 5, 6, 7, 8, 9, or 10 doses, or more) totaling from about 10 mg to about 60 mg per day (e.g., in the recited amount or in an equivalent amount of a pharmaceutically acceptable salt, such as a chloride salt), such as an amount of about 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, or 60 mg per day (e.g., in the recited amount or in an equivalent amount of a pharmaceutically acceptable salt, such as a chloride salt). In some embodiments, the compound is administered to the patient in one or more daily doses (e.g., from 1 to 10 doses, such as 1, 2, 3, 4, 5, 6, 7, 8, 9, or 10 doses, or more) totaling from about 20 mg to about 50 mg per day (e.g., in the recited amount or in an equivalent amount of a pharmaceutically acceptable salt, such as a chloride salt), such as an amount of about 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, or 50 mg per day (e.g., in the recited amount or in an equivalent amount of a pharmaceutically acceptable salt, such as a chloride salt). In some embodiments, the compound is administered to the patient in one or more daily doses (e.g., from 1 to 10 doses, such as 1, 2, 3, 4, 5, 6, 7, 8, 9, or 10 doses, or more) totaling an amount of about 40 mg per day (e.g., a single daily dose of 40 mg) (e.g., in the recited amount or in an equivalent amount of a pharmaceutically acceptable salt, such as a chloride salt).

In some embodiments, the GnRH antagonist is a compound represented by formula (XIII)

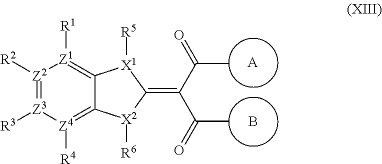

wherein R¹, R², R³ and R⁴ are the same or different, and are each independently selected from hydrogen, nitro, cyano, halogen, an optionally substituted hydrocarbon group, an optionally substituted heterocyclic group, hydroxy, alkoxy, carboxy, optionally substituted acyl-O—, optionally substituted acyl, a substituent —S(O)n₁₀₁₋(wherein n₁₀₁ is an integer of 0 to 2), H—S(O)n₁₀₁₋, optionally substituted carbamoyl, optionally substituted sulfamoyl, optionally substituted amino, and two adjacent groups selected from the group of R¹, R², R³ and R⁴ may combine to form an aryl or a carbocyclic (e.g., cycloalkenyl) group;

R⁵ and R⁶ are the same or different and are each independently selected from hydrogen, halogen, optionally substituted hydrocarbon, and optionally substituted amino,

X¹ and X² are the same or different and are each independently selected from N, S and O;

A and B are the same or different and are each independently selected from optionally substituted aryl and optionally substituted heterocyclyl, and

Z¹, Z², Z³ and Z⁴ are each independently selected from C and N; optionally provided that 1) when X¹ and X² each is S or O, one or both of the corresponding R⁵ and R⁶ are absent; and/or 2) when one to four of Z¹, Z², Z³ and/or Z⁴ are N, the corresponding R¹, R², R³ and/or R⁴ are absent;

or a pharmaceutically acceptable salt thereof. In some embodiments, the GnRH antagonist is a compound represented by formula (XIV), below.

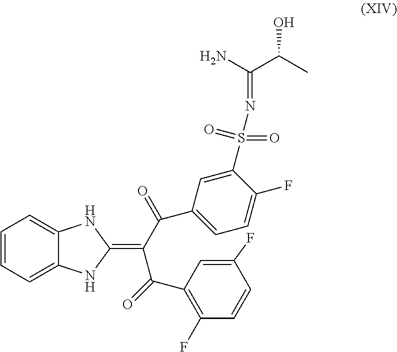 [SMILES: C[C@@H](O)/C(N)=N/S(=O)(=O)c1cc(C(=O)C(C(=O)c2cc(F)ccc2F)=C2Nc3ccccc3N2)ccc1F]

In some embodiments, the compound of any one of formulas (XIII) and (XIV), or SK12670 or BAY-784, is administered to the patient in one or more doses (i.e., one or more times) per day, week, or month, such as from 1 to 10 times per day (e.g., 1 time, 2 times, 3 times, 4 times, 5 times, 6 times, 7 times, 8 times, 9 times, or 10 times per day, such as 1 time or 2 times per day), 1 to 100 times per week (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 25 times, 30 times, 35 times 40 times, 45 times, 50 times, 55 times, 60 times, 65 times, 70 times, 75 times, 80 times, 85 times, 90 times, 95 times, or 100 times per week, such as 7 times, 8 times, 9 times, 10 times, 11 times, 12 times, 13 times, 14 times per week), or 1 to 500 times per month (e.g., 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 21 times, 22 times, 23 times, 24 times, 25 times, 26 times, 27 times, 28 times, 29 times, 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, 60 times, 61 times, 62 times, 63 times, 64 times, 65 times, 66 times, 67 times, 68 times, 69 times, 70 times, 71 times, 72 times, 73 times, 74 times, 75 times, 76 times, 77 times, 78 times, 79 times, 80 times, 81 times, 82 times, 83 times, 84 times, 85 times, 86 times, 87 times, 88 times, 89 times, 90 times, 91 times, 92 times, 93 times, 94 times, 95 times, 96 times, 97 times, 98 times, 99 times, 100 times, 200 times, 300 times, 400 times, or 500 times per month, such as 30 times, 31 times, 32 times, 33 times, 34 times, 35 times, 36 times, 37 times, 38 times, 39 times, 40 times, 41 times, 42 times, 43 times, 44 times, 45 times, 46 times, 47 times, 48 times, 49 times, 50 times, 51 times, 52 times, 53 times, 54 times, 55 times, 56 times, 57 times, 58 times, 59 times, or 60 times per month), or more.

For example, the compound of any one of formulas (XII) and (XIV), or SK12670 or BAY-784, may be administered to the patient in one or more doses every 4 hours, 6 hours, 8 hours, 10 hours, 12 hours, 14 hours, 16 hours, 18 hours, 20 hours, 22 hours, 24 hours, 28 hours, 30 hours, 32 hours, 34 hours, 36 hours, 38 hours, 40 hours, 42 hours, 44 hours, 46 hours, 48 hours, 50 hours, 52 hours, 54 hours, 56 hours, 58 hours, 60 hours, 62 hours, 64 hours, 66 hours, 68 hours, 70 hours, or 72 hours, 74 hours, 76 hours, 78 hours, 80 hours, 82 hours, 84 hours, 86 hours, 88 hours, 90 hours, 92 hours, 94 hours, 96 hours, 98 hours, 100 hour, 102 hours, 104 hours, 105 hours, 106 hours, 108 hours, 110 hours, 112 hours, 114 hours, 116 hours, 118 hours, 120 hours, 122 hours, 124 hours, 126 hours, 128 hours, 130 hours, 132 hours, 134 hours, 136 hours, 138 hours, 140 hours, 142 hours, 144 hours, 146 hours, 148 hours, 150 hours, 152 hours, 154 hours, 156 hours, 158 hours, 160 hours, 162 hours, 164 hours, 166 hours, 168 hours, or more. In some embodiments, the compound of any one of formulas (XII) and (XIV), or SK12670 or BAY-784, is administered to the patient in one or more doses per day, such as from 1 to 10 doses per 12 hours (e.g., 1 dose, 2 doses, 3 doses, 4 doses, 5 doses, 6 doses, 7 doses, 8 doses, 9 doses, 10 doses per 12 hours), from 1 to 10 doses per 24 hours (e.g., 1 dose, 2 doses, 3 doses, 4 doses, 5 doses, 6 doses, 7 doses, 8 doses, 9 doses, 10 doses per 24 hours), or from 1 to 10 doses per 48 hours (e.g., 1 dose, 2 doses, 3 doses, 4 doses, 5 doses, 6 doses, 7 doses, 8 doses, 9 doses, 10 doses per 48 hours), among others.

In some embodiments, the add-back therapy is administered to the patient concurrently with the GnRH antagonist, prior to administration of the GnRH antagonist, or following administration of the GnRH antagonist. In some embodiments, add-back therapy is administered as a fixed dose combination containing a GnRH antagonist, estrogen, and one or more additional agents, such as a progestin, in a single pharmaceutical composition. For instance, add-back therapy may be administered as a fixed dose combination of a GnRH antagonist, estrogen (e.g., in the form of p17-estradiol, ethinyl estradiol, or a conjugated estrogen, such as a conjugated equine estrogen) and/or a progestin (e.g., norethindrone or a compound that is metabolized in vivo to produce norethindrone, such as an ester of norethindrone that is de-esterified in vivo to produce norethindrone, for instance, norethindrone acetate (also referred to herein as “NETA”), among other agents, such as progesterone, norgestimate, medroxyprogesterone, and drospirenone) in the form of a single pharmaceutical composition, such as a single tablet, capsule, gel cap, powder, liquid solution, or liquid suspension. In some embodiments, the add-back therapy is administered orally, transdermally, or intravaginally.

In some embodiments, the add-back therapy is administered to the patient in one or more doses per day, week, month, or year, such as daily, for example, from 1 to 10 times daily, or more (e.g., 1, 2, 3, 4, 5, 6, 7, 8, 9, 10, or more, times daily). In some embodiments, the add-back therapy is administered to the patient once daily, for example, concurrently with the GnRH antagonist. For example, the GnRH antagonist may be administered to the patient orally, and concurrently with oral administration of the GnRH antagonist, the add-back therapy may be administered to the patient orally, transdermally, or intravaginally. In some embodiments, the add-back therapy is administered to the patient in the form of a pharmaceutical composition that further includes the GnRH antagonist, such as a single tablet, capsule, gel cap, powder, liquid solution, or liquid suspension, for instance, as described above and herein.

In some embodiments, the add-back therapy is administered to the patient once daily, following administration of the GnRH antagonist. For example, the GnRH antagonist may be administered to the patient orally, and following oral administration of the GnRH antagonist, the add-back therapy may be administered to the patient orally, transdermally, or intravaginally.

In some embodiments, the add-back therapy is administered to the patient once daily, prior to administration of the GnRH antagonist. For example, the GnRH antagonist may be administered to the patient orally, and prior to oral administration of the GnRH antagonist, the add-back therapy may be administered to the patient orally, transdermally, or intravaginally.

In some embodiments, the add-back therapy includes an estrogen. In some embodiments, the estrogen is selected from the group consisting of β17-estradiol, ethinyl estradiol, and conjugated estrogens, such as conjugated equine estrogens.

In some embodiments, the estrogen is β17-estradiol. The β17-estradiol may be administered to the patient, for example, at a dose of from about 0.1 mg to about 2.5 mg, such as at a dose of about 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, or 2.5 mg, for instance, by oral administration. In some embodiments, the β17-estradiol is administered to the patient at a dose of 1.0 mg, for instance, by oral administration. In some embodiments, the β17-estradiol is administered to the patient at a dose of 0.5 mg, for instance, by oral administration.

The β17-estradiol may be administered to the patient one or more times per day, week, or month. The β17-estradiol may be administered to the patient, for example, in an amount of from about 0.1 mg/day to about 2.5 mg/day, such as in an amount of about 0.1 mg/day, 0.2 mg/day, 0.3 mg/day, 0.4 mg/day, 0.5 mg/day, 0.6 mg/day, 0.7 mg/day, 0.8 mg/day, 0.9 mg/day, 1.0 mg/day, 1.1 mg/day, 1.2 mg/day, 1.3 mg/day, 1.4 mg/day, 1.5 mg/day, 1.6 mg/day, 1.7 mg/day, 1.8 mg/day, 1.9 mg/day, 2.0 mg/day, 2.1 mg/day, 2.2 mg/day, 2.3 mg/day, 2.4 mg/day, or 2.5 mg/day, for instance, by oral administration. In some embodiments, the β17-estradiol is administered to the patient in an amount of 1.0 mg/day, for instance, by oral administration. In some embodiments, the β17-estradiol is administered to the patient in an amount of 0.5 mg/day, for instance, by oral administration.

In some embodiments, the estrogen is ethinyl estradiol. The ethinyl estradiol may be administered to the patient, for example, at a dose of from about 1.0 μg to about 6.0 μg, such as at a dose of about 1.0 μg, 1.1 μg, 1.2 μg, 1.3 μg, 1.4 μg, 1.5 μg, 1.6 μg, 1.7 μg, 1.8 μg, 1.9 μg, 2.0 μg, 2.1 μg, 2.2 μg, 2.3 μg, 2.4 μg, 2.5 μg, 2.6 μg, 2.7 μg, 2.8 μg, 2.9 μg, 3.0 μg, 3.1 μg, 3.2 μg, 3.3 μg, 3.4 μg, 3.5 μg, 3.6 μg, 3.7 μg, 3.8 μg, 3.9 μg, 4.0 μg, 4.1 μg, 4.2 μg, 4.2 μg, 4.3 μg, 4.4 μg, 4.5 μg, 4.6 μg, 4.7 μg, 4.8 μg, 4.9 μg, 5.0 μg, 5.1 μg, 5.2 μg, 5.3 μg, 5.4 μg, 5.5 μg, 5.6 μg, 5.7 μg, 5.8 μg, 5.9 μg, or 6.0 μg, for instance, by oral administration. In some embodiments, the ethinyl estradiol is administered to the patient at a dose of 5.0 μg, for instance, by oral administration. In some embodiments, the ethinyl estradiol is administered to the patient at a dose of 2.5 μg, for instance, by oral administration.

The ethinyl estradiol may be administered to the patient one or more times per day, week, or month. The ethinyl estradiol may be administered to the patient, for example, in an amount of about 1.0 μg/day to about 6.0 μg/day, such as in an amount of about 1.0 μg/day, 1.1 μg/day, 1.2 μg/day, 1.3 μg/day, 1.4 μg/day, 1.5 μg/day, 1.6 μg/day, 1.7 μg/day, 1.8 μg/day, 1.9 μg/day, 2.0 μg/day, 2.1 μg/day, 2.2 μg/day, 2.3 μg/day, 2.4 μg/day, 2.5 μg/day, 2.6 μg/day, 2.7 μg/day, 2.8 μg/day, 2.9 μg/day, 3.0 μg/day, 3.1 μg/day, 3.2 μg/day, 3.3 μg/day, 3.4 μg/day, 3.5 μg/day, 3.6 μg/day, 3.7 μg/day, 3.8 μg/day, 3.9 μg/day, 4.0 μg/day, 4.1 μg/day, 4.2 μg/day, 4.2 μg/day, 4.3 μg/day, 4.4 μg/day, 4.5 μg/day, 4.6 μg/day, 4.7 μg/day, 4.8 μg/day, 4.9 μg/day, 5.0 μg/day, 5.1 μg/day, 5.2 μg/day, 5.3 μg/day, 5.4 μg/day, 5.5 μg/day, 5.6 μg/day, 5.7 μg/day, 5.8 μg/day, 5.9 μg/day, or 6.0 μg/day, for instance, by oral administration. In some embodiments, the ethinyl estradiol is administered to the patient in an amount of 5.0 μg/day, for instance, by oral administration. In some embodiments, the ethinyl estradiol is administered to the patient in an amount of 2.5 μg/day, for instance, by oral administration.

In some embodiments, the estrogen is a conjugated estrogen, such as a conjugated equine estrogen. The conjugated estrogen may be administered to the patient, for example, at a dose of from about 0.1 mg to about 2.0 mg, such as at a dose of about 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, or 2.0 mg, for instance, by oral administration. In some embodiments, the conjugated estrogen is administered to the patient at a dose of 0.625 mg, for instance, by oral administration. In some embodiments, the conjugated estrogen is administered to the patient at a dose of 0.45 mg, for instance, by oral administration. In some embodiments, the conjugated estrogen is administered to the patient at a dose of 0.3 mg, for instance, by oral administration.

The conjugated estrogen may be administered to the patient one or more times per day, week, or month. The conjugated estrogen may be administered to the patient, for example, in an amount of from about 0.1 mg/day to about 2.0 mg/day, such as in an amount of about 0.1 mg/day, 0.2 mg/day, 0.3 mg/day, 0.4 mg/day, 0.5 mg/day, 0.6 mg/day, 0.7 mg/day, 0.8 mg/day, 0.9 mg/day, 1.0 mg/day, 1.1 mg/day, 1.2 mg/day, 1.3 mg/day, 1.4 mg/day, 1.5 mg/day, 1.6 mg/day, 1.7 mg/day, 1.8 mg/day, 1.9 mg/day, or 2.0 mg/day, for instance, by oral administration. In some embodiments, the conjugated estrogen is administered to the patient in an amount of 0.625 mg/day, for instance, by oral administration. In some embodiments, the conjugated estrogen is administered to the patient in an amount of 0.45 mg/day, for instance, by oral administration. In some embodiments, the conjugated estrogen is administered to the patient in an amount of 0.3 mg/day, for instance, by oral administration.

In some embodiments, the add-back therapy includes a progestin. In some embodiments, the progestin is selected from the group consisting of norethindrone or an ester thereof, such as norethindrone acetate, or another agent such as progesterone, norgestimate, medroxyprogesterone, or drospirenone.

In some embodiments, the progestin is norethindrone or a compound that is metabolized in vivo to produce norethindrone, such as an ester of norethindrone that is de-esterified in vivo to produce norethindrone, for instance, norethindrone acetate.

In some embodiments, the progestin is norethindrone. The norethindrone may be administered to the patient, for example, at a dose of from about 0.05 mg to about 5.0 mg, such as at a dose of about 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, 2.5 mg, 2.6 mg, 2.7 mg, 2.8 mg, 2.9 mg, 3.0 mg, 3.1 mg, 3.2 mg, 3.3 mg, 3.4 mg, 3.5 mg, 3.6 mg, 3.7 mg, 3.8 mg, 3.9 mg, 4.0 mg, 4.1 mg, 4.2 mg, 4.3 mg, 4.4 mg, 4.5 mg, 4.6 mg, 4.7 mg, 4.8 mg, 4.9 mg, or 5.0 mg, for instance, by oral administration. In some embodiments, the norethindrone is administered to the patient at a dose of 1.0 mg, for instance, by oral administration. In some embodiments, the norethindrone is administered to the patient at a dose of 0.5 mg, for instance, by oral administration. In some embodiments, the norethindrone is administered to the patient at a dose of 0.1 mg, for instance, by oral administration.

The norethindrone may be administered to the patient one or more times per day, week, or month. The norethindrone may be administered to the patient, for example, in an amount of from about 0.05 mg/day to about 5.0 mg/day, such as in an amount of about 0.05 mg/day, 0.06 mg/day, 0.07 mg/day, 0.08 mg/day, 0.09 mg/day, 0.1 mg/day, 0.2 mg/day, 0.3 mg/day, 0.4 mg/day, 0.5 mg/day, 0.6 mg/day, 0.7 mg/day, 0.8 mg/day, 0.9 mg/day, 1.0 mg/day, 1.1 mg/day, 1.2 mg/day, 1.3 mg/day, 1.4 mg/day, 1.5 mg/day, 1.6 mg/day, 1.7 mg/day, 1.8 mg/day, 1.9 mg/day, 2.0 mg/day, 2.1 mg/day, 2.2 mg/day, 2.3 mg/day, 2.4 mg/day, 2.5 mg/day, 2.6 mg/day, 2.7 mg/day, 2.8 mg/day, 2.9 mg/day, 3.0 mg/day, 3.1 mg/day, 3.2 mg/day, 3.3 mg/day, 3.4 mg/day, 3.5 mg/day, 3.6 mg/day, 3.7 mg/day, 3.8 mg/day, 3.9 mg/day, 4.0 mg/day, 4.1 mg/day, 4.2 mg/day, 4.3 mg/day, 4.4 mg/day, 4.5 mg/day, 4.6 mg/day, 4.7 mg/day, 4.8 mg/day, 4.9 mg/day, or 5.0 mg/day, for instance, by oral administration. In some embodiments, the norethindrone is administered to the patient in an amount of 1.0 mg/day, for instance, by oral administration. In some embodiments, the norethindrone is administered to the patient in an amount of 0.5 mg/day, for instance, by oral administration. In some embodiments, the norethindrone is administered to the patient in an amount of 0.1 mg/day, for instance, by oral administration.

In some embodiments, the progestin is norethindrone acetate. The norethindrone acetate may be administered to the patient, for example, at a dose of from about 0.05 mg to about 5.0 mg, such as at a dose of about 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, 2.5 mg, 2.6 mg, 2.7 mg, 2.8 mg, 2.9 mg, 3.0 mg, 3.1 mg, 3.2 mg, 3.3 mg, 3.4 mg, 3.5 mg, 3.6 mg, 3.7 mg, 3.8 mg, 3.9 mg, 4.0 mg, 4.1 mg, 4.2 mg, 4.3 mg, 4.4 mg, 4.5 mg, 4.6 mg, 4.7 mg, 4.8 mg, 4.9 mg, or 5.0 mg, for instance, by oral administration. In some embodiments, the norethindrone acetate is administered to the patient at a dose of 1.0 mg, for instance, by oral administration. In some embodiments, the norethindrone acetate is administered to the patient at a dose of 0.5 mg, for instance, by oral administration. In some embodiments, the norethindrone acetate is administered to the patient at a dose of 0.1 mg, for instance, by oral administration.

The norethindrone acetate may be administered to the patient one or more times per day, week, or month. The norethindrone acetate may be administered to the patient, for example, in an amount of from about 0.05 mg/day to about 5.0 mg/day, such as in an amount of about 0.05 mg/day, 0.06 mg/day, 0.07 mg/day, 0.08 mg/day, 0.09 mg/day, 0.1 mg/day, 0.2 mg/day, 0.3 mg/day, 0.4 mg/day, 0.5 mg/day, 0.6 mg/day, 0.7 mg/day, 0.8 mg/day, 0.9 mg/day, 1.0 mg/day, 1.1 mg/day, 1.2 mg/day, 1.3 mg/day, 1.4 mg/day, 1.5 mg/day, 1.6 mg/day, 1.7 mg/day, 1.8 mg/day, 1.9 mg/day, 2.0 mg/day, 2.1 mg/day, 2.2 mg/day, 2.3 mg/day, 2.4 mg/day, 2.5 mg/day, 2.6 mg/day, 2.7 mg/day, 2.8 mg/day, 2.9 mg/day, 3.0 mg/day, 3.1 mg/day, 3.2 mg/day, 3.3 mg/day, 3.4 mg/day, 3.5 mg/day, 3.6 mg/day, 3.7 mg/day, 3.8 mg/day, 3.9 mg/day, 4.0 mg/day, 4.1 mg/day, 4.2 mg/day, 4.3 mg/day, 4.4 mg/day, 4.5 mg/day, 4.6 mg/day, 4.7 mg/day, 4.8 mg/day, 4.9 mg/day, or 5.0 mg/day, for instance, by oral administration. In some embodiments, the norethindrone acetate is administered to the patient in an amount of 1.0 mg/day, for instance, by oral administration. In some embodiments, the norethindrone acetate is administered to the patient in an amount of 0.5 mg/day, for instance, by oral administration. In some embodiments, the norethindrone acetate is administered to the patient in an amount of 0.1 mg/day, for instance, by oral administration.

In some embodiments, the progestin is progesterone. The progesterone may be administered to the patient, for example, at a dose of from about 50 mg to about 250 mg, such as a dose of about 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, or 250 mg, for instance, by oral administration. In some embodiments, the progesterone is administered to the patient at a dose of 200 mg, for instance, by oral administration. In some embodiments, the progesterone is administered to the patient at a dose of 100 mg, for instance, by oral administration.

The progesterone may be administered to the patient one or more times per day, week, or month. The progesterone may be administered to the patient, for example, in an amount of from about 50 mg/day to about 250 mg/day, such as a dose of about 50 mg/day, 55 mg/day, 60 mg/day, 65 mg/day, 70 mg/day, 75 mg/day, 80 mg/day, 85 mg/day, 90 mg/day, 95 mg/day, 100 mg/day, 105 mg/day, 110 mg/day, 115 mg/day, 120 mg/day, 125 mg/day, 130 mg/day, 135 mg/day, 140 mg/day, 145 mg/day, 150 mg/day, 155 mg/day, 160 mg/day, 165 mg/day, 170 mg/day, 175 mg/day, 180 mg/day, 185 mg/day, 190 mg/day, 195 mg/day, 200 mg/day, 205 mg/day, 210 mg/day, 215 mg/day, 220 mg/day, 225 mg/day, 230 mg/day, 235 mg/day, 240 mg/day, 245 mg/day, or 250 mg/day, for instance, by oral administration. In some embodiments, the progesterone is administered to the patient in an amount of 200 mg/day, for instance, by oral administration. In some embodiments, the progesterone is administered to the patient in an amount of 100 mg/day, for instance, by oral administration.

In some embodiments, the progestin is norgestimate. The norgestimate may be administered to the patient, for example, at a dose of from about 0.01 mg to about 2.0 mg, such as at a dose of about 0.01 mg, 0.02 mg, 0.03 mg, 0.04 mg, 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, or 2.0 mg, for instance, by oral administration. In some embodiments, the norgestimate is administered to the patient at a dose of 0.09 mg, for instance, by oral administration.

The norgestimate may be administered to the patient one or more times per day, week, or month. The norgestimate may be administered to the patient, for example, in an amount of from about 0.01 mg/day to about 2.0 mg/day, such as in an amount of about 0.01 mg/day, 0.02 mg/day, 0.03 mg/day, 0.04 mg/day, 0.05 mg/day, 0.06 mg/day, 0.07 mg/day, 0.08 mg/day, 0.09 mg/day, 0.1 mg/day, 0.2 mg/day, 0.3 mg/day, 0.4 mg/day, 0.5 mg/day, 0.6 mg/day, 0.7 mg/day, 0.8 mg/day, 0.9 mg/day, 1.0 mg/day, 1.1 mg/day, 1.2 mg/day, 1.3 mg/day, 1.4 mg/day, 1.5 mg/day, 1.6 mg/day, 1.7 mg/day, 1.8 mg/day, 1.9 mg/day, or 2.0 mg/day, for instance, by oral administration. In some embodiments, the norgestimate is administered to the patient in an amount of 0.09 mg/day, for instance, by oral administration.

In some embodiments, the progestin is medroxyprogesterone. The medroxyprogesterone may be administered to the patient, for example, at a dose of from about 0.5 mg to about 10.0 mg, such as at a dose of about 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2.0 mg, 2.1 mg, 2.2 mg, 2.3 mg, 2.4 mg, 2.5 mg, 2.6 mg, 2.7 mg, 2.8 mg, 2.9 mg, 3.0 mg, 3.1 mg, 3.2 mg, 3.3 mg, 3.4 mg, 3.5 mg, 3.6 mg, 3.7 mg, 3.8 mg, 3.9 mg, 4.0 mg, 4.1 mg, 4.2 mg, 4.3 mg, 4.4 mg, 4.5 mg, 4.6 mg, 4.7 mg, 4.8 mg, 4.9 mg, 5.0 mg, 5.1 mg, 5.2 mg, 5.3 mg, 5.4 mg, 5.5 mg, 5.6 mg, 5.7 mg, 5.8 mg, 5.9 mg, 6.0 mg, 6.1 mg, 6.2 mg, 6.3 mg, 6.4 mg, 6.5 mg, 6.6 mg, 6.7 mg, 6.8 mg, 6.9 mg, 7.0 mg, 7.1 mg, 7.2 mg, 7.3 mg, 7.4 mg, 7.5 mg, 7.6 mg, 7.7 mg, 7.8 mg, 7.9 mg, 8.0 mg, 8.1 mg, 8.2 mg, 8.3 mg, 8.4 mg, 8.5 mg, 8.6 mg, 8.7 mg, 8.8 mg, 8.9 mg, 9.0 mg, 9.1 mg, 9.2 mg, 9.3 mg, 9.4 mg, 9.5 mg, 9.6 mg, 9.7 mg, 9.8 mg, 9.9 mg, or 10.0 mg, for instance, by oral administration. In some embodiments, the medroxyprogesterone is administered to the patient at a dose of 5.0 mg, for instance, by oral administration. In some embodiments, the medroxyprogesterone is administered to the patient at a dose of 2.5 mg, for instance, by oral administration. In some embodiments, the medroxyprogesterone is administered to the patient at a dose of 1.5 mg, for instance, by oral administration.

The medroxyprogesterone may be administered to the patient one or more times per day, week, or month. The medroxyprogesterone may be administered to the patient, for example, in an amount of from about 0.5 mg/day to about 10.0 mg/day, such as in an amount of about 0.5 mg/day, 0.6 mg/day, 0.7 mg/day, 0.8 mg/day, 0.9 mg/day, 1.0 mg/day, 1.1 mg/day, 1.2 mg/day, 1.3 mg/day, 1.4 mg/day, 1.5 mg/day, 1.6 mg/day, 1.7 mg/day, 1.8 mg/day, 1.9 mg/day, 2.0 mg/day, 2.1 mg/day, 2.2 mg/day, 2.3 mg/day, 2.4 mg/day, 2.5 mg/day, 2.6 mg/day, 2.7 mg/day, 2.8 mg/day, 2.9 mg/day, 3.0 mg/day, 3.1 mg/day, 3.2 mg/day, 3.3 mg/day, 3.4 mg/day, 3.5 mg/day, 3.6 mg/day, 3.7 mg/day, 3.8 mg/day, 3.9 mg/day, 4.0 mg/day, 4.1 mg/day, 4.2 mg/day, 4.3 mg/day, 4.4 mg/day, 4.5 mg/day, 4.6 mg/day, 4.7 mg/day, 4.8 mg/day, 4.9 mg/day, 5.0 mg/day, 5.1 mg/day, 5.2 mg/day, 5.3 mg/day, 5.4 mg/day, 5.5 mg/day, 5.6 mg/day, 5.7 mg/day, 5.8 mg/day, 5.9 mg/day, 6.0 mg/day, 6.1 mg/day, 6.2 mg/day, 6.3 mg/day, 6.4 mg/day, 6.5 mg/day, 6.6 mg/day, 6.7 mg/day, 6.8 mg/day, 6.9 mg/day, 7.0 mg/day, 7.1 mg/day, 7.2 mg/day, 7.3 mg/day, 7.4 mg/day, 7.5 mg/day, 7.6 mg/day, 7.7 mg/day, 7.8 mg/day, 7.9 mg/day, 8.0 mg/day, 8.1 mg/day, 8.2 mg/day, 8.3 mg/day, 8.4 mg/day, 8.5 mg/day, 8.6 mg/day, 8.7 mg/day, 8.8 mg/day, 8.9 mg/day, 9.0 mg/day, 9.1 mg/day, 9.2 mg/day, 9.3 mg/day, 9.4 mg/day, 9.5 mg/day, 9.6 mg/day, 9.7 mg/day, 9.8 mg/day, 9.9 mg/day, or 10.0 mg/day, for instance, by oral administration. In some embodiments, the medroxyprogesterone is administered to the patient in an amount of 5.0 mg/day, for instance, by oral administration. In some embodiments, the medroxyprogesterone is administered to the patient in an amount of 2.5 mg/day, for instance, by oral administration. In some embodiments, the medroxyprogesterone is administered to the patient in an amount of 1.5 mg/day, for instance, by oral administration.

In some embodiments, the progestin is drospirenone. The drospirenone may be administered to the patient, for example, at a dose of from about 0.1 mg to about 1.0 mg, such as at a dose of about 0.1 mg, 0.2 mg, 0.3 mg, 0.4 mg, 0.5 mg, 0.6 mg, 0.7 mg, 0.8 mg, 0.9 mg, or 1.0 mg, for instance, by oral administration. In some embodiments, the drospirenone is administered to the patient at a dose of 0.5 mg, for instance, by oral administration. In some embodiments, the drospirenone is administered to the patient at a dose of 0.25 mg, for instance, by oral administration.

The drospirenone may be administered to the patient one or more times per day, week, or month. The drospirenone may be administered to the patient, for example, in an amount of from about 0.1 mg/day to about 1.0 mg/day, such as in an amount of about 0.1 mg/day, 0.2 mg/day, 0.3 mg/day, 0.4 mg/day, 0.5 mg/day, 0.6 mg/day, 0.7 mg/day, 0.8 mg/day, 0.9 mg/day, or 1.0 mg/day, for instance, by oral administration. In some embodiments, the drospirenone is administered to the patient in an amount of 0.5 mg/day, for instance, by oral administration. In some embodiments, the drospirenone is administered to the patient in an amount of 0.25 mg/day, for instance, by oral administration.

In some embodiments, the add-back therapy includes an estrogen and a progestin. In some embodiments, the add-back therapy includes β17-estradiol and norethindrone (or a compound that is metabolized in vivo to produce norethindrone, such as an ester of norethindrone that is de-esterified in vivo to produce norethindrone, for instance, norethindrone acetate).

In some embodiments, the add-back therapy includes from about 0.75 mg to about 1.25 mg of β17-estradiol, e.g., administered orally, and from about 0.25 mg to about 0.75 mg of norethindrone (or a compound that is metabolized in vivo to produce norethindrone, such as an ester of norethindrone that is de-esterified in vivo to produce norethindrone, for instance, norethindrone acetate), e.g., administered orally. In some embodiments, the add-back therapy includes 1.0 mg of β17-estradiol, e.g., administered orally, and 0.5 mg of norethindrone (or a compound that is metabolized in vivo to produce norethindrone, such as an ester of norethindrone that is de-esterified in vivo to produce norethindrone, for instance, norethindrone acetate), e.g., administered orally. In some embodiments, the add-back therapy includes 1.0 mg of β17-estradiol, e.g., administered orally, and, in the same pharmaceutical composition, 0.5 mg of norethindrone (or a compound that is metabolized in vivo to produce norethindrone, such as an ester of norethindrone that is de-esterified in vivo to produce norethindrone, for instance, norethindrone acetate), e.g., administered orally. In some embodiments, the add-back therapy includes 1.0 mg of β17-estradiol, e.g., administered orally, and, in a separate pharmaceutical composition, 0.5 mg of norethindrone (or a compound that is metabolized in vivo to produce norethindrone, such as an ester of norethindrone that is de-esterified in vivo to produce norethindrone, for instance, norethindrone acetate), e.g., administered orally.

In some embodiments, the GnRH antagonist is administered to the patient in a fixed-dose composition that contains the GnRH antagonist (e.g., in an amount of about 100 mg or about 200 mg of the compound of any one of formulas (I)-(VI) (e.g., in the recited amount or in an equivalent amount of a pharmaceutically acceptable salt, such as a choline salt)), from about 0.75 mg to about 1.25 mg of β17-estradiol, and from about 0.25 mg to about 0.75 mg of norethindrone (or a compound that is metabolized in vivo to produce norethindrone, such as an ester of norethindrone that is de-esterified in vivo to produce norethindrone, for instance, norethindrone acetate). In some embodiments, the GnRH antagonist is administered to the patient in a fixed-dose composition that contains the GnRH antagonist (e.g., in an amount of about 100 mg or about 200 mg of the compound of any one of formulas (I)-(VI) (e.g., in the recited amount or in an equivalent amount of a pharmaceutically acceptable salt, such as a choline salt)), about 1.0 mg of β17-estradiol (e.g., 1.0 mg of β17-estradiol), and about 0.5 mg of norethindrone (or a compound that is metabolized in vivo to produce norethindrone, such as an ester of norethindrone that is de-esterified in vivo to produce norethindrone, for instance, norethindrone acetate) (e.g., 0.5 mg of norethindrone or a compound that is metabolized in vivo to produce norethindrone, such as an ester of norethindrone that is de-esterified in vivo to produce norethindrone, for instance, norethindrone acetate).

In some embodiments, the GnRH antagonist is administered to the patient in a fixed-dose composition that contains the GnRH antagonist (e.g., in an amount of about 100 mg or about 200 mg of the compound of any one of formulas (I)-(VI) (e.g., in the recited amount or in an equivalent amount of a pharmaceutically acceptable salt, such as a choline salt)), 1.0 mg of β17-estradiol, and 0.5 mg of norethindrone acetate.

In some embodiments, the above fixed-dose composition is administered to the patient in one or more doses per 12 hours, 24 hours, 48 hours, 72 hours, week, month, or year, such as in from 1 to 10 doses per 12 hours (e.g., 1 dose every 12 hours, 2 doses every 12 hours, 3 doses every 12 hours, 4 doses every 12 hours, 5 doses every 12 hours, 6 doses every 12 hours, 7 doses every 12 hours, 8 doses every 12 hours, 9 doses every 12 hours, or 10 doses every 12 hours), from 1 to 10 doses per 24 hours (e.g., 1 dose every 24 hours, 2 doses every 24 hours, 3 doses every 24 hours, 4 doses every 24 hours, 5 doses every 24 hours, 6 doses every 24 hours, 7 doses every 24 hours, 8 doses every 24 hours, 9 doses every 24 hours, or 10 doses every 24 hours), from 1 to 10 doses per 48 hours (e.g., 1 dose every 48 hours, 2 doses every 48 hours, 3 doses every 48 hours, 4 doses every 48 hours, 5 doses every 48 hours, 6 doses every 48 hours, 7 doses every 48 hours, 8 doses every 48 hours, 9 doses every 48 hours, or 10 doses every 48 hours), from 1 to 10 doses per 72 hours (e.g., 1 dose every 72 hours, 2 doses every 72 hours, 3 doses every 72 hours, 4 doses every 72 hours, 5 doses every 72 hours, 6 doses every 72 hours, 7 doses every 72 hours, 8 doses every 72 hours, 9 doses every 72 hours, or 10 doses every 72 hours), from 1 to 10 doses per week (e.g., 1 dose every week, 2 doses every week, 3 doses every week, 4 doses every week, 5 doses every week, 6 doses every week, 7 doses every week, 8 doses every week, 9 doses every week, or 10 doses every week), or from 1 to 60 doses per month (e.g., from 30-60 doses per month, such as 1 time daily, 2 times daily, 3 times daily, 4 times daily, 5 times daily, 6 times daily, 7 times daily, 8 times daily, 9 times daily, 10 times daily, 7 times weekly, 8 times weekly, 9 times weekly, 10 times weekly, 11 times weekly, 12 times weekly, 13 times weekly, 14 times weekly, or more), among others. In some embodiments, the above fixed-dose composition is administered to the patient once daily.

In some embodiments, the add-back therapy includes from about 0.25 mg to about 0.75 mg of β17-estradiol, e.g., administered orally, and from about 0.05 mg to about 0.2 mg of norethindrone (or a compound that is metabolized in vivo to produce norethindrone, such as an ester of norethindrone that is de-esterified in vivo to produce norethindrone, for instance, norethindrone acetate), e.g., administered orally. In some embodiments, the add-back therapy includes 0.5 mg of β17-estradiol, e.g., administered orally, and 0.1 mg of norethindrone (or a compound that is metabolized in vivo to produce norethindrone, such as an ester of norethindrone that is de-esterified in vivo to produce norethindrone, for instance, norethindrone acetate), e.g., administered orally. In some embodiments, the add-back therapy includes 0.5 mg of β17-estradiol, e.g., administered orally, and, in the same pharmaceutical composition, 0.1 mg of norethindrone (or a compound that is metabolized in vivo to produce norethindrone, such as an ester of norethindrone that is de-esterified in vivo to produce norethindrone, for instance, norethindrone acetate), e.g., administered orally. In some embodiments, the add-back therapy includes 0.5 mg of β17-estradiol, e.g., administered orally, and, in a separate pharmaceutical composition, 0.1 mg of norethindrone (or a compound that is metabolized in vivo to produce norethindrone, such as an ester of norethindrone that is de-esterified in vivo to produce norethindrone, for instance, norethindrone acetate), e.g., administered orally.

In some embodiments, the GnRH antagonist is administered to the patient in a fixed-dose composition that contains the GnRH antagonist (e.g., in an amount of about 100 mg or about 200 mg of the compound of any one of formulas (I)-(VI) (e.g., in the recited amount or in an equivalent amount of a pharmaceutically acceptable salt, such as a choline salt)), from about 0.25 mg to about 0.75 mg of β17-estradiol, and from about 0.05 mg to about 0.2 mg of norethindrone (or a compound that is metabolized in vivo to produce norethindrone, such as an ester of norethindrone that is de-esterified in vivo to produce norethindrone, for instance, norethindrone acetate). In some embodiments, the GnRH antagonist is administered to the patient in a fixed-dose composition that contains the GnRH antagonist (e.g., in an amount of about 100 mg or about 200 mg of the compound of any one of formulas (I)-(VI) (e.g., in the recited amount or in an equivalent amount of a pharmaceutically acceptable salt, such as a choline salt)), about 0.5 mg of β17-estradiol (e.g., 0.5 mg of β17-estradiol), and about 0.1 mg of norethindrone (or a compound that is metabolized in vivo to produce norethindrone, such as an ester of norethindrone that is de-esterified in vivo to produce norethindrone, for instance, norethindrone acetate) (e.g., 0.1 mg of norethindrone or a compound that is metabolized in vivo to produce norethindrone, such as an ester of norethindrone that is de-esterified in vivo to produce norethindrone, for instance, norethindrone acetate).

In some embodiments, the GnRH antagonist is administered to the patient in a fixed-dose composition that contains the GnRH antagonist (e.g., in an amount of about 100 mg or about 200 mg of the compound of any one of formulas (I)-(VI) (e.g., in the recited amount or in an equivalent amount of a pharmaceutically acceptable salt, such as a choline salt)), 0.5 mg of β17-estradiol, and 0.1 mg of norethindrone acetate.

In some embodiments, the above fixed-dose composition is administered to the patient in one or more doses per 12 hours, 24 hours, 48 hours, 72 hours, week, month, or year, such as in from 1 to 10 doses per 12 hours (e.g., 1 dose every 12 hours, 2 doses every 12 hours, 3 doses every 12 hours, 4 doses every 12 hours, 5 doses every 12 hours, 6 doses every 12 hours, 7 doses every 12 hours, 8 doses every 12 hours, 9 doses every 12 hours, or 10 doses every 12 hours), from 1 to 10 doses per 24 hours (e.g., 1 dose every 24 hours, 2 doses every 24 hours, 3 doses every 24 hours, 4 doses every 24 hours, 5 doses every 24 hours, 6 doses every 24 hours, 7 doses every 24 hours, 8 doses every 24 hours, 9 doses every 24 hours, or 10 doses every 24 hours), from 1 to 10 doses per 48 hours (e.g., 1 dose every 48 hours, 2 doses every 48 hours, 3 doses every 48 hours, 4 doses every 48 hours, 5 doses every 48 hours, 6 doses every 48 hours, 7 doses every 48 hours, 8 doses every 48 hours, 9 doses every 48 hours, or 10 doses every 48 hours), from 1 to 10 doses per 72 hours (e.g., 1 dose every 72 hours, 2 doses every 72 hours, 3 doses every 72 hours, 4 doses every 72 hours, 5 doses every 72 hours, 6 doses every 72 hours, 7 doses every 72 hours, 8 doses every 72 hours, 9 doses every 72 hours, or 10 doses every 72 hours), from 1 to 10 doses per week (e.g., 1 dose every week, 2 doses every week, 3 doses every week, 4 doses every week, 5 doses every week, 6 doses every week, 7 doses every week, 8 doses every week, 9 doses every week, or 10 doses every week), from 1 to 60 doses per month (e.g., from 30-60 doses per month, such as 1 time daily, 2 times daily, 3 times daily, 4 times daily, 5 times daily, 6 times daily, 7 times daily, 8 times daily, 9 times daily, 10 times daily, 7 times weekly, 8 times weekly, 9 times weekly, 10 times weekly, 11 times weekly, 12 times weekly, 13 times weekly, 14 times weekly, or more), among others. In some embodiments, the fixed-dose composition is administered to the patient once daily.

In some embodiments of any of the foregoing aspects of the disclosure, the patient is a pre-menopausal female of from about 18 to about 48 years of age, such as a patient of 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 years of age.

In some embodiments, the patient exhibits a serum concentration of FSH of about 20 IU/L or less prior to administration of the GnRH antagonist to the patient, such as a serum concentration of FSH of from about 5 IU/L to about 20 IU/L (e.g., a serum concentration of FSH of about 5 IU/L, 6 IU/L, 7 IU/L, 8 IU/L, 9 IU/L, 10 IU/L, 11 IU/L, 12 IU/L, 13 IU/L, 14 IU/L, 15 IU/L, 16 IU/L, 17 IU/L, 18 IU/L, 19 IU/L, or 20 IU/L.

In some embodiments, the patient exhibits a rectal (type II) and/or vaginal (type III) endometriosis node of at least 2 cm prior to administration of the GnRH antagonist to the patient. The length of the type II and/or type III endometriosis node may be assessed, for example, by way of magnetic resonance imaging (MRI).

In some embodiments, the patient exhibits a junctional-zone width of about 12 mm or more prior to administration of the GnRH antagonist to the patient, such as a junctional zone width of from about 12 mm to about 20 mm, or more (e.g., a junctional zone width of from about 12 mm to about 20 mm, from about 12 mm to about 19 mm, from about 12 mm to about 18 mm, from about 12 mm to about 17 mm, from about 12 mm to about 16 mm, from about 12 mm to about 15 mm, from about 12 mm to about 14 mm, or more) prior to administration of the GnRH antagonist to the patient. The junctional zone width may be assessed, for example, by way of MRI.

In some embodiments, the patient exhibits a reduction in serum concentration of FSH, LH, and/or E2 following administration of the GnRH antagonist to the patient. The reduction in serum concentration of LH, FSH, and/or E2 may be effectuated within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient (e.g., within from about 12 weeks to about 24 weeks of the first administration of the GnRH antagonist to the patient, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of the first administration of the GnRH antagonist to the patient).

In some embodiments, the patient exhibits a reduction in uterine bleeding following administration of the GnRH antagonist to the patient. The reduction in dyspareunia may be effectuated within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient (e.g., within from about 12 weeks to about 24 weeks of the first administration of the GnRH antagonist to the patient, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of the first administration of the GnRH antagonist to the patient). The reduction in uterine bleeding may be assessed by way of an alkaline hematin method, for example, as described herein.

In some embodiments, the patient exhibits amenorrhea following administration of the GnRH antagonist to the patient. The amenorrhea may be effectuated within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient (e.g., within from about 12 weeks to about 24 weeks of the first administration of the GnRH antagonist to the patient, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of the first administration of the GnRH antagonist to the patient).

In some embodiments, the patient exhibits a reduction in the volume of one or more rectovaginal endometriosis nodes following administration of the GnRH antagonist to the patient. The reduction in the volume of the one or more rectovaginal endometriosis nodes may be effectuated within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient (e.g., within from about 12 weeks to about 24 weeks of the first administration of the GnRH antagonist to the patient, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of the first administration of the GnRH antagonist to the patient). The reduction in the volume of the one or more rectovaginal endometriosis nodes may be assessed, for example, by way of MRI and/or TVUS.

In some embodiments, the patient exhibits a reduction in bowel involvement of one or more type III endometriosis nodes following administration of the GnRH antagonist to the patient. The reduction in bowel involvement of one or more type III endometiosis nodes may be effectuated within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient (e.g., within from about 12 weeks to about 24 weeks of the first administration of the GnRH antagonist to the patient, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of the first administration of the GnRH antagonist to the patient). The reduction in bowel involvement of one or more type IlIl endometriosis nodes may be assessed, for example, by way of MRI.

In some embodiments, the patient exhibits a reduction in pelvic pain following administration of the GnRH antagonist to the patient. The reduction in pelvic pain may be effectuated within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient (e.g., within from about 12 weeks to about 24 weeks of the first administration of the GnRH antagonist to the patient, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of the first administration of the GnRH antagonist to the patient). The reduction in pelvic pain may be assessed by way of a modified Biberoglu & Behrman (mB&B) score, Numerical Rating Scale (NRS) score, or Verbal Rating Scale (VRS) score.

In some embodiments, the patient exhibits a reduction in dysmenorrhea following administration of the GnRH antagonist to the patient. The reduction in dysmenorrhea may be effectuated within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient (e.g., within from about 12 weeks to about 24 weeks of the first administration of the GnRH antagonist to the patient, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of the first administration of the GnRH antagonist to the patient). The reduction in dysmenorrhea may be assessed by way of an mB&B score, NRS score, or VRS score.

In some embodiments, the patient exhibits a reduction in dyspareunia following administration of the GnRH antagonist to the patient. The reduction in dyspareunia may be effectuated within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient (e.g., within from about 12 weeks to about 24 weeks of the first administration of the GnRH antagonist to the patient, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of the first administration of the GnRH antagonist to the patient). The reduction in dyspareunia may be assessed by way of an mB&B score, NRS score, or VRS score.

In some embodiments, the patient exhibits a reduction in dyschezia following administration of the GnRH antagonist to the patient. The reduction in dyschezia may be effectuated within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient (e.g., within from about 12 weeks to about 24 weeks of the first administration of the GnRH antagonist to the patient, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of the first administration of the GnRH antagonist to the patient). The reduction in dyschezia may be assessed by way of an mB&B score, NRS score, or VRS score.

In some embodiments, the patient exhibits a reduction in uterine volume following administration of the GnRH antagonist to the patient. The reduction in uterine volume may be effectuated within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient (e.g., within from about 12 weeks to about 24 weeks of the first administration of the GnRH antagonist to the patient, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of the first administration of the GnRH antagonist to the patient). The reduction in uterine volume may be assessed, for example, by way of MRI or transvaginal ultrasound (TVUS).

In some embodiments, the patient exhibits a reduction in the thickness of the anterior and/or posterior region of the uterine myometrium following administration of the GnRH antagonist to the patient. The reduction in the thickness of the anterior and/or posterior region of the uterine myometrium may be effectuated within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient (e.g., within from about 12 weeks to about 24 weeks of the first administration of the GnRH antagonist to the patient, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of the first administration of the GnRH antagonist to the patient).

In some embodiments, the patient exhibits a reduction in uterine tenderness following administration of the GnRH antagonist to the patient. The reduction in uterine tenderness may be effectuated within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient (e.g., within from about 12 weeks to about 24 weeks of the first administration of the GnRH antagonist to the patient, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of the first administration of the GnRH antagonist to the patient).

In some embodiments, the patient exhibits a reduction in the diameter of a junctional zone of adenomyosis following administration of the GnRH antagonist to the patient. The reduction in the diameter of a junctional zone of adenomyosis may be effectuated within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient (e.g., within from about 12 weeks to about 24 weeks of the first administration of the GnRH antagonist to the patient, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of the first administration of the GnRH antagonist to the patient).

In some embodiments, the patient exhibits an improvement in Endometriosis Health Profile questionnaire (EHP-30) score following administration of the GnRH antagonist to the patient. The improvement in the EHP-30 score may be effectuated within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient (e.g., within from about 12 weeks to about 24 weeks of the first administration of the GnRH antagonist to the patient, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of the first administration of the GnRH antagonist to the patient).

In some embodiments, the patient exhibits a positive Patient Global Impression of Change (PGIC) score following administration of the GnRH antagonist to the patient. The positive PGIC score may be effectuated within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient (e.g., within from about 12 weeks to about 24 weeks of the first administration of the GnRH antagonist to the patient, such as within about 12 weeks, 13 weeks, 14 weeks, 15 weeks, 16 weeks, 17 weeks, 18 weeks, 19 weeks, 20 weeks, 21 weeks, 22 weeks, 23 weeks, or 24 weeks of the first administration of the GnRH antagonist to the patient).

In some embodiments, the patient does not exhibit a reduction in bone mineral density (BMD) of greater than 5% following administration of the GnRH antagonist to the patient. In some embodiments, the patient does not exhibit a reduction in BMD of greater than 4% following administration of the GnRH antagonist to the patient. In some embodiments, the patient does not exhibit a reduction in BMD of greater than 3% following administration of the GnRH antagonist to the patient. In some embodiments, the patient does not exhibit a reduction in BMD of greater than 2% following administration of the GnRH antagonist to the patient. In some embodiments, the patient does not exhibit a reduction in BMD of greater than 1% following administration of the GnRH antagonist to the patient. BMBD may be assessed, for example, by dual energy X-ray absorptiometry, such as in the spine and/or femur of the patient. In some embodiments, the BMD is assessed by comparing the concentration of bone specific alkaline phosphatase (BAP) in a sample isolated from the patient following the administration to the concentration of BAP in a sample isolated from the patient prior to the administration. In some embodiments, the BMD is assessed by comparing the concentration of deoxypyridinoline (DPD) in a sample isolated from the patient following the administration to the concentration of DPD in a sample isolated from the patient prior to the administration. In some embodiments, the BMD is assessed by comparing the concentration of type I collagen C-terminal telopeptide (CTX) in a sample isolated from the patient following the administration to the concentration of CTX in a sample isolated from the patient prior to the administration. In some embodiments, the BMD is assessed by comparing the concentration of procollagen 1 N-terminal peptide (P1NP) in a sample isolated from the patient following the administration to the concentration of P1NP in a sample isolated from the patient prior to the administration.

In another aspect, the disclosure features a kit containing a GnRH antagonist, such as a GnRH antagonist of any of the above aspects or embodiments of the disclosure. The kit may further contain a package insert, such as a package insert instructing a user of the kit to administer the GnRH antagonist to a patient in accordance with the method of any one of the foregoing aspects or embodiments of the disclosure. In some embodiments, the GnRH antagonist contained within the kit is a compound represented by formula (I)

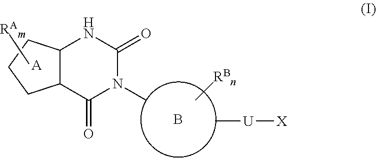

wherein ring A is a thiophene ring;

each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;

n is an integer from 0 to 2;

U is a single bond;

X is a group represented by —S-L-Y, —O-L-Y, —CO-L-Y, or —SO₂-L-Y, wherein L is an optionally substituted lower alkylene group;

Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and

Z is an optionally fused and optionally substituted cycloalkyl group, an optionally fused and optionally substituted heterocycloalkyl group, an optionally fused and optionally substituted aryl group, or an optionally fused and optionally substituted heteroaryl group;

or a pharmaceutically acceptable salt thereof.

In some embodiments of formula (I), the ring A is a thiophene ring represented by formula (IIa)

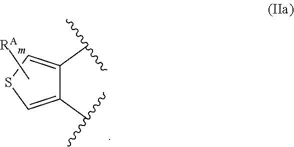

In some embodiments of formula (I) or (IIa), m is 1.

In some embodiments of formula (I) or (IIa), the ring A is an optionally substituted thiophene ring represented by formula (IIb)

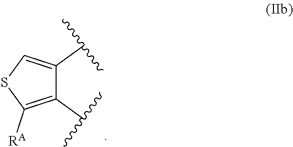

In some embodiments of formula (I), (IIa), or (IIb), each R^{A} is independently a halogen atom, an optionally substituted lower alkyl group, COOW¹, or CONW²W³, wherein W¹ to W³ independently are a hydrogen atom or an optionally substituted lower alkyl group, or W² and W³ may bind together with the neighboring nitrogen atom to form an optionally substituted cyclic amino group.

In some embodiments of formula (I), (IIa), or (IIb), each R^{A} is COOH or pharmaceutically acceptable salt thereof.

In some embodiments of formula (I), (IIa), or (IIb), the ring B is an optionally substituted benzene ring, pyridine ring, or thiophene ring.

In some embodiments of formula (I), (IIa), or (IIb), the ring B is represented by a formula selected from the group consisting of:

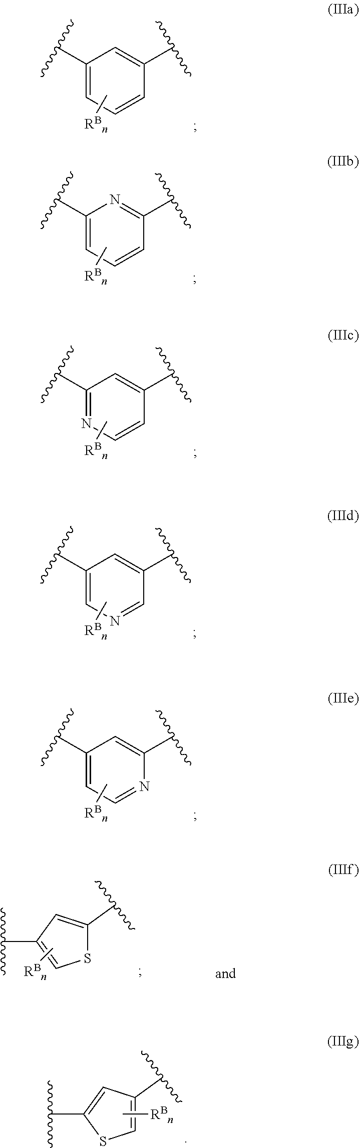

In some embodiments of formula (I), (IIa), (Ib), or any one of (IIIa)-(IIIg), n is 2.

In some embodiments of formula (I), (IIa), (IIb), or any one of (IIIa)-(IIIg), the ring B is represented by a formula selected from the group consisting of:

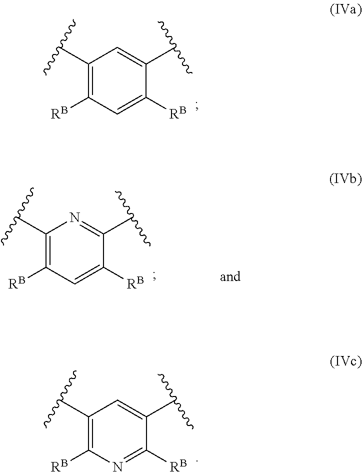

In some embodiments of formula (I), (IIa), (IIb), any one of (IIIa)-(IIIg), or any one of (IVa)-(IVc), each R^{B} is independently a halogen atom, an optionally substituted lower alkyl group, or OW⁴, wherein each W⁴ is independently a hydrogen atom or an optionally substituted lower alkyl group.

In some embodiments of formula (I), (IIa), (IIb), any one of (IIIa)-(IIIg), or any one of (IVa)-(IVc), each R^{B} is independently a fluorine atom, chlorine atom, bromine atom, methyl group, or methoxy group.

In some embodiments of formula (I), (IIa), (IIb), any one of (IIIa)-(IIIg), or any one of (IVa)-(IVc), U is a single bond.

In some embodiments of formula (I), (IIa), (IIb), any one of (IIIa)-(IIIg), or any one of (IVa)-(IVc), X is a group represented by —O-L-Y.

In some embodiments of formula (I), (IIa), (IIb), any one of (IIIa)-(IIIg), or any one of (IVa)-(IVc), L is a methylene group.

In some embodiments of formula (I), (IIa), (IIb), any one of (IIIa)-(IIIg), or any one of (IVa)-(IVc), Y is an optionally substituted benzene ring represented by formula (V)

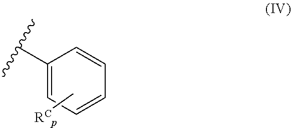

wherein each R^{C} is independently a halogen atom, an optionally substituted lower alkyl group, or OW⁹, wherein each W⁹ is independently a hydrogen atom or an optionally substituted lower alkyl group; and

p is an integer from 0 to 3.

In some embodiments of formula (I), (IIa), (IIb), any one of (IIIa)-(IIIg), any one of (IVa)-(IVc), or (V), Y is a substituted benzene ring represented by formula (Va)

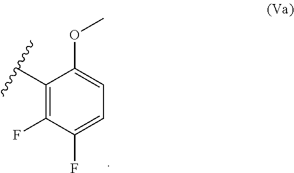 [SMILES: *c1c(OC)ccc(F)c1F]

In some embodiments, the compound is represented by formula (Ia)

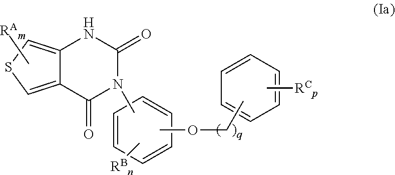

wherein each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;

m is an integer from 0 to 3;

each R^{B} is independently a halogen atom, a cyano group, an optionally substituted lower alkyl group, OW⁴, COW⁴, COOW⁴, or CONW⁵W⁶, wherein W⁴ to W⁸ independently are a hydrogen atom or an optionally substituted lower alkyl group, or W⁵ and W⁶ may bind together with the neighboring nitrogen atom to form an optionally substituted cyclic amino group;

n is an integer from 0 to 2;

q is an integer from 0 to 3;

each R^{C} is independently a halogen atom, an optionally substituted lower alkyl group, or OW⁹, wherein each W⁹ is independently a hydrogen atom or an optionally substituted lower alkyl group; and

p is an integer from 0 to 3;

or a pharmaceutically acceptable salt thereof.

In some embodiments, the compound is represented by formula (Ib)

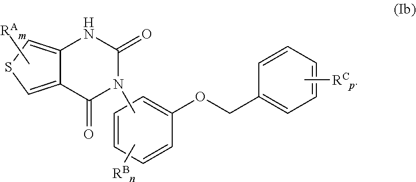

In some embodiments, the compound is represented by formula (Ic)

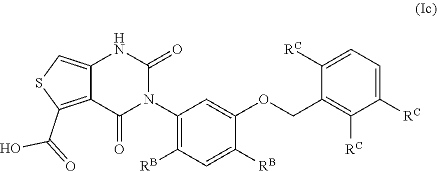

or a pharmaceutically acceptable salt thereof.

In some embodiments, the compound is 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid, represented by formula (VI)

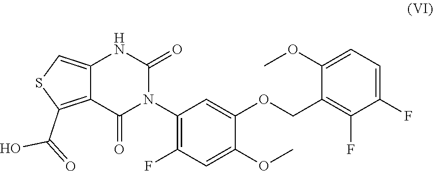 [SMILES: COc1cc(F)c(-n2c(=O)[nH]c3csc(C(=O)O)c3c2=O)cc1OCc1c(OC)ccc(F)c1F]

or a pharmaceutically acceptable salt thereof. In some embodiments, the compound 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid is administered to the patient in the form of an electrostatically neutral carboxylic acid. In some embodiments, the compound 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid is administered to the patient in the form of a pharmaceutically acceptable salt. In some embodiments, the compound 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid is administered to the patient in the form of the choline salt, choline 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate.

In some embodiments, the compound is the choline salt of the compound represented by formula (VI), choline 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate.

In some embodiments, the choline 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate is in a crystalline state.

In some embodiments, the choline 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate exhibits characteristic X-ray powder diffraction (XRPD) peaks at about 7.1° 2θ, about 11.5° 2θ, about 19.4° 2θ, about 21.5° 2θ, about 22.0° 2θ, about 22.6° 2θ, about 23.5° 2θ, and about 26.2° 2θ.

In some embodiments, the choline 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate exhibits ¹³C solid-state nuclear magnetic resonance (NMR) peaks centered at about 55.5 ppm, about 57.1 ppm, about 58.7 ppm, about 69.8 ppm, about 98.1 ppm, about 110.3 ppm, about 111.6 ppm, about 113.7 ppm, about 118.0 ppm, about 145.3 ppm, about 149.8 ppm, and about 155.8 ppm.

In some embodiments, the choline 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate exhibits ¹⁹F solid-state NMR peaks centered at about −151.8 ppm, −145.2 ppm, and −131.6 ppm.

In some embodiments, the GnRH antagonist contained within the kit is a compound represented by formula (VII)

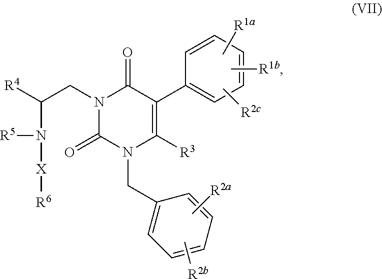

wherein R₁ₐ, R_{1b} and R_{1c} are the same or different and are each independently hydrogen, halogen, C₁₋₄alkyl, hydroxy or alkoxy, or R₁ₐ and R_{1b} taken together form —OCH₂O— or —OCH₂CH₂—;

R₂ₐ and R_{2b} are the same or different and are each independently hydrogen, halogen, trifluoromethyl, cyano or —SO₂CH₃;

R₃ is hydrogen or methyl;

R₄ is phenyl or C₃₋₇alkyl;

R₅ is hydrogen or C₁₋₄alkyl;

R⁶ is —COOH or an acid isostere; and

X is C₁₋₆alkanediyl optionally substituted with from 1 to 3 C₁₋₆alkyl groups;

or a pharmaceutically acceptable salt thereof.

In some embodiments, the GnRH antagonist is a compound represented by formula (VIII)

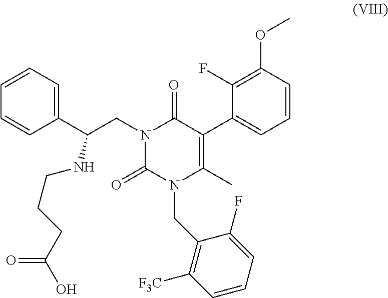 [SMILES: COc1cccc(-c2c(C)n(Cc3c(F)cccc3C(F)(F)F)c(=O)n(C[C@H](NCCCC(=O)O)c3ccccc3)c2=O)c1F]

or a pharmaceutically acceptable salt thereof. In some embodiments, the GnRH antagonist is the sodium salt of the compound represented by formula (VIII), which is represented by formula (IX), below.

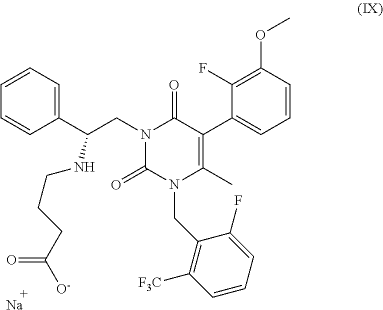

In some embodiments, the GnRH antagonist contained within the kit is a compound represented by formula (X)

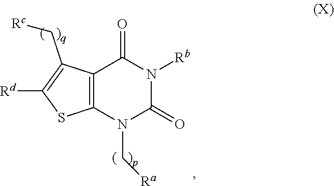

wherein R^{a} is a hydrogen atom, an optionally substituted aryl group (such as an aryl group that may have 1 to 5 substituents selected from halogen, nitro, cyano, amino, a carboxyl group that may be esterified or amidated, an alkylenedioxy, alkyl, alkoxy, alkylthio, alkylsulfinyl, and alkylsulfonyl), an optionally substituted cycloalkyl group, or an optionally substituted heterocyclic group;

R^{b} is an optionally substituted nitrogen-containing heterocyclic group;

R^{c} is an optionally substituted amino group;

R^{d} is an optionally substituted aryl group;

p is an integer from 0 to 3; and

q is an integer from 0 to 3;

or a pharmaceutically acceptable salt thereof.

In some embodiments, the GnRH antagonist is a compound represented by formula (XI)

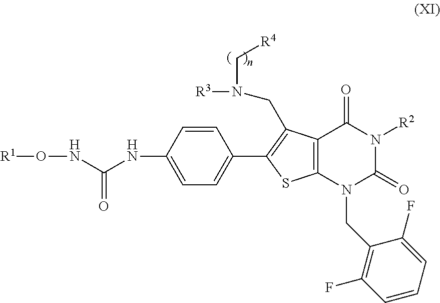

wherein R¹ is C₁₋₄alkyl;

R² is (1) a C₁₋₄alkyl which may have a substituent selected from the group consisting of (1′) a hydroxy group, (2′) a C₁₋₄alkoxy, (3′) a C₁₋₄alkoxy-carbonyl, (4′) a di-C₁-4alkyl-carbamoyl, (5′) a 5- to 7-membered nitrogen-containing heterocyclic group, (6′) a C₁₋₄alkyl-carbonyl and (7′) a halogen, (2) a C₃₋₈ cycloalkyl which may have (1′) a hydroxy group or (2′) a mono-C₁₋₄alkyl-carbonylamino, (3) a 5- to 7-membered nitrogen-containing heterocyclic group which may have a substituent selected from the group consisting of (1′) a halogen, (2′) a hydroxy group, (3′) a C₁₋₄alkyl and (4′) a C₁₋₄alkoxy, (4) a phenyl which may have a substituent selected from the group consisting of (1′) a halogen, (2′) a C₁₋₄alkoxy-C₁₋₄alkyl, (3′) a mono-C₁₋₄alkyl-carbamoyl-C₁₋₄alkyl, (4′) a C₁₋₄alkoxy and (5′) a mono-C₁₋₄alkylcarbamoyl-C₁₋₄alkoxy, or (5) a C₁₋₄alkoxy;

R³ is C₁₋₄alkyl;

R⁴ is (1) hydrogen, (2) C₁₋₄alkoxy, (3) C₆₋₁₀aryl, (4)N—C₁₋₄alkyl-N—C₁₋₄alkylsulfonylamino, (5) hydroxyl, or (6) a 5- to 7-membered nitrogen-containing heterocyclic group which may have a substituent selected from the group consisting of (1′) oxo, (2′) a C₁₋₄alkyl, (3′) a hydroxy-C₁₋₄alkyl, (4′) a C₁₋₄alkoxy-carbonyl, (5′) a mono-C₁₋₄alkyl-carbamoyl and (6′) a C₁₋₄alkylsulfonyl; and

n is an integer from 1 to 4;

optionally provided that when R² is a phenyl which may have a substituent, R⁴ is a 5- to 7-membered nitrogen-containing heterocyclic group which may have a substituent selected from the group consisting of (1) oxo, (2) hydroxy-C₁₋₄alkyl, (3) C₁₋₄alkoxy-carbonyl, (4) mono-C₁₋₄alkyl-carbamoyl and (5) C₁₋₄alkylsulfonyl;

or a pharmaceutically acceptable salt thereof. In some embodiments, the GnRH antagonist is a compound represented by formula (XII), below, or a pharmaceutically acceptable salt thereof, such as a chloride salt thereof.

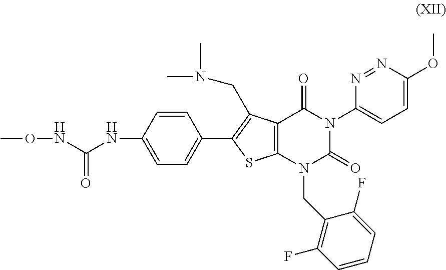

In some embodiments, the GnRH antagonist contained within the kit is a compound represented by formula (XIII)

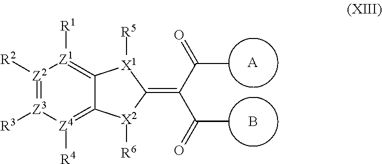

wherein R¹, R², R³ and R⁴ are the same or different, and are each independently selected from hydrogen, nitro, cyano, halogen, an optionally substituted hydrocarbon group, an optionally substituted heterocyclic group, hydroxy, alkoxy, carboxy, optionally substituted acyl-O—, optionally substituted acyl, a substituent —S(O)n₁₀₁₋ (wherein n₁₀₁ is an integer of 0 to 2), H—S(O)n₁₀₁₋, optionally substituted carbamoyl, optionally substituted sulfamoyl, optionally substituted amino, and two adjacent groups selected from the group of R¹, R², R³ and R⁴ may combine to form an aryl or a carbocyclic (e.g., cycloalkenyl) group;

R⁵ and R⁶ are the same or different and are each independently selected from hydrogen, halogen, optionally substituted hydrocarbon, and optionally substituted amino,

X¹ and X² are the same or different and are each independently selected from N, S and O;

A and B are the same or different and are each independently selected from optionally substituted aryl and optionally substituted heterocyclyl, and

Z¹, Z², Z³ and Z⁴ are each independently selected from C and N; optionally provided that 1) when X¹ and X² each is S or O, one or both of the corresponding R⁵ and R⁶ are absent; and/or 2) when one to four of Z¹, Z², Z³ and/or Z⁴ are N, the corresponding R¹, R², R³ and/or R⁴ are absent;

or a pharmaceutically acceptable salt thereof. In some embodiments, the GnRH antagonist is a compound represented by formula (XIV), below.

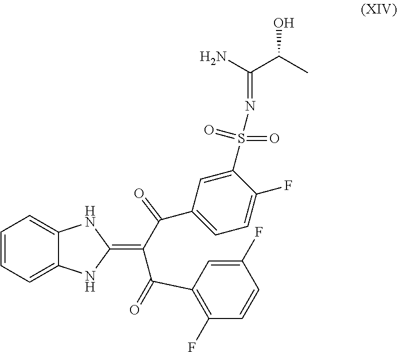 [SMILES: C[C@@H](O)/C(N)=N/S(=O)(=O)c1cc(C(=O)C(C(=O)c2cc(F)ccc2F)=C2Nc3ccccc3N2)ccc1F]

In some embodiments, the GnRH antagonist contained within the kit is SK12670 or BAY-784, or a variant or derivative thereof.

In a further aspect, the disclosure provides a GnRH antagonist, such as a GnRH antagonist described herein, for use in any of the methods described herein, such as those set forth above.

In another aspect, the disclosure provides uses of a GnRH antagonist, such as a GnRH antagonist described herein, in the manufacture of a medicament for treating a disease or condition described herein, for example, by way of any of the methods set forth above.

### Definitions

As used herein, the term “about” refers to a value that is within 10% above or below the value being described. For instance, a value of “about 5 mg” refers to a quantity that is from 4.5 mg to 5.5 mg.

As used herein, the term “abnormal uterine bleeding” refers to uterine blood loss that occurs either at an inappropriate time during a patient's menstrual cycle or in an amount that exceeds typical menstrual blood loss, such as “heavy menstrual blood loss” and “menorrhagia,” which refer to menstrual blood loss of 80 ml or more (e.g., 80 ml, 90 ml, 100 ml, 110 ml, 120 ml, 130 ml, 140 ml, 150 ml, 160 ml, 170 ml, 180 ml, 190 ml, 200 ml, or more) per menstrual cycle (The Menorrhagia Research Group. Quantification of menstrual blood loss. The Obstetrician & Gynaecologist 6:88-92 (2004)).

As used herein, the term “add-back therapy” refers to the administration of estrogen during a treatment regimen, such as treatment with a GnRH antagonist (e.g., 3-[2-fluoro-5-(2,3-difluoro-6-methoxybenzyloxy)-4-methoxyphenyl]-2,4-dioxo-1,2,3,4-tetrahydrothieno [3,4d]pyrimidine-5-carboxylic acid or a pharmaceutically acceptable salt thereof, such as the choline salt thereof, as described herein), so as to counteract side effects that may otherwise be associated with excessive suppression of estradiol. Such side effects may include, for example, a reduction in bone mineral density (BMD). A patient's BMD may be assessed by dual energy X-ray absorptiometry, for instance, in the spine or femur of the patient. Add-back therapy may be administered to a patient according to the methods described herein so as to mitigate a reduction in BMD caused by the administration of a GnRH antagonist. For instance, add-back therapy may be administered to a patient undergoing GnRH antagonist therapy such that the patient does not exhibit a reduction in BMD of greater than 5% (e.g., no greater than 5%, 4%, 3%, 2%, 1%, 0.9%, 0.8%, 0.7%, 0.6%, 0.5%, 0.4%, 0.3%, 0.2%, 0.1%, or less). Add-back therapy may include estrogen in the form of β17-estradiol, ethinyl estrogen, or a conjugated estrogen, such as a conjugated equine estrogen, and may further include one or more additional agents, such as a progestin (e.g., norethindrone or a compound that is metabolized in vivo to produce norethindrone, such as an ester of norethindrone that is de-esterified in vivo to produce norethindrone, for instance, norethindrone acetate, among other progestins such as progesterone, norgestimate, medroxyprogesterone, and drospirenone). Add-back therapy may be formulated for oral administration, such as in the form of a tablet, capsule, gel cap, powder, liquid solution, or liquid suspension. Add-back therapy may feature a co-formulation containing estrogen (e.g., in the form of β17-estradiol) and an additional agent such as a progestin (e.g., norethindrone or a compound that is metabolized in vivo to produce norethindrone, such as an ester of norethindrone that is de-esterified in vivo to produce norethindrone, for instance, norethindrone acetate). For instance, add-back therapy may be administered to a patient in the form of a single tablet, capsule, gel cap, powder, liquid solution, or liquid suspension that contains both estrogen (e.g., in the form of β17-estradiol) and a progestin (e.g., norethindrone or a compound that is metabolized in vivo to produce norethindrone, such as an ester of norethindrone that is de-esterified in vivo to produce norethindrone, for instance, norethindrone acetate). In some embodiments, add-back therapy is administered as a fixed dose combination containing a GnRH antagonist, estrogen, and one or more additional agents, such as a progestin, in a single pharmaceutical composition. For instance, add-back therapy may be administered as a fixed dose combination of a GnRH antagonist, estrogen (e.g., in the form of E2) and a progestin (e.g., norethindrone or a compound that is metabolized in vivo to produce norethindrone, such as an ester of norethindrone that is de-esterified in vivo to produce norethindrone, for instance, norethindrone acetate) in the form of a single pharmaceutical composition, such as a single tablet, capsule, gel cap, powder, liquid solution, or liquid suspension.

As used herein, a compound, such as a GnRH antagonist, estrogen, or progestin, among others, that is “administered” to a patient, such as a patient having an estrogen-dependent disease described herein, may be administered in an electrostatically neutral and/or nonionized form (e.g., in the form of a neutral carboxylic acid, a neutral amine, and the like) and/or in the form of a pharmaceutically acceptable salt, particularly if the compound contains a substituent that readily ionizes at physiological pH. For example, a compound containing a carboxylic acid substituent may be administered to a patient (e.g., a patient suffering from an estrogen-dependent disease described herein) in the form of the neutral, uncharged carboxylic acid and/or in the form of a carboxylate salt containing a pharmaceutically acceptable cation. Similarly, a compound containing an amine substituent may be administered to the patient in the form of the neutral, uncharged amine and/or in the form of an ammonium salt containing a pharmaceutically acceptable anion.

For example, as used herein, a GnRH antagonist of the disclosure, such as an optionally substituted thieno[3,4d]pyrimidine compound containing a carboxylic acid substituent, may be “administered” to a patient in the form of the neutral, uncharged carboxylic acid and/or in the form of a pharmaceutically acceptable salt (e.g., a salt containing the corresponding carboxylate anion and a pharmaceutically acceptable cation). Accordingly, as used herein, a GnRH antagonist of the formula 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid may be “administered” to a patient in the form of the neutral, uncharged carboxylic acid and/or in the form of a pharmaceutically acceptable salt (e.g., a salt containing the corresponding 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate anion and a pharmaceutically acceptable cation). For example, a GnRH antagonist of the formula 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid may be “administered” to a patient in the form of the neutral, uncharged carboxylic acid and/or in the form of a choline salt (i.e., a salt containing the corresponding 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate anion and a choline cation).

For example, as used herein, a GnRH antagonist of the disclosure, such as an optionally substituted 3-aminoalkyl pyrimidine-2,4(1H,3H)-dione compound containing a carboxylic acid substituent, may be “administered” to a patient in the form of the neutral, uncharged carboxylic acid and/or in the form of a pharmaceutically acceptable salt (e.g., a salt containing the corresponding carboxylate anion and a pharmaceutically acceptable cation). Accordingly, as used herein, a GnRH antagonist of the formula 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoic acid may be “administered” to a patient in the form of the neutral, uncharged carboxylic acid and/or in the form of a pharmaceutically acceptable salt (e.g., a salt containing the corresponding 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoate anion and a pharmaceutically acceptable cation). For example, a GnRH antagonist of the formula 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethy}amino)butanoic acid may be “administered” to a patient in the form of the neutral, uncharged carboxylic acid and/or in the form of a sodium salt (i.e., a salt containing the corresponding 4-({(1R)-2-[5-(2-fluoro-3-methoxyphenyl)-3-{[2-fluoro-6-(trifluoromethyl)phenyl]methy}-4-methyl-2,6-dioxo-3,6-dihydropyrimidin-1(2H)-yl]-1-phenylethyl}amino)butanoate anion and a sodium cation).

For example, as used herein, a GnRH antagonist of the disclosure, such as an optionally substituted thieno[2,3d]pyrimidine compound containing an amine substituent, may be “administered” to a patient in the form of the neutral, uncharged amine and/or in the form of a pharmaceutically acceptable salt (e.g., a salt containing the corresponding ammonium cation and a pharmaceutically acceptable anion). Accordingly, as used herein, a GnRH antagonist of the formula N-(4-(1-(2,6-difluorobenzyl)-5-((dimethylamino)methyl)-3-(6-metoxy-3-pyridazinyl)-2,4-dioxo-1,2,3,4-tetrahydrothieno[2,3-d]pyrimidin-6-yl)phenyl)-N′-methoxyurea may be “administered” to a patient in the form of the neutral, uncharged amine and/or in the form of a pharmaceutically acceptable salt (e.g., a salt containing the corresponding, protonated N-(4-(1-(2,6-difluorobenzyl)-5-((dimethylamino)methyl)-3-(6-methoxy-3-pyridazinyl)-2,4-dioxo-1,2,3,4-tetrahydrothieno[2,3-d]pyrimidin-6yl)phenyl)N′-methoxyurea cation and a pharmaceutically acceptable anion). For example, a GnRH antagonist of the formula N-(4-(1-(2,6-difluorobenzyl)-5-((dimethylamino)methyl)-3-(6-methoxy-3-pyridazinyl)-2,4-dioxo-1,2,3,4-tetrhydrothieno[2,3-d]pyrimidin-6-yl)phenyl)-N′-methoxyurea may be “administered” to a patient in the form of the neutral, uncharged amine and/or in the form of a chloride salt (i.e., a salt containing the corresponding, protonated N-(4-(1-(2,6-difluorobenzyl)5-((dimethylamino)methyl)-3-(6-methoxy-3-pyridazinyl)-2,4-dioxo-1,2,3,4-tetrahydrothieno[2,3-d]pyrimidin-6-yl)phenyl)-N′-methoxyurea cation and a chloride anion).

As used herein, a compound, such as a GnRH antagonist, estrogen, or progestin, among others, that is “administered” to a patient (e.g., a patient having an estrogen-dependent disease described herein) in a recited amount (e.g., per dose, per day, per week, per month, etc.) may be administered to the patient in the recited amount of an electrostatically neutral and/or nonionized form of the compound or in an equivalent amount of a pharmaceutically acceptable salt of the compound. As used herein, an amount of a pharmaceutically acceptable salt of a compound that is “equivalent” to a recited amount of the compound is an amount of the pharmaceutically acceptable salt that contains the same molar quantity of the compound as that contained by the recited amount of the compound. One can readily calculate the amount of a pharmaceutically acceptable salt of a compound that is “equivalent” to a recited amount of the compound using standard stoichiometry calculations known in the art.

Accordingly, as used herein, a GnRH antagonist of the disclosure, such as an optionally substituted thieno[3,4d]pyrimidine compound containing a carboxylic acid substituent, that is “administered” to a patient in a recited amount (e.g., per dose, per day, per week, per month, etc.) may be administered to the patient in the recited amount of an electrostatically neutral and/or nonionized of the compound or in an equivalent amount of a pharmaceutically acceptable salt of the compound. For example, as an optionally substituted thieno[3,4d]pyrimidine compound containing a carboxylic acid substituent, such as 3-[2-fluoro-5-(2,3-difluoro-methoxybenzyloxy)4-methoxyphenyl]-2,4-dioxo-1,2,3,4-tetrahydrothieno [3,4d]pyrimidine-5-carboxylic acid, that is “administered” to a patient in a recited amount may be administered to the patient in the recited amount of the neutral, uncharged form of the compound or in an equivalent amount of a pharmaceutically acceptable salt of the compound (e.g., a salt containing the corresponding 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate anion and a pharmaceutically acceptable cation, such as a choline cation). Accordingly, an optionally substituted thieno[3,4d]pyrimidine compound containing a carboxylic acid substituent, such as 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid, that is “administered” to a patient in a recited amount, such as a recited amount of from 25 mg to 400 mg (e.g., 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, or 400 mg), may be administered to the patient in the recited amount of the neutral, uncharged form of the compound or in an equivalent amount of a pharmaceutically acceptable salt of the compound (e.g., a salt containing the corresponding 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate anion and a pharmaceutically acceptable cation, such as a choline cation).

Accordingly, as used herein, a GnRH antagonist of the disclosure, such as an optionally substituted 3-aminoalkyl pyrimidine-2,4(1H,3H)-dione compound containing a carboxylic acid substituent, that is “administered” to a patient in a recited amount (e.g., per dose, per day, per week, per month, etc.) may be administered to the patient in the recited amount of an electrostatically neutral and/or nonionized form of the compound or in an equivalent amount of a pharmaceutically acceptable salt of the compound. For example, as an optionally substituted 3-aminoalkyl pyrimidine-2,4(1H,3H)-dione compound containing a carboxylic acid substituent, such as 4-({(1R)-2-[5-(2-fluoro-3-methoxyphenyl)-3-{[2-fluoro-8-(trifluoromethyl)phenyl]methyl}-4-methyl-2,6-dioxo-3,6-dihydropyrimidin-1(2H)-yl]-1-phenylethyl}amino)butanoic acid, that is “administered” to a patient in a recited amount may be administered to the patient in the recited amount of the neutral, uncharged form of the compound or in an equivalent amount of a pharmaceutically acceptable salt of the compound (e.g., a salt containing the corresponding 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoate anion and a pharmaceutically acceptable cation, such as a sodium cation). Accordingly, an optionally substituted 3-aminoalkyl pyrimidine-2,4(1H,3H)-dione compound containing a carboxylic acid substituent, such as 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoic acid, that is “administered” to a patient in a recited amount, such as a recited amount of from 50 mg to 650 mg (e.g., 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, 400 mg, 401 mg, 402 mg, 403 mg, 404 mg, 405 mg, 406 mg, 407 mg, 408 mg, 409 mg, 410 mg, 411 mg, 412 mg, 413 mg, 414 mg, 415 mg, 416 mg, 417 mg, 418 mg, 419 mg, 420 mg, 421 mg, 422 mg, 423 mg, 424 mg, 425 mg, 426 mg, 427 mg, 428 mg, 429 mg, 430 mg, 431 mg, 432 mg, 433 mg, 434 mg, 435 mg, 436 mg, 437 mg, 438 mg, 439 mg, 440 mg, 441 mg, 442 mg, 443 mg, 444 mg, 445 mg, 446 mg, 447 mg, 448 mg, 449 mg, 450 mg, 451 mg, 452 mg, 453 mg, 454 mg, 455 mg, 456 mg, 457 mg, 458 mg, 459 mg, 460 mg, 461 mg, 462 mg, 463 mg, 464 mg, 465 mg, 466 mg, 467 mg, 468 mg, 469 mg, 470 mg, 471 mg, 472 mg, 473 mg, 474 mg, 475 mg, 476 mg, 477 mg, 478 mg, 479 mg, 480 mg, 481 mg, 482 mg, 483 mg, 484 mg, 485 mg, 486 mg, 487 mg, 488 mg, 489 mg, 490 mg, 491 mg, 492 mg, 493 mg, 494 mg, 495 mg, 496 mg, 497 mg, 498 mg, 499 mg, 500 mg, 501 mg, 502 mg, 503 mg, 504 mg, 505 mg, 506 mg, 507 mg, 508 mg, 509 mg, 510 mg, 511 mg, 512 mg, 513 mg, 514 mg, 515 mg, 516 mg, 517 mg, 518 mg, 519 mg, 520 mg, 521 mg, 522 mg, 523 mg, 524 mg, 525 mg, 526 mg, 527 mg, 528 mg, 529 mg, 530 mg, 531 mg, 532 mg, 533 mg, 534 mg, 535 mg, 536 mg, 537 mg, 538 mg, 539 mg, 540 mg, 541 mg, 542 mg, 543 mg, 544 mg, 545 mg, 546 mg, 547 mg, 548 mg, 549 mg, 550 mg, 551 mg, 552 mg, 553 mg, 554 mg, 555 mg, 556 mg, 557 mg, 558 mg, 559 mg, 560 mg, 561 mg, 562 mg, 563 mg, 564 mg, 565 mg, 566 mg, 567 mg, 568 mg, 569 mg, 570 mg, 571 mg, 572 mg, 573 mg, 574 mg, 575 mg, 576 mg, 577 mg, 578 mg, 579 mg, 580 mg, 581 mg, 582 mg, 583 mg, 584 mg, 585 mg, 586 mg, 587 mg, 588 mg, 589 mg, 590 mg, 591 mg, 592 mg, 593 mg, 594 mg, 595 mg, 596 mg, 597 mg, 598 mg, 599 mg, 600 mg, 601 mg, 602 mg, 603 mg, 604 mg, 605 mg, 606 mg, 607 mg, 608 mg, 609 mg, 610 mg, 611 mg, 612 mg, 613 mg, 614 mg, 615 mg, 616 mg, 617 mg, 618 mg, 619 mg, 620 mg, 621 mg, 622 mg, 623 mg, 624 mg, 625 mg, 626 mg, 627 mg, 628 mg, 629 mg, 630 mg, 631 mg, 632 mg, 633 mg, 634 mg, 635 mg, 636 mg, 637 mg, 638 mg, 639 mg, 640 mg, 641 mg, 642 mg, 643 mg, 644 mg, 645 mg, 646 mg, 647 mg, 648 mg, 649 mg, or 650 mg), may be administered to the patient in the recited amount of the neutral, uncharged form of the compound or in an equivalent amount of a pharmaceutically acceptable salt of the compound (e.g., a salt containing the corresponding 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoate anion and a pharmaceutically acceptable cation, such as a sodium cation).

Accordingly, as used herein, a GnRH antagonist of the disclosure, such as an optionally substituted thieno[2,3d]pyrimidine compound containing an amine substituent, that is “administered” to a patient in a recited amount (e.g., per dose, per day, per week, per month, etc.) may be administered to the patient in the recited amount of an electrostatically neutral and/or nonionized form of the compound or in an equivalent amount of a pharmaceutically acceptable salt of the compound. For example, as an optionally substituted thieno[2,3d]pyrimidine compound containing an amine substituent, such as N-(4-(1-(2,6-difluorobenzyl)-5-((dimethylamino)methyl)-3-(6-methoxy-3-pyridazinyl)-2,4-dioxo-1,2,3,4-tetrahydrothieno[2,3-d]pyrimidin-6-yl)phenyl)-N′-methoxyurea, that is “administered” to a patient in a recited amount may be administered to the patient in the recited amount of the neutral, uncharged form of the compound or in an equivalent amount of a pharmaceutically acceptable salt of the compound (e.g., a salt containing the corresponding, protonated N-(4-(1-(2,6-difluorobenzyl)-5-((dimethylamino)methyl)-3-(6-methoxy-3-pyridazinyl)-2,4-dioxo-1,2,3,4-tetrahydrothieno[2,3-d]pyrimidin-6-yl)phenyl)-N′-methoxyurea cation and a pharmaceutically acceptable anion, such as a chloride anion). Accordingly, an optionally substituted thieno[2,3d]pyrimidine compound containing an amine substituent, such as N-(4-(1-(2,6-difluorobenzyl)5-((dimethylamino)methyl)-3-(6-methoxy-3-pyridazinyl)-2,4-dioxo-1,2,3,4-tetrahydrothieno[2,3-d]pyrimidin-6-yl)phenyl)-N′-methoxyurea, that is “administered” to a patient in a recited amount, such as a recited amount of from 10 mg to 60 mg (e.g., 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, or 60 mg), may be administered to the patient in the recited amount of the neutral, uncharged form of the compound or in an equivalent amount of a pharmaceutically acceptable salt of the compound (e.g., a salt containing the corresponding, protonated N-(4-(1-(2,6-difluorobenzyl)-5-((dimethylamino)methyl)-3-(6-methoxy-3-pyridazinyl)-2,4-dioxo-1,2,3,4-tetrahydrothieno[2,3-d]pyrimidin-6-yl)phenyl)-N′-methoxyurea cation and a pharmaceutically acceptable anion, such as a chloride anion).

As used herein, the term “affinity” refers to the strength of a binding interaction between two molecules, such as a ligand and a receptor. The term “K”, as used herein, is intended to refer to the inhibition constant of an antagonist for a particular molecule of interest, and can be expressed as a molar concentration (M). K values for antagonist-target interactions can be determined, e.g., using methods established in the art. Methods that can be used to determine the K of an antagonist for a molecular target include competitive binding experiments, e.g., as described in U.S. Pat. No. 9,040,693. The term “K_{d}”, as used herein, is intended to refer to the dissociation constant, which can be obtained, e.g., from the ratio of the rate constant for the dissociation of the two molecules (k_{d}) to the rate constant for the association of the two molecules (kₐ) and is expressed as a molar concentration (M). K_{d} values for receptor-ligand interactions can be determined, e.g., using methods established in the art. Methods that can be used to determine the K_{d} of a receptor-ligand interaction include surface plasmon resonance, e.g., through the use of a biosensor system such as a BIACORE® system.

As used herein, the term “amenorrhea” refers to the absence or near absence of uterine blood loss in a female patient, such as a human female patient undergoing GnRH antagonist treatment according to a dosing regimen described herein. As such, amenorrhea is a clinical indicator of reduced menstrual blood loss, such as reduced menstrual blood loss in a patient suffering from an estrogen-dependent disease (e.g., uterine fibroids, endometriosis (such as rectovaginal endometriosis), and adenomyosis, among others described herein) and undergoing GnRH antagonist treatment according to a dosing regimen described herein.

As used herein, the terms “benefit” and “response” are used interchangeably in the context of a subject undergoing therapy for the treatment of an estrogen-dependent disease described herein. These terms refers to any clinical improvement in the subject's condition. For example, clinical benefits in the context of a subject administered a gonadotropin-releasing hormone (GnRH) antagonist for the treatment of uterine fibroids, one of the estrogen-dependent diseases described herein, include, without limitation, (i) a reduction in serum concentration of FSH, LH, and/or E2 following administration of the GnRH antagonist to the patient, (ii) a reduction in uterine bleeding following administration of the GnRH antagonist to the patient; (iii) achievement of amenorrhea following administration of the GnRH antagonist to the patient; (iv) a reduction in the volume of one or more uterine fibroids following administration of the GnRH antagonist to the patient, (v) a reduction in pelvic pain following administration of the GnRH antagonist to the patient; (vi) a reduction in dysmenorrhea following administration of the GnRH antagonist to the patient; (vii) a reduction in dyspareunia following administration of the GnRH antagonist to the patient; (viii) a reduction in dyschezia following administration of the GnRH antagonist to the patient; and (ix) an improvement in the patient's overall well-being as determined, for example, by an improvement in the patient's Endometriosis Health Profile questionnaire (EHP-30) score following administration of the GnRH antagonist to the patient and/or by way of a positive Patient Global Impression of Change (PGIC) score following administration of the GnRH antagonist to the patient.

Similarly, exemplary clinical benefits in the context of a subject administered a GnRH antagonist for the treatment of endometriosis (e.g., rectovaginal endometriosis) include, without limitation, (i) a reduction in serum concentration of FSH, LH, and/or E2 following administration of the GnRH antagonist to the patient, (ii) a reduction in the volume of one or more endometriosis nodes (e.g., one or more rectovaginal endometriosis nodes) following administration of the GnRH antagonist to the patient, (iii) a reduction in bowel involvement of one or more type III endometriosis nodes following administration of the GnRH antagonist to the patient, (iv) a reduction in pelvic pain following administration of the GnRH antagonist to the patient; (v) a reduction in dysmenorrhea following administration of the GnRH antagonist to the patient; (vi) a reduction in dyspareunia following administration of the GnRH antagonist to the patient; (vii) a reduction in dyschezia following administration of the GnRH antagonist to the patient; (viii) a reduction in uterine bleeding following administration of the GnRH antagonist to the patient; (ix) achievement of amenorrhea following administration of the GnRH antagonist to the patient; and (x) an improvement in the patient's overall well-being as determined by an improvement in the patient's EHP-30 score following administration of the GnRH antagonist to the patient and/or by way of a positive PGIC score following administration of the GnRH antagonist to the patient

Exemplary clinical benefits in the context of a subject administered a GnRH antagonist for the treatment of adenomyosis, another estrogen-dependent disease described herein, include, without limitation, (i) a reduction in serum concentration of FSH, LH, and/or E2 following administration of the GnRH antagonist to the patient, (ii) a reduction in uterine volume following administration of the GnRH antagonist to the patient, (iii) a reduction in the thickness of the anterior and/or posterior region of the uterine myometrium following administration of the GnRH antagonist to the patient, (iv) a reduction in pelvic pain following administration of the GnRH antagonist to the patient; (v) a reduction in dysmenorrhea following administration of the GnRH antagonist to the patient; (vi) a reduction in dyspareunia following administration of the GnRH antagonist to the patient; (vii) a reduction in dyschezia following administration of the GnRH antagonist to the patient; (viii) a reduction in uterine tenderness following administration of the GnRH antagonist to the patient; (ix) a reduction in uterine bleeding following administration of the GnRH antagonist to the patient; (x) achievement of amenorrhea following administration of the GnRH antagonist to the patient; (xi) a reduction in the diameter of a junctional zone of adenomyosis following administration of the GnRH antagonist to the patient; and (xii) an improvement in the patient's overall well-being as determined by an improvement in the patient's EHP-30 score following administration of the GnRH antagonist to the patient and/or by way of a positive PGIC score following administration of the GnRH antagonist to the patient.

As used herein, the term “Biberoglu and Behrman scale” or “B&B scale” or a modification thereof, such as a “modified Biberoglu and Behrman scale” refers to a multi-point scale that can be used to indicate the severity of one or more symptoms experienced by patient suffering from an estrogen-dependent disease, such as endometriosis, among others. A B&B score can be assessed by verbally prompting the patient to indicate the degree of function or quality of life being experienced. A B&B score can be used, e.g., to assess the severity of such symptoms as dysmenorrhea, dyspareunia, chronic pelvic pain, pelvic tenderness, and induration, among others. Methods of determining a B&B score are described in detail, e.g., in Biberoglu and Behrman, Am. J. Obstet. Gynecol. 139:645 (1981).

As used herein, the term “crystalline” or “crystalline form” means having a physical state that is a regular three-dimensional array of atoms, ions, molecules or molecular assemblies. Crystalline forms have lattice arrays of building blocks called asymmetric units that are arranged according to well-defined symmetries into unit cells that are repeated in three-dimensions. In contrast, the term “amorphous” or “amorphous form” refers to an unorganized (no orderly) structure. The physical state of a therapeutic compound may be determined by exemplary techniques such as x-ray diffraction, polarized light microscopy and/or differential scanning calorimetry.

As used herein, the term “dose” refers to the quantity of a therapeutic agent, such as a GnRH antagonist described herein, that is administered to a subject for the treatment of a disorder or condition, such as to treat or ameliorate one or more symptoms of an estrogen-dependent disease described herein (e.g., uterine fibroids, endometriosis, such as rectovaginal endometriosis, and/or uterine fibroids). A therapeutic agent as described herein may be administered in a single dose or in multiple doses. In each case, the therapeutic agent may be administered using one or more “unit dosage forms” of the therapeutic agent, a term that refers to a one or more discrete compositions containing a therapeutic agent that collectively constitute a single “dose” of the agent. For instance, a single dose of 200 mg of a therapeutic agent may be administered using, e.g., two 100 mg unit dosage forms of the therapeutic agent. The unit dosage forms may be, for example, solid unit dosage forms, such as tablets or capsules, among others.

As used herein, the term “dual energy X-ray absorptiometry” (DEXA) refers to a spectroscopic method of measuring bone mineral density in a patient (e.g., a human patient) in which X-ray radiation of two distinct frequencies are transmitted towards a target bone of the patient. The absorption of the transmitted radiation can subsequently be correlated with a measure of the bone mineral density within the target bone. Methods of determining bone mineral density using DEXA are described in detail, e.g., in Mazess et al., American Journal of Clinical Nutrition 51:1106-1112 (1990).

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “Endometriosis Health Profile-30” or “EHP-30” refers to a questionnaire that can be used to evaluate quality of life in patient suffering from an estrogen-dependent disease, such as endometriosis, among others. A score obtained from this questionnaire (i.e., an “EHP-30 score”) may provide an indication of the patient's degree of pain, feeling of control and powerlessness, emotional well-being, social support, and/or self-image. Exemplary methods that can be used to perform an EHP-30 questionnaire and procedures for interpreting the scores obtained therefrom are known in the art are described, e.g., in Renouvel et al., Journal de Gynécologie Obstétrique et Biologie de la Reproduction 38:404-410 (2009), the disclosure of which is incorporated herein by reference as it pertains to methods for conducting and evaluating an EHP-30 questionnaire.

As used herein, the term “estrogen-dependent disease” refers to a disease or condition that is exacerbated or caused by excessive, inappropriate, or unregulated estrogen (e.g., β17-estradiol) production and/or an aberrant physiological response to estrogen. Estrogen-dependent diseases include those exacerbated or caused by circulating β17-estradiol levels in excess of, for example, 50 pg/ml. Examples of such diseases include uterine fibroids, endometriosis (e.g., rectovaginal endometriosis), and adenomyosis. Additional examples of estrogen-dependent diseases include, without limitation, benign prostatic hypertrophy, precocious puberty, premenstrual syndrome, polycystic ovary syndrome, hirsutism, short stature, sleep disorders, acne, baldness, and irritable bowel syndrome, among others.

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted there from.

As used herein, the term “gonadotropin-releasing hormone antagonist” or “GnRH antagonist” refers to a compound that specifically binds the GnRH receptor and is capable of inhibiting receptor signalling, e.g., such that release of one or more gonadotropins (such as follicle-stimulating hormone and luteinizing hormone) is inhibited. GnRH antagonists for use with the compositions and methods described herein include thieno[3,4d]pyrimidine derivatives and variants, such as those described in U.S. Pat. No. 9,040,693, the disclosure of which is incorporated herein by reference in its entirety. Exemplary GnRH antagonists include 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid or a pharmaceutically acceptable salt thereof, such as the choline salt thereof, e.g., as described in U.S. Pat. No. 9,169,266, the disclosure of which is incorporated herein by reference in its entirety. Additional examples of GnRH antagonists that may be used in conjunction with the compositions and methods described herein include optionally substituted 3-aminoalkyl pyrimidine-2,4(1H,3H)-dione derivatives, such as sodium 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoate, also referred to as elagolix, or the carboxylic acid conjugate thereof, and related compounds described in U.S. Pat. No. 7,056,927, the disclosure of which is incorporated herein by reference in its entirety. Further examples of GnRH antagonists that may be used in conjunction with the compositions and methods described herein include optionally substituted thieno[2,3d]pyrimidine derivatives, such as N-(4-(1-(2,6-difluorobenzyl)-5-((dimethylamino)methyl)-3-(6-methoxy-3-pyridazinyl)-2,4-dioxo-1,2,3,4-tetrahydrothieno[2,3-d]pyrimidin-6-yl)phenyl)-N′-methoxyurea, also referred to as relugolix, or a pharmaceutically acceptable salt thereof, and related compounds described in U.S. Pat. No. 7,300,935, the disclosure of which is incorporated herein by reference in its entirety. Additional examples of GnRH antagonists that may be used in conjunction with the compositions and methods described herein include optionally substituted propane-1,3-dione derivatives, such as (2R)—N-{5-[3-(2,5-difluorophenyl)-2-(1,3-dihydro-2H-benzimidazol-2-ylidene)-3-oxopropanoyl]-2-fluorobenzene-1-sulfonyl}-2-hydroxypropanimidamide, also referred to as opigolix or ASP-1707, and related compounds described in U.S. Pat. No. 6,960,591, the disclosure of which is incorporated herein by reference in its entirety.

As used herein, the term “IC₅₀” refers to the concentration of a substance (antagonist) that reduces the efficacy of a reference agonist or the constitutive activity of a biological target by 50%, e.g., as measured in a competitive ligand binding assay. Exemplary competitive ligand binding assays include competitive radioligand binding assays, competitive enzyme-linked immunosorbant assays (ELISA), and fluorescence anisotropy-based assays, among others known in the art.

As used herein in the context of a GnRH antagonist and add-back therapy, the term “in combination with” refers to administration of the GnRH antagonist and add-back therapy agent(s) such that the later-administered of these substances is provided to the patient at a time when there is still a detectable concentration in the patient's blood of the earlier-administered of these substances. The GnRH antagonist and add-back therapy need not be administered at the exact same moment for these substances to be administered “in combination with” one another.

As used herein, the term “menstrual cycle” refers to a recurring cycle of physiological changes in females, such as human females, that is associated with reproductive fertility. While the cycle length may vary from woman to woman, 28 days is generally taken as representative of the average ovulatory cycle in human females.

As used herein, the term “Numerical Rating Score” (NRS) refers to a score within an 11-point numerical scale of 0-10 that indicates the degree of pain experienced by a patient, such as a patient having an estrogen-dependent disease described herein. For instance, a score of 0 may indicate the patient is experiencing no pain, while scores from 1-3 may indicate that the patient is experiencing mild pain. A score of from 4-6 may indicate that the patient is experiencing moderate pain, and a score of from 7-10 may indicate that the patient is experiencing severe pain. Typically, to determine a NRS score, the patient is asked to indicate the level of pain currently being experienced, as well as the pain experienced at its most intense and least intense occurrences. Methods for determining a NRS are described in detail, e.g., in McCaffery et al., Pain: Clinical Manual for Nursing Practice. Baltimore (1993), the disclosure of which is incorporated herein by reference as it pertains to methods for obtaining and evaluating an NRS.

As used herein, the term “pharmaceutical composition” means a mixture containing a therapeutic compound to be administered to a patient, such as a mammal, e.g., a human, in order to prevent, treat or control a particular disease or condition affecting the mammal, such as preterm labor or dysmenorrhea.

As used herein, the term “pharmaceutically acceptable” refers to those compounds, materials, compositions and/or dosage forms, which are suitable for contact with the tissues of a patient, such as a mammal (e.g., a human) without excessive toxicity, irritation, allergic response and other problem complications commensurate with a reasonable benefit/risk ratio.

As used herein in the context of administration of a therapeutic agent, the term “periodically” refers to administration of the agent two or more times over the course of a treatment period (e.g., two or more times daily, weekly, monthly, or yearly).

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) isolated from a patient.

As used herein, the phrases “specifically binds” and “binds” refer to a binding reaction which is determinative of the presence of a particular protein in a heterogeneous population of proteins and other biological molecules that is recognized, e.g., by a ligand with particularity. A ligand (e.g., a protein, proteoglycan, or glycosaminoglycan) that specifically binds to a protein will bind to the protein, e.g., with a K_{D} of less than 100 nM. For example, a ligand that specifically binds to a protein may bind to the protein with a K_{D} of up to 100 nM (e.g., between 1 pM and 100 nM). A ligand that does not exhibit specific binding to a protein or a domain thereof may exhibit a K_{D} of greater than 100 nM (e.g., greater than 200 nM, 300 nM, 400 nM, 500 nM, 600 nm, 700 nM, 800 nM, 900 nM, 1 μM, 100 μM, 500 μM, or 1 mM) for that particular protein or domain thereof. A variety of assay formats may be used to determine the affinity of a ligand for a specific protein. For example, solid-phase ELISA assays are routinely used to identify ligands that specifically bind a target protein. See, e.g., Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), for a description of assay formats and conditions that can be used to determine specific protein binding.

As used herein, the terms “subject” and “patient” are used interchangeably and refer to an organism, such as a mammal (e.g., a human) that receives treatment for an estrogen-dependent disease described herein, such as uterine fibroids, endometriosis (e.g., rectovaginal endometriosis) and/or adenomyosis using the compositions and methods described herein and/or that is diagnosed as having one or more of these diseases in accordance with the methods described herein. Examples of patients include pre-menopausal female human patients. For instance, uterine fibroids patients in need of treatment in accordance with the compositions and methods described herein include those experiencing heavy menstrual bleeding (i.e., blood loss in excess of 80 ml per menstrual cycle). Examples of adenomyosis patients in need of treatment using a GnRH antagonist according to the methods described herein include, e.g., adenomyosis patients diagnosed as having a junctional-zone width of about 12 mm or more prior to administration of the GnRH antagonist to the patient (e.g., a junctional zone width of from about 12 mm to about 20 mm, from about 12 mm to about 19 mm, from about 12 mm to about 18 mm, from about 12 mm to about 17 mm, from about 12 mm to about 16 mm, from about 12 mm to about 15 mm, from about 12 mm to about 14 mm, or more, prior to administration of the GnRH antagonist to the patient). Examples of endometriosis patients (e.g., rectovaginal endometriosis patients) in need of treatment using a GnRH antagonist according to the methods described herein include, e.g., rectovaginal endometriosis patients exhibiting a rectal (type II) and/or vaginal (type III) endometriosis node of at least 2 cm prior to administration of the GnRH antagonist to the patient, such as a rectal (type II) and/or vaginal (type III) endometriosis node of from about 2 cm to about 10 cm, or more (e.g., a rectal (type II) and/or vaginal (type III) endometriosis node of from about 2 cm to about 9 cm, from about 2 cm to about 8 cm, from about 2 cm to about 7 cm, from about 2 cm to about 6 cm, from about 2 cm to about 5 cm, or from about 2 cm to about 4 cm, or more) prior to administration of the GnRH antagonist to the patient.

As used herein in the context of administration of a GnRH antagonist and add-back therapy, the term “substantially the same time” refers to administration of the later-provided agent to a patient at a time such that there is still a detectable concentration of the earlier-provided agent in the patient's circulating blood. Depending on the half-life of the GnRH antagonist, for example, add-back therapy may be provided to a patient at “substantially the same time” as the GnRH antagonist if the two agents are administered to the patient within one or more hours of one another, provided that there remains a detectable concentration of the earlier-administered agent in the patient's blood when the later-administered agent is provided to the patient. The compound represented by formula (VI) herein, and its pharmaceutically acceptable salt forms, such as the choline salt thereof (represented by formula (VIa), herein) has a half-life of about 14-15 hours in a human patient, such as a human patient having an estrogen-dependent disease described herein. Elagolix, in contrast, has a half-life of from about 2-6 hours in vivo, while relugolix has a half-life of about 37-42 hours in vivo. Given these half-lives, one of skill in the art will appreciate that the add-back therapy and GnRH antagonist need not be administered to a patient at precisely the same moment in order for these agents to be considered administered to a patient at “substantially the same time” in accordance with the compositions and methods of the present disclosure.

As used herein in the context of a GnRH antagonist and add-back therapy, the term “therapeutically effective amounts” refers to the combined quantities of a GnRH antagonist and add-back therapy agent(s) (e.g., an estrogen and/or progestin described herein) that, when administered in combination with one another, are capable of promoting a reduction in endogenous β17-estradiol levels to physiologically healthy concentrations, such as a concentration of less than 50 pg/ml in circulating blood, without permitting endogenous β17-estradiol to be depleted to an extent so low that the patient exhibits an undesirable side effect, such as bone mineral density loss, for example, in excess of 5%. Exemplary “therapeutically effective amounts” of a GnRH antagonist include, without limitation, from about 50 mg to about 200 mg (e.g., from about 100 mg to about 200 mg) of a compound represented by any one of formulas (I)-(VIa) herein (e.g., in the recited amount or in an equivalent amount of a pharmaceutically acceptable salt, such as a choline salt), when administered, for instance, in combination with add back therapy, such as β17-estradiol in an amount of about 1.0 mg and norethindrone acetate in an amount of about 0.5 mg, among other dosage quantities described herein.

As used herein, the terms “treat” or “treatment” refer to therapeutic treatment, in which the object is to prevent or slow down (lessen) an undesired physiological change or disorder in a human patient, such as the progression of an estrogen-dependent disease described herein, including uterine fibroids, endometriosis (e.g., rectovaginal endometriosis) and adenomyosis, among others. Beneficial or desired clinical results include, but are not limited to, alleviation of symptoms, such as a reduction in pelvic pain, a reduction in dysmenorrhea, a reduction in dyspareunia, a reduction in dyschezia, and a reduction in uterine bleeding, among other desired benefits described herein. As a non-limiting example, a patient, such as a female human patient, suffering from uterine fibroids may considered to be treated using a GnRH antagonist described herein if the patient exhibits (i) a reduction in uterine blood loss (e.g., elimination of heavy menstrual blood loss) following administration of the GnRH antagonist to the patient (e.g., within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient); and/or (ii) induction of amenorrhea following administration of the GnRH antagonist to the patient (e.g., within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient).

Similarly, clinical indicators of successful treatment of a patient having endometriosis (e.g., rectovaginal endometriosis) using a GnRH antagonist described herein include (i) a reduction in the volume of one or more endometriosis nodes (e.g., rectovaginal endometriosis nodes) following administration of the GnRH antagonist to the patient (e.g., within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient); (ii) a reduction in bowel involvement of one or more type III endometriosis nodes following administration of the GnRH antagonist to the patient (e.g., within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient); (iii) a reduction in pelvic pain following administration of the GnRH antagonist to the patient (e.g., within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient); (iv) a reduction in dysmenorrhea following administration of the GnRH antagonist to the patient (e.g., within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient); (v) a reduction in dyspareunia following administration of the GnRH antagonist to the patient (e.g., within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient); (vi) a reduction in dyschezia following administration of the GnRH antagonist to the patient (e.g., within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient); (vii) a reduction in uterine tenderness following administration of the GnRH antagonist to the patient (e.g., within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient); (viii) a reduction in uterine bleeding following administration of the GnRH antagonist to the patient (e.g., within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient); (ix) achievement of amenorrhea following administration of the GnRH antagonist to the patient (e.g., within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient); and/or (x) an improvement in the patient's overall well-being as determined by an improvement in the patient's EHP-30 score following administration of the GnRH antagonist to the patient (e.g., within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient) and/or the observation of a positive PGIC score following administration of the GnRH antagonist to the patient (e.g., within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient).

Clinical indicators of successful treatment of a patient having adenomyosis, another estrogen-dependent disease described herein, include, without limitation, (i) a reduction in uterine volume following administration of the GnRH antagonist to the patient (e.g., within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient); (ii) a reduction in the thickness of the anterior and/or posterior region of the uterine myometrium following administration of the GnRH antagonist to the patient (e.g., within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient); (iii) a reduction in pelvic pain following administration of the GnRH antagonist to the patient (e.g., within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient); (iv) a reduction in dysmenorrhea following administration of the GnRH antagonist to the patient (e.g., within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient); (v) a reduction in dyspareunia following administration of the GnRH antagonist to the patient (e.g., within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient); (vi) a reduction in dyschezia following administration of the GnRH antagonist to the patient (e.g., within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient); (vii) a reduction in uterine tenderness following administration of the GnRH antagonist to the patient (e.g., within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient); (viii) a reduction in uterine bleeding following administration of the GnRH antagonist to the patient (e.g., within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient); (ix) achievement of amenorrhea following administration of the GnRH antagonist to the patient (e.g., within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient); (x) a reduction in the diameter of a junctional zone of adenomyosis following administration of the GnRH antagonist to the patient (e.g., within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient); and/or (xi) an improvement in the patient's overall well-being as determined by an improvement in the patient's EHP-30 score following administration of the GnRH antagonist to the patient (e.g., within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient) and/or the observation of a positive PGIC score following administration of the GnRH antagonist to the patient (e.g., within from about one day to about 36 weeks of the first administration of the GnRH antagonist to the patient, such as within about 1 day, 2 days, 3 days, 4 days, 4 days, 6 days, 7 days, 2 weeks, 3 weeks, 4 weeks, 5 weeks, 6 weeks, 7 weeks, 8 weeks, 9 weeks, 10 weeks, 11 week, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, or 36 weeks, of the first administration of the GnRH antagonist to the patient).

As used herein, the term “treatment period” refers to a duration of time over which a patient may be periodically administered a therapeutic agent, such as a GnRH antagonist described herein. Treatment periods as described herein may have a duration of several days, weeks, months, or years. For instance, a treatment period for administration of a thieno[3,4d]pyrimidine derivative, such as 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid or a pharmaceutically acceptable salt thereof, such as the choline salt thereof, may last for from about four weeks to about six months (e.g., from about 28 days to about 180 days, about 30 days to about 175 days, about 35 days to about 170 days, about 40 days to about 165 days, about 45 days to about 160 days, about 50 days to about 155 days, about 55 days to about 150 days, about 60 days to about 145 days, about 65 days to about 140 days, about 70 days to about 135 days, about 75 days, to about 130 days, about 80 days to about 125 days, about 85 days, to about 120 days, or about 90 days to about 115 days). In some embodiments, the GnRH antagonist is periodically administered to the patient over a treatment period of from about eight weeks to about sixteen weeks (e.g., from about 60 days to about 110 days, about 65 days to about 105 days, about 70 days to about 100 days, about 75 days to about 95 days, or about 80 days, to about 90 days). In some embodiments, the GnRH antagonist is periodically administered to the patient over a treatment period of about twelve weeks. In some embodiments, the GnRH antagonist is periodically administered to the patient over a treatment period of from about 20 weeks to about 30 weeks (e.g., from about 140 days to about 210 days, about 150 days to about 100 days, about 60 days to about 90 days, about 65 days to about 85 days, or about 68 days). In some embodiments, the GnRH antagonist is periodically administered to the patient over a treatment period of about 24 weeks.

As used herein, the term “Verbal Rating Score” (VRS) refers to a subjective multi-point scale used to indicate the level of pain being experienced by a patient undergoing therapy or that has previously undergone therapy for a disease or condition, such as an estrogen-dependent disease described herein. The VRS may be a five-point scale and can be assessed by prompting the patient with one or more questions in order to determine the level of pain currently being experienced by the patient. Methods for assessing a VRS are described in detail, e.g., in Jensen et al., Journal of Pain and Symptom Management 41:1073-1093 (2011), the disclosure of which is incorporated herein by reference as it pertains to methods for obtaining and evaluating a VRS.

As used herein, the term “aryl” refers to an unsaturated aromatic carbocyclic group of from 6 to 14 carbon atoms having a single ring (e.g., optionally substituted phenyl) or multiple condensed rings (e.g., optionally substituted naphthyl). Exemplary aryl groups include phenyl, naphthyl, phenanthrenyl, and the like.

As used herein, the term “cycloalkyl” refers to a monocyclic cycloalkyl group having from 3 to 8 carbon atoms, such as cyclopropyl, cyclobutyl, cyclopentyl, cyclohexyl, cycloheptyl, cyclooctyl, and the like.

As used herein, the term “halogen atom” refers to a fluorine atom, a chlorine atom, a bromine atom, or an iodine atom.

As used herein, the term “heteroaryl” refers to a monocyclic heteroaromatic, or a bicyclic or a tricyclic fused-ring heteroaromatic group. Exemplary heteroaryl groups include optionally substituted pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadia-zolyl, 1,2,5-oxadiazolyl, 1,3,4-oxadiazolyl, 1,3,4-triazinyl, 1,2,3-triazinyl, benzofuryl, [2,3-dihydro]benzofuryl, isobenzofuryl, benzothienyl, benzotriazolyl, isobenzothienyl, indolyl, isoindolyl, 3H-indolyl, benzimidazolyl, imidazo[1,2-a]pyridyl, benzothiazolyl, benzoxazolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 5,6,7,8-tetrahydroquinolyl, 5,6,7,8-tetrahydroisoquinolyl, purinyl, pteridinyl, carbazolyl, xanthenyl, benzoquinolyl, and the like.

As used herein, the term “heterocycloalkyl” refers to a 3 to 8-membered heterocycloalkyl group having 1 or more heteroatoms, such as a nitrogen atom, an oxygen atom, a sulfur atom, and the like, and optionally having 1 or 2 oxo groups such as pyrrolidinyl, piperidinyl, oxopiperidinyl, morpholinyl, piperazinyl, oxopiperazinyl, thiomorpholinyl, azepanyl, diazepanyl, oxazepanyl, thiazepanyl, dioxothiazepanyl, azokanyl, tetrahydrofuranyl, tetrahydropyranyl, and the like.

As used herein, the terms “lower alkyl” and “C₁₋₆alkyl” refer to an optionally branched alkyl moiety having from 1 to 6 carbon atoms, such as methyl, ethyl, propyl, isopropyl, butyl, isobutyl, sec-butyl, tert-butyl, pentyl, isopentyl, neopentyl, tert-pentyl, hexyl, and the like.

As used herein, the term “lower alkylene” refers to an optionally branched alkylene group having from 1 to 6 carbon atoms, such as methylene, ethylene, methylmethylene, trimethylene, dimethylmethylene, ethylmethylene, methylethylene, propylmethylene, isopropylmethylene, dimethylethylene, butylmethylene, ethylmethylmethylene, pentamethylene, diethylmethylene, dimethyltrimethylene, hexamethylene, diethylethylene and the like.

As used herein, the term “lower alkenyl” refers to an optionally branched alkenyl moiety having from 2 to 6 carbon atoms, such as vinyl, allyl, 1-propenyl, isopropenyl, 1-butenyl, 2-butenyl, 2-methylallyl, and the like.

As used herein, the term “lower alkynyl” refers to an optionally branched alkynyl moiety having from 2 to 6 carbon atoms, such as ethynyl, 2-propynyl, and the like.

As used herein, the term “optionally fused” refers to a cyclic chemical group that may be fused with a ring system, such as cycloalkyl, heterocycloalkyl, aryl, or heteroaryl. Exemplary ring systems that may be fused to an optionally fused chemical group include, e.g., indolyl, isoindolyl, benzofuranyl, isobenzofuranyl, benzothiophenyl, benzoxazolyl, benzothiazolyl, benzoisoxazolyl, benzoisothiazolyl, indazolyl, benzimidazolyl, quinolinyl, isoquinolinyl, phthalazinyl, quinoxalinyl, quinazolinyl, cinnolinyl, indolizinyl, naphthyridinyl, pteridinyl, indanyl, naphtyl, 1,2,3,4-tetrahydronaphthyl, indolinyl, isoindolinyl, 2,3,4,5-tetrahydrobenzo[b]oxepinyl, 6,7,8,9-tetrahydro-5H-benzocycloheptenyl, chromanyl, and the like.

As used herein, the term “optionally substituted” refers to a chemical moiety that may have one or more (e.g., 1, 2, 3, 4, 5, 6, 7, 8, 9, 10, or more chemical substituents, such as lower alkyl, lower alkenyl, lower alkynyl, cycloalkyl, heterocyclolalkyl, aryl, alkylaryl, heteroaryl, alkylheteroaryl, amino, ammonium, acyl, acyloxy, acylamino, aminocarbonyl, alkoxycarbonyl, ureido, carbamate, sulfinyl, sulfonyl, alkoxy, sulfanyl, halogen, carboxy, trihalomethyl, cyano, hydroxy, mercapto, nitro, and the like. An optionally substituted chemical moiety may contain, e.g., neighboring substituents that have undergone ring closure, such as ring closure of vicinal functional substituents, thus forming, e.g., lactams, lactones, cyclic anhydrides, acetals, thioacetals, or aminals formed by ring closure, for instance, in order to generate protecting group.

As used herein, the term “sulfinyl” refers to the chemical moiety “—S(O)—R” in which R represents, e.g., hydrogen, aryl, heteroaryl, optionally substituted alkyl, optionally substituted alkenyl, or optionally substituted alkynyl.

As used herein, the term “sulfonyl” refers to the chemical moiety “—SO₂—” in which R represents, e.g., hydrogen, aryl, heteroaryl, optionally substituted alkyl, optionally substituted alkenyl, or optionally substituted alkynyl.

One of skill in the art will appreciate that certain compounds described herein can exist in one or more different isomeric (e.g., stereoisomers, geometric isomers, tautomers) and/or isotopic (e.g., in which one or more atoms has been substituted with a different isotope of the atom, such as hydrogen substituted for deuterium) forms. Unless otherwise indicated or clear from context, a depicted structure is to be understood as representing any such isomeric or isotopic form, individually or in combination.

### BRIEF DESCRIPTION OF THE FIGURES

FIG. 1A is a graph characterizing the uterine bleeding patterns of a cohort of female human subjects pre-treated for four weeks with the GnRH antagonist represented by formula (VI), herein (administered as a choline salt), followed by combined administration of the GnRH antagonist and hormonal add-back therapy (“Delayed-ABT”), as described in Example 1, below. Uterine bleeding was classified as belonging to one of the following four categories: “no bleeding,” “spotting,” “bleeding,” or “heavy bleeding.” Values along the x-axis represent the time point, in weeks, during which the uterine blood loss measurement was made.

FIG. 1B is a graph characterizing the uterine bleeding patterns of a cohort of female human subjects that were provided with a simultaneous onset of administration of GnRH antagonist therapy (using the GnRH antagonist represented by formula (VI), herein (administered as a choline salt)) and hormonal add-back therapy administration (“Concurrent-ABT”), as described in Example 1, below. Uterine bleeding was classified as belonging to one of the following four categories: “no bleeding,” “spotting,” “bleeding,” or “heavy bleeding.” Values along the x-axis represent the time point, in weeks, during which the uterine blood loss measurement was made.

FIG. 2 is a graph characterizing the quantity of patients that exhibited no bleeding at various time points over the course of administration of GnRH antagonist therapy (using the GnRH antagonist represented by formula (VI), herein (administered as a choline salt)), as described in Example 1, below. Particularly, the graph compares (p=0.0299) the proportion of patients in the “no bleeding” category among patients that were provided with a simultaneous onset of administration of GnRH antagonist therapy and hormonal add-back therapy administration (“Concurrent-ABT”) to the proportion of patients in the “no bleeding” category among patients that were pre-treated for four weeks with the GnRH antagonist followed by combined administration of the GnRH antagonist and hormonal add-back therapy (“Delayed-ABT”).

FIG. 3A is a graph showing the reduction in endogenous β17-estradiol (E2) levels induced by GnRH antagonist therapy in combination with add-back therapy using either a “Delayed-ABT” dosing regimen or “Concurrent-ABT” dosing regimen, as described in Example 1, below.

FIG. 3B is a graph showing the reduction in endogenous progesterone levels induced by GnRH antagonist therapy in combination with add-back therapy, as described in Example 1, below.

### DETAILED DESCRIPTION

The compositions and methods of the present disclosure can be used to treat a variety of estrogen-dependent disorders, including uterine fibroids and endometriosis, among others. For example, using the compositions and methods described herein, a patient, such as a female human patient, may be administered a gonadotropin-releasing hormone (GnRH) antagonist in order to not only treat the cause of the estrogen-dependent pathology, but also to alleviate one or more symptoms associated these conditions. Exemplary disease that are induced by elevated estrogen production, and that can be treated using the compositions and methods described herein, include, without limitation, uterine fibroids, endometriosis (e.g., rectovaginal endometriosis), and adenomyosis, among others.

GnRH antagonists that may be used for the treatment of an estrogen-dependent disorder as described herein include optionally substituted thieno[3,4d]pyrimidine derivatives, such as 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid or the choline salt thereof. Other GnRH antagonists useful in conjunction with the compositions and methods of the disclosure include optionally substituted 3-aminoalkyl pyrimidine-2,4(1H,3H)-dione derivatives, such as sodium 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoate, also referred to as elagolix, or the carboxylic acid conjugate thereof. In some embodiments, the GnRH antagonist is an optionally substituted thieno[2,3d]pyrimidine derivative, such as N-(4-(1-(2,6-difluorobenzyl)-5-((dimethylamino)methyl)-3-(6-methoxy-3-pyridazinyl)-2,4-dioxo-1,2,3,4-tetrahydrothieno[2,3-d]pyrimidin-6-yl)phenyl)-N′-methoxyurea, also referred to as relugolix, or a pharmaceutically acceptable salt thereof. In some embodiments, the GnRH antagonist is an optionally substituted propane-1,3-dione derivative, such as (2R)—N-{5-[3-(2,5-difluorophenyl)-2-(1,3-dihydro-2H-benzimidazol-2-ylidene)-3-oxopropanoyl]-2-fluorobenzene-1-sulfonyl}-2-hydroxypropanimidamide, also referred to as opigolix or ASP-1707. Additional GnRH antagonists that may be used in conjunction with the compositions and methods described herein include SK12670 and BAY-784, as well as derivatives and variants thereof, among others.

Estrogen-dependent diseases, such as uterine fibroids, endometriosis (e.g., rectovaginal endometriosis), and adenomyosis, among others described herein, may be effectuated by circulating concentrations of β17-estradiol (E2) in excess of 50 pg/ml. Using the compositions and methods described herein, a GnRH antagonist may be administered to the patient so as to suppress E2 production to healthy levels, such as a concentration of from about 20 pg/ml to about 50 pg/ml, in order to correct the underlying physiological cause of the disease and ameliorate one or more symptoms associated therewith. However, excessive depletion of endogenous E2 (for example, to levels of less than 20 pg/ml) may lead to undesirable side effects, such as a reduction in bone mineral density. To prevent hypoestrogenemia, the GnRH antagonists of the disclosure may be administered to a patient in combination with add-back therapy, which provides the patient with an exogenous source of estrogen. Dual administration of a GnRH antagonist and add-back therapy may thus have the effect of partially compensating for the reduction in endogenous E2 induced by the GnRH antagonist. In this way, endogenous E2 can be reduced to a level sufficient to treat an estrogen-dependent disease (e.g., uterine fibroids, endometriosis, such as rectovaginal endometriosis, and adenomyosis, among others) without permitting bone mineral density loss.

The compositions and methods of the disclosure are based, in part, on the surprising discovery that the simultaneous onset of GnRH antagonist administration and add-back therapy administration reduces endogenous E2 levels to a greater extent than dosing schedules in which the patent is first treated with a GnRH antagonist alone before receiving add-back therapy. In uterine fibroids patients, the enhanced reduction in E2 achieved by concurrent onset of GnRH antagonist and add-back therapy administration manifests as a marked improvement in uterine bleeding pattern. One of the discoveries underlying the present disclosure is the unexpected observation that patients administered a GnRH antagonist for the treatment of uterine fibroids with a concurrent onset of the antagonist and add-back therapy exhibit substantially diminished uterine blood loss relative to patients that are pre-treated with the GnRH antagonist alone before receiving add-back therapy. It has been observed that patients pre-treated with a GnRH antagonist for several weeks have endogenous E2 concentrations that mirror those of post-menopausal subjects (Pohl et al., J Clin Endocrinol Metab. 103:497-504 (2018)). When post-menopausal subjects are administered add-back therapy, the uterine blood loss exhibited by these subjects remains low (Archer et al., Fertil. Steril. 108:152-160 (2017)). Thus, one might conclude that subjects pre-treated with a GnRH antagonist before receiving add-back therapy would similarly exhibit minimal uterine bleeding. The present disclosure is based, in part, on the surprising discovery that subjects that are provided a simultaneous onset of GnRH antagonist administration and add-back therapy administration actually exhibit significantly less uterine bleeding relative to subjects that are pre-treated with a GnRH antagonist alone before initiating add-back therapy.

The sections that follow provide a detailed description of GnRH antagonists and add-back therapy agents that may be used in conjunction with the compositions and methods of the disclosure, as well as a description of various estrogen-dependent diseases that may be treated using these therapeutics.

GnRH Antagonists

### Thieno[3,4d]pyrimidines

GnRH antagonists for use with the compositions and methods described herein include thieno[3,4d]pyrimidine derivatives and variants, such as those described in U.S. Pat. No. 9,040,693, the disclosure of which is incorporated herein by reference in its entirety. Exemplary GnRH antagonists include those represented by formula (I)

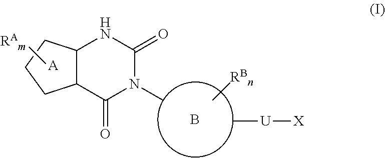

wherein ring A is a thiophene ring;

each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;

m is an integer from 0 to 3;

ring B is an aryl group or a monocyclic heteroaryl group;

each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;

n is an integer from 0 to 2;

U is a single bond;

X is a group represented by —S-L-Y, —O-L-Y, —CO-L-Y, or —SO₂-L-Y, wherein L is an optionally substituted lower alkylene group;

Y is a group represented by Z or —NW⁷W⁶, wherein W⁷ and W⁸ independently are a hydrogen atom, an optionally substituted lower alkyl group, or Z with the proviso that W⁷ and W⁶ are not simultaneously hydrogen atoms, or W⁷ and W⁸ may bind together with the neighboring nitrogen atom to form an optionally substituted cyclic amino group;

Z is an optionally fused and optionally substituted cycloalkyl group, an optionally fused and optionally substituted heterocycloalkyl group, an optionally fused and optionally substituted aryl group, or an optionally fused and optionally substituted heteroaryl group;

or a pharmaceutically acceptable salt thereof.

In some embodiments, the ring A is a thiophene ring represented by formula (IIa)

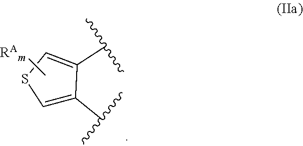

In some embodiments, m is 1 or 2. In some embodiments, m is 1. For instance, the ring A may be an optionally substituted thiophene ring represented by formula (IIb)

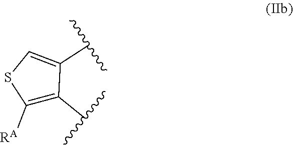

Each R^{A} may independently be, for example, a halogen atom (e.g., fluorine, chlorine, bromine, or iodine), an optionally substituted lower alkyl group, COOW¹, or CONW²W³, wherein W¹ to W³ independently are a hydrogen atom or an optionally substituted lower alkyl group, or W² and W³ may bind together with the neighboring nitrogen atom to form an optionally substituted cyclic amino group. In some embodiments, each R^{A} is COOH or pharmaceutically acceptable salt thereof.

In some embodiments, ring B is an optionally substituted benzene ring, pyridine ring, or thiophene ring. For instance, ring B may be represented by a formula selected from the group consisting of:

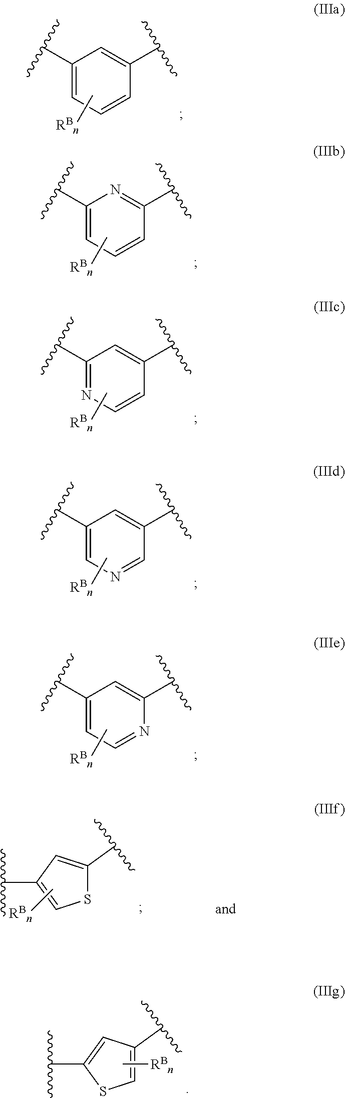

In some embodiments, n is 1 or 2. For instance, in some embodiments, n is 1. Ring B may be, for example, represented by a formula selected from the group consisting of:

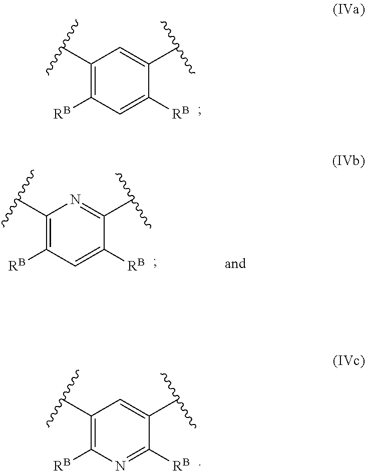

In some embodiments, each R^{B} is independently a halogen atom, an optionally substituted lower alkyl group, or OW⁴, wherein each W⁴ is independently a hydrogen atom or an optionally substituted lower alkyl group. For instance, each R^{B} may be independently a fluorine atom, chlorine atom, bromine atom, methyl group, or methoxy group.

In some embodiments, U is a single bond. X may be, for example, a group represented by —O-L-Y. L may be, for example, a methylene group. In some embodiments, Y is an optionally substituted benzene ring represented by formula (V)

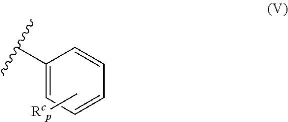

wherein each R is independently a halogen atom, an optionally substituted lower alkyl group, or OW⁹ wherein each W⁸ is independently a hydrogen atom or an optionally substituted lower alkyl group; and

p is an integer from 0 to 3.

In some embodiments, Y is a substituted benzene ring represented by formula (Va)

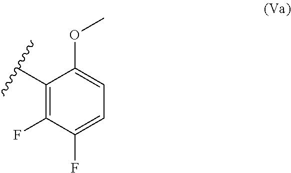 [SMILES: *c1c(OC)ccc(F)c1F]

For example, GnRH antagonists that may be used for the treatment of the endometrial growth disorders described herein include those thieno[3,4d]pyrimidine compounds described in Table 1, below. The synthesis and characterization of these compounds is reported, for instance, in U.S. Pat. No. 9,040,693, incorporated herein by reference.

TABLE 1
Exemplary Thieno[3,4d]pyrimidine GnRH Antagonists Useful for the Treatment of Estrogen-Dependent Diseases

| No. | Compound | Reported ¹H NMR spectral properties |
|---|---|---|
| 1 | 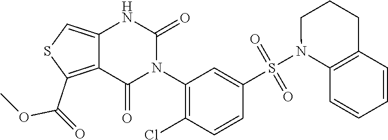 [SMILES: COC(=O)c1scc2[nH]c(=O)n(-c3cc(S(=O)(=O)N4CCCc5ccccc54)ccc3Cl)c(=O)c12] | (CDCl₃) 1.6-1.75 (2H, m), 2.45-2.6 (2H, m), 3.7-3.85 (2H, m), 3.94 (3H, s), 6.82 (1H, s), 7.0-7.15 (2H, m), 7.15-7.25 (1H, m), 7.49 (1H, dd, J = 8.4 Hz, 2.0 Hz), 7.54 (1H, d, J = 8.4 Hz), 7.6 (1H, d, J = 2.0 Hz), 7.7-7.8 (1H, m), 9.47 (1H, s) |
| 2 | 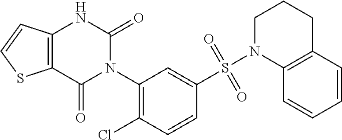 [SMILES: O=c1[nH]c2ccsc2c(=O)n1-c1cc(S(=O)(=O)N2CCCc3ccccc32)ccc1Cl] | (CDCl₃) 1.6-1.75 (2H, m), 2.4-2.6 (2H, m), 3.7-3.8 (1H, m), 3.8-3.9 (1H, m), 6.82 (1H, d, J = 5.2 Hz), 7.0-7.15 (2H, m), 7.15-7.25 (1H, m), 7.46 (1H, dd, J = 8.3 Hz, 2.1 Hz), 7.54 (1H, d, J = 8.3 Hz), 7.65 (1H, d, J = 2.1 Hz), 7.7-7.8 (2H, m), 9.65 (1H, s) |
| 3 | 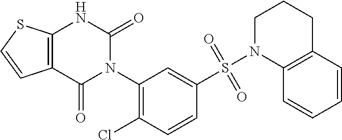 [SMILES: O=c1[nH]c2sccc2c(=O)n1-c1cc(S(=O)(=O)N2CCCc3ccccc32)ccc1Cl] | (CDCl₃) 1.6-1.75 (2H, m), 2.4-2.6 (2H, m), 3.7-3.9 (2H, m), 6.89 (1H d, J = 5.8 Hz), 7.0-7.15 (2H, m), 7.15-7.25 (1H, m), 7.32 (1H, d, J = 5.8 Hz), 7.46 (1H, dd, J = 8.4 Hz, 2.1 Hz), 7.55 (1H, d, J = 8.4 Hz), 7.64 (1H, d, J = 2.1 Hz), 7.75- 7.8 (1H, m), 9.54 (1H, brs) |
| 4 | 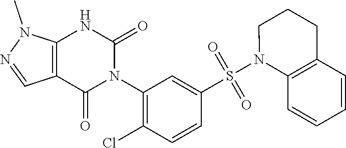 [SMILES: Cn1ncc2c(=O)n(-c3cc(S(=O)(=O)N4CCCc5ccccc54)ccc3Cl)c(=O)[nH]c21] | (CDCl₃) 1.6-1.8 (2H, m), 2.4-2.6 (2H, m), 3.65-3.8 (4H, m), 3.8-3.9 (1H, m), 7.0-7.15 (2H, m), 7.15-7.25 (1H, m), 7.49 (1H, dd, J = 8.4 Hz, 1.9 Hz), 7.55 (1H, d, J = 8.4 Hz), 7.59 (1H, d, J = 1.9 Hz), 7.75 (1H, d, J = 7.5 Hz), 7.91 (1H, s) |
| 5 | 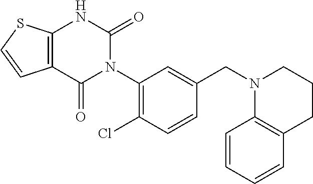 [SMILES: O=c1[nH]c2sccc2c(=O)n1-c1cc(CN2CCCc3ccccc32)ccc1Cl] | (CDCl₃) 1.95-2.05 (2H, m), 2.75-2.85 (2H, m), 3.3-3.35 (2H, m), 4.48 (2H, s), 6.5 (1H, d, J = 8.4 Hz), 6.55-6.65 (1H, m), 6.81 (1H, d, J = 5.6 Hz), 6.9-7.0 (2H, m), 7.2-7.35 (3H, m), 7.51 (1H, d, J = 8.4 Hz), 9.94 (1H, brs) |
| 6 | 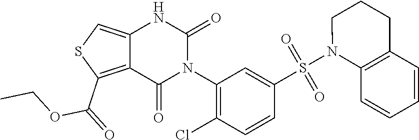 [SMILES: CCOC(=O)c1scc2[nH]c(=O)n(-c3cc(S(=O)(=O)N4CCCc5ccccc54)ccc3Cl)c(=O)c12] | (CDCl₃) 1.38 (3H, t, J = 7.1 Hz), 1.6- 1.75 (2H, m), 2.4-2.6 (2H, m), 3.7-3.85 (2H, m), 4.41 (2H, q, J = 7.1 Hz), 6.82 (1H, s), 7.0-7.15 (2H, m), 7.15-7.25 (1H, m), 7.47 (1H, dd, J = 8.5 Hz, 2.2 Hz), 7.53 (1H, d, J = 8.5 Hz), 7.6 (1H, d, J = 2.2 Hz), 7.7-7.8 (1H, m), 9.11 (1H, s) |
| 7 | 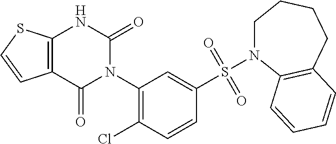 [SMILES: O=c1[nH]c2sccc2c(=O)n1-c1cc(S(=O)(=O)N2CCCCc3ccccc32)ccc1Cl] | (CDCl₃) 1.2-1.35 (2H, m), 1.75-1.9 (2H, m), 2.4-2.55 (2H, m), 3.2-4.3 (2H, br), 6.87 (1H, d, J = 5.3 Hz), 7.1-7.2 (3H, m), 7.25-7.35 (2H, m), 7.63 (1H, d, J = 8.5 Hz), 7.69 (1H, dd, J = 8.5 Hz, 1.9 Hz), 7.78 (1H, d, J = 1.9 Hz), 9.5-10.2 (1H, br) |
| 8 | 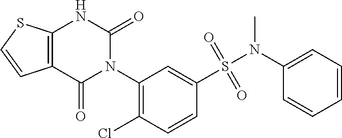 [SMILES: CN(c1ccccc1)S(=O)(=O)c1ccc(Cl)c(-n2c(=O)[nH]c3sccc3c2=O)c1] | (CDCl₃) 3.2 (3H, s), 6.87 (1H, d, J = 5.4 Hz), 7.1-7.15 (2H, m), 7.2-7.35 (4H, m), 7.54 (1H, dd, J = 8.4 Hz, 2.0 Hz), 7.6 (1H, d, J = 2.0 Hz), 7.64 (1H, d, J = 8.4 Hz), 9.5-11.0 (1H, br) |
| 9 | 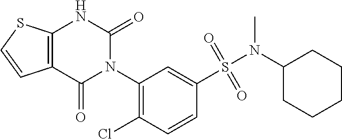 [SMILES: CN(C1CCCCC1)S(=O)(=O)c1ccc(Cl)c(-n2c(=O)[nH]c3sccc3c2=O)c1] | (CDCl₃) 0.9-1.8 (10H, m), 2.77 (3H, s), 3.7-3.8 (1H, m), 6.87 (1H, d, J = 5.7 Hz), 7.3 (1H, d, J = 5.7 Hz), 7.69 (1H, d, J = 8.1 Hz), 7.8-7.9 (2H, m), 9.5-10.5 (1H, br) |
| 10 | 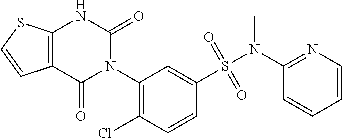 [SMILES: CN(c1ccccn1)S(=O)(=O)c1ccc(Cl)c(-n2c(=O)[nH]c3sccc3c2=O)c1] | (DMSO-d₆) 3.26 (3H, s), 7.19 (1H, d, J = 5.7 Hz), 7.22 (1H, d, J = 5.7 Hz), 7.25- 7.3 (1H, m), 7.45-7.55 (1H, m), 7.62 (1H, dd, J = 8.5 Hz, 2.1 Hz), 7.8-7.9 (2H, m), 8.04 (1H, d, J = 2.1 Hz), 8.3-8.4 (1H, m), 12.54 (1H, s) |
| 11 | 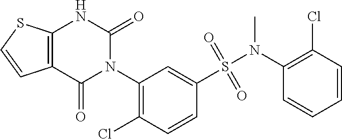 [SMILES: CN(c1ccccc1Cl)S(=O)(=O)c1ccc(Cl)c(-n2c(=O)[nH]c3sccc3c2=O)c1] | (DMSO-d₆) 3.16 (3H, s), 7.05 (1H, d, J = 7.8 Hz), 7.15-7.45 (4H, m), 7.5-7.6 (1H, m), 7.75-8.1 (3H, m), 12.55 (1H, s) |
| 12 | 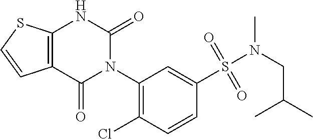 [SMILES: CC(C)CN(C)S(=O)(=O)c1ccc(Cl)c(-n2c(=O)[nH]c3sccc3c2=O)c1] | (DMSO-d₆) 0.8-0.9 (6H, m), 1.75-1.95 (1H, m), 2.6-2.8 (5H, m), 7.15-7.25 (2H, m), 7.8-7.95 (2H, m), 8.0-8.1 (1H, m), 12.53 (1H, s) |
| 13 | 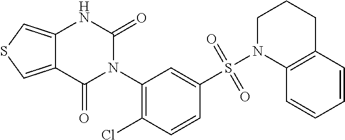 [SMILES: O=c1[nH]c2cscc2c(=O)n1-c1cc(S(=O)(=O)N2CCCc3ccccc32)ccc1Cl] | (DMSO-d₆) 1.6-1.75 (2H, m), 2.4-2.55 (2H, m), 3.7-3.85 (2H, m), 6.95 (1H, d, J = 3.1 Hz), 7.05-7.25 (3H, m), 7.52 (1H, dd, J = 8.5 Hz, 2.0 Hz), 7.56 (1H, d, J = 8.1 Hz), 7.78 (1H, d, J = 8.5 Hz), 8.04 (1H, d, J = 2.0 Hz), 8.52 (1H, d, J = 3.1 Hz), 11.53 (1H, s) |
| 14 | 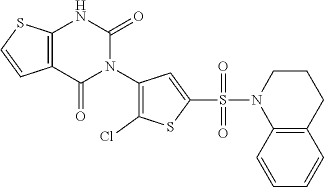 [SMILES: O=c1[nH]c2sccc2c(=O)n1-c1cc(S(=O)(=O)N2CCCc3ccccc32)sc1Cl] | (CDCl₃) 1.75-1.9 (2H, m), 2.55-2.7 (2H, m), 3.75-3.95 (2H, m), 6.9 (1H, d, J = 5.5 Hz), 7.05-7.25 (4H, m), 7.29 (1H, d, J = 5.5 Hz), 7.74 (1H, d, J = 8.2 Hz), 10.17 (1H, s) |
| 15 | 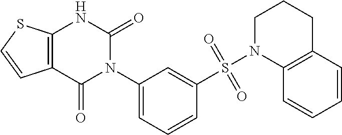 [SMILES: O=c1[nH]c2sccc2c(=O)n1-c1cccc(S(=O)(=O)N2CCCc3ccccc32)c1] | (DMSO-d₆) 1.6-1.7 (2H, m), 2.4-2.5 (2H, m), 3.7-3.8 (2H, m), 7.05-7.1 (2H, m), 7.1-7.25 (3H, m), 7.45-7.65 (4H, m), 7.74 (1H, d, J = 1.0 Hz), 12.37 (1H, s) |
| 16 | 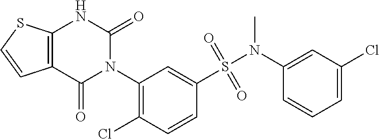 [SMILES: CN(c1cccc(Cl)c1)S(=O)(=O)c1ccc(Cl)c(-n2c(=O)[nH]c3sccc3c2=O)c1] | (DMSO-d₆) 3.18 (3H, s), 7.0-7.1 (1H, m), 7.15-7.25 (3H, m), 7.35-7.4 (2H, m), 7.6 (1H, dd, J = 8.5 Hz, 2.3 Hz), 7.87 (1H, d, J = 8.5 Hz), 7.96 (1H, d, J = 2.3 Hz), 12.54 (1H, s) |
| 17 | 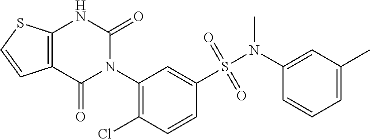 [SMILES: Cc1cccc(N(C)S(=O)(=O)c2ccc(Cl)c(-n3c(=O)[nH]c4sccc4c3=O)c2)c1] | (DMSO-d₆) 2.26 (3H, s), 3.15 (3H, s), 6.8-6.95 (2H, m), 7.05-7.25 (4H, m), 7.6 (1H, dd, J = 8.4 Hz, 2.3 Hz), 7.8-7.9 (2H, m), 12.54 (1H, s) |
| 18 | 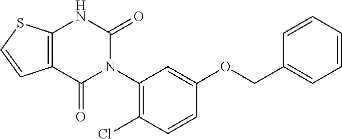 [SMILES: O=c1[nH]c2sccc2c(=O)n1-c1cc(OCc2ccccc2)ccc1Cl] | (DMSO-d₆) 5.1 (2H, s), 7.14 (1H, dd, J = 9.0 Hz, 2.9 Hz), 7.18 (1H, d, J = 5.6 Hz), 7.22 (1H, d, J = 5.6 Hz), 7.25 (1H, d, J = 2.9 Hz), 7.3-7.5 (5H, m), 7.53 (1H, d, J = 9.0 Hz), 12.48 (1H, s) |
| 19 | 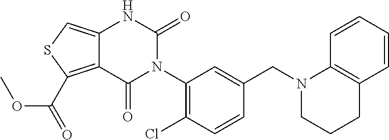 [SMILES: COC(=O)c1scc2[nH]c(=O)n(-c3cc(CN4CCCc5ccccc54)ccc3Cl)c(=O)c12] | (DMSO-d₆) 1.85-2.0 (2H, m), 2.7-2.8 (2H, m), 3.3-3.4 (2H, m), 3.81 (3H, s), 4.5 (2H, s), 6.4-6.5 (2H, m), 6.85-6.95 (2H, m), 7.2 (1H, s), 7.31 (1H, dd, J = 8.2 Hz, 2.1 Hz), 7.4 (1H, d, J = 2.1 Hz), 7.56 (1H, d, J = 8.2 Hz), 11.61 (1H, s) |
| 20 | 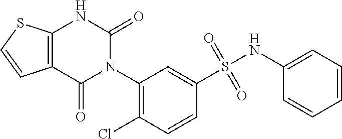 [SMILES: O=c1[nH]c2sccc2c(=O)n1-c1cc(S(=O)(=O)Nc2ccccc2)ccc1Cl] | (DMSO-d₆) 7.05-7.3 (7H, m), 7.75-7.9 (2H, m), 7.95-8.05 (1H, m), 10.48 (1H, s), 12.56 (1H, s) |
| 21 | 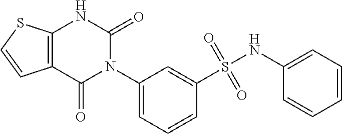 [SMILES: O=c1[nH]c2sccc2c(=O)n1-c1cccc(S(=O)(=O)Nc2ccccc2)c1] | (DMSO-d₆) 7.0-7.3 (7H, m), 7.55-7.7 (2H, m), 7.75-7.85 (2H, m), 10.4 (1H, s), 12.4 (1H, s), |
| 22 | 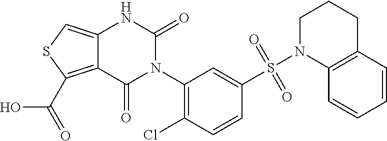 [SMILES: O=C(O)c1scc2[nH]c(=O)n(-c3cc(S(=O)(=O)N4CCCc5ccccc54)ccc3Cl)c(=O)c12] | (DMSO-d₆) 1.6-1.7 (2H, m), 2.4-2.55 (2H, m), 3.7-3.8 (2H, m), 7.05-7.25 (3H, m), 7.39 (1H, s), 7.55 (1H, d, J = 8.2 Hz), 7.6 (1H, dd, J = 8.5 Hz, 2.4 Hz), 7.83 (1H, d, J = 8.5 Hz), 8.09 (1H, d, J = 2.4 Hz), 12.03 (1H, s), 14.23 (1H, s) |
| 23 | 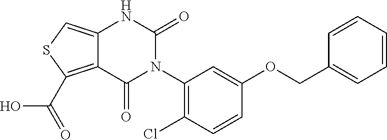 [SMILES: O=C(O)c1scc2[nH]c(=O)n(-c3cc(OCc4ccccc4)ccc3Cl)c(=O)c12] | (DMSO-d₆) 5.11 (2H, s), 7.19 (1H, dd, J = 9.0 Hz, 2.9 Hz), 7.3-7.45 (5H, m), 7.45-7.5 (2H, m), 7.57 (1H, d, J = 9.0 Hz), 12.04 (1H, s), 14.45 (1H, brs) |
| 24 | 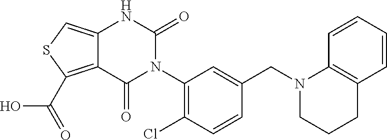 [SMILES: O=C(O)c1scc2[nH]c(=O)n(-c3cc(CN4CCCc5ccccc54)ccc3Cl)c(=O)c12] | (DMSO-d₆) 1.85-2.0 (2H, m), 2.7-2.8 (2H, m), 3.3-3.4 (2H, m), 4.52 (2H, s), 6.44 (1H, d, J = 8.2 Hz), 6.45-6.5 (1H, m), 6.85-6.95 (2H, m), 7.36 (1H, d, J = 8.2 Hz), 7.38 (1H, s), 7.48 (1H, d, J = 2.1 Hz), 7.61 (1H, d, J = 8.2 Hz), 12.0 (1H, s), 14.45 (1H, brs) |
| 25 | 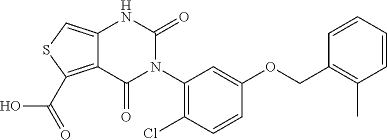 [SMILES: Cc1ccccc1COc1ccc(Cl)c(-n2c(=O)[nH]c3csc(C(=O)O)c3c2=O)c1] | (DMSO-d₆) 2.33 (3H, s), 5.09 (2H, s), 7.15-7.3 (4H, m), 7.35 (1H, d, J = 2.7 Hz), 7.41 (1H, s), 7.43 (1H, d, J = 7.7 Hz), 7.58 (1H, d, J = 9.0 Hz), 12.04 (1H, s), 14.44 (1H, s) |
| 26 | 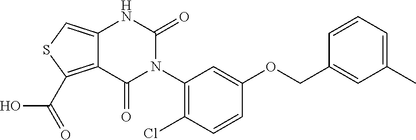 [SMILES: Cc1cccc(COc2ccc(Cl)c(-n3c(=O)[nH]c4csc(C(=O)O)c4c3=O)c2)c1] | (DMSO-d₆) 2.32 (3H, s), 5.07 (2H, s), 7.1-7.35 (6H, m), 7.41 (1H, s), 7.57 (1H, d, J = 8.7 Hz), 12.04 (1H, s), 14.45 (1H, brs) |
| 27 | 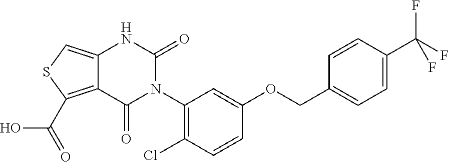 [SMILES: O=C(O)c1scc2[nH]c(=O)n(-c3cc(OCc4ccc(C(F)(F)F)cc4)ccc3Cl)c(=O)c12] | (DMSO-d₆) 5.24 (2H, s), 7.2 (1H, dd, J = 8.8 Hz, 3.0 Hz), 7.35 (1H, d, J = 3.0 Hz), 7.41 (1H, s), 7.59 (1H, d, J = 8.8 Hz), 7.69 (2H, d, J = 8.2 Hz), 7.78 (2H, d, J = 8.2 Hz), 12.04 (1H, s), 14.43 (1H, s) |
| 28 | 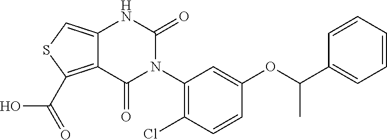 [SMILES: CC(Oc1ccc(Cl)c(-n2c(=O)[nH]c3csc(C(=O)O)c3c2=O)c1)c1ccccc1] | (DMSO-d₆) 1.5-1.6 (3H, m), 5.45-5.55 (1H, m), 7.0-7.1 (1H, m), 7.23 (1H, dd, J = 6.1 Hz, 2.9 Hz), 7.25-7.5 (7H, m), 11.95-12.1 (1H, m), 14.42 (1H, s) |
| 29 | 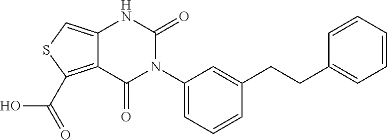 [SMILES: O=C(O)c1scc2[nH]c(=O)n(-c3cccc(CCc4ccccc4)c3)c(=O)c12] | (DMSO-d₆) 2.85-3.0 (4H, m), 7.15-7.35 (8H, m), 7.38 (1H, s), 7.41 (1H, t, J = 7.7 Hz), 11.92 (1H, s), 14.94 (1H, brs) |
| 30 | 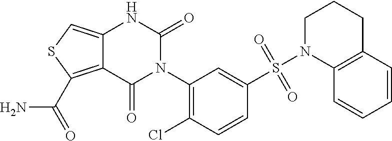 [SMILES: NC(=O)c1scc2[nH]c(=O)n(-c3cc(S(=O)(=O)N4CCCc5ccccc54)ccc3Cl)c(=O)c12] | (DMSO-d₆) 1.6-1.7 (2H, m), 2.4-2.55 (2H, m), 3.7-3.85 (2H, m), 7.0-7.2 (3H, m), 7.24 (1H, s), 7.56 (1H, d, J = 8.3 Hz), 7.59 (1H, dd, J = 8.5 Hz, 2.1 Hz), 7.81 (1H, d, J = 8.5 Hz), 8.05-8.15 (2H, m), 9.55 (1H, s), 11.81 (1H, s) |
| 31 | 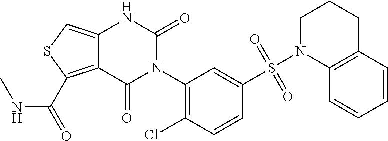 [SMILES: CNC(=O)c1scc2[nH]c(=O)n(-c3cc(S(=O)(=O)N4CCCc5ccccc54)ccc3Cl)c(=O)c12] | (CDCl₃) 1.65-1.75 (2H, m), 2.4-2.6 (2H, m), 2.99 (3H, d, J = 4.7 Hz), 3.7-3.9 (2H, m), 6.91 (1H, s), 6.95-7.15 (2H, m), 7.15-7.25 (1H, m), 7.5-7.65 (3H, m), 7.7-7.8 (1H, m), 8.91 (1H, s), 10.05- 10.15 (1H, m) |
| 32 | 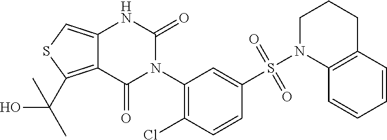 [SMILES: CC(C)(O)c1scc2[nH]c(=O)n(-c3cc(S(=O)(=O)N4CCCc5ccccc54)ccc3Cl)c(=O)c12] | (CDCl₃) 1.55 (6H, s), 1.6-1.75 (2H, m), 2.4-2.55 (2H, m), 3.7-3.9 (2H, m), 6.04 (1H, s), 6.49 (1H, s), 7.0-7.15 (2H, m), 7.15-7.25 (2H, m), 7.5-7.55 (1H, m), 7.55-7.6 (2H, m), 7.76 (1H, d, J = 8.3 Hz), 8.41 (1H, s) |
| 33 | 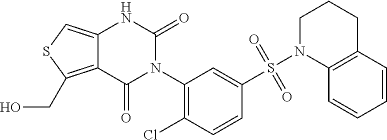 [SMILES: O=c1[nH]c2csc(CO)c2c(=O)n1-c1cc(S(=O)(=O)N2CCCc3ccccc32)ccc1Cl] | (DMSO-d₆) 1.6-1.75 (2H, m), 2.45-2.55 (2H, m), 3.7-3.85 (2H, m), 4.95-5.05 (2H, m), 5.99 (1H, t, J = 5.5 Hz), 6.73 (1H, s), 7.05-7.15 (2H, m), 7.15-7.25 (1H, m), 7.5-7.6 (2H, m), 7.78 (1H, d, J = 8.5 Hz), 7.97 (1H, d, J = 2.3 Hz), 11.41 (1H, s) |
| 34 | 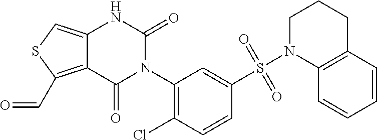 [SMILES: O=Cc1scc2[nH]c(=O)n(-c3cc(S(=O)(=O)N4CCCc5ccccc54)ccc3Cl)c(=O)c12] | (DMSO-d₆) 1.6-1.75 (2H, m), 2.45-2.55 (2H, m), 3.7-3.8 (2H, m), 7.05-7.15 (2H, m), 7.15-7.25 (1H, m), 7.5-7.6 (3H, m), 7.82 (1H, d, J = 8.5 Hz), 8.09 (1H, d, J = 2.1 Hz), 10.5-10.55 (1H, m), 11.88 (1H, s) |
| 35 | 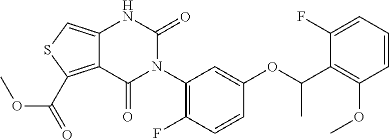 [SMILES: COC(=O)c1scc2[nH]c(=O)n(-c3cc(OC(C)c4c(F)cccc4OC)ccc3F)c(=O)c12] | (DMSO-d₆) 1.64 (3H, d, J = 6.5 Hz), 3.8- 3.9 (6H, m), 5.79 (1H, q, J = 6.5 Hz), 6.7-6.8 (1H, m), 6.85-6.95 (2H, m), 6.95- 7.05 (1H, m), 7.15-7.25 (2H, m), 7.25- 7.35 (1H, m), 11.63 (1H, s) |
| 36 | 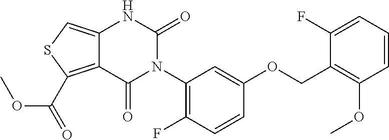 [SMILES: COC(=O)c1scc2[nH]c(=O)n(-c3cc(OCc4c(F)cccc4OC)ccc3F)c(=O)c12] | (DMSO-d₆) 3.83 (3H, s), 3.85 (3H, s), 4.99 (2H, s), 6.85-7.0 (2H, m), 7.1-7.2 (2H, m), 7.21 (1H, s), 7.25-7.35 (1H, m), 7.4-7.5 (1H, m), 11.68 (1H, s) |
| 37 | 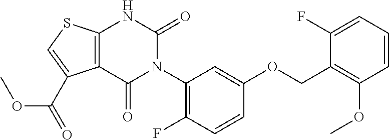 [SMILES: COC(=O)c1csc2[nH]c(=O)n(-c3cc(OCc4c(F)cccc4OC)ccc3F)c(=O)c12] | (DMSO-d₆) 3.75 (3H, s), 3.85 (3H, s), 4.99 (2H, s), 6.85-7.0 (2H, m), 7.1-7.2 (2H, m), 7.25-7.35 (1H, m), 7.4-7.5 (1H, m), 7.65 (1H, s), 12.64 (1H, s) |
| 38 | 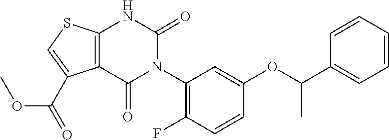 [SMILES: COC(=O)c1csc2[nH]c(=O)n(-c3cc(OC(C)c4ccccc4)ccc3F)c(=O)c12] | (DMSO-d₆) 1.54 (3H, d, J = 6.3 Hz), 3.8- 3.85 (3H, m), 5.46 (1H, q, J = 6.3 Hz), 6.9-7.0 (1H, m), 7.05-7.1 (1H, m), 7.15- 7.45 (7H, m), 11.63 (1H, s) |
| 39 | 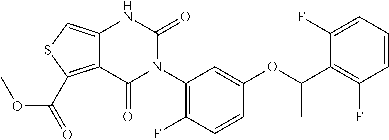 [SMILES: COC(=O)c1scc2[nH]c(=O)n(-c3cc(OC(C)c4c(F)cccc4F)ccc3F)c(=O)c12] | (DMSO-d₆) 1.7 (3H, d, J = 6.8 Hz), 3.8- 3.85 (3H, m), 5.7-5.8 (1H, m), 6.9-7.0 (1H, m), 7.05-7.15 (3H, m), 7.15-7.3 (2H, m), 7.35-7.5 (1H, m), 11.63 (1H, s) |
| 40 | 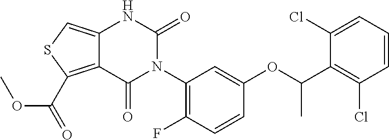 [SMILES: COC(=O)c1scc2[nH]c(=O)n(-c3cc(OC(C)c4c(Cl)cccc4Cl)ccc3F)c(=O)c12] | (DMSO-d₆) 1.71 (3H, d, J = 6.6 Hz), 3.82 (3H, s), 5.95-6.05 (1H, m), 6.8-6.9 (1H, m), 7.0-7.05 (1H, m), 7.15-7.3 (2H, m), 7.3-7.4 (1H, m), 7.45-7.5 (2H, m), 11.63 (1H, s) |
| 41 | 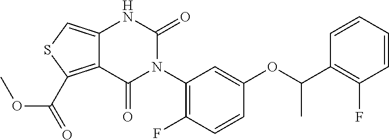 [SMILES: COC(=O)c1scc2[nH]c(=O)n(-c3cc(OC(C)c4ccccc4F)ccc3F)c(=O)c12] | (DMSO-d₆) 1.58 (3H, d, J = 6.3 Hz), 3.8- 3.85 (3H, m), 5.66 (1H, q, J = 6.3 Hz), 6.9-7.0 (1H, m), 7.05-7.15 (1H, m), 7.15-7.3 (4H, m), 7.3-7.4 (1H, m), 7.45- (1H, m), 11.63 (1H, s) |
| 42 | 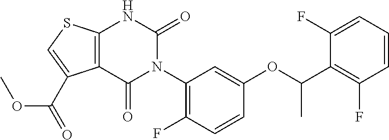 [SMILES: COC(=O)c1csc2[nH]c(=O)n(-c3cc(OC(C)c4c(F)cccc4F)ccc3F)c(=O)c12] | (DMSO-d₆) 1.69 (3H, d, J = 6.4 Hz), 3.7- 3.75 (3H, m), 5.7-5.8 (1H, m), 6.9-7.0 (1H, m), 7.05-7.15 (3H, m), 7.2-7.3 (1H, m), 7.35-7.5 (1H, m), 7.6-7.65 (1H, m), 12.59 (1H, s) |
| 43 | 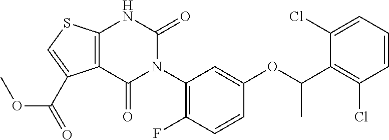 [SMILES: COC(=O)c1csc2[nH]c(=O)n(-c3cc(OC(C)c4c(Cl)cccc4Cl)ccc3F)c(=O)c12] | (DMSO-d₆) 1.71 (3H, d, J = 6.6 Hz), 3.74 (3H, s), 5.95-6.05 (1H, m), 6.8-6.9 (1H, m), 6.95-7.05 (1H, m), 7.2-7.3 (1H, m), 7.3-7.4 (1H, m), 7.4-7.5 (2H, m), 7.6-7.65 (1H, m), 12.59 (1H, s) |
| 44 | 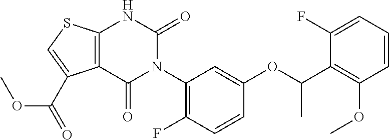 [SMILES: COC(=O)c1csc2[nH]c(=O)n(-c3cc(OC(C)c4c(F)cccc4OC)ccc3F)c(=O)c12] | (DMSO-d₆) 1.64 (3H, d, J = 6.7 Hz), 3.7- 3.8 (3H, m), 3.8-3.9 (3H, m), 5.75-5.85 (1H, m), 6.7-6.8 (1H, m), 6.85-6.95 (2H, m), 6.95-7.05 (1H, m), 7.15-7.25 (1H, m), 7.25-7.35 (1H, m), 7.63 (1H, s), 12.58 (1H, s) |
| 45 | 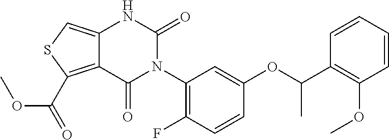 [SMILES: COC(=O)c1scc2[nH]c(=O)n(-c3cc(OC(C)c4ccccc4OC)ccc3F)c(=O)c12] | (DMSO-d₆) 1.51 (3H, d, J = 6.3 Hz), 3.8- 3.9 (6H, m), 5.6-5.7 (1H, m), 6.8-6.9 (1H, m), 6.9-7.0 (1H, m), 7.0-7.1 (2H, m), 7.15-7.3 (3H, m), 7.3-7.4 (1H, m), 11.6 (1H, s) |
| 46 | 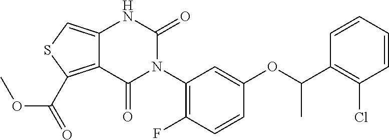 [SMILES: COC(=O)c1scc2[nH]c(=O)n(-c3cc(OC(C)c4ccccc4Cl)ccc3F)c(=O)c12] | (DMSO-d₆) 1.57 (3H, d, J = 6.3 Hz), 3.8- 3.85 3H, m), 5.6-5.7 (1H, m), 6.8-6.9 (1H, m), 7.05-7.1 (1H, m), 7.15-7.3 (2H, m), 7.3-7.4 m), 7.45-7.5 (1H, m), 7.5-7.6 (1H, m), 11.61 (1H, s) |
| 47 | 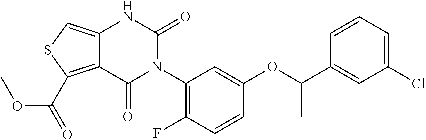 [SMILES: COC(=O)c1scc2[nH]c(=O)n(-c3cc(OC(C)c4cccc(Cl)c4)ccc3F)c(=O)c12] | (DMSO-d₆) 1.54 (3H, d, J = 6.2 Hz), 3.75-3.85 (3H, m), 5.45-5.55 (1H, m), 6.95-7.05 (1H, m), 7.05-7.15 (1H, m), 7.15-7.25 (2H, m), 7.3-7.45 (3H, m), 7.49 (1H, s), 11.61 (1H, s) |
| 48 | 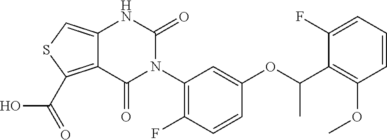 [SMILES: COc1cccc(F)c1C(C)Oc1ccc(F)c(-n2c(=O)[nH]c3csc(C(=O)O)c3c2=O)c1] | (DMSO-d₆) 1.65 (3H, d, J = 6.5 Hz), 3.8- 3.9 3H, m), 5.75-5.85 (1H, m), 6.7-6.8 (1H, m), 6.85-7.0 (2H, m), 7.05-7.1 (1H, m), 7.2-7.35 (2H, m), 7.37 (1H, d, J = 3.6 Hz), 12.01 (1H, s), 14.43 (1H, s) |
| 49 | 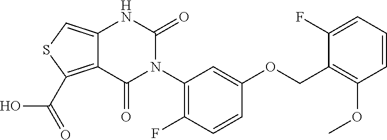 [SMILES: COc1cccc(F)c1COc1ccc(F)c(-n2c(=O)[nH]c3csc(C(=O)O)c3c2=O)c1] | (DMSO-d₆) 3.85 (3H, s), 5.0 (2H, s), 6.88 (1H, t, J = 8.7 Hz), 6.95 (1H, d, J = 8.7 Hz), 7.15-7.25 (2H, m), 7.3-7.5 (3H, m), 12.06 (1H, s), 14.43 (1H, s) |
| 50 | 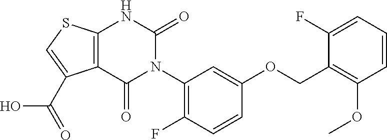 [SMILES: COc1cccc(F)c1COc1ccc(F)c(-n2c(=O)[nH]c3scc(C(=O)O)c3c2=O)c1] | (DMSO-d₆) 3.85 (3H, s), 5.0 (2H, s), 6.88 (1H, t, J = 8.6 Hz), 6.95 (1H, d, J = 8.6 Hz), 7.1-7.2 (1H, m), 7.2-7.25 (1H, m), 7.3-7.4 (1H, m), 7.4-7.5 (1H, m), 7.94 (1H, s), 13.04 (1H, s), 13.93 (1H, s) |
| 51 | 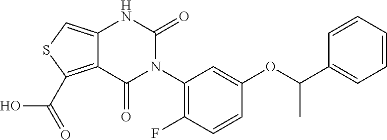 [SMILES: CC(Oc1ccc(F)c(-n2c(=O)[nH]c3csc(C(=O)O)c3c2=O)c1)c1ccccc1] | (DMSO-d₆) 1.5-1.6 (3H, m), 5.4-5.5 (1H, m), 6.95-7.05 (1H, m), 7.1-7.2 (1H, m), 7.2-7.4 (5H, m), 7.4-7.45 (2H, m), 11.95-12.05 (1H, m), 14.42 (1H, s) |
| 52 | 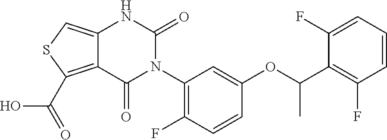 [SMILES: CC(Oc1ccc(F)c(-n2c(=O)[nH]c3csc(C(=O)O)c3c2=O)c1)c1c(F)cccc1F] | (DMSO-d₆) 1.7 (3H, d, J = 6.6 Hz), 5.76 (1H, q, J = 6.6 Hz), 7.0-7.2 (4H, m), 7.25-7.35 (1H, m), 7.35-7.5 (2H, m), 11.95-12.05 (1H, m), 14.42 (1H, s) |
| 53 | 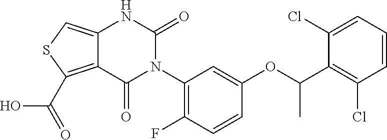 [SMILES: CC(Oc1ccc(F)c(-n2c(=O)[nH]c3csc(C(=O)O)c3c2=O)c1)c1c(Cl)cccc1Cl] | (DMSO-d₆) 1.65-1.75 (3H, m), 6.03 (1H, q, J = 6.6 Hz), 6.85-6.95 (1H, m), 7.05- 7.15 (1H, m), 7.25-7.4 (3H, m), 7.4-7.5 (2H, m), 11.95-12.05 (1H, m), 14.4 (1H, s) |
| 54 | 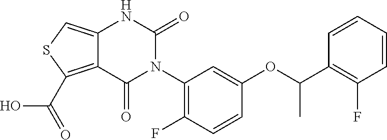 [SMILES: CC(Oc1ccc(F)c(-n2c(=O)[nH]c3csc(C(=O)O)c3c2=O)c1)c1ccccc1F] | (DMSO-d₆) 1.55-1.65 (3H, m), 5.65 (1H, q, J = 6.5 Hz), 7.0-7.05 (1H, m), 7.15- 7.25 (3H, m), 7.25-7.4 (3H, m), 7.45- 7.55 (1H, m), 11.95-12.05 (1H, m), 14.42 (1H, s) |
| 55 | 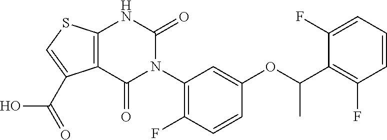 [SMILES: CC(Oc1ccc(F)c(-n2c(=O)[nH]c3scc(C(=O)O)c3c2=O)c1)c1c(F)cccc1F] | (DMSO-d₆) 1.65-1.75 (3H, m), 5.7-5.8 (1H, m), 6.95-7.2 (4H, m), 7.2-7.5 (2H, m), 7.93 (1H, d, J = 7.2 Hz),12.98 (1H, s), 13.85-14.0 (1H, m) |
| 56 | 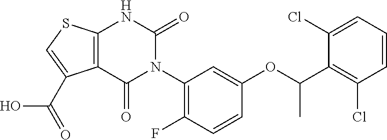 [SMILES: CC(Oc1ccc(F)c(-n2c(=O)[nH]c3scc(C(=O)O)c3c2=O)c1)c1c(Cl)cccc1Cl] | (DMSO-d₆) 1.72 (3H, d, J = 6.6 Hz), 6.0- 6.1 (1H, m), 6.85-6.95 (1H, m), 7.05-7.1 (1H, m), 7.25-7.4 (2H, m), 7.45-7.5 (2H, m), 7.92 (1H, d, J = 11.1 Hz), 12.98 (1H, brs), 13.85-14.0 (1H, m) |
| 57 | 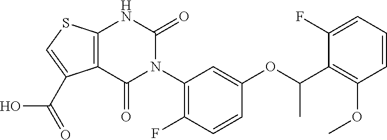 [SMILES: COc1cccc(F)c1C(C)Oc1ccc(F)c(-n2c(=O)[nH]c3scc(C(=O)O)c3c2=O)c1] | (DMSO-d₆) 1.65 (3H, d, J = 6.6 Hz), 3.8- 3.9 (3H, m), 5.79 (1H, q, J = 6.6 Hz), 6.7-6.8 (1H, m), 6.85-6.95 (2H, m), 7.0- 7.1 (1H, m), 7.2-7.35 (2H, m), 7.85-8.0 (1H, m), 12.98 (1H, brs), 13.85-14.05 (1H, m) |
| 58 | 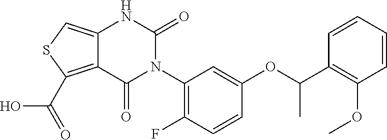 [SMILES: COc1ccccc1C(C)Oc1ccc(F)c(-n2c(=O)[nH]c3csc(C(=O)O)c3c2=O)c1] | (DMSO-d₆) 1.52 (3H, d, J = 6.3 Hz), 3.8- 3.9 (3H, m), 5.64 (1H, q, J = 6.3 Hz), 6.85-7.0 (2H, m), 7.0-7.15 (2H, m), 7.2- 7.4 (4H, m), 11.95-12.0 (1H, m), 14.41 (1H, s) |
| 59 | 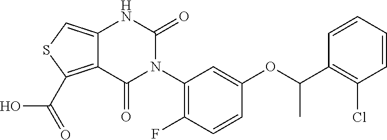 [SMILES: CC(Oc1ccc(F)c(-n2c(=O)[nH]c3csc(C(=O)O)c3c2=O)c1)c1ccccc1Cl] | (DMSO-d₆) 1.45-1.65 (3H, m), 5.55-5.8 (1H, m), 6.8-7.7 (8H, m), 11.98 (1H, s), 14.39 (1H, s) |
| 60 | 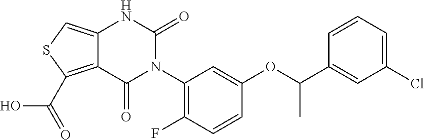 [SMILES: CC(Oc1ccc(F)c(-n2c(=O)[nH]c3csc(C(=O)O)c3c2=O)c1)c1cccc(Cl)c1] | (DMSO-d₆) 1.45-1.65 (3H, m), 5.4-5.6 (1H, m), 6.95-7.6 (8H, m), 11.99 (1H, s), 14.39 (1H, s) |
| 61 | 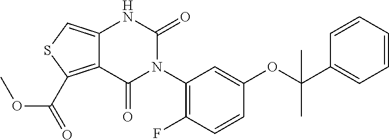 [SMILES: COC(=O)c1scc2[nH]c(=O)n(-c3cc(OC(C)(C)c4ccccc4)ccc3F)c(=O)c12] | (DMSO-d₆) 1.56 (3H, s), 1.57 (3H, s), 3.82 (3H, s), 7.15-7.45 (8H, m), 7.8-7.9 (1H, m), 11.68 (1H, s) |
| 62 | 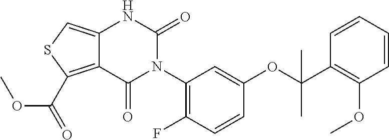 [SMILES: COC(=O)c1scc2[nH]c(=O)n(-c3cc(OC(C)(C)c4ccccc4OC)ccc3F)c(=O)c12] | (DMSO-d₆) 1.5-1.6 (6H, m), 3.34 (3H, s), 3.82 (3H, s), 6.84 (1H, d, J = 8.2 Hz), 7.0-7.1 (1H, m), 7.1-7.3 (3H, m), 7.3-7.4 (1H, m), 7.5-7.6 (1H, m), 7.8-7.9 (1H, m), 11.63 (1H, s) |
| 63 | 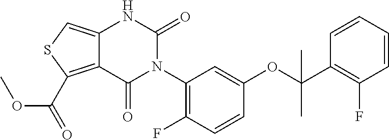 [SMILES: COC(=O)c1scc2[nH]c(=O)n(-c3cc(OC(C)(C)c4ccccc4F)ccc3F)c(=O)c12] | (DMSO-d₆) 1.59 (3H, s), 1.6 (3H, s), 3.82 (3H, s), 7.05-7.15 (1H, m), 7.19 (1H, s), 7.2-7.4 (3H, m), 7.4-7.5 (1H, m), 7.65-7.75 (1H, m), 7.9-8.0 (1H, m), 11.7 (1H, s) |
| 64 | 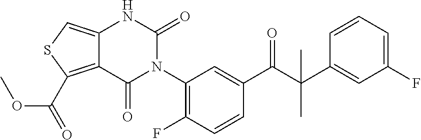 [SMILES: COC(=O)c1scc2[nH]c(=O)n(-c3cc(C(=O)C(C)(C)c4cccc(F)c4)ccc3F)c(=O)c12] | (DMSO-d₆) 1.57 (3H, s), 1.58 (3H, s), 3.82 (3H, s), 7.06 (1H, d, J = 8.4 Hz), 7.1-7.25 (3H, m), 7.25-7.35 (1H, m), 7.35-7.45 (2H, m), 7.8-7.9 (1H, m), 11.68 (1H, s) |
| 65 | 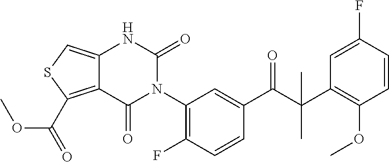 [SMILES: COC(=O)c1scc2[nH]c(=O)n(-c3cc(C(=O)C(C)(C)c4cc(F)ccc4OC)ccc3F)c(=O)c12] | (DMSO-d₆) 1.54 (6H, s), 3.31 (3H, s), 3.82 (3H, s), 6.8-6.9 (1H, m), 7.0-7.1 (1H, m), 7.15-7.25 (2H, m), 7.3-7.45 (2H, m), 7.8-7.9 (1H, m), 11.63 (1H, s) |
| 66 | 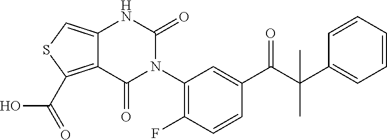 [SMILES: CC(C)(C(=O)c1ccc(F)c(-n2c(=O)[nH]c3csc(C(=O)O)c3c2=O)c1)c1ccccc1] | (DMSO-d₆) 1.55-1.6 (6H, m), 7.25-7.45 (8H, m), 7.9-8.0 (1H, m), 12.0 (1H, s), 14.29 (1H, s) |
| 67 | 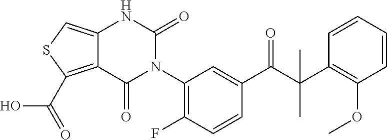 [SMILES: COc1ccccc1C(C)(C)C(=O)c1ccc(F)c(-n2c(=O)[nH]c3csc(C(=O)O)c3c2=O)c1] | (DMSO-d₆) 1.55 (3H, s), 1.56 (3H, s), 3.33 (3H, s), 6.84 (1H, d, J = 8.2 Hz), 7.0-7.1 (1H, m), 7.15-7.3 (2H, m), 7.37 (1H, s), 7.4-7.5 (1H, m), 7.5-7.55 (1H, m), 7.9-7.95 (1H, m), 11.99 (1H, s), 14.35 (1H, s) |
| 68 | 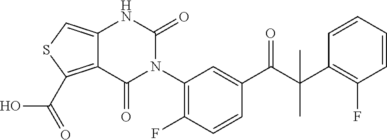 [SMILES: CC(C)(C(=O)c1ccc(F)c(-n2c(=O)[nH]c3csc(C(=O)O)c3c2=O)c1)c1ccccc1F] | (DMSO-d₆) 1.59 (3H, s), 1.62 (3H, s), 7.0-7.1 (1H, m), 7.25-7.4 (4H, m), 7.5- 7.6 (1H, m), 7.65-7.75 (1H, m), 7.95- 8.05 (1H, m), 12.01 (1H, s), 14.29 (1H, s) |
| 69 | 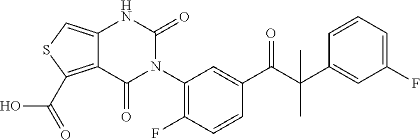 [SMILES: CC(C)(C(=O)c1ccc(F)c(-n2c(=O)[nH]c3csc(C(=O)O)c3c2=O)c1)c1cccc(F)c1] | (DMSO-d₆) 1.58 (3H, s), 1.59 (3H, s), 7.0-7.25 (3H, m), 7.3-7.45 (3H, m), 7.45-7.55 (1H, m), 7.9-7.95 (1H, m), 12.02 (1H, s), 14.29 (1H, s) |
| 70 | 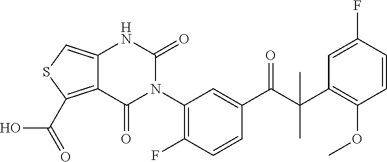 [SMILES: COc1ccc(F)cc1C(C)(C)C(=O)c1ccc(F)c(-n2c(=O)[nH]c3csc(C(=O)O)c3c2=O)c1] | (DMSO-d₆) 1.54 (3H, s), 1.56 (3H, s), 3.31 (3H, s), 6.8-6.9 (1H, m), 7.0-7.1 (1H, m), 7.26 (1H, t, J = 9.2 Mz), 7.3-7.4 (2H, m), 7.5-7.6 (1H, m), 7.85-7.95 (1H, m), 11.99 (1H, s), 14.36 (1H, s) |
| 71 | 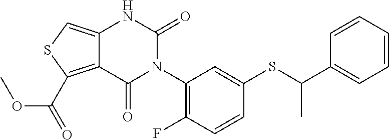 [SMILES: COC(=O)c1scc2[nH]c(=O)n(-c3cc(SC(C)c4ccccc4)ccc3F)c(=O)c12] | (DMSO-d₆) 1.55-1.6 (3H, m), 3.82 (3H, s), 4.6-4.7 (1H, m), 7.1-7.2 (2H, m), 7.2- 7.45 (8H, m), 11.49 (1H, s) |
| 72 | 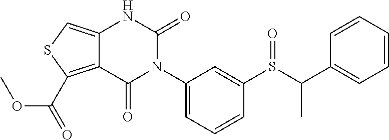 [SMILES: COC(=O)c1scc2[nH]c(=O)n(-c3cccc(S(=O)C(C)c4ccccc4)c3)c(=O)c12] | (DMSO-d₆) 1.36 (3H, d, J = 7.2 Hz), 3.82 (3H, s), 4.05-4.15 (1H, m), 7.15- 7.65 (10H, m), 11.54 (1H, s) |
| 73 | 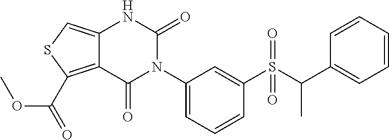 [SMILES: COC(=O)c1scc2[nH]c(=O)n(-c3cccc(S(=O)(=O)C(C)c4ccccc4)c3)c(=O)c12] | (DMSO-d₆) 1.56 (3H, d, J = 7.1 Hz), 3.83 (3H, s), 4.71 (1H, q, J = 7.1 Hz), 7.18 (1H, s), 7.2-7.35 (5H, m), 7.6-7.7 (3H, m), 7.75-7.8 (1H, m), 11.56 (1H, s) |
| 74 | 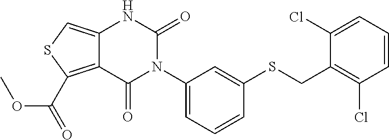 [SMILES: COC(=O)c1scc2[nH]c(=O)n(-c3cccc(SCc4c(Cl)cccc4Cl)c3)c(=O)c12] | (DMSO-d₆) 3.82 (3H, s), 4.44 (2H, s), 7.1-7.25 (2H, m), 7.3-7.4 (1H, m), 7.4- 7.45 (3H, m), 7.45-7.55 (2H, m), 11.5 (1H, s) |
| 75 | 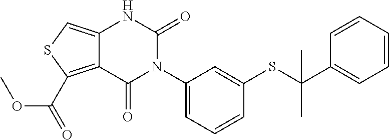 [SMILES: COC(=O)c1scc2[nH]c(=O)n(-c3cccc(SC(C)(C)c4ccccc4)c3)c(=O)c12] | (DMSO-d₆) 1.64 (6H, s), 3.82 (3H, s), 7.05-7.25 (4H, m), 7.25-7.4 (4H, m), 7.4-7.5 (2H, m), 11.45 (1H, s) |
| 76 | 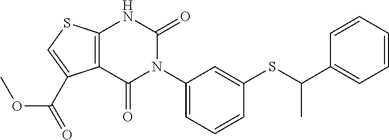 [SMILES: COC(=O)c1csc2[nH]c(=O)n(-c3cccc(SC(C)c4ccccc4)c3)c(=O)c12] | (DMSO-d₆) 1.57 (3H, d, J = 6.9 Hz), 3.74 (3H, s), 4.66 (1H, q, J = 6.9 Hz), 7.1-7.15 (1H, m), 7.2-7.45 (8H, m), 7.59 (1H, s), 12.44 (1H, s) |
| 77 | 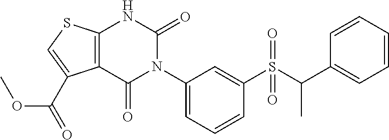 [SMILES: COC(=O)c1csc2[nH]c(=O)n(-c3cccc(S(=O)(=O)C(C)c4ccccc4)c3)c(=O)c12] | (DMSO-d₆) 1.56 (3H, d, J = 6.8 Hz), 3.76 (3H, s), 4.65-4.75 (1H, m), 7.2-7.35 (5H, m), 7.6-7.75 (4H, m), 7.78 (1H, s), 12.52 (1H, s) |
| 78 | 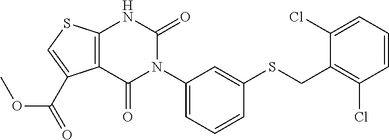 [SMILES: COC(=O)c1csc2[nH]c(=O)n(-c3cccc(SCc4c(Cl)cccc4Cl)c3)c(=O)c12] | (DMSO-d₆) 3.74 (3H, s), 4.44 (2H, s), 7.15-7.25 (1H, m), 7.3-7.55 (6H, m), 7.59 (1H, s), 12.45 (1H, s) |
| 79 | 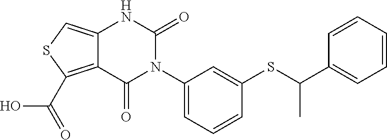 [SMILES: CC(Sc1cccc(-n2c(=O)[nH]c3csc(C(=O)O)c3c2=O)c1)c1ccccc1] | (DMSO-d₆) 1.58 (3H, d, J = 6.9 Hz), 4.65 (1H, q, J = 6.9 Hz), 7.2-7.5 (10H, m), 11.93 (1H, s), 14.87 (1H, s) |
| 80 | 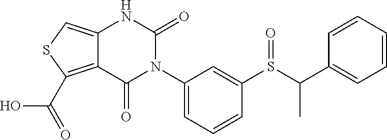 [SMILES: CC(c1ccccc1)S(=O)c1cccc(-n2c(=O)[nH]c3csc(C(=O)O)c3c2=O)c1] | (DMSO-d₆) 1.39 (3H, d, J = 7.3 Hz), 4.05-4.15 (1H, m), 7.2-7.8 (10H, m), 11.95 (1H, s), 14.8 (1H, s) |
| 81 | 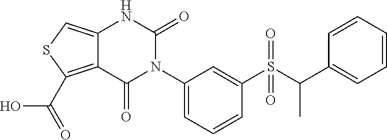 [SMILES: CC(c1ccccc1)S(=O)(=O)c1cccc(-n2c(=O)[nH]c3csc(C(=O)O)c3c2=O)c1] | (DMSO-d₆) 1.59 (3H, d, J = 7.7 Hz), 4.72 (1H, q, J = 7.7 Hz), 7.2-7.35 (5H, m), 7.39 (1H, s), 7.65-7.9 (4H, m), 11.96 (1H, s), 14.73 (1H, s) |
| 82 | 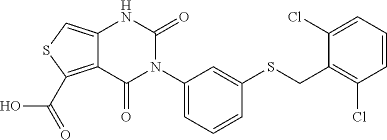 [SMILES: O=C(O)c1scc2[nH]c(=O)n(-c3cccc(SCc4c(Cl)cccc4Cl)c3)c(=O)c12] | (DMSO-d₆) 4.44 (2H, s), 7.25-7.45 (3H, m), 7.45-7.55 (5H, m), 11.93 (1H, s), 14.87 (1H, s) |
| 83 | 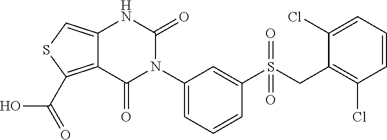 [SMILES: O=C(O)c1scc2[nH]c(=O)n(-c3cccc(S(=O)(=O)Cc4c(Cl)cccc4Cl)c3)c(=O)c12] | (DMSO-d₆) 4.94 (2H, s), 7.35-7.55 (4H, m), 7.75-7.85 (3H, m), 7.95-8.0 (1H, m), 11.96 (1H, s), 14.75 (1H, s) |
| 84 | 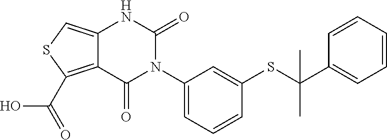 [SMILES: CC(C)(Sc1cccc(-n2c(=O)[nH]c3csc(C(=O)O)c3c2=O)c1)c1ccccc1] | (DMSO-d₆) 1.65 (6H, s), 7.1-7.5 (10H, m), 11.88 (1H, s), 14.84 (1H, s) |
| 85 | 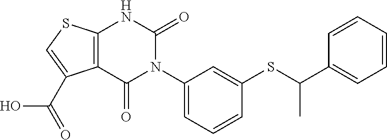 [SMILES: CC(Sc1cccc(-n2c(=O)[nH]c3scc(C(=O)O)c3c2=O)c1)c1ccccc1] | (DMSO-d₆) 1.58 (3H, d, J = 7.0 Hz), 4.66 (1H, q, J = 7.0 Hz), 7.15-7.45 (9H, m), 8.0 (1H, s), 12.94 (1H, s), 14.43 (1H, s) |
| 86 | 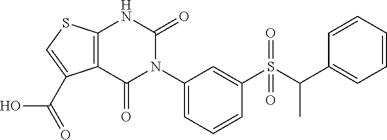 [SMILES: CC(c1ccccc1)S(=O)(=O)c1cccc(-n2c(=O)[nH]c3scc(C(=O)O)c3c2=O)c1] | (DMSO-d₆) 1.58 (3H, d, J = 7.0 Hz), 4.72 (1H, q, J = 7.0 Hz), 7.2-7.35 (5H, m), 7.65-7.8 (3H, m), 7.84 (1H, s), 7.98 (1H, s), 12.96 (1H, s), 14.26 (1H, s) |
| 87 | 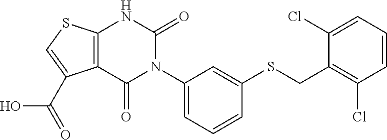 [SMILES: O=C(O)c1csc2[nH]c(=O)n(-c3cccc(SCc4c(Cl)cccc4Cl)c3)c(=O)c12] | (DMSO-d₆) 4.45 (2H, s), 7.25-7.3 (1H, m), 7.3-7.4 (1H, m), 7.45-7.55 (5H, m), 8.0 (1H, s), 12.94 (1H, s), 14.42 (1H, s) |
| 88 | 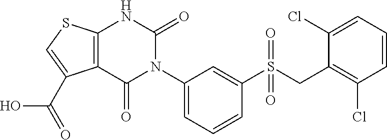 [SMILES: O=C(O)c1csc2[nH]c(=O)n(-c3cccc(S(=O)(=O)Cc4c(Cl)cccc4Cl)c3)c(=O)c12] | (DMSO-d₆) 4.94 (2H, s), 7.35-7.45 (1H, m), 7.45-7.55 (2H, m), 7.7-7.85 (3H, m), 7.95-8.0 (2H, m), 12.96 (1H, s), 14.3 (1H, s) |
| 89 | 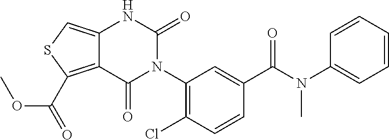 [SMILES: COC(=O)c1scc2[nH]c(=O)n(-c3cc(C(=O)N(C)c4ccccc4)ccc3Cl)c(=O)c12] | (DMSO-d₆) 3.37 (3H, s), 3.83 (3H, s), 7.15-7.25 (5H, m), 7.25-7.35 (2H, m), 7.44 (1H, d, J = 8.1 Hz), 7.56 (1H, d, J = 1.9 Hz), 11.63 (1H, s) |
| 90 | 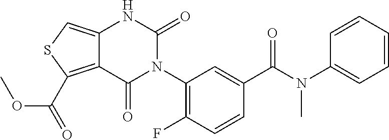 [SMILES: COC(=O)c1scc2[nH]c(=O)n(-c3cc(C(=O)N(C)c4ccccc4)ccc3F)c(=O)c12] | (DMSO-d₆) 3.37 (3H, s), 3.83 (3H, s), 7.15-7.35 (8H, m), 7.53 (1H, dd, J = 7.3 Hz, 1.9 Hz), 11.63 (1H, s) |
| 91 | 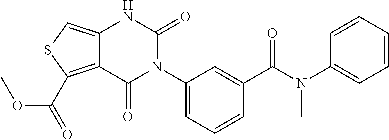 [SMILES: COC(=O)c1scc2[nH]c(=O)n(-c3cccc(C(=O)N(C)c4ccccc4)c3)c(=O)c12] | (DMSO-d₆) 3.37 (3H, s), 3.82 (3H, s), 7.1-7.4 10H, m), 11.46 (1H, s) |
| 92 | 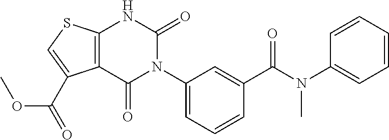 [SMILES: COC(=O)c1csc2[nH]c(=O)n(-c3cccc(C(=O)N(C)c4ccccc4)c3)c(=O)c12] | (DMSO-d₆) 3.37 (3H, s), 3.74 (3H, s), 7.1-7.4 (9H, m), 7.58 (1H, s), 12.42 (1H, s) |
| 93 | 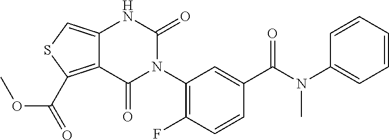 [SMILES: COC(=O)c1scc2[nH]c(=O)n(-c3cc(C(=O)N(C)c4ccccc4)ccc3F)c(=O)c12] | (DMSO-d₆) 3.38 (3H, s), 7.15-7.4 (8H, m), 7.5-7.6 (1H, m), 11.96 (1H, s), 14.34 (1H, s) |
| 94 | 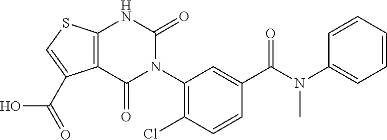 [SMILES: CN(C(=O)c1ccc(Cl)c(-n2c(=O)[nH]c3scc(C(=O)O)c3c2=O)c1)c1ccccc1] | (DMSO-d₆) 3.37 (3H, s), 7.15-7.4 (7H, m), 7.44 (1H, d, J = 8.7 Hz), 7.72 (1H, s) |
| 95 | 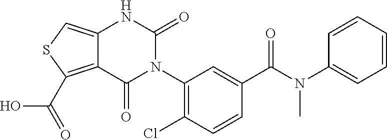 [SMILES: CN(C(=O)c1ccc(Cl)c(-n2c(=O)[nH]c3csc(C(=O)O)c3c2=O)c1)c1ccccc1] | (DMSO-d₆) 3.38 (3H, s), 7.15-7.25 (3H, m), 7.25-7.35 (3H, m), 7.38 (1H, s), 7.5 (1H, d, J = 7.5 Hz), 7.58 (1H, d, J = 1.9 Hz), 11.98 (1H, s), 14.33 (1H, s) |
| 96 | 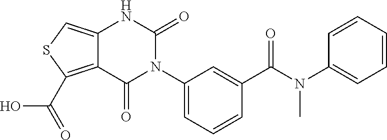 [SMILES: CN(C(=O)c1cccc(-n2c(=O)[nH]c3csc(C(=O)O)c3c2=O)c1)c1ccccc1] | (DMSO-d₆) 3.37 (3H, s), 7.15-7.4 (10H, m), 11.89 (1H, s), 14.81 (1H, s) |
| 97 | 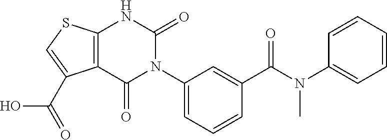 [SMILES: CN(C(=O)c1cccc(-n2c(=O)[nH]c3scc(C(=O)O)c3c2=O)c1)c1ccccc1] | (DMSO-d₆) 3.37 (3H, s), 7.15-7.4 (9H, m), 7.99 (1H, s), 12.9 (1H, s), 14.37 (1H, s) |
| 98 | 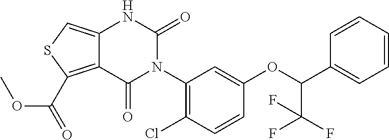 [SMILES: COC(=O)c1scc2[nH]c(=O)n(-c3cc(OC(c4ccccc4)C(F)(F)F)ccc3Cl)c(=O)c12] | (DMSO-d₆) 3.75-3.85 (3H, m), 6.3-6.4 (1H, m), 7.05-7.15 (1H, m), 7.2 (1H, s), 7.32 (1H, dd, J = 7.6 Hz, 2.9 Hz), 7.45- 7.55 (4H, m), 7.55-7.65 (2H, m), 11.66 (1H, s) |
| 99 | 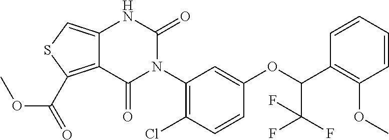 | (DMSO-d₆) 3.75-3.95 (6H, m), 6.2-6.35 (1H, m), 6.9-7.0 (1H, m), 7.0-7.1 (1H, m), 7.17 (1H, d, J = 8.0 Hz), 7.21 (1H, s), 7.31 (1H, dd, J = 6.4 Hz, 3.0 Hz), 7.4-7.55 (3H, m), 11.64 (1H, s) |
| 100 | 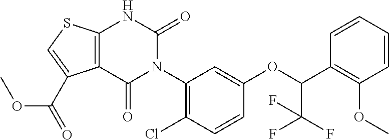 | (DMSO-d₆) 3.7-3.75 (3H, m), 3.85-3.95 (3H, m), 6.2-6.35 (1H, m), 6.9-7.0 (1H, m), 7.04 (1H, t, J = 7.6 Hz), 7.15-7.2 (1H, m), 7.25-7.35 (1H, m), 7.4-7.55 (3H, m), 7.64 (1H, s), 12.6 (1H, s) |
| 101 | 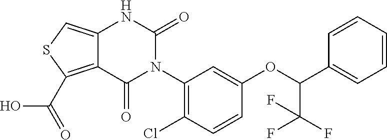 [SMILES: O=C(O)c1scc2[nH]c(=O)n(-c3cc(OC(c4ccccc4)C(F)(F)F)ccc3Cl)c(=O)c12] | (DMSO-d₆) 6.3-6.4 (1H, m), 7.1-7.2 (1H, m), 7.35-7.4 (2H, m), 7.45-7.55 (3H, m), 7.55-7.65 (3H, m), 11.95-12.1 (1H, m), 14.32 (1H, s) |
| 102 | 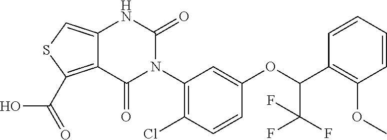 [SMILES: COc1ccccc1C(Oc1ccc(Cl)c(-n2c(=O)[nH]c3csc(C(=O)O)c3c2=O)c1)C(F)(F)F] | (DMSO-d₆) 3.85-3.95 (3H, m), 6.25-6.35 (1H, m), 6.95-7.1 (2H, m), 7.15-7.2 (1H, m), 7.35-7.5 (4H, m), 7.56 (1H, d, J = 8.9 Hz), 12.0-12.1 (1H, m), 14.34 (1H, s) |
| 103 | 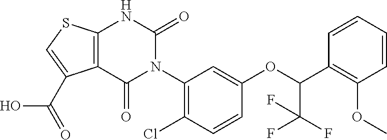 [SMILES: COc1ccccc1C(Oc1ccc(Cl)c(-n2c(=O)[nH]c3scc(C(=O)O)c3c2=O)c1)C(F)(F)F] | (DMSO-d₆) 3.85-3.95 (3H, m), 6.25-6.35 (1H, m), 6.95-7.1 (2H, m), 7.15-7.2 (1H, m), 7.3-7.4 (1H, m), 7.4-7.5 (2H, m), 7.55 (1H, d, J = 9.0 Hz), 7.92 (1H, d, J = 4.5 Hz), 13.0 (1H, brs), 13.8-13.95 (1H, m) |
| 104 | 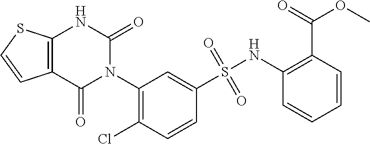 [SMILES: COC(=O)c1ccccc1NS(=O)(=O)c1ccc(Cl)c(-n2c(=O)[nH]c3sccc3c2=O)c1] | (CDCl3) 3.89 (3H, s), 6.84 (1H, d, J = 5.8 Hz), 7.05-7.1 (1H, m), 7.28 (1H, d, J = 5.8 Hz), 7.45-7.55 (1H, m), 7.59 (1H, d, J = 8.5 Hz), 7.65-7.75 (1H, m), 7.8- 7.85 (1H, m), 7.85-8.0 (2H, m), 10.06 (1H, s), 10.75 (1H, s) |
| 105 | 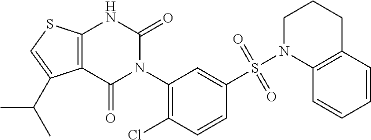 [SMILES: CC(C)c1csc2[nH]c(=O)n(-c3cc(S(=O)(=O)N4CCCc5ccccc54)ccc3Cl)c(=O)c12] | (CDCl3) 1.6-1.75 (2H, m), 2.45-2.55 (2H, m), 3.45-3.6 (1H, m), 3.7-3.85 (2H, m), 6.45 (1H, d, J = 0.5 Hz), 7.01 (1H, d, J = 7.0 Hz), 7.05-7.25 (2H, m), 7.5-7.6 (3H, m), 7.76 (1H, d, J = 7.7 Hz), 10.68 (1H, s) |
| 106 | 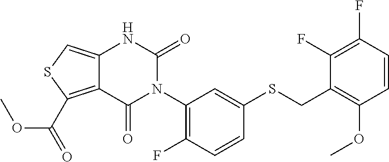 [SMILES: COC(=O)c1scc2[nH]c(=O)n(-c3cc(SCc4c(OC)ccc(F)c4F)ccc3F)c(=O)c12] | (DMSO-d6) 3.74 (3H, s), 3.83 (3H, s), 4.15-4.2 (2H, m), 6.75-6.85 (1H, m), 7.21 (1H, s), 7.25-7.4 (2H, m) 7.4-7.5 (1H, m), 7.5-7.55 (1H, m), 11.65 (1H, s) |
| 107 | 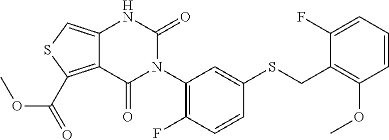 [SMILES: COC(=O)c1scc2[nH]c(=O)n(-c3cc(SCc4c(F)cccc4OC)ccc3F)c(=O)c12] | (DMSO-d6) 3.77 (3H, s), 3.83 (3H, s), 4.15 (2H, s), 6.75-6.9 (2H, m), 7.15-7.55 (5H, m), 11.65 (1H, s) |
| 108 | 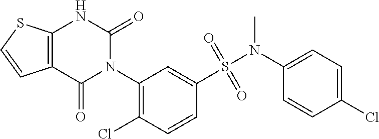 [SMILES: CN(c1ccc(Cl)cc1)S(=O)(=O)c1ccc(Cl)c(-n2c(=O)[nH]c3sccc3c2=O)c1] | (CDCl3) 3.16 (3H, s), 6.88 (1H, d, J = 5.8 Hz), 7.0-7.1 (2H, m), 7.25-7.35 (3H, m), 7.5-7.6 (2H, m), 7.6-7.7 (1H, m), 9.5-11.0 (1H, br) |
| 109 | 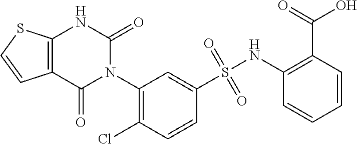 [SMILES: O=C(O)c1ccccc1NS(=O)(=O)c1ccc(Cl)c(-n2c(=O)[nH]c3sccc3c2=O)c1] | (DMSO-d6) 7.1-7.25 (3H, m), 7.44 (1H, d, J = 8.5 Hz), 7.5-7.6 (1H, m), 7.87 (1H, d, J = 8.5 Hz), 7.9-8.0 (2H, m), 8.18 (1H, J = 2.2 Hz), 11.0-12.0 (1H, br), 12.52 (1H, s) |
| 110 | 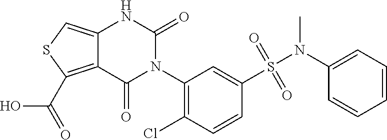 [SMILES: CN(c1ccccc1)S(=O)(=O)c1ccc(Cl)c(-n2c(=O)[nH]c3csc(C(=O)O)c3c2=O)c1] | (CDCl3) 3.22 (3H, s), 7.05-7.15 (3H, m), 7.25-7.35 (3H, m), 7.45-7.55 (1H, m), 7.71 (1H, s), 9.22 (1H, s), 14.14 (1H, s) |
| 111 | 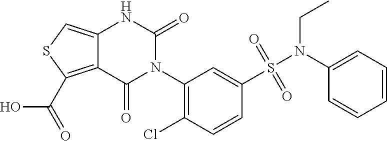 [SMILES: CCN(c1ccccc1)S(=O)(=O)c1ccc(Cl)c(-n2c(=O)[nH]c3csc(C(=O)O)c3c2=O)c1] | (CDCl3) 1.11 (3H, t, J = 7.1 Hz), 3.5-3.6 (1H, m), 3.65-3.8 (1H, m), 7.05-7.1 (2H, m), 7.13 (1H, s), 7.25-7.35 (3H, m), 7.55 (1H, d, J = 2.2 Hz), 7.7 (1H, d, J = 8.4 Hz), 7.77 (1H, dd, J = 8.4 Hz, 2.2 Hz), 9.22 (1H, s), 14.17 (1H, s) |
| 112 | 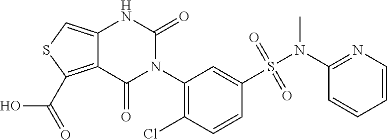 [SMILES: CN(c1ccccn1)S(=O)(=O)c1ccc(Cl)c(-n2c(=O)[nH]c3csc(C(=O)O)c3c2=O)c1] | (CDCl3) 3.33 (3H, s), 7.1 (1H, s), 7.15- 7.2 (1H, m), 7.57 (1H, d, J = 8.2 Hz), 7.6-7.75 (4H, m), 8.3-8.4 (1H, m), 9.05 (1H, s), 14.09 (1H, s) |
| 113 | 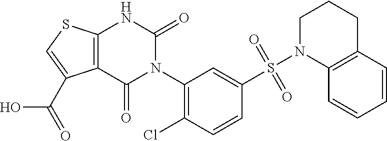 [SMILES: O=C(O)c1csc2[nH]c(=O)n(-c3cc(S(=O)(=O)N4CCCc5ccccc54)ccc3Cl)c(=O)c12] | (DMSO-d6) 1.6-1.75 (2H, m), 2.45-2.55 (2H, m), 3.7-3.85 (2H, m), 6.9-7.0 (1H, m), 7.05-7.25 (3H, m), 7.5-7.6 (2H, m), 7.78 (1H, d, J = 8.5 Hz), 8.0-8.05 (1H, m), 8.5-8.55 (1H, m), 11.52 (1H, s) |
| 114 | 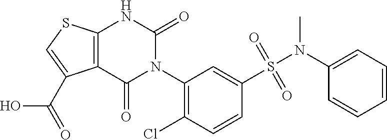 [SMILES: CN(c1ccccc1)S(=O)(=O)c1ccc(Cl)c(-n2c(=O)[nH]c3scc(C(=O)O)c3c2=O)c1] | (DMSO-d6) 3.17 (3H, s), 7.05-7.15 (2H, m), 7.25-7.4 (3H, m), 7.63 (1H, dd, J = 8.6 Hz, 2.2 Hz), 7.85-7.95 (3H, m), 12.5- 13.5 (1H, br), 13.79 (1H, s) |
| 115 | 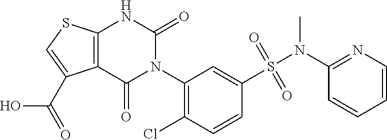 [SMILES: CN(c1ccccn1)S(=O)(=O)c1ccc(Cl)c(-n2c(=O)[nH]c3scc(C(=O)O)c3c2=O)c1] | (DMSO-d6) 3.26 (3H, s), 7.25-7.3 (1H, m), 7.45-7.55 (1H, m), 7.65-7.75 (1H, m), 7.8-7.95 (3H, m), 8.05-8.1 (1H, m), 8.3-8.4 (1H, m) |
| 116 | 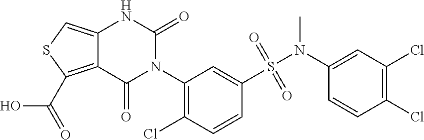 [SMILES: CN(c1ccc(Cl)c(Cl)c1)S(=O)(=O)c1ccc(Cl)c(-n2c(=O)[nH]c3csc(C(=O)O)c3c2=O)c1] | (CDCl3) 3.17 (3H, s), 6.9-7.0 (1H, m), 7.13 (1H, s), 7.29 (1H, d, J = 2.5 Hz), 7.39 (1H, d, J = 8.4 Hz), 7.51 (1H, d, J = 1.2 Hz), 7.7-7.8 (2H, m), 9.12 (1H, s), 14.05 (1H, s) |
| 117 | 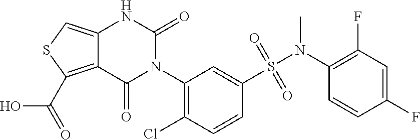 [SMILES: CN(c1ccc(F)cc1F)S(=O)(=O)c1ccc(Cl)c(-n2c(=O)[nH]c3csc(C(=O)O)c3c2=O)c1] | (CDCl3) 3.25 (3H, s), 6.75-6.9 (2H, m), 7.13 (1H, s), 7.2-7.3 (1H, m), 7.63 (1H, d, J = 2.1 Hz), 7.75 (1H, d, J = 8.6 Hz), 7.8-7.85 (1H, m), 9.03 (1H, s), 14.11 (1H, s) |
| 118 | 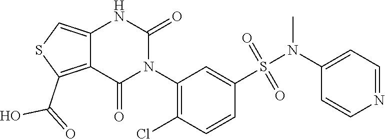 [SMILES: CN(c1ccncc1)S(=O)(=O)c1ccc(Cl)c(-n2c(=O)[nH]c3csc(C(=O)O)c3c2=O)c1] | (CDCl3) 3.22 (3H, s), 7.08 (1H, s), 7.15- 7.25 (2H, m), 7.38 (1H, d, J = 1.7 Hz), 7.7-7.8 (2H, m), 8.55-8.65 (2H, m) |
| 119 | 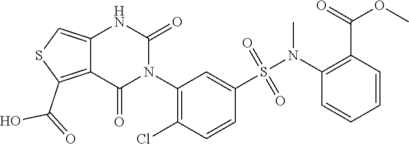 [SMILES: COC(=O)c1ccccc1N(C)S(=O)(=O)c1ccc(Cl)c(-n2c(=O)[nH]c3csc(C(=O)O)c3c2=O)c1] | (DMSO-d6) 3.23 (3H, s), 3.8 (3H, s), 6.9-6.95 (1H, m), 7.35-7.55 (3H, m), 7.7-7.85 (2H, m), 7.9-8.0 (2H, m), 12.03 (1H, s), 14.29 (1H, s) |
| 120 | 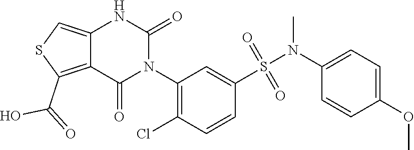 [SMILES: COc1ccc(N(C)S(=O)(=O)c2ccc(Cl)c(-n3c(=O)[nH]c4csc(C(=O)O)c4c3=O)c2)cc1] | (DMSO-d6) 3.12 (3H, s), 3.73 (3H, s), 6.85-6.9 (2H, m), 6.95-7.0 (2H, m), 7.39 (1H, s), 7.68 (1H, dd, J = 8.4 Hz, 2.3 Hz), 7.85-7.95 (2H, m), 12.03 (1H, s), 14.3 (1H, s) |
| 121 | 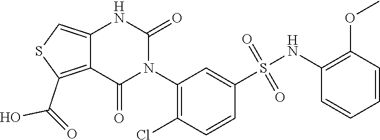 [SMILES: COc1ccccc1NS(=O)(=O)c1ccc(Cl)c(-n2c(=O)[nH]c3csc(C(=O)O)c3c2=O)c1] | (DMSO-d6) 3.51 (3H, s), 6.85-6.95 (2H, m), 7.15-7.25 (2H, m), 7.38 (1H, s), 7.7- 7.8 (1H, m), 7.85-7.95 (1H, m), 7.95-8.0 (1H, m), 9.74 (1H, s), 12.02 (1H, s), 13.5-15.0 (1H, br) |
| 122 | 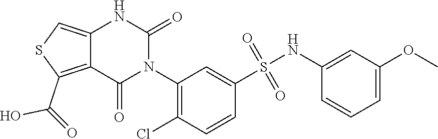 [SMILES: COc1cccc(NS(=O)(=O)c2ccc(Cl)c(-n3c(=O)[nH]c4csc(C(=O)O)c4c3=O)c2)c1] | (DMSO-d6) 3.67 (3H, s), 6.6-6.75 (3H, m), 7.1-7.2 (1H, m), 7.38 (1H, s), 7.84 (1H, dd, J = 8.5 Hz, 2.2 Hz), 7.89 (1H, d, J = 8.5 Hz), 8.13 (1H, d, J = 2.2 Hz), 10.49 (1H, s), 12.04 (1H, s), 14.22 (1H, s) |
| 123 | 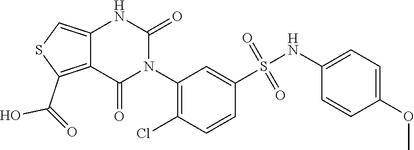 [SMILES: COc1ccc(NS(=O)(=O)c2ccc(Cl)c(-n3c(=O)[nH]c4csc(C(=O)O)c4c3=O)c2)cc1] | (DMSO-d6) 3.69 (3H, s), 6.83 (2H, d, J = 8.9 Hz), 6.99 (2H, d, J = 8.9 Hz), 7.38 (1H, s), 7.75 (1H, dd, J = 8.5 Hz, 2.0 Hz), 7.88 (1H, d, J = 8.5 Hz), 7.99 (1H, d, J = 2.0 Hz), 10.08 (1H, s), 12.03 (1H, s), 14.24 (1H, s) |
| 124 | 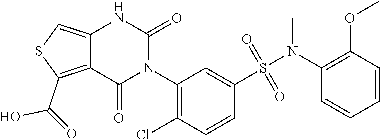 [SMILES: COc1ccccc1N(C)S(=O)(=O)c1ccc(Cl)c(-n2c(=O)[nH]c3csc(C(=O)O)c3c2=O)c1] | (DMSO-d6) 3.17 (3H, s), 3.47 (3H, s), 6.9-7.0 (2H, m), 7.15-7.2 (1H, m), 7.25- 7.35 (1H, m), 7.39 (1H, s), 7.71 (1H, dd, J = 8.6 Hz, 2.2 Hz), 7.9 (1H, d, J = 8.6 Hz), 8.02 (1H, d, J = 2.2 Hz), 12.02 (1H, s), 14.31 (1H, s) |
| 125 | 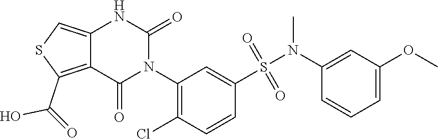 [SMILES: COc1cccc(N(C)S(=O)(=O)c2ccc(Cl)c(-n3c(=O)[nH]c4csc(C(=O)O)c4c3=O)c2)c1] | (DMSO-d6) 3.18 (3H, s), 3.69 (3H, s), 6.55-6.7 (2H, m), 6.8-6.9 (1H, m), 7.2- 7.3 (1H, m), 7.35-7.4 (1H, m), 7.6-7.7 (1H, m), 7.85-8.05 (2H, m), 12.04 (1H, s), 14.26 (1H, s) |
| 126 | 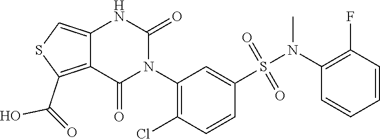 [SMILES: CN(c1ccccc1F)S(=O)(=O)c1ccc(Cl)c(-n2c(=O)[nH]c3csc(C(=O)O)c3c2=O)c1] | (DMSO-d6) 3.18 (3H, s), 7.1-7.35 (3H, m), 7.35-7.45 (2H, m), 7.81 (1H, d, J = 2.3 Hz), 7.95 (1H, d, J = 8.5 Hz), 8.03 (1H, d, J = 2.3 Hz), 12.03 (1H, s), 14.29 (1H, s) |
| 127 | 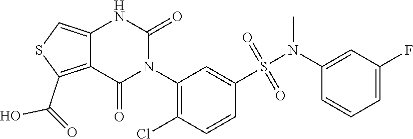 [SMILES: CN(c1cccc(F)c1)S(=O)(=O)c1ccc(Cl)c(-n2c(=O)[nH]c3csc(C(=O)O)c3c2=O)c1] | (DMSO-d6) 3.19 (3H, s), 6.95-7.05 (2H, m), 7.1-7.2 (1H, m), 7.35-7.45 (2H, m), 7.7-7.75 (1H, m), 7.9-8.0 (2H, m), 12.04 (1H, s), 14.27 (1H, s) |
| 128 | 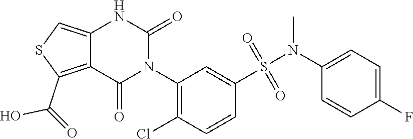 [SMILES: CN(c1ccc(F)cc1)S(=O)(=O)c1ccc(Cl)c(-n2c(=O)[nH]c3csc(C(=O)O)c3c2=O)c1] | (DMSO-d6) 3.15 (3H, s), 7.05-7.25 (4H, m), 7.38 (1H, s), 7.69 (1H, dd, J = 8.5 Hz, 2.5 Hz), 7.9 (1H, d, J = 2.5 Hz), 7.93 (1H, d, J = 8.5 Hz), 12.05 (1H, s), 14.29 (1H, s) |
| 129 | 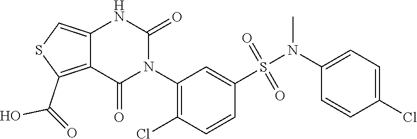 [SMILES: CN(c1ccc(Cl)cc1)S(=O)(=O)c1ccc(Cl)c(-n2c(=O)[nH]c3csc(C(=O)O)c3c2=O)c1] | (DMSO-d6) 3.15 (3H, s), 7.1-7.15 (2H, m), 7.35-7.45 (3H, m), 7.7 (1H, dd, J = 8.6 Hz, 2.1 Hz), 7.9-7.95 (2H, m), 12.05 (1H, s), 14.28 (1H, s) |
| 130 | 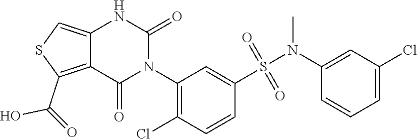 [SMILES: CN(c1cccc(Cl)c1)S(=O)(=O)c1ccc(Cl)c(-n2c(=O)[nH]c3csc(C(=O)O)c3c2=O)c1] | (DMSO-d6) 3.19 (3H, s), 7.05-7.1 (1H, m), 7.2-7.25 (1H, m), 7.35-7.45 (3H, m), 7.72 (1H, dd, J = 8.5 Hz, 2.1 Hz), 7.9- 8.0 (2H, m), 12.06 (1H, s), 14.3 (1H, s) |
| 131 | 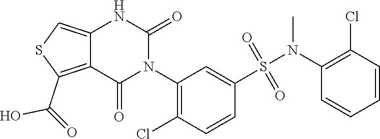 [SMILES: CN(c1ccccc1Cl)S(=O)(=O)c1ccc(Cl)c(-n2c(=O)[nH]c3csc(C(=O)O)c3c2=O)c1] | (DMSO-d6) 3.16 (3H, s), 7.0-7.1 (1H, m), 7.25-7.45 (3H, m), 7.55-7.6 (1H, m), 7.85-7.9 (1H, m), 7.97 (1H, d, J = 8.5 Hz), 8.1 (1H, s), 12.06 (1H, s), 14.31 (1H, s) |
| 132 | 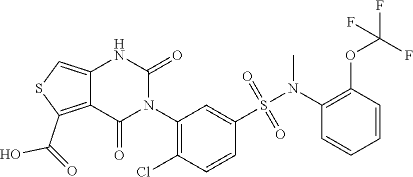 [SMILES: CN(c1ccccc1OC(F)(F)F)S(=O)(=O)c1ccc(Cl)c(-n2c(=O)[nH]c3csc(C(=O)O)c3c2=O)c1] | (DMSO-d6) 3.13 (3H, s), 7.0-7.1 (1H, m), 7.3-7.4 (2H, m), 7.45-7.55 (2H, m), 7.86 (1H, dd, J = 8.5 Hz, 2.4 Hz), 7.98 (1H, d, J = 8.5 Hz), 8.02 (1H, d, J = 2.4 Hz), 12.03 (1H, s), 14.3 (1H, s) |
| 133 | 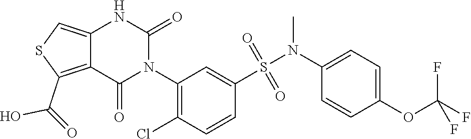 [SMILES: CN(c1ccc(OC(F)(F)F)cc1)S(=O)(=O)c1ccc(Cl)c(-n2c(=O)[nH]c3csc(C(=O)O)c3c2=O)c1] | (DMSO-d6) 3.17 (3H, s), 7.2-7.3 (2H, m), 7.3-7.4 (3H, m), 7.71 (1H, dd, J = 8.5 Hz, 2.1 Hz), 7.9 (1H, d, J = 2.1 Hz), 7.93 (1H, d, J = 8.5 Hz), 12.0 (1H, s), 14.25 (1H, s) |
| 134 | 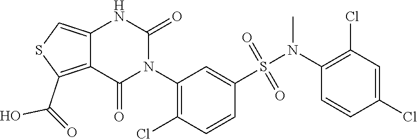 [SMILES: CN(c1ccc(Cl)cc1Cl)S(=O)(=O)c1ccc(Cl)c(-n2c(=O)[nH]c3csc(C(=O)O)c3c2=O)c1] | (DMSO-d6) 3.15 (3H, s), 7.06 (1H, d, J = 8.5 Hz), 7.35-7.45 (2H, m), 7.76 (1H, d, J = 2.4 Hz), 7.85-7.9 (1H, m), 7.97 (1H, d, J = 8.54 Hz), 8.07 (1H, d, J = 2.2 Hz), 12.03 (1H, s), 14.27 (1H, s) |
| 135 | 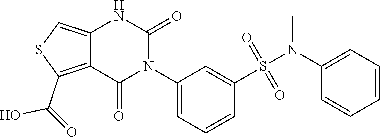 [SMILES: CN(c1ccccc1)S(=O)(=O)c1cccc(-n2c(=O)[nH]c3csc(C(=O)O)c3c2=O)c1] | (DMSO-d6) 3.16 (3H, s), 7.05-7.1 (2H, m), 7.25-7.4 (4H, m), 7.55-7.65 (1H, m), 7.7-7.8 (3H, m), 11.95 (1H, s), 14.77 (1H, s) |
| 136 | 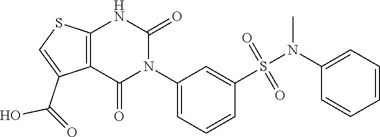 [SMILES: CN(c1ccccc1)S(=O)(=O)c1cccc(-n2c(=O)[nH]c3scc(C(=O)O)c3c2=O)c1] | (DMSO-d6) 3.16 (3H, s), 7.05-7.1 (2H, m), 7.25-7.4 (3H, m), 7.55-7.65 (1H, m), 7.7-7.8 (3H, m), 7.98 (1H, s), 12.95 (1H, s), 14.31 (1H, s) |
| 137 | 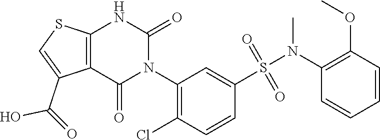 [SMILES: COc1ccccc1N(C)S(=O)(=O)c1ccc(Cl)c(-n2c(=O)[nH]c3scc(C(=O)O)c3c2=O)c1] | (DMSO-d6) 3.16 (3H, s), 3.47 (3H, s), 6.85-7.0 (2H, m), 7.1-7.2 (1H, m), 7.25- 7.35 (1H, m), 7.69 (1H, dd, J = 8.4 Hz, 2.1 Hz), 7.85-7.95 (2H, m), 7.99 (1H, d, J = 2.1 Hz), 12.5-13.5 (1H, br), 13.83 (1H, brs) |
| 138 | 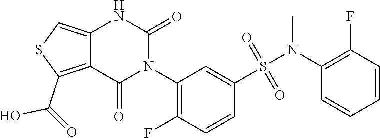 [SMILES: CN(c1ccccc1F)S(=O)(=O)c1ccc(F)c(-n2c(=O)[nH]c3csc(C(=O)O)c3c2=O)c1] | (DMSO-d6) 3.17 (3H, s), 7.1-7.25 (2H, m), 7.25-7.35 (1H, m), 7.35-7.45 (2H, m), 7.65-7.75 (1H, m), 7.8-7.9 (1H, m), 8.02 (1H, dd, J = 6.6 Hz, 2.3 Hz), 12.03 (1H, s), 14.31 (1H, s) |
| 139 | 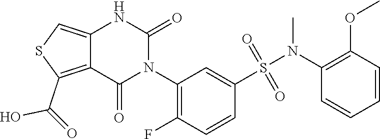 [SMILES: COc1ccccc1N(C)S(=O)(=O)c1ccc(F)c(-n2c(=O)[nH]c3csc(C(=O)O)c3c2=O)c1] | (DMSO-d6) 3.16 (3H, s), 3.47 (3H, s), 6.9-7.0 (2H, m), 7.19 (1H, dd, J = 8.0 Hz, 1.5 Hz), 7.3-7.35 (1H, m), 7.37 (1H, s), 7.6-7.7 (1H, m), 7.7-7.8 (1H, m), 8.0 (1H, dd, J = 6.6 Hz, 2.5 Hz), 12.02 (1H, s), 14.32 (1H, s) |
| 140 | 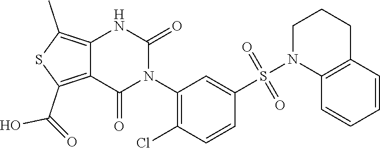 [SMILES: Cc1sc(C(=O)O)c2c(=O)n(-c3cc(S(=O)(=O)N4CCCc5ccccc54)ccc3Cl)c(=O)[nH]c12] | (DMSO-d6) 1.6-1.7 (2H, m), 2.45-2.55 (5H, m), 3.7-3.8 (2H, m), 7.05-7.25 (3H, m), 7.56 (1H, d, J = 8.5 Hz), 7.6-7.65 (1H, m), 7.84 (1H, d, J = 8.5 Hz), 8.1 (1H, d, J = 2.4 Hz), 11.94 (1H, s), 14.22 (1H, s) |
| 141 | 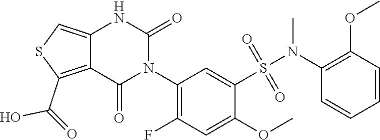 [SMILES: COc1ccccc1N(C)S(=O)(=O)c1cc(-n2c(=O)[nH]c3csc(C(=O)O)c3c2=O)c(F)cc1OC] | (CD3OD) 3.36 (3H, s), 3.54 (3H, s), 4.01 (3H, s), 6.85-6.95 (2H, m), 7.15- 7.35 (4H, m), 7.69 (1H, d, J = 8.4 Hz) |
| 142 | 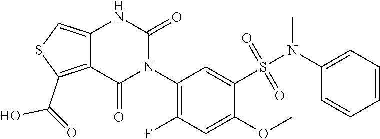 [SMILES: COc1cc(F)c(-n2c(=O)[nH]c3csc(C(=O)O)c3c2=O)cc1S(=O)(=O)N(C)c1ccccc1] | (DMSO-d6) 3.3 (3H, s), 3.87 (3H, s), 7.15-7.4 (6H, m), 7.42 (1H, d, J = 11.7 Hz), 7.92 (1H, d, J = 8.3 Hz), 11.95 (1H, s), 14.39 (1H, s) |
| 143 | 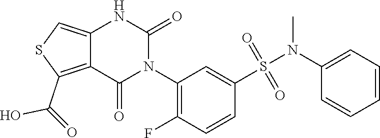 [SMILES: CN(c1ccccc1)S(=O)(=O)c1ccc(F)c(-n2c(=O)[nH]c3csc(C(=O)O)c3c2=O)c1] | (DMSO-d6) 3.17 (3H, s), 7.05-7.15 (2H, m), 7.25-7.4 (4H, m), 7.6-7.75 (2H, m), 7.9-8.0 (1H, m), 12.02 (1H, s), 14.29 (1H, s) |
| 144 | 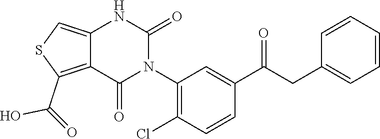 [SMILES: O=C(Cc1ccccc1)c1ccc(Cl)c(-n2c(=O)[nH]c3csc(C(=O)O)c3c2=O)c1] | (DMSO-d6) 4.42 (2H, s), 7.2-7.45 (6H, m), 7.86 (1H, d, J = 8.5 Hz), 8.2 (1H, dd, J = 8.5 Hz, 2.2 Hz), 8.29 (1H, d, J = 2.2 Hz), 12.06 (1H, brs), 14.32 (1H, brs) |
| 145 | 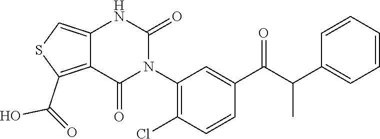 [SMILES: CC(C(=O)c1ccc(Cl)c(-n2c(=O)[nH]c3csc(C(=O)O)c3c2=O)c1)c1ccccc1] | (DMSO-d6) 1.4-1.5 (3H, m), 4.9-5.0 (1H, m), 7.15-7.45 (6H, m), 7.7-7.8 (1H, m), 8.1-8.15 (1H, m), 8.2-8.3 (1H, m), 12.0-12.1 (1H, m), 14.2-14.35 (1H, m) |
| 146 | 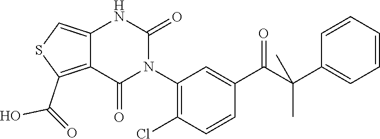 [SMILES: CC(C)(C(=O)c1ccc(Cl)c(-n2c(=O)[nH]c3csc(C(=O)O)c3c2=O)c1)c1ccccc1] | (DMSO-d6) 1.57 (6H, s), 7.25-7.45 (7H, m), 7.5-7.6 (1H, m), 7.9-8.0 (1H, m), 11.9-12.1 (1H, br), 14.2-14.4 (1H, br) |
| 147 | 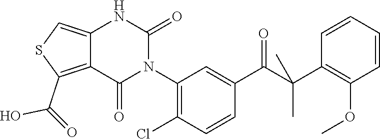 [SMILES: COc1ccccc1C(C)(C)C(=O)c1ccc(Cl)c(-n2c(=O)[nH]c3csc(C(=O)O)c3c2=O)c1] | (DMSO-d6) 1.55 (3H, s), 1.57 (3H, s), 3.33 (3H, s), 6.8-6.85 (1H, m), 7.0-7.1 (1H, m), 7.2-7.3 (1H, m), 7.3-7.5 (3H, m), 7.5-7.55 (1H, m), 7.94 (1H, d, J = 2.4 Hz), 12.0 (1H, s), 14.37 (1H, brs) |
| 148 | 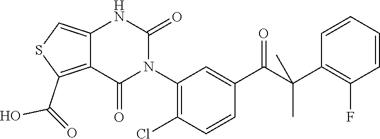 [SMILES: CC(C)(C(=O)c1ccc(Cl)c(-n2c(=O)[nH]c3csc(C(=O)O)c3c2=O)c1)c1ccccc1F] | (DMSO-d6) 1.6 (3H, s), 1.62 (3H, s), 7.0-7.15 (1H, m), 7.25-7.4 (3H, m), 7.47 (1H, dd, J = 8.7 Hz, 2.0 Hz), 7.57 (1H, d, J = 8.7 Hz), 7.65-7.75 (1H, m), 8.01 (1H, d, J = 2.0 Hz), 12.03 (1H, s), 14.3 (1H, brs) |
| 149 | 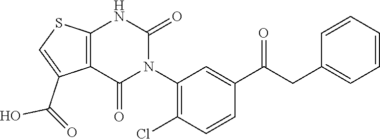 [SMILES: O=C(Cc1ccccc1)c1ccc(Cl)c(-n2c(=O)[nH]c3scc(C(=O)O)c3c2=O)c1] | (DMSO-d6) 4.42 (2H, s), 7.2-7.35 (5H, m), 7.86 (1H, d, J = 8.5 Hz), 7.94 (1H, s), 8.15-8.3 (2H, m), 13.84 (1H, s) |
| 150 | 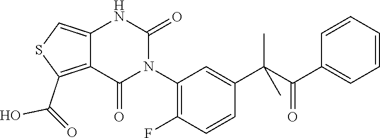 [SMILES: CC(C)(C(=O)c1ccccc1)c1ccc(F)c(-n2c(=O)[nH]c3csc(C(=O)O)c3c2=O)c1] | (DMSO-d6) 1.55 (6H, s), 7.25-7.55 (8H, m), 7.6-7.65 (1H, m), 11.99 (1H, s), 14.46 (1H, brs) |
| 151 | 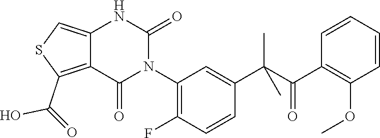 [SMILES: COc1ccccc1C(=O)C(C)(C)c1ccc(F)c(-n2c(=O)[nH]c3csc(C(=O)O)c3c2=O)c1] | (DMSO-d6) 1.49 (3H, s), 1.5 (3H, s), 3.66 (3H, s), 6.55-6.65 (1H, m), 6.75- 6.85 (1H, m), 6.95-7.05 (1H, m), 7.25- 7.5 (4H, m), 7.5-7.6 (1H, m), 11.99 (1H, s), 14.5 (1H, s) |
| 152 | 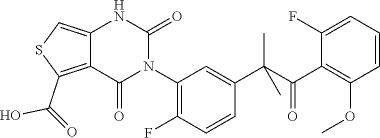 [SMILES: COc1cccc(F)c1C(=O)C(C)(C)c1ccc(F)c(-n2c(=O)[nH]c3csc(C(=O)O)c3c2=O)c1] | (DMSO-d6) 1.536 (3H, s), 1.543 (3H, s), 3.61 (3H, s), 6.75-6.85 (2H, m), 7.3-7.4 (3H, m), 7.45-7.6 (2H, m), 11.98 (1H, s), 14.5 (1H, s) |
| 153 | 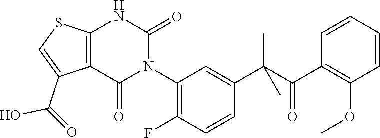 [SMILES: COc1ccccc1C(=O)C(C)(C)c1ccc(F)c(-n2c(=O)[nH]c3scc(C(=O)O)c3c2=O)c1] | (DMSO-d6) 1.49 (3H, s), 1.5 (3H, s), 3.66 (3H, s), 6.55-6.65 (1H, m), 6.75- 6.85 (1H, m), 6.95-7.05 (1H, m), 7.25- 7.55 (4H, m), 7.94 (1H, s), 12.8-13.1 (1H, br), 14.01 (1H, s) |
| 154 | 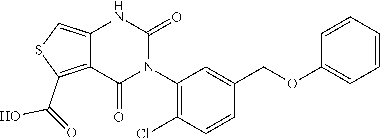 [SMILES: O=C(O)c1scc2[nH]c(=O)n(-c3cc(COc4ccccc4)ccc3Cl)c(=O)c12] | (DMSO-d6) 5.15 (2H, s), 6.9-7.1 (3H, m), 7.25-7.35 (2H, m), 7.4 (1H, s), 7.55- 7.65 (1H, m), 7.65-7.75 (2H, m), 12.05 (1H, s), 14.42 (1H, s) |
| 155 | 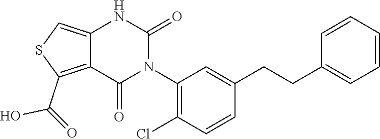 [SMILES: O=C(O)c1scc2[nH]c(=O)n(-c3cc(CCc4ccccc4)ccc3Cl)c(=O)c12] | (DMSO-d6) 2.85-2.95 (4H, m), 7.1-7.6 (9H, m), 12.04 (1H, s), 14.46 (1H, s) |
| 156 | 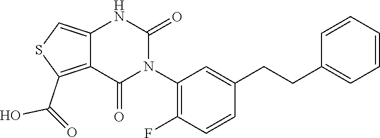 [SMILES: O=C(O)c1scc2[nH]c(=O)n(-c3cc(CCc4ccccc4)ccc3F)c(=O)c12] | (DMSO-d6) 2.8-3.0 (4H, m), 7.15-7.45 (9H, m), 12.03 (1H, s), 14.46 (1H, brs) |
| 157 | 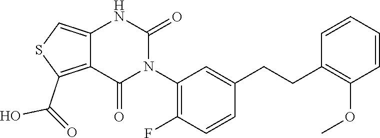 [SMILES: COc1ccccc1CCc1ccc(F)c(-n2c(=O)[nH]c3csc(C(=O)O)c3c2=O)c1] | (DMSO-d6) 2.8-2.9 (4H, m), 3.79 (3H, s), 6.8-6.9 (1H, m), 6.9-7.0 (1H, m), 7.1-7.25 (2H, m), 7.25-7.45 (4H, m), 12.02 (1H, s), 14.48 (1H, brs) |
| 158 | 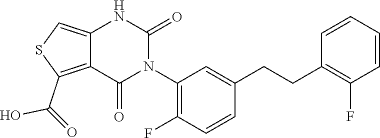 [SMILES: O=C(O)c1scc2[nH]c(=O)n(-c3cc(CCc4ccccc4F)ccc3F)c(=O)c12] | (DMSO-d6) 2.85-3.0 (4H, m), 7.05-7.2 (2H, m), 7.2-7.45 (6H, m), 12.03 (1H, s), 14.46 (1H, brs) |
| 159 | 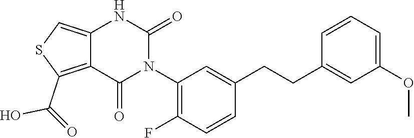 [SMILES: COc1cccc(CCc2ccc(F)c(-n3c(=O)[nH]c4csc(C(=O)O)c4c3=O)c2)c1] | (DMSO-d6) 2.8-2.95 (4H, m), 3.72 (3H, s), 6.7-6.85 (3H, m), 7.15-7.25 (1H, m), 7.25-7.45 (4H, m), 11.95-12.1 (1H, br), 14.35-14.55 (1H, br) |
| 160 | 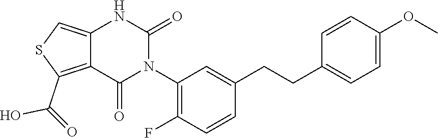 [SMILES: COc1ccc(CCc2ccc(F)c(-n3c(=O)[nH]c4csc(C(=O)O)c4c3=O)c2)cc1] | (DMSO-d6) 2.75-2.95 (4H, m), 3.71 (3H, s), 6.8-6.9 (2H, m), 7.1-7.2 (2H, m), 7.25-7.45 (4H, m), 12.01 (1H, brs), 14.4- 14.55 (1H, br) |
| 161 | 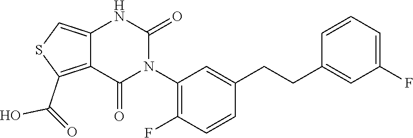 [SMILES: O=C(O)c1scc2[nH]c(=O)n(-c3cc(CCc4cccc(F)c4)ccc3F)c(=O)c12] | (DMSO-d6) 2.85-3.0 (4H, m), 6.95-7.05 (1H, m), 7.05-7.15 (2H, m), 7.25-7.45 (5H, m), 12.03 (1H, brs), 14.3-14.6 (1H, br) |
| 162 | 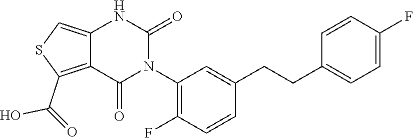 [SMILES: O=C(O)c1scc2[nH]c(=O)n(-c3cc(CCc4ccc(F)cc4)ccc3F)c(=O)c12] | (DMSO-d6) 2.8-2.95 (4H, m), 7.05-7.15 (2H, m), 7.2-7.45 (6H, m), 12.03 (1H, brs), 14.3-14.6 (1H, br) |
| 163 | 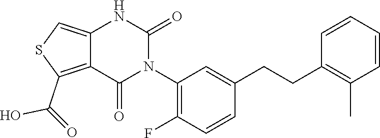 [SMILES: Cc1ccccc1CCc1ccc(F)c(-n2c(=O)[nH]c3csc(C(=O)O)c3c2=O)c1] | (DMSO-d6) 2.28 (3H, s), 2.8-2.9 (4H, m), 7.05-7.25 (4H, m), 7.25-7.5 (4H, m), 12.04 (1H, brs), 14.47 (1H, brs) |
| 164 | 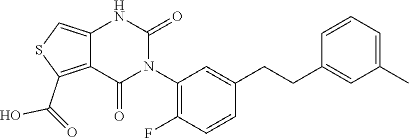 [SMILES: Cc1cccc(CCc2ccc(F)c(-n3c(=O)[nH]c4csc(C(=O)O)c4c3=O)c2)c1] | (DMSO-d6) 2.27 (3H, s), 2.8-2.95 (4H, m), 6.95-7.1 (3H, m), 7.1-7.2 (1H, m), 7.25-7.45 (4H, m), 12.03 (1H, brs), 14.47 (1H, brs) |
| 165 | 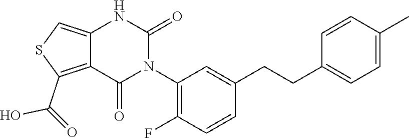 [SMILES: Cc1ccc(CCc2ccc(F)c(-n3c(=O)[nH]c4csc(C(=O)O)c4c3=O)c2)cc1] | (DMSO-d6) 2.25 (3H, s), 2.8-2.95 (4H, m), 7.05-7.15 (4H, m), 7.25-7.45 (4H, m), 12.03 (1H, brs), 14.35-14.6 (1H, br) |
| 166 | 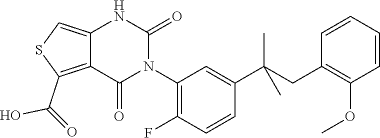 [SMILES: COc1ccccc1CC(C)(C)c1ccc(F)c(-n2c(=O)[nH]c3csc(C(=O)O)c3c2=O)c1] | (DMSO-d6) 1.23 (3H, s), 1.25 (3H, s), 2.85 (2H, s), 3.67 (3H, s), 6.7-6.8 (2H, m), 6.85-6.95 (1H, m), 7.1-7.2 (1H, m), 7.25-7.35 (1H, m), 7.35-7.45 (2H, m), 7.5-7.6 (1H, m), 12.0 (1H, s), 14.55 (1H, s) |
| 167 | 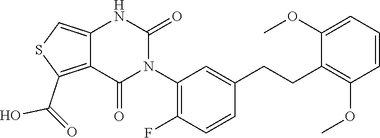 [SMILES: COc1cccc(OC)c1CCc1ccc(F)c(-n2c(=O)[nH]c3csc(C(=O)O)c3c2=O)c1] | (DMSO-d6) 2.65-2.75 (2H, m), 2.8-2.9 (2H, m), 3.75 (6H, s), 6.55-6.7 (2H, m), 7.1-7.2 (1H, m), 7.25-7.45 (4H, m), 12.0 (1H, s), 14.48 (1H, brs) |
| 168 | 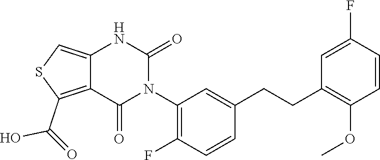 [SMILES: COc1ccc(F)cc1CCc1ccc(F)c(-n2c(=O)[nH]c3csc(C(=O)O)c3c2=O)c1] | (DMSO-d6) 2.8-2.95 (4H, m), 3.77 (3H, s), 6.9-7.1 (3H, m), 7.25-7.45 (4H, m), 12.01 (1H, s), 14.46 (1H, brs) |
| 169 | 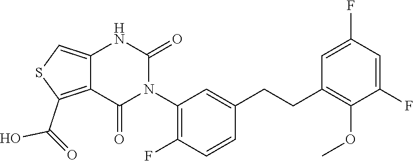 [SMILES: COc1c(F)cc(F)cc1CCc1ccc(F)c(-n2c(=O)[nH]c3csc(C(=O)O)c3c2=O)c1] | (DMSO-d6) 2.85-2.95 (4H, m), 3.75 (3H, s), 6.9-7.05 (1H, m), 7.05-7.2 (1H, m), 7.3-7.45 (4H, m), 12.01 (1H, s), 14.45 (1H, brs) |
| 170 | 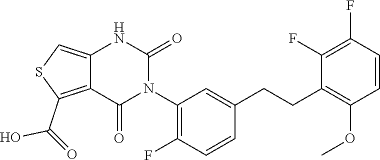 [SMILES: COc1ccc(F)c(F)c1CCc1ccc(F)c(-n2c(=O)[nH]c3csc(C(=O)O)c3c2=O)c1] | (DMSO-d6) 2.75-2.85 (2H, m), 2.85- 2.95 (2H, m), 3.77 (3H, s), 6.75-6.85 (1H, m), 7.15-7.35 (4H, m), 7.37 (1H, s), 11.99 (1H, s), 14.46 (1H, brs) |
| 171 | 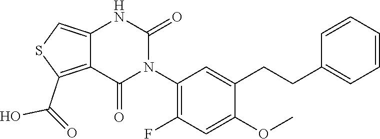 [SMILES: COc1cc(F)c(-n2c(=O)[nH]c3csc(C(=O)O)c3c2=O)cc1CCc1ccccc1] | (DMSO-d6) 2.75-2.9 (4H, m), 3.86 (3H, s), 7.08 (1H, d, J = 12.2 Hz), 7.15-7.35 (6H, m), 7.38 (1H, s), 11.97 (1H, s), 14.55 (1H, brs) |
| 172 | 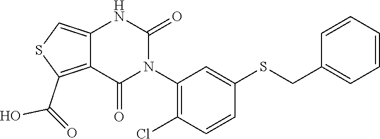 [SMILES: O=C(O)c1scc2[nH]c(=O)n(-c3cc(SCc4ccccc4)ccc3Cl)c(=O)c12] | (DMSO-d6) 4.28 (2H, s), 7.2-7.35 (3H, m), 7.35-7.5 (4H, m), 7.58 (1H, d, J = 8.5 Hz), 7.66 (1H, d, J = 2.2 Hz), 12.06 (1H, s), 14.41 (1H, s) |
| 173 | 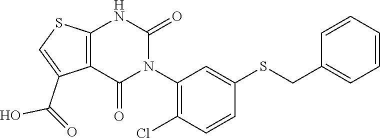 [SMILES: O=C(O)c1csc2[nH]c(=O)n(-c3cc(SCc4ccccc4)ccc3Cl)c(=O)c12] | (DMSO-d6) 4.29 (2H, s), 7.2-7.45 (6H, m), 7.57 (1H, d, J = 8.5 Hz), 7.64 (1H, d, J = 2.3 Hz), 7.94 (1H, s), 13.03 (1H, s), 13.94 (1H, s) |
| 174 | 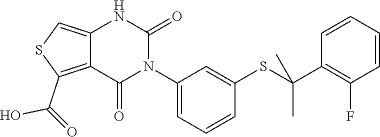 [SMILES: CC(C)(Sc1cccc(-n2c(=O)[nH]c3csc(C(=O)O)c3c2=O)c1)c1ccccc1F] | (DMSO-d6) 1.69 (6H, s), 7.0-7.2 (4H, m), 7.25-7.4 (5H, m), 11.89 (1H, s), 14.86 (1H, s) |
| 175 | 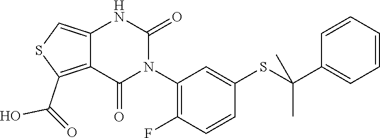 [SMILES: CC(C)(Sc1ccc(F)c(-n2c(=O)[nH]c3csc(C(=O)O)c3c2=O)c1)c1ccccc1] | (DMSO-d6) 4.24 (2H, s), 7.2-7.5 (8H, m), 7.62 (1H, dd, J = 6.7 Hz, 2.2 Hz), 12.05 (1H, s), 14.41 (1H, s) |
| 176 | 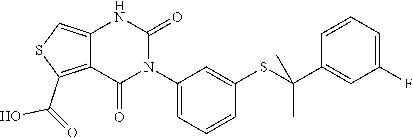 [SMILES: CC(C)(Sc1cccc(-n2c(=O)[nH]c3csc(C(=O)O)c3c2=O)c1)c1cccc(F)c1] | (DMSO-d6) 1.65 (6H, s), 7.0-7.1 (1H, m), 7.1-7.45 (8H, m), 11.88 (1H, s), 14.83 (1H, s) |
| 177 | 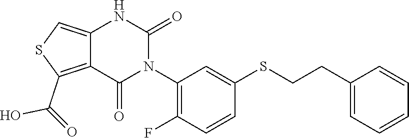 [SMILES: O=C(O)c1scc2[nH]c(=O)n(-c3cc(SCCc4ccccc4)ccc3F)c(=O)c12] | (DMSO-d6) 2.8-2.9 (2H, m), 3.15-3.25 (2H, m), 7.15-7.55 (8H, m), 7.58 (1H, dd, J = 6.8 Hz, 2.3 Hz) |
| 178 | 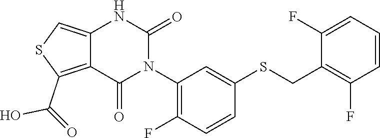 [SMILES: O=C(O)c1scc2[nH]c(=O)n(-c3cc(SCc4c(F)cccc4F)ccc3F)c(=O)c12] | (DMSO-d6) 4.21 (2H, s), 7.0-7.15 (2H, m), 7.3-7.45 (3H, m), 7.5-7.65 (2H, m), 12.0 (1H, s), 14.37 (1H, s) |
| 179 | 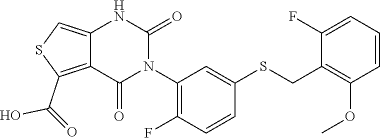 [SMILES: COc1cccc(F)c1CSc1ccc(F)c(-n2c(=O)[nH]c3csc(C(=O)O)c3c2=O)c1] | (DMSO-d6) 3.76 (3H, s), 4.15 (2H, s), 6.75-6.9 (2H, m), 7.25-7.35 (2H, m), 7.35-7.45 (1H, m), 7.45-7.6 (2H, m) |
| 180 | 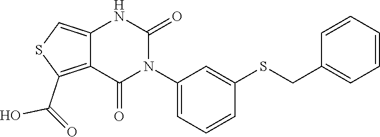 [SMILES: O=C(O)c1scc2[nH]c(=O)n(-c3cccc(SCc4ccccc4)c3)c(=O)c12] | (DMSO-d6) 4.27 (2H, s), 7.15-7.5 (10H, m), 11.93 (1H, s), 14.88 (1H, s) |
| 181 | 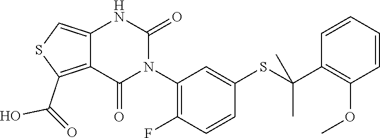 [SMILES: COc1ccccc1C(C)(C)Sc1ccc(F)c(-n2c(=O)[nH]c3csc(C(=O)O)c3c2=O)c1] | (DMSO-d6) 1.65-1.7 (6H, m), 3.86 (3H, s) 6.75-6.85 (1H, m), 7.0-7.15 (3H, m), 7.2-7.3 (2H, m), 7.37 (1H, s), 7.42 (1H, dd, J = 6.9 Hz, 2.2 Hz), 11.98 (1H, s), 14.38 (1H, s) |
| 182 | 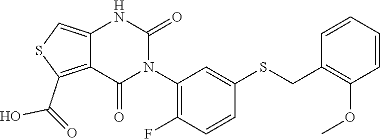 [SMILES: COc1ccccc1CSc1ccc(F)c(-n2c(=O)[nH]c3csc(C(=O)O)c3c2=O)c1] | (DMSO-d6) 3.79 (3H, s), 4.16 (2H, s), 6.8-6.9 (1H, m), 6.95-7.05 (1H, m), 7.2- 7.3 (2H, m), 7.3-7.5 (3H, m), 7.59 (1H, dd, J = 7.0 hz, 2.2 Hz), 12.02 (1H, s), 14.39 (1H, s) |
| 183 | 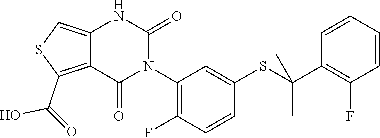 [SMILES: CC(C)(Sc1ccc(F)c(-n2c(=O)[nH]c3csc(C(=O)O)c3c2=O)c1)c1ccccc1F] | (DMSO-d6) 1.69 (6H, s), 7.0-7.35 (6H, m), 7.37 (1H, s), 7.46 (1H, dd, J = 6.7 Hz, 2.2 Hz), 11.98 (1H, s), 14.37 (1H, brs) |
| 184 | 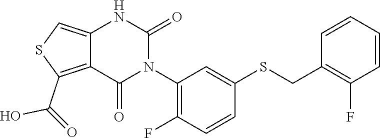 [SMILES: O=C(O)c1scc2[nH]c(=O)n(-c3cc(SCc4ccccc4F)ccc3F)c(=O)c12] | (DMSO-d6) 4.24 (2H, s), 7.05-7.25 (2H, m), 7.25-7.45 (4H, m), 7.45-7.55 (1H, m), 7.63 (1H, dd, J = 6.8 Hz, 2.5 Hz), 12.03 (1H, s), 14.38 (1H, s) |
| 185 | 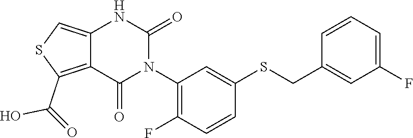 [SMILES: O=C(O)c1scc2[nH]c(=O)n(-c3cc(SCc4cccc(F)c4)ccc3F)c(=O)c12] | (DMSO-d6) 4.25 (2H, s), 7.0-7.1 (1H, m), 7.15-7.2 (2H, m), 7.25-7.5 (4H, m), 7.62 (1H, dd, J = 6.7 Hz, 2.3 Hz), 12.03 (1H, s), 14.37 (1H, s) |
| 186 | 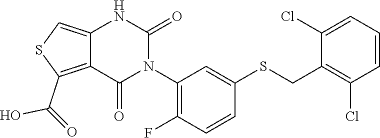 [SMILES: O=C(O)c1scc2[nH]c(=O)n(-c3cc(SCc4c(Cl)cccc4Cl)ccc3F)c(=O)c12] | (DMSO-d6) 4.4 (2H, s), 7.3-7.6 (6H, m), 7.65-7.75 (1H, m), 12.01 (1H, s), 14.37 (1H, s) |
| 187 | 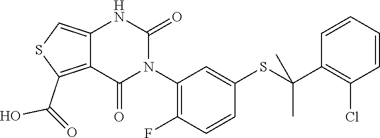 [SMILES: CC(C)(Sc1ccc(F)c(-n2c(=O)[nH]c3csc(C(=O)O)c3c2=O)c1)c1ccccc1Cl] | (DMSO-d6) 1.75-1.8 (6H, m), 7.05-7.15 (1H, m), 7.15-7.3 (4H, m), 7.37 (1H, s), 7.4-7.5 (2H, m), 11.97 (1H, s), 14.37 (1H, s) |
| 188 | 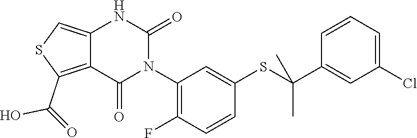 [SMILES: CC(C)(Sc1ccc(F)c(-n2c(=O)[nH]c3csc(C(=O)O)c3c2=O)c1)c1cccc(Cl)c1] | (DMSO-d6) 1.65 (6H, s), 7.15-7.25 (1H, m), 7.25-7.45 (6H, m), 7.5-7.6 (1H, m), 12.0 (1H, s), 14.38 (1H, s) |
| 189 | 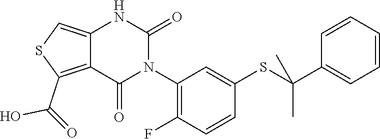 [SMILES: CC(C)(Sc1ccc(F)c(-n2c(=O)[nH]c3csc(C(=O)O)c3c2=O)c1)c1ccccc1] | (DMSO-d6) 1.64 (6H, s), 7.15-7.35 (5H, m), 7.37 (1H, s), 7.4-7.45 (2H, m), 7.52 (1H, dd, J = 7.3 Hz, 2.3 Hz), 11.99 (1H, s), 14.37 (1H, brs) |
| 190 | 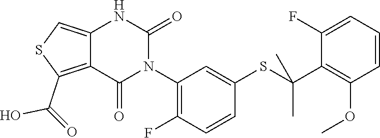 [SMILES: COc1cccc(F)c1C(C)(C)Sc1ccc(F)c(-n2c(=O)[nH]c3csc(C(=O)O)c3c2=O)c1] | (DMSO-d6) 1.75-1.85 (6H, m), 3.77 (3H, s), 6.6-6.7 (1H, m), 6.85 (1H, d, J = 8.1 Hz), 7.05-7.15 (1H, m), 7.2-7.35 (2H, m), 7.37 (1H, s), 7.4-7.45 (1H, m), 11.97 (1H, s), 14.4 (1H, brs) |
| 191 | 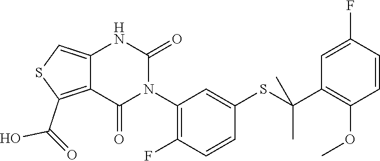 [SMILES: COc1ccc(F)cc1C(C)(C)Sc1ccc(F)c(-n2c(=O)[nH]c3csc(C(=O)O)c3c2=O)c1] | (DMSO-d6) 1.65-1.7 (6H, m), 3.85 (3H, s), 6.75-6.8 (1H, m), 7.0-7.1 (2H, m), 7.1-7.2 (1H, m), 7.25-7.35 (1H, m), 7.37 (1H, s), 7.4 (1H, dd, J = 7.3 Hz, 2.2 hz), 11.98 (1H, s), 14.39 (1H, brs) |
| 192 | 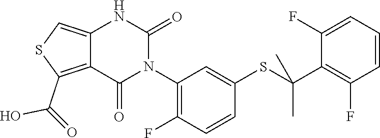 [SMILES: CC(C)(Sc1ccc(F)c(-n2c(=O)[nH]c3csc(C(=O)O)c3c2=O)c1)c1c(F)cccc1F] | (DMSO-d6) 1.81 (6H, s), 6.9-7.05 (2H, m), 7.1-7.2 (1H, m), 7.25-7.4 (3H, m), 7.45-7.5 (1H, m), 11.97 (1H, s), 14.38 (1H, brs) |
| 193 | 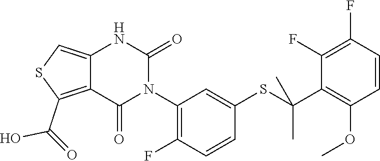 [SMILES: COc1ccc(F)c(F)c1C(C)(C)Sc1ccc(F)c(-n2c(=O)[nH]c3csc(C(=O)O)c3c2=O)c1] | (DMSO-d6) 1.8-1.85 (6H, m), 3.76 (3H, s), 6.75-6.85 (1H, m), 7.15-7.45 (5H, m), 11.98 (1H, s), 14.38 (1H, brs) |
| 194 | 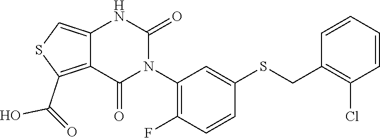 [SMILES: O=C(O)c1scc2[nH]c(=O)n(-c3cc(SCc4ccccc4Cl)ccc3F)c(=O)c12] | (DMSO-d6) 4.29 (2H, s), 7.2-7.35 (2H, m), 7.35-7.55 (5H, m), 7.6-7.7 (1H, m), 12.02 (1H, s), 14.38 (1H, brs) |
| 195 | 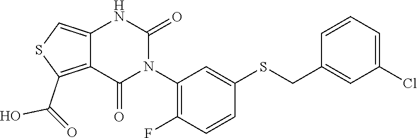 [SMILES: O=C(O)c1scc2[nH]c(=O)n(-c3cc(SCc4cccc(Cl)c4)ccc3F)c(=O)c12] | (DMSO-d6) 4.24 (2H, s), 7.25-7.5 (7H, m), 7.6-7.65 (1H, m), 12.03 (1H, s), 14.38 (1H, brs) |
| 196 | 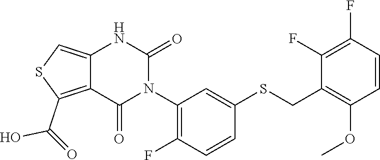 [SMILES: COc1ccc(F)c(F)c1CSc1ccc(F)c(-n2c(=O)[nH]c3csc(C(=O)O)c3c2=O)c1] | (DMSO-d6) 3.73 (3H, s), 4.16 (2H, s), 6.75-6.85 (1H, m), 7.1-7.6 (5H, m) |
| 197 | 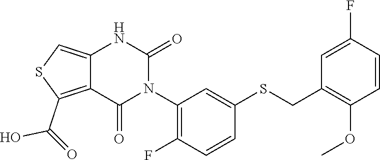 [SMILES: COc1ccc(F)cc1CSc1ccc(F)c(-n2c(=O)[nH]c3csc(C(=O)O)c3c2=O)c1] | (DMSO-d6) 3.77 (3H, s), 4.15 (2H, s), 6.95-7.15 (3H, m), 7.35-7.5 (3H, m), 7.6-7.65 (1H, m), 12.06 (1H, s), 14.41 (1H, s) |
| 198 | 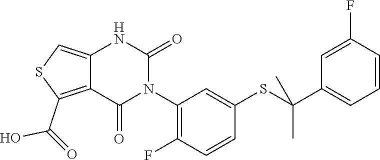 [SMILES: CC(C)(Sc1ccc(F)c(-n2c(=O)[nH]c3csc(C(=O)O)c3c2=O)c1)c1cccc(F)c1] | (DMSO-d6) 1.6-1.7 (6H, m), 7.0-7.1 (1H, m), 7.15-7.25 (3H, m), 7.3-7.4 (3H, m), 7.5-7.55 (1H, m), 12.01 (1H, s), 14.4 (1H, s) |
| 199 | 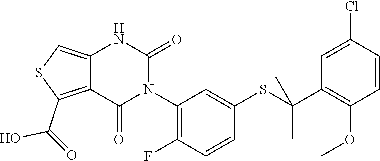 [SMILES: COc1ccc(Cl)cc1C(C)(C)Sc1ccc(F)c(-n2c(=O)[nH]c3csc(C(=O)O)c3c2=O)c1] | (DMSO-d6) 1.66 (3H, s), 1.67 (3H, s), 3.86 (3H, s), 7.0 (1H, d, J = 2.5 Hz), 7.06 (1H, d, J = 8.8 Hz), 7.1-7.2 (1H, m), 7.25-7.35 (2H, m), 7.38 (1H, s), 7.4-7.5 (1H, m), 12.01 (1H, s), 14.41 (1H, brs) |
| 200 | 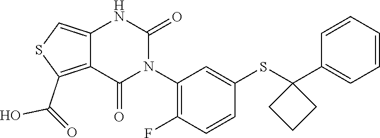 [SMILES: O=C(O)c1scc2[nH]c(=O)n(-c3cc(SC4(c5ccccc5)CCC4)ccc3F)c(=O)c12] | (DMSO-d6) 1.75-1.9 (1H, m), 2.15-2.3 (1H, m), 2.4-2.65 (4H, m), 7.0-7.2 (4H, m), 7.2-7.35 (3H, m), 7.37 (1H, s), 7.45- 7.5 (1H, m), 11.98 (1H, s), 14.41 (1H, brs) |
| 201 | 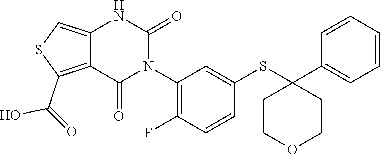 [SMILES: O=C(O)c1scc2[nH]c(=O)n(-c3cc(SC4(c5ccccc5)CCOCC4)ccc3F)c(=O)c12] | (DMSO-d6) 2.05-2.25 (4H, m), 3.5-3.65 (2H, m), 3.85-3.95 (2H, m), 6.85-6.95 (1H, m), 7.15-7.4 (8H, m), 11.98 (1H, s), 14.38 (1H, brs) |
| 202 | 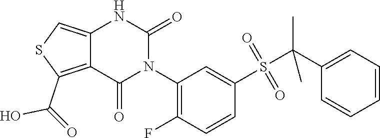 [SMILES: CC(C)(c1ccccc1)S(=O)(=O)c1ccc(F)c(-n2c(=O)[nH]c3csc(C(=O)O)c3c2=O)c1] | (DMSO-d6) 1.73 (6H, s), 7.3-7.4 (7H, m), 7.5-7.6 (1H, m), 7.95 (1H, dd, J = 6.7 Hz, 2.2 Hz), 12.01 (1H, s), 14.25 (1H, s) |
| 203 | 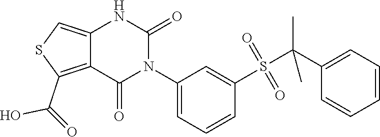 [SMILES: CC(C)(c1ccccc1)S(=O)(=O)c1cccc(-n2c(=O)[nH]c3csc(C(=O)O)c3c2=O)c1] | (DMSO-d6) 1.7 (6H, s), 6.89 (1H, s), 7.2-7.4 (6H, m), 7.5-7.7 (3H, m) |
| 204 | 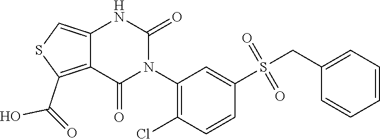 [SMILES: O=C(O)c1scc2[nH]c(=O)n(-c3cc(S(=O)(=O)Cc4ccccc4)ccc3Cl)c(=O)c12] | (DMSO-d6) 4.77 (2H, s), 7.15-7.25 (2H, m), 7.25-7.35 (3H, m), 7.39 (1H, s), 7.87 (1H, dd, J = 8.6 Hz, 2.2 Hz), 7.95 (1H, d, J = 8.36 Hz), 8.09 (1H, d, J = 2.2 Hz), 12.08 (1H, s), 14.26 (1H, s) |
| 205 | 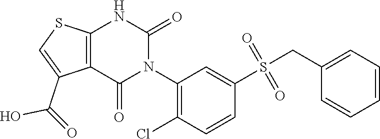 [SMILES: O=C(O)c1csc2[nH]c(=O)n(-c3cc(S(=O)(=O)Cc4ccccc4)ccc3Cl)c(=O)c12] | (DMSO-d6) 4.77 (2H, s), 7.15-7.25 (2H, m), 7.25-7.35 (3H, m), 7.8-8.0 (3H, m), 8.08 (1H, d, J = 2.3 Hz), 13.78 (1H, s) |
| 206 | 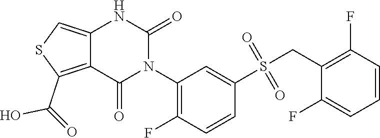 [SMILES: O=C(O)c1scc2[nH]c(=O)n(-c3cc(S(=O)(=O)Cc4c(F)cccc4F)ccc3F)c(=O)c12] | (DMSO-d6) 4.76 (2H, s), 7.05-7.15 (2H, m), 7.36 (1H, s), 7.4-7.55 (1H, m), 7.7- 7.8 (1H, m), 7.95-8.1 (2H, m), 12.02 (1H, s), 14.26 (1H, s) |
| 207 | 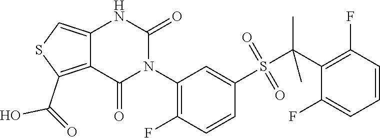 [SMILES: CC(C)(c1c(F)cccc1F)S(=O)(=O)c1ccc(F)c(-n2c(=O)[nH]c3csc(C(=O)O)c3c2=O)c1] | (DMSO-d6) 1.89 (6H, s), 7.0-7.1 (2H, m), 7.36 (1H, s), 7.4-7.5 (1H, m), 7.6-7.7 (2H, m), 8.0-8.1 (1H, m), 12.0 (1H, s), 14.28 (1H, s) |
| 208 | 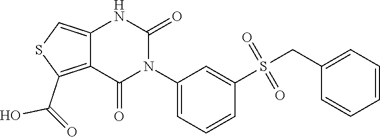 [SMILES: O=C(O)c1scc2[nH]c(=O)n(-c3cccc(S(=O)(=O)Cc4ccccc4)c3)c(=O)c12] | (DMSO-d6) 4.72 (2H, s), 7.15-7.25 (2H, m), 7.25-7.35 (3H, m), 7.39 (1H, s), 7.7- 7.95 (4H, m), 11.97 (1H, s), 14.72 (1H, s) |
| 209 | 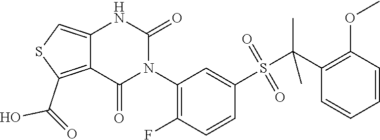 [SMILES: COc1ccccc1C(C)(C)S(=O)(=O)c1ccc(F)c(-n2c(=O)[nH]c3csc(C(=O)O)c3c2=O)c1] | (DMSO-d6) 1.86 (6H, s), 3.35 (3H, s), 6.83 (1H, d, J = 8.6 Hz), 6.9-7.0 (1H, m), 7.25-7.35 (2H, m), 7.37 (1H, s), 7.4-7.45 (1H, m), 7.5-7.6 (1H, m), 7.85-7.95 (1H, m), 12.0 (1H, s), 14.29 (1H, s) |
| 210 | 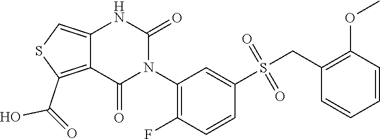 [SMILES: COc1ccccc1CS(=O)(=O)c1ccc(F)c(-n2c(=O)[nH]c3csc(C(=O)O)c3c2=O)c1] | (DMSO-d6) 3.46 (3H, s), 4.63 (2H, s), 6.85-6.95 (2H, m), 7.2-7.35 (2H, m), 7.37 (1H, s), 7.6-7.75 (2H, m), 7.9-8.0 (1H, m), 12.02 (1H, s), 14.26 (1H, s) |
| 211 | 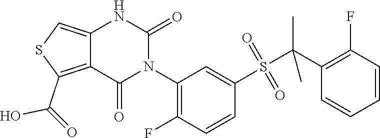 [SMILES: CC(C)(c1ccccc1F)S(=O)(=O)c1ccc(F)c(-n2c(=O)[nH]c3csc(C(=O)O)c3c2=O)c1] | (DMSO-d6) 1.81 (6H, s), 7.05-7.2 (2H, m), 7.3-7.45 (3H, m), 7.5-7.65 (2H, m), 7.98 (1H, dd, J = 6.7 Hz, 2.3 Hz), 12.01 (1H, s), 14.27 (1H, s) |
| 212 | 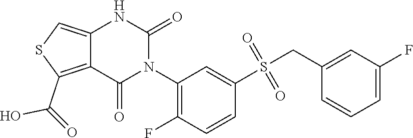 [SMILES: O=C(O)c1scc2[nH]c(=O)n(-c3cc(S(=O)(=O)Cc4cccc(F)c4)ccc3F)c(=O)c12] | (DMSO-d6) 4.81 (2H, s), 6.95-7.05 (2H, m), 7.1-7.2 (1H, m), 7.3-7.4 (2H, m), 7.7-7.75 (1H, m), 7.9-8.0 (1H, m), 8.04 (1H, dd, J = 6.8 Hz, 2.6 Hz), 12.05 (1H, s), 14.24 (1H, s) |
| 213 | 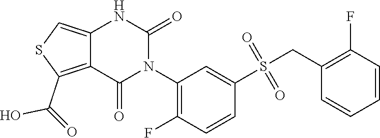 [SMILES: O=C(O)c1scc2[nH]c(=O)n(-c3cc(S(=O)(=O)Cc4ccccc4F)ccc3F)c(=O)c12] | (DMSO-d6) 4.7-4.8 (2H, m), 7.1-7.3 (3H, m), 7.35-7.45 (2H, m), 7.65-7.75 (1H, m), 7.9-8.0 (1H, m), 8.05 (1H, dd, J = 6.8 Hz, 2.5 Hz), 12.03 (1H, s), 14.25 (1H, s) |
| 214 | 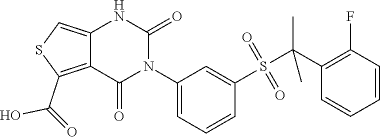 [SMILES: CC(C)(c1ccccc1F)S(=O)(=O)c1cccc(-n2c(=O)[nH]c3csc(C(=O)O)c3c2=O)c1] | (DMSO-d6) 1.75-1.85 (6H, m), 7.05-7.2 (2H, m), 7.3-7.5 (4H, m), 7.6-7.8 (3H, m), 11.91 (1H, s), 14.72 (1H, s) |
| 215 | 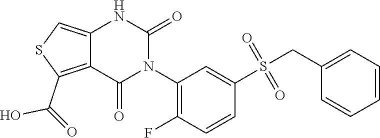 [SMILES: O=C(O)c1scc2[nH]c(=O)n(-c3cc(S(=O)(=O)Cc4ccccc4)ccc3F)c(=O)c12] | (DMSO-d6) 4.74 (2H, s), 7.15-7.2 (2H, m), 7.25-7.35 (3H, m), 7.37 (1H, s), 7.65-7.75 (1H, m), 7.85-7.95 (1H, m), 8.07 (1H, dd, J = 6.6 Hz, 2.5 Hz), 12.04 (1H, s), 14.24 (1H, s) |
| 216 | 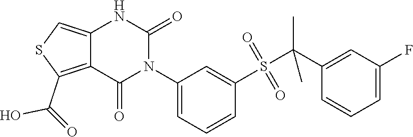 [SMILES: CC(C)(c1cccc(F)c1)S(=O)(=O)c1cccc(-n2c(=O)[nH]c3csc(C(=O)O)c3c2=O)c1] | (DMSO-d6) 1.72 (6H, s), 7.1-7.25 (3H, m), 7.3-7.45 (3H, m), 7.6-7.7 (1H, m), 7.7-7.8 (2H, m), 11.92 (1H, s), 14.7 (1H, s) |
| 217 | 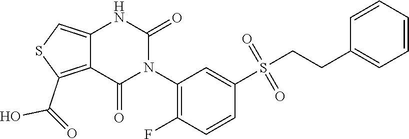 [SMILES: O=C(O)c1scc2[nH]c(=O)n(-c3cc(S(=O)(=O)CCc4ccccc4)ccc3F)c(=O)c12] | (DMSO-d6) 2.85-2.95 (2H, m), 3.65- 3.75 (2H, m), 7.15-7.3 (5H, m), 7.37 (1H, s), 7.7-7.8 (1H, m), 8.1-8.15 (1H, m), 8.27 (1H, dd, J = 6.6 Hz, 2.6 Hz), 12.05 (1H, s), 14.23 (1H, brs) |
| 218 | 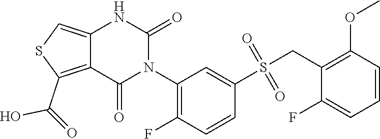 [SMILES: COc1cccc(F)c1CS(=O)(=O)c1ccc(F)c(-n2c(=O)[nH]c3csc(C(=O)O)c3c2=O)c1] | (DMSO-d6) 3.47 (3H, s), 4.62 (2H, s), 6.75 (1H, d, J = 8.4 Hz), 6.8-6.9 (1H, m), 7.3-7.4 (2H, m), 7.65-7.75 (1H, m), 7.8- 7.9 (1H, m), 7.95-8.05 (1H, m), 12.0 (1H, s), 14.27 (1H, brs) |
| 219 | 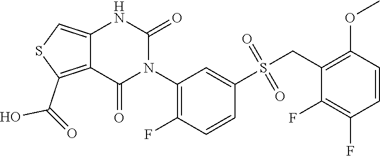 [SMILES: COc1ccc(F)c(F)c1CS(=O)(=O)c1ccc(F)c(-n2c(=O)[nH]c3csc(C(=O)O)c3c2=O)c1] | (DMSO-d6) 3.45 (3H, s), 4.68 (2H, s), 6.7-6.75 (1H, m), 7.35-7.45 (2H, m), 7.65-7.75 (1H, m), 7.85-8.0 (2H, m), 12.01 (1H, s), 14.25 (1H, brs) |
| 220 | 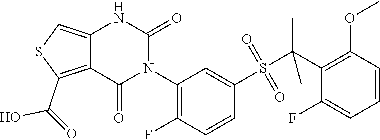 [SMILES: COc1cccc(F)c1C(C)(C)S(=O)(=O)c1ccc(F)c(-n2c(=O)[nH]c3csc(C(=O)O)c3c2=O)c1] | (DMSO-d6) 1.9-2.0 (6H, m), 3.38 (3H, s), 6.65-6.8 (2H, m), 7.3-7.4 (2H, m), 7.4-7.5 (1H, m), 7.55-7.6 (1H, m), 7.95- 8.0 (1H, m), 12.0 (1H, s), 14.28 (1H, brs) |
| 221 | 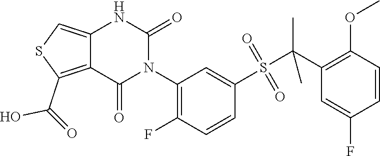 [SMILES: COc1ccc(F)cc1C(C)(C)S(=O)(=O)c1ccc(F)c(-n2c(=O)[nH]c3csc(C(=O)O)c3c2=O)c1] | (DMSO-d6) 1.85 (6H, s), 3.34 (3H, s), 6.84 (1H, dd, J = 9.1 Hz, 5.2 Hz), 7.1- 7.25 (2H, m), 7.37 (1H, s), 7.4-7.45 (1H, m), 7.55-7.65 (1H, m), 7.9-8.0 (1H, m), 12.0 (1H, s), 14.29 (1H, brs) |
| 222 | 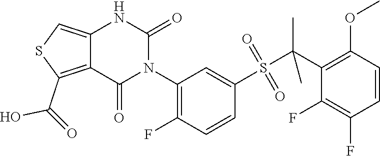 [SMILES: COc1ccc(F)c(F)c1C(C)(C)S(=O)(=O)c1ccc(F)c(-n2c(=O)[nH]c3csc(C(=O)O)c3c2=O)c1] | (DMSO-d6) 1.9-2.0 (6H, m), 3.36 (3H, s), 6.65-6.7 (1H, m), 7.35-7.45 (2H, m), 7.5-7.65 (2H, m), 7.95-8.0 (1H, m), 12.0 (1H, s), 14.28 (1H, brs) |
| 223 | 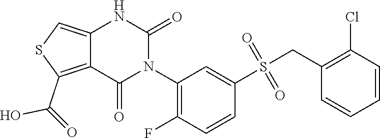 [SMILES: O=C(O)c1scc2[nH]c(=O)n(-c3cc(S(=O)(=O)Cc4ccccc4Cl)ccc3F)c(=O)c12] | (DMSO-d6) 4.85 (2H, s), 7.3-7.45 (5H, m), 7.65-7.75 (1H, m), 7.8-7.9 (1H, m), 8.05-8.1 (1H, m), 12.03 (1H, s), 14.25 (1H, brs) |
| 224 | 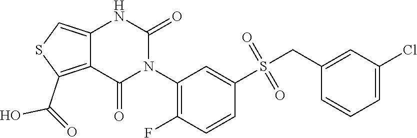 [SMILES: O=C(O)c1scc2[nH]c(=O)n(-c3cc(S(=O)(=O)Cc4cccc(Cl)c4)ccc3F)c(=O)c12] | (DMSO-d6) 4.81 (2H, s), 7.05-7.15 (1H, m), 7.25-7.45 (4H, m), 7.7-7.8 (1H, m), 7.9-8.0 (1H, m), 8.05-8.1 (1H, m), 12.05 (1H, s), 14.24 (1H, brs) |
| 225 | 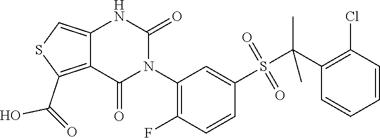 [SMILES: CC(C)(c1ccccc1Cl)S(=O)(=O)c1ccc(F)c(-n2c(=O)[nH]c3csc(C(=O)O)c3c2=O)c1] | (DMSO-d6) 1.95 (6H, s), 7.25-7.45 (5H, m), 7.5-7.65 (2H, m), 8.03 (1H, dd, J = 6.8 Hz, 2.4 Hz), 12.0 (1H, s), 14.28 (1H, s) |
| 226 | 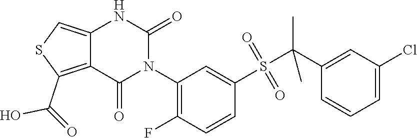 [SMILES: CC(C)(c1cccc(Cl)c1)S(=O)(=O)c1ccc(F)c(-n2c(=O)[nH]c3csc(C(=O)O)c3c2=O)c1] | (DMSO-d6) 1.72 (6H, s), 7.3-7.5 (6H, m), 7.55-7.65 (1H, m), 8.01 (1H, dd, J = 6.7 Hz, 2.3 Hz), 12.02 (1H, s), 14.25 (1H, s) |
| 227 | 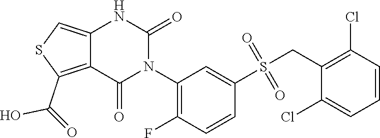 [SMILES: O=C(O)c1scc2[nH]c(=O)n(-c3cc(S(=O)(=O)Cc4c(Cl)cccc4Cl)ccc3F)c(=O)c12] | (DMSO-d6) 4.96 (2H, s), 7.35-7.45 (2H, m), 7.45-7.55 (2H, m), 7.65-7.75 (1H, m), 7.85-7.95 (1H, m), 8.15-8.2 (1H, m), 12.05 (1H, s), 14.28 (1H, s) |
| 228 | 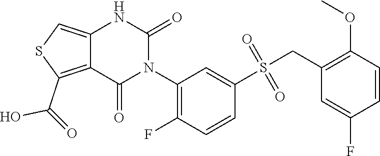 [SMILES: COc1ccc(F)cc1CS(=O)(=O)c1ccc(F)c(-n2c(=O)[nH]c3csc(C(=O)O)c3c2=O)c1] | (DMSO-d6) 3.43 (3H, s), 4.6-4.7 (2H, m), 6.85-6.9 (1H, m), 7.1-7.2 (2H, m), 7.37 (1H, s), 7.65-7.7 (1H, m), 7.75-7.85 (1H, m), 7.9-8.0 (1H, m), 12.04 (1H, s), 14.29 (1H, s) |
| 229 | 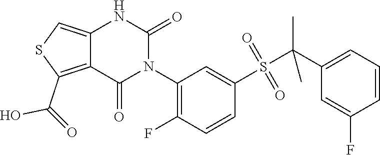 [SMILES: CC(C)(c1cccc(F)c1)S(=O)(=O)c1ccc(F)c(-n2c(=O)[nH]c3csc(C(=O)O)c3c2=O)c1] | (DMSO-d6) 1.72 (3H, s), 1.73 (3H, s), 7.15-7.25 (3H, m), 7.35-7.45 (2H, m), 7.45-7.55 (1H, m), 7.6 (1H, t, J = 9.1 Hz), 7.9-8.0 (1H, m), 12.04 (1H, s), 14.28 (1H, brs) |
| 230 | 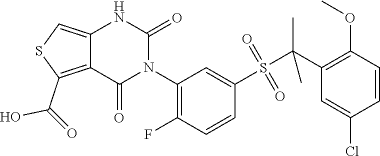 [SMILES: COc1ccc(Cl)cc1C(C)(C)S(=O)(=O)c1ccc(F)c(-n2c(=O)[nH]c3csc(C(=O)O)c3c2=O)c1] | (DMSO-d6) 1.86 (6H, s), 6.87 (1H, d, J = 8.8 Hz), 7.35-7.45 (4H, m), 7.55-7.65 (1H, m), 7.95-8.0 (1H, m), 12.03 (1H, s), 14.3 (1H, brs) |
| 231 | 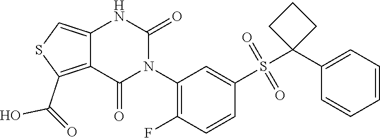 [SMILES: O=C(O)c1scc2[nH]c(=O)n(-c3cc(S(=O)(=O)C4(c5ccccc5)CCC4)ccc3F)c(=O)c12] | (DMSO-d6) 1.8-1.95 (1H, m), 2.0-2.15 (1H, m), 2.55-2.7 (2H, m), 3.0-3.15 (2H, m), 6.95-7.05 (2H, m), 7.2-7.35 (4H, m), 7.37 (1H, s), 7.45-7.55 (1H, m), 7.9- 8.0 (1H, m), 12.0 (1H, s), 14.28 (1H, brs) |
| 232 | 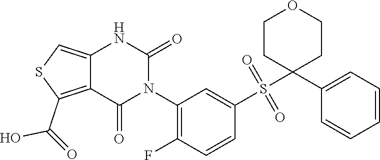 [SMILES: O=C(O)c1scc2[nH]c(=O)n(-c3cc(S(=O)(=O)C4(c5ccccc5)CCOCC4)ccc3F)c(=O)c12] | (DMSO-d6) 2.2-2.35 (2H, m), 3.05-3.2 (2H, m), 3.8-3.9 (2H, m), 7.2-7.4 (7H, m), 7.45-7.55 (1H, m), 7.8-7.9 (1H, m), 12.02 (1H, s), 14.25 (1H, brs) |
| 233 | 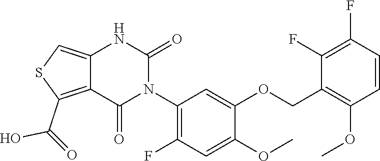 [SMILES: COc1cc(F)c(-n2c(=O)[nH]c3csc(C(=O)O)c3c2=O)cc1OCc1c(OC)ccc(F)c1F] | (DMSO-d6) 3.75-3.85 (6H, m), 4.96 (2H, s), 6.85-6.95 (1H, m), 7.13 (1H, d, J = 11.3 Hz), 7.26 (1H, d, J = 7.2 Hz), 7.39 (1H, s), 7.4-7.55 (1H, m), 12.0 (1H, s), 14.53 (1H, s) |
| 234 | 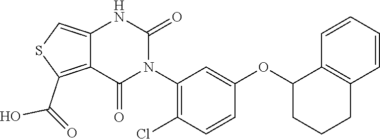 [SMILES: O=C(O)c1scc2[nH]c(=O)n(-c3cc(OC4CCCc5ccccc54)ccc3Cl)c(=O)c12] | (DMSO-d6) 1.7-2.1 (4H, m), 2.65-2.9 (2H, m), 5.45-5.5 (1H, m), 7.1-7.45 (7H, m), 7.57 (1H, d, J = 9.0 Hz), 12.0-12.1 (1H, m), 14.45 (1H, s) |
| 235 | 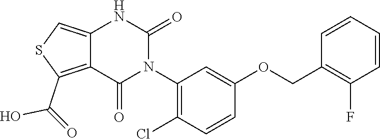 [SMILES: O=C(O)c1scc2[nH]c(=O)n(-c3cc(OCc4ccccc4F)ccc3Cl)c(=O)c12] | (DMSO-d6) 5.14 (2H, s), 7.15-7.5 (6H, m), 7.55-7.65 (2H, m), 12.05 (1H, s), 14.43 (1H, s) |
| 236 | 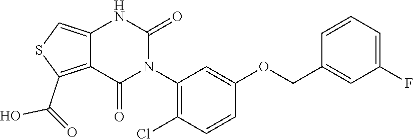 [SMILES: O=C(O)c1scc2[nH]c(=O)n(-c3cc(OCc4cccc(F)c4)ccc3Cl)c(=O)c12] | (DMSO-d6) 5.14 (2H, s), 7.15-7.25 (2H, m), 7.25-7.5 (5H, m), 7.58 (1H, d, J = 9.1 Hz), 12.05 (1H, s), 14.43 (1H, s) |
| 237 | 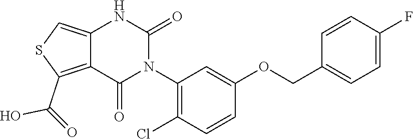 [SMILES: O=C(O)c1scc2[nH]c(=O)n(-c3cc(OCc4ccc(F)cc4)ccc3Cl)c(=O)c12] | (DMSO-d6) 5.09 (2H, s), 7.15-7.3 (3H, m), 7.32 (1H, d, J = 2.9 Hz), 7.41 (1H, s), 7.5-7.6 (3H, m), 12.04 (1H, s), 14.44 (1H, s) |
| 238 | 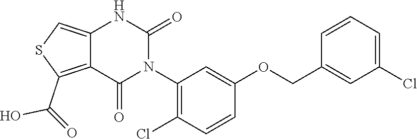 [SMILES: O=C(O)c1scc2[nH]c(=O)n(-c3cc(OCc4cccc(Cl)c4)ccc3Cl)c(=O)c12] | (DMSO-d6) 5.13 (2H, s), 7.19 (1H, dd, J = 9.0 Hz, 3.0 Hz), 7.3-7.5 (5H, m), 7.5- 7.6 (2H, m), 12.05 (1H, s), 14.44 (1H, s) |
| 239 | 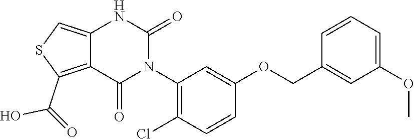 [SMILES: COc1cccc(COc2ccc(Cl)c(-n3c(=O)[nH]c4csc(C(=O)O)c4c3=O)c2)c1] | (DMSO-d6) 3.76 (3H, s), 5.08 (2H, s), 6.85-6.95 (1H, m), 7.0-7.05 (2H, m), 7.18 (1H, dd, J = 9.1 Hz, 3.1 Hz), 7.25- 7.35 (2H, m), 7.41 (1H, s), 7.57 (1H, d, J = 9.1 Hz), 12.04 (1H, s), 14.44 (1H, s) |
| 240 | 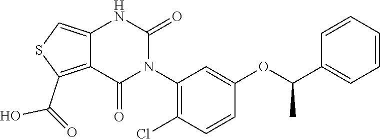 [SMILES: C[C@@H](Oc1ccc(Cl)c(-n2c(=O)[nH]c3csc(C(=O)O)c3c2=O)c1)c1ccccc1] | (DMSO-d6) 1.5-1.6 (3H, m), 5.45-5.55 (1H, m), 7.0-7.1 (1H, m), 7.23 (1H dd, J = 6.1 Hz, 2.9 Hz), 7.25-7.5 (7H, m), 11.95-12.1 (1H, m), 14.42 (1H, s) |
| 241 | 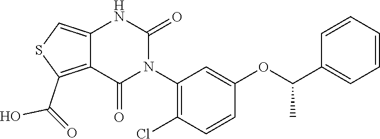 [SMILES: C[C@H](Oc1ccc(Cl)c(-n2c(=O)[nH]c3csc(C(=O)O)c3c2=O)c1)c1ccccc1] | (DMSO-d6) 1.5-1.6 (3H, m), 5.45-5.55 (1H, m), 7.0-7.1 (1H, m), 7.23 (1H, dd, J = 6.1 Hz, 2.9 Hz), 7.25-7.5 (7H, m), 11.95-12.1 (1H, m), 14.42 (1H, s) |
| 242 | 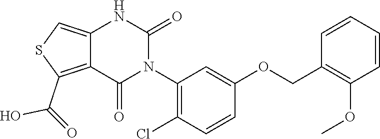 [SMILES: COc1ccccc1COc1ccc(Cl)c(-n2c(=O)[nH]c3csc(C(=O)O)c3c2=O)c1] | (DMSO-d6) 3.82 (3H, s), 5.04 (2H, s), 6.95-7.0 (1H, m), 7.06 (1H, d, J = 7.9 Hz), 7.17 (1H, dd, J = 9.1 Hz, 2.9 Hz), 7.3-7.45 (4H, m), 7.56 (1H, d, J = 9.1 Hz), 12.04 (1H, s), 14.46 (1H, s) |
| 243 | 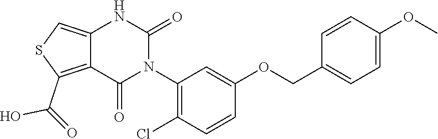 [SMILES: COc1ccc(COc2ccc(Cl)c(-n3c(=O)[nH]c4csc(C(=O)O)c4c3=O)c2)cc1] | (DMSO-d6) 3.76 (3H, s), 5.02 (2H, s), 6.9-7.0 (2H, m), 7.15-7.2 (1H, m), 7.3 (1H, d, J = 3.3 Hz), 7.35-7.45 (3H, m), 7.56 (1H, d, J = 9.0 Hz), 12.04 (1H, s), 14.46 (1H, s) |
| 244 | 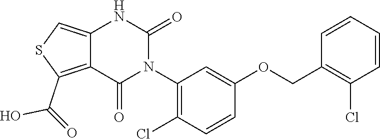 [SMILES: O=C(O)c1scc2[nH]c(=O)n(-c3cc(OCc4ccccc4Cl)ccc3Cl)c(=O)c12] | (DMSO-d6) 5.18 (2H, s), 7.02 (1H, s), 7.16 (1H, dd, J = 9.0 Hz, 2.7 Hz), 7.25- 7.4 (2H, m), 7.5-7.6 (2H, m), 7.8-7.9 (1H, m), 8.58 (1H, d, J = 4.5 Hz), 11.0- 12.5 (1H, br) |
| 245 | 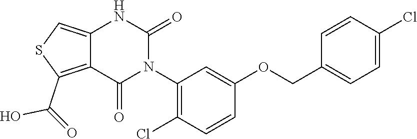 [SMILES: O=C(O)c1scc2[nH]c(=O)n(-c3cc(OCc4ccc(Cl)cc4)ccc3Cl)c(=O)c12] | (DMSO-d6) 5.11 (2H, s), 7.15-7.2 (1H, m), 7.32 (1H, d, J = 2.9 Hz), 7.41 (1H, s), 7.45-7.55 (4H, m), 7.57 (1H, d, J = 8.6 Hz), 12.06 (1H, s), 14.43 (1H, s) |
| 246 | 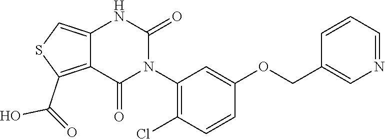 [SMILES: O=C(O)c1scc2[nH]c(=O)n(-c3cc(OCc4cccnc4)ccc3Cl)c(=O)c12] | (DMSO-d6) 5.16 (2H, s), 7.1-7.25 (2H, m), 7.31 (1H, d, J = 2.9 Hz), 7.4-7.5 (1H, m), 7.56 (1H, d, J = 9.0 Hz), 7.85-7.95 (1H, m), 8.5-8.6 (1H, m), 8.69 (1H, s), 11.0-13.0 (1H, br) |
| 247 | 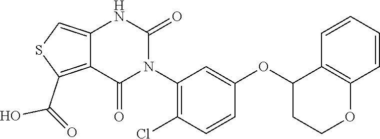 [SMILES: O=C(O)c1scc2[nH]c(=O)n(-c3cc(OC4CCOc5ccccc54)ccc3Cl)c(=O)c12] | (DMSO-d6) 2.1-2.25 (2H, m), 4.1-4.2 (1H, m), 4.25-4.35 (1H, m), 5.45-5.55 (1H, m), 6.8-6.95 (2H, m), 7.2-7.35 (3H, m), 7.35-7.45 (2H, m), 7.59 (1H, d, J = 8.7 Hz), 12.0-12.05 (1H, m), 14.42 (1H, s) |
| 248 | 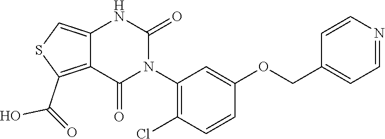 [SMILES: O=C(O)c1scc2[nH]c(=O)n(-c3cc(OCc4ccncc4)ccc3Cl)c(=O)c12] | (DMSO-d6) 5.2 (2H, s), 7.1-7.2 (2H, m), 7.3 (1H, d, J = 2.9 Hz), 7.4-7.5 (2H, m), 7.56 (1H, d, J = 8.8 Hz), 8.55-8.65 (2H, m), 11.0-13.0 (1H, br) |
| 249 | 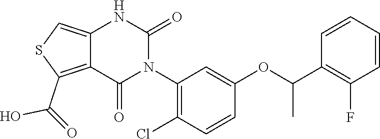 [SMILES: CC(Oc1ccc(Cl)c(-n2c(=O)[nH]c3csc(C(=O)O)c3c2=O)c1)c1ccccc1F] | (DMSO-d6) 1.6 (3H, d, J = 6.3 Hz), 5.65-5.75 (1H, m), 7.0-7.1 (1H, m), 7.15-7.3 (3H, m), 7.3-7.45 (2H, m), 7.45-7.55 (2H, m), 11.95-12.1 (1H, m), 14.42 (1H, s) |
| 250 | 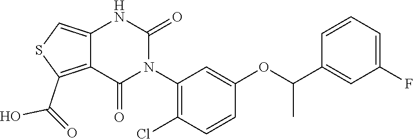 [SMILES: CC(Oc1ccc(Cl)c(-n2c(=O)[nH]c3csc(C(=O)O)c3c2=O)c1)c1cccc(F)c1] | (DMSO-d6) 1.5-1.6 (3H, m), 5.5-5.6 (1H, m), 7.0-7.15 (2H, m), 7.2-7.3 (3H, m), 7.35-7.55 (3H, m), 11.95-12.0 (1H, m), 14.41 (1H, s) |
| 251 | 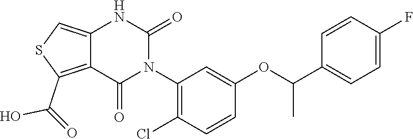 [SMILES: CC(Oc1ccc(Cl)c(-n2c(=O)[nH]c3csc(C(=O)O)c3c2=O)c1)c1ccc(F)cc1] | (DMSO-d6) 1.5-1.6 (3H, m), 5.45-5.6 (1H, m), 7.0-7.1 (1H, m), 7.15-7.25 (3H, m), 7.4 (1H, d, J = 4.1 Hz), 7.45-7.55 (3H, m), 11.95-12.05 (1H, m), 14.42 (1H, s) |
| 252 | 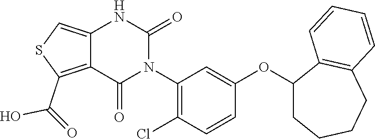 [SMILES: O=C(O)c1scc2[nH]c(=O)n(-c3cc(OC4CCCCc5ccccc54)ccc3Cl)c(=O)c12] | (DMSO-d6) 1.35-1.5 (1H, m), 1.65-2.05 (5H, m), 2.8-3.0 (2H, m), 5.45-5.6 (1H, m), 7.05-7.2 (4H, m), 7.25-7.35 (2H, m), 7.39 (1H, d, J = 3.8 Hz), 7.52 (1H, d, J = 8.8 Hz), 12.02 (1H, s), 14.43 (1H, s) |
| 253 | 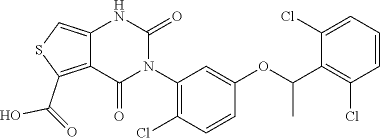 [SMILES: CC(Oc1ccc(Cl)c(-n2c(=O)[nH]c3csc(C(=O)O)c3c2=O)c1)c1c(Cl)cccc1Cl] | (DMSO-d6) 1.73 (3H, d, J = 6.6 Hz), 6.07 (1H, q, J = 6.6 Hz), 6.89 (1H, dd, J = 9.0 Hz, 3.0 Hz), 7.15-7.2 (1H, m), 7.3- 7.4 (2H, m), 7.45-7.55 (3H, m), 12.0 (1H, s), 14.4 (1H, s) |
| 254 | 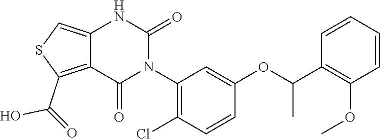 [SMILES: COc1ccccc1C(C)Oc1ccc(Cl)c(-n2c(=O)[nH]c3csc(C(=O)O)c3c2=O)c1] | (DMSO-d6) 1.53 (3H, d, J = 6.2 Hz), 5.68 (1H, q, J = 6.2 Hz), 6.85-7.0 (2H, m), 7.05 (1H, d, J = 8.4 Hz), 7.2 (1H, t, J = 3.2 Hz), 7.25-7.4 (3H, m), 7.47 (1H, d, J = 9.3 Hz), 12.01 (1H, s), 14.45 (1H, s) |
| 255 | 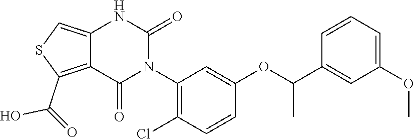 [SMILES: COc1cccc(C(C)Oc2ccc(Cl)c(-n3c(=O)[nH]c4csc(C(=O)O)c4c3=O)c2)c1] | (DMSO-d6) 1.5-1.6 (3H, m), 3.7-3.75 (3H, m), 5.4-5.5 (1H, m), 6.8-6.9 (1H, m), 6.95-7.1 (3H, m), 7.2-7.3 (2H, m), 7.4 (1H, d, J = 3.4 Hz), 7.47 (1H, dd, J = 9.0 Hz, 1.6 Hz), 11.95-12.05 (1H, m), 14.43 (1H, s) |
| 256 | 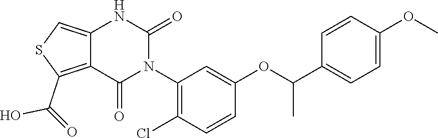 [SMILES: COc1ccc(C(C)Oc2ccc(Cl)c(-n3c(=O)[nH]c4csc(C(=O)O)c4c3=O)c2)cc1] | (DMSO-d6) 1.5-1.6 (3H, m), 3.73 (3H, s), 5.4-5.5 (1H, m), 6.85-6.95 (2H, m), 7.0-7.05 (1H, m), 7.15-7.25 (1H, m), 7.3-7.5 (4H, m), 11.95-12.05 (1H, m), 14.44 (1H, s) |
| 257 | 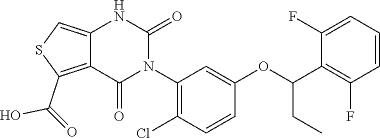 [SMILES: CCC(Oc1ccc(Cl)c(-n2c(=O)[nH]c3csc(C(=O)O)c3c2=O)c1)c1c(F)cccc1F] | (DMSO-d6) 0.93 (3H, t, J = 7.5 Hz), 1.9- 2.05 (1H, m), 2.1-2.25 (1H, m), 5.55 (1H, t, J = 7.3 Hz), 6.95-7.25 (4H, m), 7.35-7.5 (2H, m), 7.52 (1H, d, J = 8.7 Hz), 11.95-12.05 (1H, m), 14.4 (1H, s) |
| 258 | 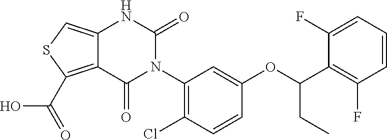 [SMILES: CCC(Oc1ccc(Cl)c(-n2c(=O)[nH]c3csc(C(=O)O)c3c2=O)c1)c1c(F)cccc1F] | (DMSO-d6) 0.93 (3H, t, J = 7.4 Hz), 1.9- 2.05 (1H, m), 6.95-7.25 (4H, m), 7.35-7.5 (1H, m), 6.95-7.25 (4H, m), 7.35-7.5 (1H, m), 7.52 (1H, d, J = 9.2 Hz), 7.95 (1H, d, J = 6.7 Hz), 12.99 (1H, s), 13.8- 14.0 (1H, m) |
| 259 | 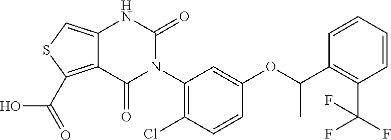 [SMILES: CC(Oc1ccc(Cl)c(-n2c(=O)[nH]c3csc(C(=O)O)c3c2=O)c1)c1ccccc1C(F)(F)F] | (DMSO-d6) 1.55-1.65 (3H, m), 5.65-5.8 (1H, m), 6.9-7.0 (1H, m), 7.2-7.3 (1H, m), 7.35-7.45 (1H, m), 7.45-7.6 (2H, m), 7.7-7.85 (3H, m), 12.03 (1H, s), 14.41 (1H, s) |
| 260 | 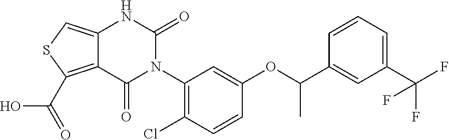 [SMILES: CC(Oc1ccc(Cl)c(-n2c(=O)[nH]c3csc(C(=O)O)c3c2=O)c1)c1cccc(C(F)(F)F)c1] | (DMSO-d6) 1.55-1.65 (3H, m), 5.6-5.7 (1H, m), 7.05-7.15 (1H, m), 7.25-7.3 (1H, m), 7.4 (1H, d, J = 5.6 Hz), 7.45- 7.55 (1H, m), 7.55-7.85 (4H, m), 11.95- 12.1 (1H, m), 14.4 (1H, s) |
| 261 | 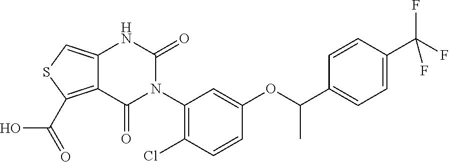 [SMILES: CC(Oc1ccc(Cl)c(-n2c(=O)[nH]c3csc(C(=O)O)c3c2=O)c1)c1ccc(C(F)(F)F)cc1] | (DMSO-d6) 1.55-1.65 (3H, m), 5.6-5.7 (1H, m), 7.0-7.1 (1H, m), 7.25 (1H, dd, J = 6.6 Hz, 3.0 Hz), 7.4 (1H, d, J = 5.3 Hz), 7.45-7.55 (1H, m), 7.6-7.7 (2H, m), 7.7-7.8 (2H, m), 11.95-12.1 (1H, m), 14.4 (1H, s) |
| 262 | 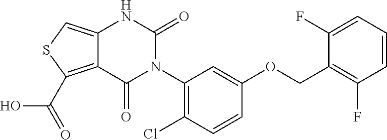 [SMILES: O=C(O)c1scc2[nH]c(=O)n(-c3cc(OCc4c(F)cccc4F)ccc3Cl)c(=O)c12] | (DMSO-d6) 5.13 (2H, s), 7.15-7.3 (3H, m), 7.34 (1H, d, J = 2.9 Hz), 7.4 (1H, s), 7.5-7.65 (2H, m), 12.03 (1H, s), 14.4 (1H, s) |
| 263 | 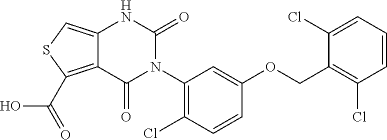 [SMILES: O=C(O)c1scc2[nH]c(=O)n(-c3cc(OCc4c(Cl)cccc4Cl)ccc3Cl)c(=O)c12] | (DMSO-d6) 5.24 (2H, s), 7.2-7.65 (7H, m), 12.02 (1H, s), 14.39 (1H, s) |
| 264 | 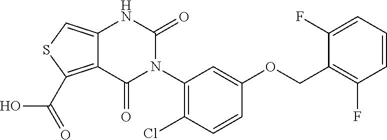 [SMILES: O=C(O)c1scc2[nH]c(=O)n(-c3cc(OCc4c(F)cccc4F)ccc3Cl)c(=O)c12] | (DMSO-d6) 5.12 (2H, s), 7.15-7.25 (3H, m), 7.32 (1H, d, J = 3.1 Hz), 7.5-7.65 (2H, m), 7.94 (1H, s), 12.8-13.2 (1H, br), 13.93 (1H, s) |
| 265 | 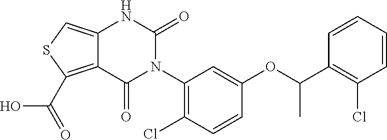 [SMILES: CC(Oc1ccc(Cl)c(-n2c(=O)[nH]c3csc(C(=O)O)c3c2=O)c1)c1ccccc1Cl] | (DMSO-d6) 1.59 (3H, d, J = 6.3 Hz), 5.71 (1H, q, J = 6.3 Hz), 6.85-6.95 (1H, m), 7.2-7.25 (1H, m), 7.3-7.45 (3H, m), 7.45-7.55 (3H, m), 11.95-12.05 (1H, m), 14.39 (1H, s) |
| 266 | 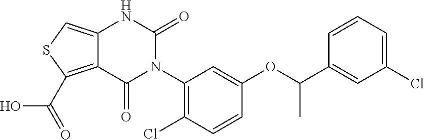 [SMILES: CC(Oc1ccc(Cl)c(-n2c(=O)[nH]c3csc(C(=O)O)c3c2=O)c1)c1cccc(Cl)c1] | (DMSO-d6) 1.56 (3H, d, J = 6.2 Hz), 5.45-5.6 (1H, m), 7.0-7.1 (1H, m), 7.2- 7.55 (7H, m), 11.95-12.05 (1H, m), 14.39 (1H, s) |
| 267 | 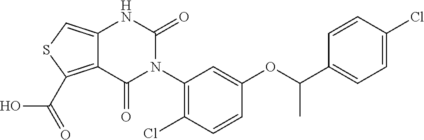 [SMILES: CC(Oc1ccc(Cl)c(-n2c(=O)[nH]c3csc(C(=O)O)c3c2=O)c1)c1ccc(Cl)cc1] | (DMSO-d6) 1.5-1.6 (3H, m), 5.45-5.6 (1H, m), 7.0-7.05 (1H, m), 7.22 (1H, dd, J = 8.9 Hz, 3.0 Hz), 7.35-7.5 (6H, m), 11.9-12.05 (1H, m), 14.39 (1H, s) |
| 268 | 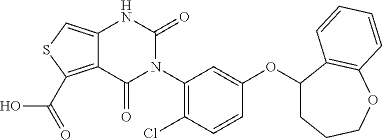 [SMILES: O=C(O)c1scc2[nH]c(=O)n(-c3cc(OC4CCCOc5ccccc54)ccc3Cl)c(=O)c12] | (DMSO-d6) 1.9-2.15 (4H, m), 3.75-3.85 (1H, m), 4.1-4.25 (1H, m), 5.45-5.55 (1H, m), 6.95-7.45 (7H, m), 7.53 (1H, d, J = 8.9 Hz), 11.95-12.05 (1H, m), 14.42 (1H, s) |
| 269 | 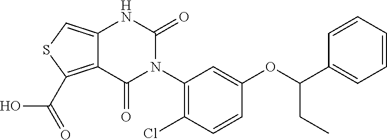 [SMILES: CCC(Oc1ccc(Cl)c(-n2c(=O)[nH]c3csc(C(=O)O)c3c2=O)c1)c1ccccc1] | (DMSO-d6) 0.91 (3H, t, J = 7.5 Hz), 1.75-2.0 (2H, m), 5.2-5.3 (1H, m), 6.95- 7.05 (1H, m), 7.15-7.5 (8H, m), 11.95- 12.05 (1H, m), 14.39 (1H, s) |
| 270 | 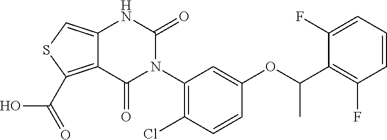 [SMILES: CC(Oc1ccc(Cl)c(-n2c(=O)[nH]c3csc(C(=O)O)c3c2=O)c1)c1c(F)cccc1F] | (DMSO-d6) 1.71 (3H, d, J = 6.5 Hz), 5.81 (1H, q, J = 6.5 Hz), 7.0-7.15 (3H, m), 7.2-7.25 (1H, m), 7.35-7.5 (2H, m), 7.53 (1H, d, J = 9.2 Hz), 11.95-12.05 (1H, m), 14.41 (1H, s) |
| 271 | 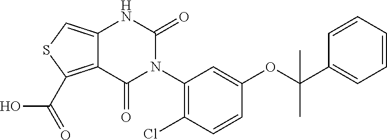 [SMILES: CC(C)(Oc1ccc(Cl)c(-n2c(=O)[nH]c3csc(C(=O)O)c3c2=O)c1)c1ccccc1] | (DMSO-d6) 1.65-1.75 (6H, m), 6.64 (1H, dd, J = 8.7 Hz, 2.8 Hz), 7.07 (1H, d, J = 2.8 Hz), 7.25-7.5 (7H, m), 11.97 (1H, s), 14.42 (1H, s) |
| 272 | 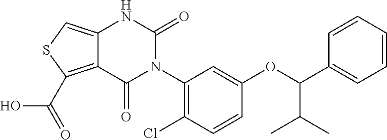 [SMILES: CC(C)C(Oc1ccc(Cl)c(-n2c(=O)[nH]c3csc(C(=O)O)c3c2=O)c1)c1ccccc1] | (DMSO-d6) 0.82 (3H, d, J = 6.4 Hz), 0.95-1.05 (3H, m), 2.05-2.15 (1H, m), 5.0-5.1 (1H, m), 6.95-7.05 (1H, m), 7.15-7.5 (8H, m), 11.95-12.05 (1H, m), 14.4 (1H, s) |
| 273 | 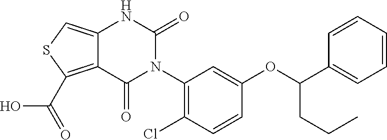 [SMILES: CCCC(Oc1ccc(Cl)c(-n2c(=O)[nH]c3csc(C(=O)O)c3c2=O)c1)c1ccccc1] | (DMSO-d6) 0.91 (3H, t, J = 7.4 Hz), 1.25-1.5 (2H, m), 1.7-1.8 (1H, m), 1.85- 2.0 (1H, m), 5.3-5.35 (1H, m), 6.95-7.05 (1H, m), 7.15-7.5 (8H, m), 11.95-12.05 (1H, m), 14.41 (1H, brs) |
| 274 | 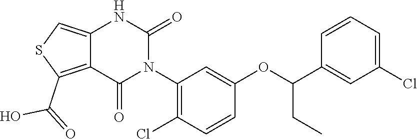 [SMILES: CCC(Oc1ccc(Cl)c(-n2c(=O)[nH]c3csc(C(=O)O)c3c2=O)c1)c1cccc(Cl)c1] | (DMSO-d6) 0.91 (3H, t, J = 7.4 Hz), 1.75-2.0 (2H, m), 5.25-5.35 (1H, m), 7.0-7.1 (1H, m), 7.2-7.25 (1H, m), 7.3- 7.55 (6H, m), 11.95-12.05 (1H, m), 14.4 (1H, s) |
| 275 | 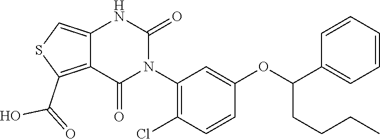 [SMILES: CCCCC(Oc1ccc(Cl)c(-n2c(=O)[nH]c3csc(C(=O)O)c3c2=O)c1)c1ccccc1] | (DMSO-d6) 0.85 (3H, t, J = 7.1 Hz), 1.2- 1.45 (4H, m), 1.7-1.85 (1H, m), 1.9-2.0 (1H, m), 5.25-5.35 (1H, m), 6.95-7.05 (1H, m), 7.15-7.5 (8H, m), 11.95-12.05 (1H, m), 14.41 (1H, s) |
| 276 | 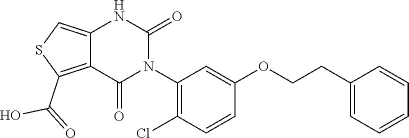 [SMILES: O=C(O)c1scc2[nH]c(=O)n(-c3cc(OCCc4ccccc4)ccc3Cl)c(=O)c12] | (DMSO-d6) 3.05 (2H, t, J = 7.0 Hz), 4.19 (2H, t, J = 7.0 Hz), 7.05-7.15 (1H, m), 7.2-7.35 (6H, m), 7.4 (1H, s), 7.54 (1H, d, J = 9.2 Hz), 12.03 (1H, s), 14.45 (1H, s) |
| 277 | 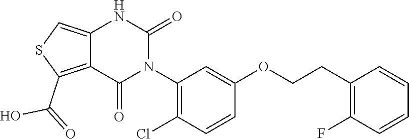 [SMILES: O=C(O)c1scc2[nH]c(=O)n(-c3cc(OCCc4ccccc4F)ccc3Cl)c(=O)c12] | (DMSO-d6) 3.09 (2H, t, J = 6.8 Hz), 4.19 (2H, t, J = 6.8 Hz), 7.05-7.35 (5H, m), 7.35-7.45 (2H, m), 7.54 (1H, d, J = 8.9 Hz), 12.03 (1H, s), 14.45 (1H, s) |
| 278 | 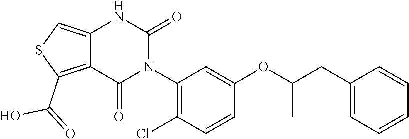 [SMILES: CC(Cc1ccccc1)Oc1ccc(Cl)c(-n2c(=O)[nH]c3csc(C(=O)O)c3c2=O)c1] | (DMSO-d6) 1.22 (3H, d, J = 6.0 Hz), 2.8-2.9 (1H, m), 2.95-3.05 (1H, m), 4.6- 4.75 (1H, m), 7.09 (1H, dd, J = 9.0 Hz, 3.0 Hz), 7.15-7.35 (6H, m), 7.35-7.45 (1H, m), 7.52 (1H, d, J = 9.2 Hz), 12.02 (1H, s), 14.46 (1H, s) |
| 279 | 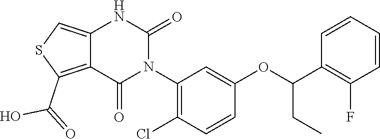 [SMILES: CCC(Oc1ccc(Cl)c(-n2c(=O)[nH]c3csc(C(=O)O)c3c2=O)c1)c1ccccc1F] | (DMSO-d6) 0.93 (3H, t, J = 7.5 Hz), 1.8- 2.05 (2H, m), 5.47 (1H, t, J = 6.1 Hz), 6.95-7.05 (1H, m), 7.15-7.25 (3H, m), 7.3-7.55 (4H, m), 11.95-12.05 (1H, m), 14.35-14.45 (1H, m) |
| 280 | 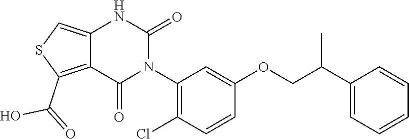 [SMILES: CC(COc1ccc(Cl)c(-n2c(=O)[nH]c3csc(C(=O)O)c3c2=O)c1)c1ccccc1] | (DMSO-d6) 1.32 (3H, d, J = 6.9 Hz), 3.15-3.3 (1H, m), 4.0-4.15 (2H, m), 7.09 (1H, dd, J = 8.9 Hz, 3.1 Hz), 7.15-7.45 (7H, m), 7.52 (1H, d, J = 8.9 Hz), 12.02 (1H, s), 14.45 (1H, s) |
| 281 | 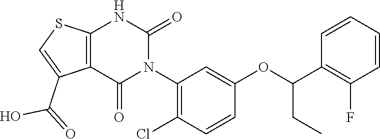 [SMILES: CCC(Oc1ccc(Cl)c(-n2c(=O)[nH]c3scc(C(=O)O)c3c2=O)c1)c1ccccc1F] | (DMSO-d6) 0.93 (3H, t, J = 7.5 Hz), 1.8- 2.05 (2H, m), 5.48 (1H, t, J = 6.5 Hz), 6.95-7.05 (1H, m), 7.15-7.25 (3H, m), 7.3-7.4 (1H, m), 7.4-7.55 (2H, m), 7.94 (1H, d, J = 3.4 Hz), 12.98 (1H, s), 13.93 (1H, s) |
| 282 | 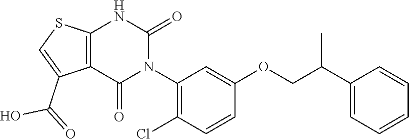 [SMILES: CC(COc1ccc(Cl)c(-n2c(=O)[nH]c3scc(C(=O)O)c3c2=O)c1)c1ccccc1] | (DMSO-d6) 1.32 (3H, d, J = 6.9 Hz), 3.15-3.3 (1H, m), 4.0-4.15 (2H, m), 7.05-7.1 (1H, m), 7.15-7.4 (6H, m), 7.52 (1H, d, J = 9.0 Hz), 7.95 (1H, s), 13.0 (1H, s), 13.98 (1H, s) |
| 283 | 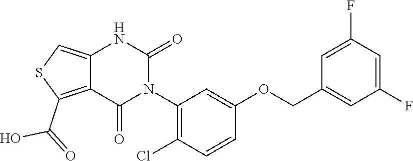 [SMILES: O=C(O)c1scc2[nH]c(=O)n(-c3cc(OCc4cc(F)cc(F)c4)ccc3Cl)c(=O)c12] | (DMSO-d6) 5.15 (2H, s), 7.15-7.3 (4H, m), 7.35 (1H, d, J = 2.8 Hz), 7.46 (1H, s), 7.59 (1H, d, J = 8.8 Hz), 12.19 (1H, s) |
| 284 | 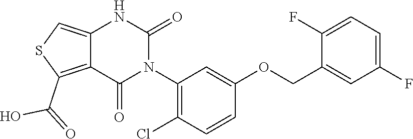 [SMILES: O=C(O)c1scc2[nH]c(=O)n(-c3cc(OCc4cc(F)ccc4F)ccc3Cl)c(=O)c12] | (DMSO-d6) 5.14 (2H, s), 7.2-7.5 (6H, m), 7.6 (1H, d, J = 8.7 Hz), 12.11 (1H, s), 14.44 (1H, s) |
| 285 | 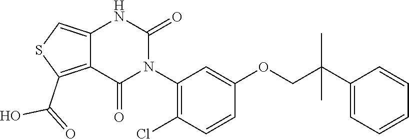 [SMILES: CC(C)(COc1ccc(Cl)c(-n2c(=O)[nH]c3csc(C(=O)O)c3c2=O)c1)c1ccccc1] | (DMSO-d6) 1.4 (6H, s), 4.01 (2H, s), 7.05-7.15 (1H, m), 7.15-7.25 (2H, m), 7.3-7.35 (2H, m), 7.39 (1H, s), 7.4-7.5 (2H, m), 7.52 (1H, d, J = 9.2 Hz), 12.0 (1H, s), 14.44 (1H, s) |
| 286 | 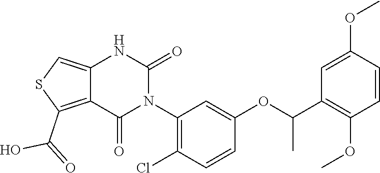 [SMILES: COc1ccc(OC)c(C(C)Oc2ccc(Cl)c(-n3c(=O)[nH]c4csc(C(=O)O)c4c3=O)c2)c1] | (DMSO-d6) 1.52 (3H, d, J = 6.3 Hz), 3.67 (3H, s), 3.75-3.85 (3H, m), 5.65 (1H, q, J = 6.3 Hz), 6.8-7.0 (4H, m), 7.15-7.25 (1H, m), 7.39 (1H, d, J = 1.9 Hz), 7.48 (1H, d, J = 8.9 Hz), 11.99 (1H, s), 14.42 (1H, s) |
| 287 | 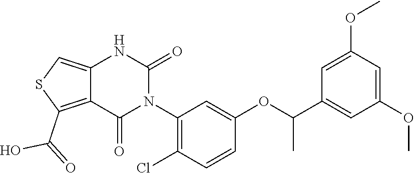 [SMILES: COc1cc(OC)cc(C(C)Oc2ccc(Cl)c(-n3c(=O)[nH]c4csc(C(=O)O)c4c3=O)c2)c1] | (DMSO-d6) 1.55 (3H, d, J = 6.3 Hz), 3.31 (3H, s), 3.7-3.75 (3H, m), 5.35-5.45 (1H, m), 6.35-6.45 (1H, m), 6.5-6.6 (2H, m), 7.0-7.1 (1H, m), 7.2-7.3 (1H, m), 7.39 (1H, d, J = 2.0 Hz), 7.45-7.5 (1H, m), 11.95-12.05 (1H, m), 14.41 (1H, s) |
| 288 | 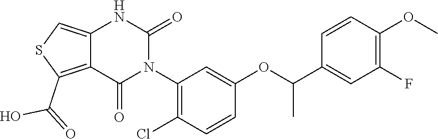 [SMILES: COc1ccc(C(C)Oc2ccc(Cl)c(-n3c(=O)[nH]c4csc(C(=O)O)c4c3=O)c2)cc1F] | (DMSO-d6) 1.5-1.6 (3H, m), 3.81 (3H, s), 5.4-5.5 (1H, m), 7.0-7.3 (5H, m), 7.4 (1H, d, J = 3.9 Hz), 7.48 (1H, dd, J = 8.9 Hz, 1.6 Hz), 11.95-12.05 (1H, m), 14.41 (1H, s) |
| 289 | 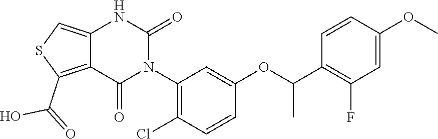 [SMILES: COc1ccc(C(C)Oc2ccc(Cl)c(-n3c(=O)[nH]c4csc(C(=O)O)c4c3=O)c2)c(F)c1] | (DMSO-d6) 1.58 (3H, d, J = 6.4 Hz), 3.75 (3H, s), 5.55-5.7 (1H, m), 6.75-6.9 (2H, m), 6.95-7.05 (1H, m), 7.15-7.55 (4H, m), 11.95-12.05 (1H, m), 14.42 (1H, s) |
| 290 | 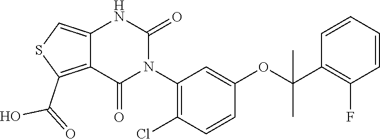 [SMILES: CC(C)(Oc1ccc(Cl)c(-n2c(=O)[nH]c3csc(C(=O)O)c3c2=O)c1)c1ccccc1F] | (DMSO-d6) 1.75 (6H, s), 6.78 (1H, dd, J = 9.0 Hz, 3.0 Hz), 7.0-7.55 (7H, m), 11.91 (1H, brs), 14.0-14.8 (1H, br) |
| 291 | 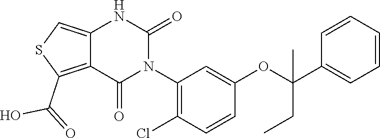 [SMILES: CCC(C)(Oc1ccc(Cl)c(-n2c(=O)[nH]c3csc(C(=O)O)c3c2=O)c1)c1ccccc1] | (DMSO-d6) 0.8 (3H, t, J = 7.3 Hz), 1.65- 1.7 (3H, m), 1.85-2.05 (2H, m), 6.55- 6.65 (1H, m), 7.08 (1H, dd, J = 6.6 Hz, 3.0 Hz), 7.25-7.45 (7H, m), 11.97 (1H, s), 14.43 (1H, s) |
| 292 | 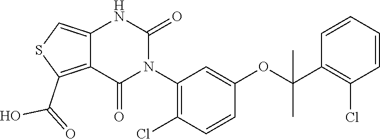 [SMILES: CC(C)(Oc1ccc(Cl)c(-n2c(=O)[nH]c3csc(C(=O)O)c3c2=O)c1)c1ccccc1Cl] | (DMSO-d6) 1.83 (6H, s), 6.69 (1H, dd, J = 9.1 Hz, 3.1 Hz), 7.06 (1H, d, J = 3.1 Hz), 7.3-7.5 (5H, m), 7.61 (1H, dd, J = 7.7 Hz, 1.8 Hz), 11.96 (1H, s), 14.42 (1H, s) |
| 293 | 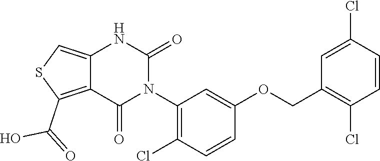 [SMILES: O=C(O)c1scc2[nH]c(=O)n(-c3cc(OCc4cc(Cl)ccc4Cl)ccc3Cl)c(=O)c12] | (DMSO-d6) 5.15 (2H, s), 7.15-7.75 (7H, m), 12.03 (1H, s), 14.41 (1H, s) |
| 294 | 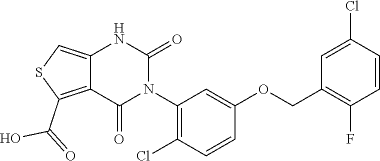 [SMILES: O=C(O)c1scc2[nH]c(=O)n(-c3cc(OCc4cc(Cl)ccc4F)ccc3Cl)c(=O)c12] | (DMSO-d6) 5.14 (2H, s), 7.23 (1H, dd, J = 8.8 Hz, 3.2 Hz), 7.3-7.45 (3H, m), 7.45-7.55 (1H, m), 7.59 (1H, d, J = 9.1 Hz), 7.65-7.7 (1H, m), 12.03 (1H, s), 14.41 (1H, s) |
| 295 | 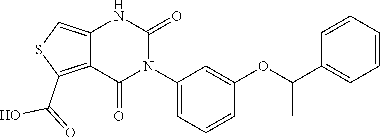 [SMILES: CC(Oc1cccc(-n2c(=O)[nH]c3csc(C(=O)O)c3c2=O)c1)c1ccccc1] | (DMSO-d6) 1.56 (3H, d, J = 6.3 Hz), 5.45-5.55 (1H, m), 6.85-6.9 (1H, m), 6.95-7.05 (2H, m), 7.25-7.45 (7H, m), 11.9 (1H, s) |
| 296 | 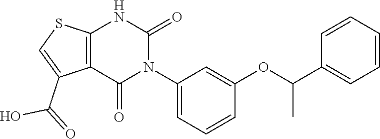 [SMILES: CC(Oc1cccc(-n2c(=O)[nH]c3scc(C(=O)O)c3c2=O)c1)c1ccccc1] | (DMSO-d6) 1.56 (3H, d, J = 6.4 Hz), 5.5 (1H, q, J = 6.4 Hz), 6.85-6.9 (1H, m), 6.9-7.05 (2H, m), 7.25-7.4 (4H, m), 7.4- 7.45 (2H, m), 7.99 (1H, s), 12.86 (1H, s), 14.43 (1H, s) |
| 297 | 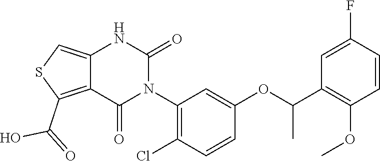 [SMILES: COc1ccc(F)cc1C(C)Oc1ccc(Cl)c(-n2c(=O)[nH]c3csc(C(=O)O)c3c2=O)c1] | (DMSO-d6) 1.52 (3H, d, J = 6.0 Hz), 3.84 (3H, s), 5.6-5.7 (1H, m), 6.85-7.55 (7H, m), 11.99 (1H, s), 14.42 (1H, s) |
| 298 | 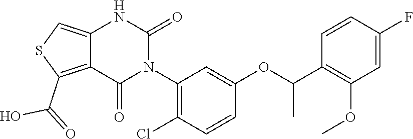 [SMILES: COc1cc(F)ccc1C(C)Oc1ccc(Cl)c(-n2c(=O)[nH]c3csc(C(=O)O)c3c2=O)c1] | (DMSO-d6) 1.52 (3H, d, J = 6.4 Hz), 3.85-3.9 (3H, m), 5.6-5.7 (1H, m), 6.75- 6.8 (1H, m), 6.85-7.0 (2H, m), 7.15-7.25 (1H, m), 7.3-7.45 (2H, m), 7.48 (1H, d, J = 9.2 Hz), 12.0 (1H, s), 14.44 (1H, s) |
| 299 | 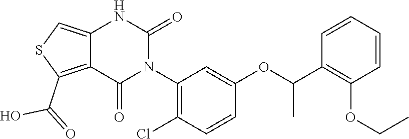 [SMILES: CCOc1ccccc1C(C)Oc1ccc(Cl)c(-n2c(=O)[nH]c3csc(C(=O)O)c3c2=O)c1] | (DMSO-d6) 1.3-1.4 (3H, m), 1.54 (3H, d, J = 6.3 Hz), 4.05-4.2 (2H, m), 5.65-5.75 (1H, m), 6.85-7.0 (2H, m), 7.0-7.05 (1H, m), 7.15-7.35 (3H, m), 7.39 (1H, d, J = 4.2 Hz), 7.48 (1H, d, J = 9.1 Hz), 11.95- 12.05 (1H, m), 14.42 (1H, s) |
| 300 | 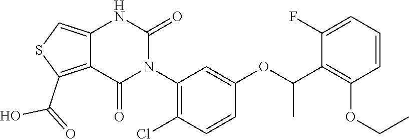 [SMILES: CCOc1cccc(F)c1C(C)Oc1ccc(Cl)c(-n2c(=O)[nH]c3csc(C(=O)O)c3c2=O)c1] | (DMSO-d6) 1.66 (3H, d, J = 6.6 Hz), 3.85-3.9 (3H, m), 5.8-5.9 (1H, m), 6.75- 6.85 (1H, m), 6.85-7.0 (2H, m), 7.17 (1H, d, J = 2.6 Hz), 7.25-7.4 (2H, m), 7.48 (1H, d, J = 8.6 Hz), 12.0 (1H, s), 14.42 (1H, s) |
| 301 | 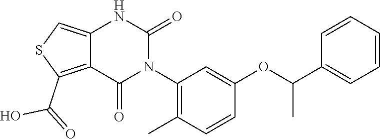 [SMILES: Cc1ccc(OC(C)c2ccccc2)cc1-n1c(=O)[nH]c2csc(C(=O)O)c2c1=O] | (DMSO-d6) 1.5-1.6 (3H, m), 1.95 (3H, s), 5.4-5.5 (1H, m), 6.85-6.95 (1H, m), 6.98 (1H, dd, J = 5.8 Hz, 2.6 Hz), 7.15- 7.45 (7H, m), 11.85-11.95 (1H, m), 14.76 (1H, s) |
| 302 | 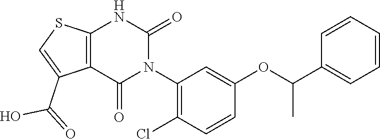 [SMILES: CC(Oc1ccc(Cl)c(-n2c(=O)[nH]c3scc(C(=O)O)c3c2=O)c1)c1ccccc1] | (DMSO-d6) 1.56 (3H, d, J = 6.3 Hz), 5.52 (1H, q, J = 6.3 Hz), 7.03 (1H, dd, J = 9.0 Hz, 3.0 Hz), 7.15-7.5 (7H, m), 7.9- 8.0 (1H, m), 12.99 (1H, brs), 13.95 (1H, s) |
| 303 | 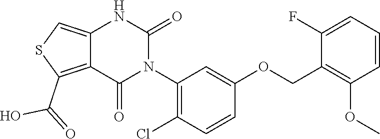 [SMILES: COc1cccc(F)c1COc1ccc(Cl)c(-n2c(=O)[nH]c3csc(C(=O)O)c3c2=O)c1] | (DMSO-d6) 3.85 (3H, s), 5.03 (2H, s), 6.85-7.0 (2H, m), 7.21 (1H, dd, J = 8.9 Hz, 3.1 Hz), 7.32 (1H, d, J = 3.1 Hz), 7.4-7.5 (2H, m), 7.58 (1H, d, J = 8.9 Hz), 12.06 (1H, s), 14.43 (1H, s) |
| 304 | 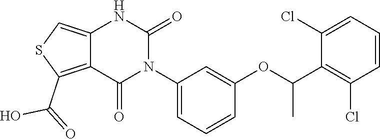 [SMILES: CC(Oc1cccc(-n2c(=O)[nH]c3csc(C(=O)O)c3c2=O)c1)c1c(Cl)cccc1Cl] | (DMSO-d6) 1.73 (3H, d, J = 6.7 Hz), 6.0-6.1 (1H, m), 6.8-6.85 (1H, m), 6.9- 7.0 (2H, m), 7.3-7.4 (3H, m), 7.45-7.5 (2H, m), 11.89 (1H, s), 14.86 (1H, s) |
| 305 | 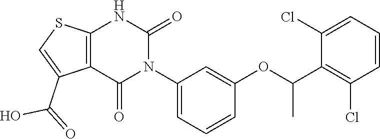 [SMILES: CC(Oc1cccc(-n2c(=O)[nH]c3scc(C(=O)O)c3c2=O)c1)c1c(Cl)cccc1Cl] | (DMSO-d6) 1.73 (3H, d, J = 6.7 Hz), 6.05 (1H, q, J = 6.7 Hz), 6.8-7.0 (3H, m), 7.3-7.4 (2H, m), 7.45-7.5 (2H, m), 7.99 (1H, s), 12.92 (1H, s), 14.43 (1H, s) |
| 306 | 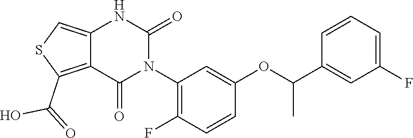 [SMILES: CC(Oc1ccc(F)c(-n2c(=O)[nH]c3csc(C(=O)O)c3c2=O)c1)c1cccc(F)c1] | (DMSO-d6) 1.56 (3H, d, J = 5.7 Hz), 5.45-5.55(1H, m), 7.0-7.5 (8H, m), 11.95-12.05 (1H, m), 14.4 (1H, brs) |
| 307 | 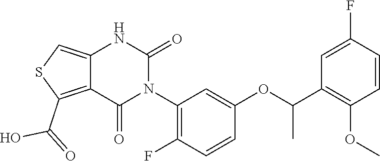 [SMILES: COc1ccc(F)cc1C(C)Oc1ccc(F)c(-n2c(=O)[nH]c3csc(C(=O)O)c3c2=O)c1] | (DMSO-d6) 1.52 (3H, d, J = 6.2 Hz), 3.84 (3H, s), 5.55-5.65 (1H, m), 6.9-7.0 (1H, m), 7.0-7.15 (4H, m), 7.2-7.3 (1H, m), 7.35-7.4 (1H, m), 11.98 (1H, s), 14.4 (1H, s) |
| 308 | 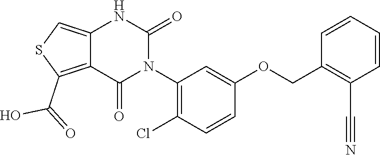 [SMILES: N#Cc1ccccc1COc1ccc(Cl)c(-n2c(=O)[nH]c3csc(C(=O)O)c3c2=O)c1] | (DMSO-d6) 5.2-5.35 (2H, m), 7.25 (1H, dd, J = 9.1 Hz, 3.0 Hz), 7.38 (1H, d, J = 3.0 Hz), 7.41 (1H, s), 7.55-7.65 (2H, m), 7.75-7.8 (2H, m), 7.94 (1H, d, J = 7.7 Hz), 12.05 (1H, s), 14.43 (1H, s) |
| 309 | 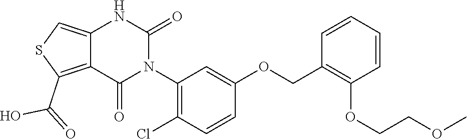 [SMILES: COCCOc1ccccc1COc1ccc(Cl)c(-n2c(=O)[nH]c3csc(C(=O)O)c3c2=O)c1] | (DMSO-d6) 3.26 (3H, s), 3.6-3.7 (2H, m), 4.1-4.2 (2H, m), 5.07 (2H, s), 6.95- 7.0 (1H, m), 7.07 (1H, d, J = 8.0 Hz), 7.15-7.2 (1H, m), 7.3-7.4 (2H, m), 7.4- 7.45 (2H, m), 7.55 (1H, d, J = 9.3 Hz), 12.04 (1H, s), 14.46 (1H, s) |
| 310 | 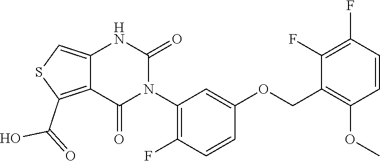 [SMILES: COc1ccc(F)c(F)c1COc1ccc(F)c(-n2c(=O)[nH]c3csc(C(=O)O)c3c2=O)c1] | (DMSO-d6) 3.83 (3H, s), 5.0-5.1 (2H, m), 6.9-6.95 (1H, m), 7.15-7.25 (2H, m), 7.3-7.4 (2H, m), 7.45-7.55 (1H, m), 12.02 (1H, s), 14.4 (1H, s) |
| 311 | 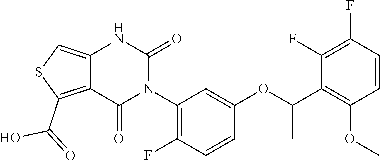 [SMILES: COc1ccc(F)c(F)c1C(C)Oc1ccc(F)c(-n2c(=O)[nH]c3csc(C(=O)O)c3c2=O)c1] | (DMSO-d6) 1.66 (3H, d, J = 6.5 Hz), 3.8-3.9 (3H, m), 5.75-5.85 (1H, m), 6.8- 7.0 (2H, m), 7.05-7.15 (1H, m), 7.2-7.4 (3H, m), 11.95-12.05 (1H, m), 14.39 (1H, s) |
| 312 | 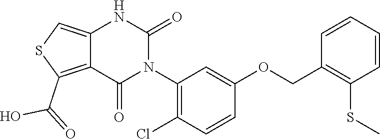 [SMILES: CSc1ccccc1COc1ccc(Cl)c(-n2c(=O)[nH]c3csc(C(=O)O)c3c2=O)c1] | (DMSO-d6) 2.48 (3H, s), 5.09 (2H, s), 7.15-7.25 (2H, m), 7.34 (1H, d, J = 2.9 Hz), 7.35-7.45 (3H, m), 7.49 (1H, d, J = 7.2 Hz), 7.58 (1H, d, J = 9.0 Hz), 12.04 (1H, s), 14.45 (1H, s) |
| 313 | 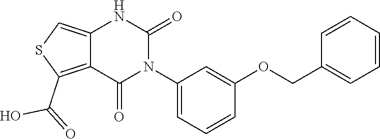 [SMILES: O=C(O)c1scc2[nH]c(=O)n(-c3cccc(OCc4ccccc4)c3)c(=O)c12] | (DMSO-d6) 5.1 (2H, s), 6.95-7.0 (1H, m), 7.05-7.15 (2H, m), 7.3-7.5 (7H, m), 11.88 (1H, s), 14.89 (1H, s) |
| 314 | 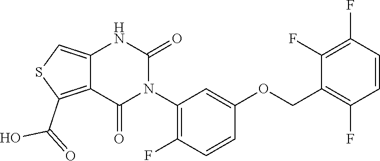 [SMILES: O=C(O)c1scc2[nH]c(=O)n(-c3cc(OCc4c(F)ccc(F)c4F)ccc3F)c(=O)c12] | (DMSO-d6) 5.14 (2H, s), 7.2-7.3 (3H, m), 7.35-7.45 (2H, m), 7.55-7.65 (1H, m), 12.02 (1H s), 14.38 (1H, s) |
| 315 | 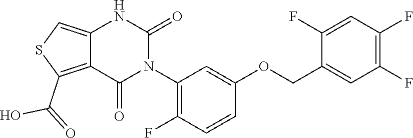 [SMILES: O=C(O)c1scc2[nH]c(=O)n(-c3cc(OCc4cc(F)c(F)cc4F)ccc3F)c(=O)c12] | (DMSO-d6) 5.08 (2H, s), 7.15-7.25 (1H, m), 7.25-7.3 (1H, m), 7.35-7.4 (2H, m), 7.55-7.7 (1H, m), 7.7-7.8 (1H, m), 12.0 (1H, s), 14.4 (1H, s) |
| 316 | 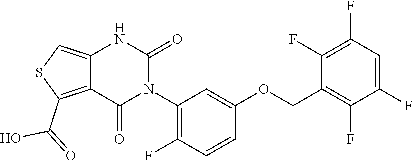 [SMILES: O=C(O)c1scc2[nH]c(=O)n(-c3cc(OCc4c(F)c(F)cc(F)c4F)ccc3F)c(=O)c12] | (DMSO-d6) 5.2 (2H, s), 7.2-7.3 (2H, m), 7.35-7.45 (2H, m), 7.95-8.05 (1H, m), 12.02 (1H, s), 14.38 (1H, s) |
| 317 | 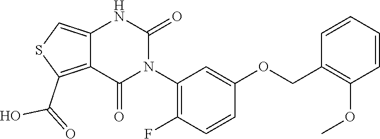 [SMILES: COc1ccccc1COc1ccc(F)c(-n2c(=O)[nH]c3csc(C(=O)O)c3c2=O)c1] | (DMSO-d6) 3.82 (3H, s), 5.02 (2H, s), 6.95-7.0 (1H, m), 7.06 (1H, d, J = 8.2 Hz), 7.1-7.2 (1H, m), 7.24 (1H, dd, J = 6.0 Hz, 3.2 Hz), 7.3-7.45 (4H, m), 12.01 (1H, s), 14.44 (1H, s) |
| 318 | 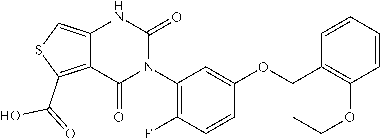 [SMILES: CCOc1ccccc1COc1ccc(F)c(-n2c(=O)[nH]c3csc(C(=O)O)c3c2=O)c1] | (DMSO-d6) 1.31 (3H, t, J = 6.9 Hz), 4.09 (2H, q, J = 6.9 Hz), 5.03 (2H, s), 6.9-7.0 (1H, m), 7.04 (1H, d, J = 8.2 Hz), 7.1-7.2 (1H, m), 7.2-7.25 (1H, m), 7.3- 7.45 (4H, m), 12.01 (1H, s), 14.43 (1H, s) |
| 319 | 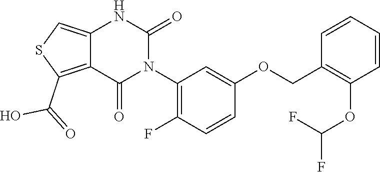 [SMILES: O=C(O)c1scc2[nH]c(=O)n(-c3cc(OCc4ccccc4OC(F)F)ccc3F)c(=O)c12] | (DMSO-d6) 5.08 (2H, s), 7.05-7.5 (7H, m), 7.55-7.65 (1H, m), 12.01 (1H, s), 14.42 (1H, s) |
| 320 | 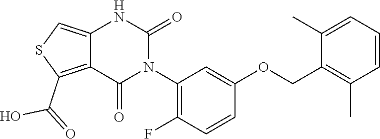 [SMILES: Cc1cccc(C)c1COc1ccc(F)c(-n2c(=O)[nH]c3csc(C(=O)O)c3c2=O)c1] | (DMSO-d6) 2.34 (6H, s), 5.03 (2H, s), 7.0-7.45 (7H, m), 12.0 (1H, s), 14.4 (1H, s) |
| 321 | 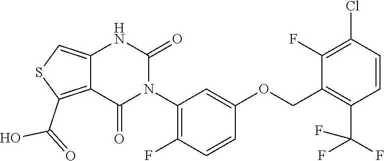 [SMILES: O=C(O)c1scc2[nH]c(=O)n(-c3cc(OCc4c(C(F)(F)F)ccc(Cl)c4F)ccc3F)c(=O)c12] | (DMSO-d6) 5.17 (2H, s), 7.2-7.3 (2H, m), 7.35-7.45 (2H, m), 7.74 (1H, d, J = 8.2 Hz), 7.9-8.0 (1H, m), 12.02 (1H, s), 14.38 (1H, s) |
| 322 | 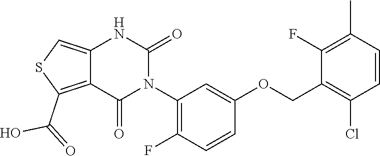 [SMILES: Cc1ccc(Cl)c(COc2ccc(F)c(-n3c(=O)[nH]c4csc(C(=O)O)c4c3=O)c2)c1F] | (DMSO-d6) 2.2-2.3 (3H, m), 5.05-5.15 (2H, m), 7.2-7.3 (2H, m), 7.32 (1H, d, J = 8.6 Hz), 7.35-7.45 (3H, m), 12.02 (1H, s), 14.39 (1H, s) |
| 323 | 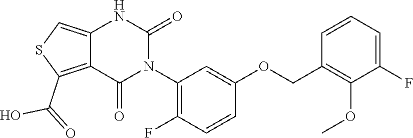 [SMILES: COc1c(F)cccc1COc1ccc(F)c(-n2c(=O)[nH]c3csc(C(=O)O)c3c2=O)c1] | (DMSO-d6) 3.85-3.95 (3H, m), 5.07 (2H, s), 7.1-7.4 (7H, m), 12.01 (1H, s), 14.42 (1H, s) |
| 324 | 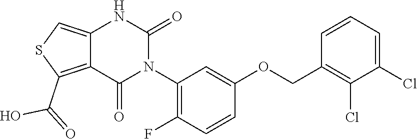 [SMILES: O=C(O)c1scc2[nH]c(=O)n(-c3cc(OCc4cccc(Cl)c4Cl)ccc3F)c(=O)c12] | (DMSO-d6) 5.18 (2H, s), 7.15-7.25 (1H, m), 7.25-7.35 (1H, m), 7.35-7.5 (3H, m), 7.55-7.7 (2H, m), 12.01 (1H, s), 14.41 (1H, s) |
| 325 | 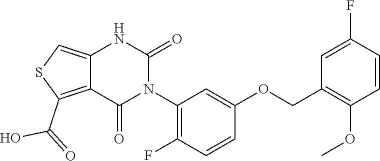 [SMILES: COc1ccc(F)cc1COc1ccc(F)c(-n2c(=O)[nH]c3csc(C(=O)O)c3c2=O)c1] | (DMSO-d6) 3.81 (3H, s), 5.02 (2H, s), 7.0-7.1 (1H, m), 7.1-7.2 (2H, m), 7.2- 7.4 (4H, m), 12.01 (1H, s), 14.42 (1H, s) |
| 326 | 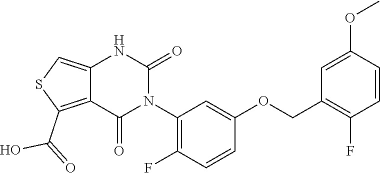 [SMILES: COc1ccc(F)c(COc2ccc(F)c(-n3c(=O)[nH]c4csc(C(=O)O)c4c3=O)c2)c1] | (DMSO-d6) 3.75 (3H, s), 5.08 (2H, s), 6.9-7.0 (1H, m), 7.1-7.25 (3H, m), 7.27 (1H, dd, J = 6.1 Hz, 3.2 Hz), 7.3-7.4 (2H, m), 12.02 (1H, s), 14.41 (1H, s) |
| 327 | 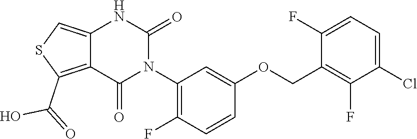 [SMILES: O=C(O)c1scc2[nH]c(=O)n(-c3cc(OCc4c(F)ccc(Cl)c4F)ccc3F)c(=O)c12] | (DMSO-d6) 5.14 (2H, s), 7.15-7.35 (3H, m), 7.35-7.45 (2H, m), 7.7-7.8 (1H, m), 12.02 (1H, s), 14.39 (1H, s) |
| 328 | 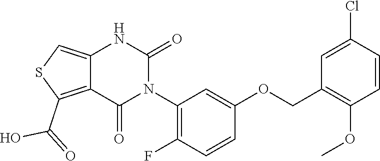 [SMILES: COc1ccc(Cl)cc1COc1ccc(F)c(-n2c(=O)[nH]c3csc(C(=O)O)c3c2=O)c1] | (DMSO-d6) 3.83 (3H, s), 5.01 (2H, s), 7.09 (1H, d, J = 8.8 Hz), 7.15-7.2 (1H, m), 7.25-7.35 (1H, m), 7.3-7.45 (3H, m), 7.48 (1H, d, J = 2.6 Hz), 12.02 (1H, s), 14.43 (1H, s) |
| 329 | 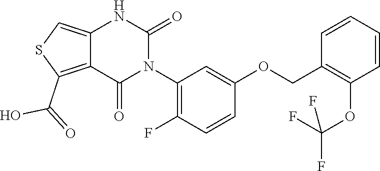 [SMILES: O=C(O)c1scc2[nH]c(=O)n(-c3cc(OCc4ccccc4OC(F)(F)F)ccc3F)c(=O)c12] | (DMSO-d6) 5.12 (2H, s), 7.15-7.25 (1H, m), 7.27 (1H, dd, J = 6.0 Hz, 3.2 Hz), 7.3-7.4 (2H, m), 7.4-7.5 (2H, m), 7.5-7.6 (1H, m), 7.7 (1H, dd, J = 7.4 Hz, 1.6 Hz), 12.01 (1H, s), 14.41 (1H, s) |
| 330 | 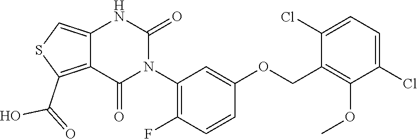 [SMILES: COc1c(Cl)ccc(Cl)c1COc1ccc(F)c(-n2c(=O)[nH]c3csc(C(=O)O)c3c2=O)c1] | (DMSO-d6) 3.85 (3H, s), 5.1 (2H, s), 7.2-7.35 (2H, m), 7.35-7.45 (3H, m), 7.63 (1H, d, J = 8.7 Hz), 12.01 (1H, s), 14.38 (1H, s) |
| 331 | 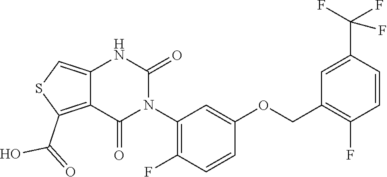 [SMILES: O=C(O)c1scc2[nH]c(=O)n(-c3cc(OCc4cc(C(F)(F)F)ccc4F)ccc3F)c(=O)c12] | (DMSO-d6) 5.21 (2H, s), 7.2-7.35 (2H, m), 7.35-7.45 (2H, m), 7.53 (1H, t, J = 9.2 Hz), 7.8-7.9 (1H, m), 8.02 (1H, d, J = 6.1 Hz), 12.02 (1H, s), 14.4 (1H, s) |
| 332 | 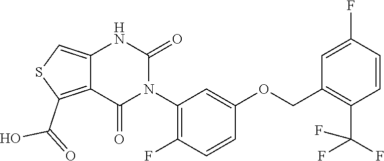 [SMILES: O=C(O)c1scc2[nH]c(=O)n(-c3cc(OCc4cc(F)ccc4C(F)(F)F)ccc3F)c(=O)c12] | (DMSO-d6) 5.21 (2H, s), 7.15-7.25 (1H, m), 7.3 (1H, dd, J = 5.8 Hz, 3.0 Hz), 7.35-7.5 (3H, m), 7.65-7.75 (1H, m), 7.89 (1H, dd, J = 8.7 Hz, 5.4 Hz), 12.01 (1H, s), 14.4 (1H, s) |
| 333 | 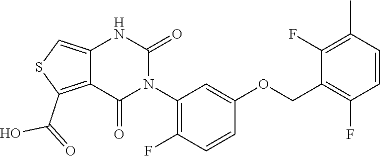 [SMILES: Cc1ccc(F)c(COc2ccc(F)c(-n3c(=O)[nH]c4csc(C(=O)O)c4c3=O)c2)c1F] | (DMSO-d6) 2.24 (3H, s), 5.08 (2H, s), 7.05-7.15 (1H, m), 7.15-7.3 (2H, m), 7.35-7.45 (3H, m), 12.02 (1H, s), 14.4 (1H, s) |
| 334 | 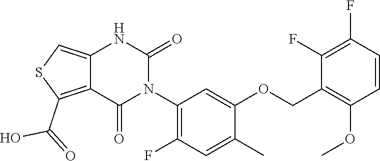 [SMILES: COc1ccc(F)c(F)c1COc1cc(-n2c(=O)[nH]c3csc(C(=O)O)c3c2=O)c(F)cc1C] | (DMSO-d6) 2.13 (3H, s), 3.82 (3H, s), 5.0 (2H, s), 6.85-6.95 (1H, m), 7.25 (1H, d, J = 10.0 Hz), 7.32 (1H, d, J = 6.4 Hz), 7.39 (1H, s), 7.4-7.55 (1H, m), 12.01 (1H, s), 14.47 (1H, s) |
| 335 | 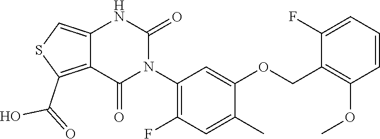 [SMILES: COc1cccc(F)c1COc1cc(-n2c(=O)[nH]c3csc(C(=O)O)c3c2=O)c(F)cc1C] | (DMSO-d6) 2.12 (3H, s), 3.84 (3H, s), 4.96 (2H, s), 6.8-7.0 (2H, m), 7.24 (1H, d, J = 10.2 Hz), 7.32 (1H, d, J = 6.3 Hz), 7.35-7.5 (2H, m), 12.04 (1H, s), 14.48 (1H, s) |
| 336 | 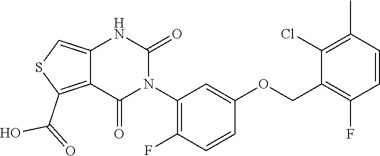 [SMILES: Cc1ccc(F)c(COc2ccc(F)c(-n3c(=O)[nH]c4csc(C(=O)O)c4c3=O)c2)c1Cl] | (DMSO-d6) 2.36 (3H, s), 5.1-5.15 (2H, m), 7.2-7.3 (3H, m), 7.35-7.45 (2H, m), 7.5 (1H, dd, J = 8.7 Hz, 6.2 Hz), 12.03 (1H, s), 14.4 (1H, s) |
| 337 | 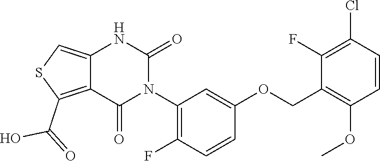 [SMILES: COc1ccc(Cl)c(F)c1COc1ccc(F)c(-n2c(=O)[nH]c3csc(C(=O)O)c3c2=O)c1] | (DMSO-d6) 3.86 (3H, s), 5.04 (2H, s), 7.0 (1H, d, J = 9.3 Hz), 7.15-7.25 (2H, m), 7.3-7.4 (2H, m), 7.55-7.65 (1H, m), 12.03 (1H, s), 14.41 (1H, s) |
| 338 | 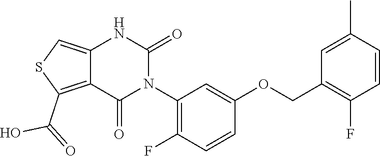 [SMILES: Cc1ccc(F)c(COc2ccc(F)c(-n3c(=O)[nH]c4csc(C(=O)O)c4c3=O)c2)c1] | (DMSO-d6) 2.3 (3H, s), 5.06 (2H, s), 7.1-7.45 (7H, m), 12.03 (1H, s), 14.43 (1H, s) |
| 339 | 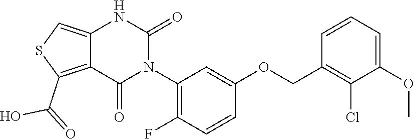 [SMILES: COc1cccc(COc2ccc(F)c(-n3c(=O)[nH]c4csc(C(=O)O)c4c3=O)c2)c1Cl] | (DMSO-d6) 3.88 (3H, s), 5.13 (2H, s), 7.15-7.25 (3H, m), 7.27 (1H, dd, J = 6.0 Hz, 3.2 Hz), 7.3-7.4 (3H, m), 12.02 (1H, s), 14.42 (1H, s) |
| 340 | 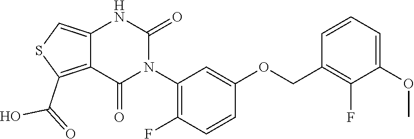 [SMILES: COc1cccc(COc2ccc(F)c(-n3c(=O)[nH]c4csc(C(=O)O)c4c3=O)c2)c1F] | (DMSO-d6) 3.85 (3H, s), 5.11 (2H, s), 7.05-7.3 (5H, m), 7.3-7.4 (2H, m), 12.02 (1H, s), 14.42 (1H, s) |
| 341 | 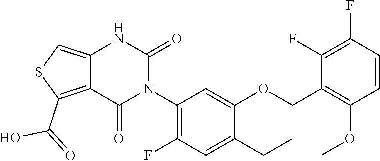 [SMILES: CCc1cc(F)c(-n2c(=O)[nH]c3csc(C(=O)O)c3c2=O)cc1OCc1c(OC)ccc(F)c1F] | (DMSO-d6) 1.1 (3H, t, J = 7.5 Hz), 2.45- 2.6 (2H, m), 3.82 (3H, s), 5.0 (2H, s), 6.9-6.95 (1H, m), 7.23 (1H, d, J = 10.3 Hz), 7.34 (1H, d, J = 6.3 Hz), 7.39 (1H, s), 7.4-7.55 (1H, m), 12.03 (1H, s), 14.47 (1H, s) |
| 342 | 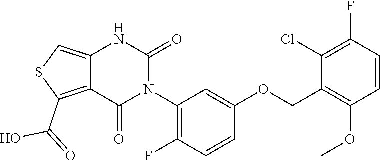 [SMILES: COc1ccc(F)c(Cl)c1COc1ccc(F)c(-n2c(=O)[nH]c3csc(C(=O)O)c3c2=O)c1] | (DMSO-d6) 3.84 (3H, s), 5.1 (2H, s), 7.13 (1H, dd, J = 9.2 Hz, 4.0 Hz), 7.15- 7.3 (2H, m), 7.3-7.4 (2H, m), 7.45-7.55 (1H, m), 12.03 (1H, s) |
| 343 | 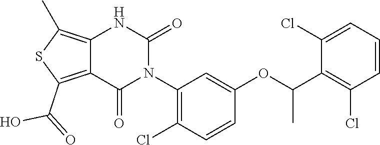 [SMILES: Cc1sc(C(=O)O)c2c(=O)n(-c3cc(OC(C)c4c(Cl)cccc4Cl)ccc3Cl)c(=O)[nH]c12] | (DMSO-d6) 1.73 (3H, d, J = 6.7 Hz), 2.5-2.55 (3H, m), 6.07 (1H, q, J = 6.7 Hz), 6.85-6.95 (1H, m), 7.15-7.2 (1H, m), 7.3-7.4 (1H, m), 7.45-7.55 (3H, m), 11.89 (1H, s), 14.37 (1H, s) |
| 344 | 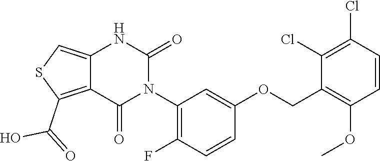 [SMILES: COc1ccc(Cl)c(Cl)c1COc1ccc(F)c(-n2c(=O)[nH]c3csc(C(=O)O)c3c2=O)c1] | (DMSO-d6) 3.86 (3H, s), 5.12 (2H, s), 7.1-7.3 (3H, m), 7.3-7.4 (2H, m), 7.69 (1H, d, J = 8.7 Hz), 12.03 (1H, s), 14.41 (1H, s) |
| 345 | 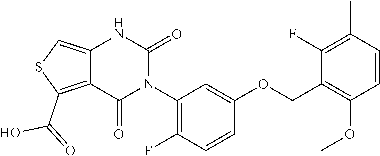 [SMILES: COc1ccc(C)c(F)c1COc1ccc(F)c(-n2c(=O)[nH]c3csc(C(=O)O)c3c2=O)c1] | (DMSO-d6) 2.15-2.2 (3H, m), 3.81 (3H, s), 5.0 (2H, s), 6.84 (1H, d, J = 8.5 Hz), 7.15-7.25 (2H, m), 7.25-7.4 (3H, m), 12.03 (1H, s), 14.42 (1H, s) |
| 346 | 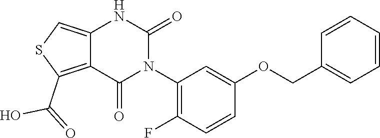 [SMILES: O=C(O)c1scc2[nH]c(=O)n(-c3cc(OCc4ccccc4)ccc3F)c(=O)c12] | (DMSO-d6) 5.08 (2H, s), 7.1-7.5 (9H, m), 12.01 (1H, s), 14.42 (1H, s) |
| 347 | 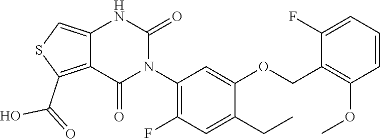 [SMILES: CCc1cc(F)c(-n2c(=O)[nH]c3csc(C(=O)O)c3c2=O)cc1OCc1c(F)cccc1OC] | (DMSO-d6) 1.1 (3H, t, J = 7.5 Hz), 2.45- 2.55 (2H, m), 3.84 (3H, s), 4.97 (2H, s), 6.85-7.0 (2H, m), 7.22 (1H, d, J = 10.2 Hz), 7.33 (1H, d, J = 6.4 Hz), 7.35-7.5 (2H, m), 12.02 (1H, s), 14.47 (1H, brs) |
| 348 | 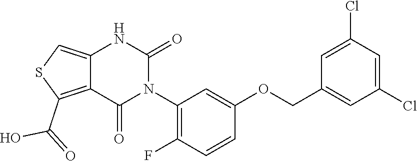 [SMILES: O=C(O)c1scc2[nH]c(=O)n(-c3cc(OCc4cc(Cl)cc(Cl)c4)ccc3F)c(=O)c12] | (DMSO-d6) 5.11 (2H, s), 7.1-7.3 (2H, m), 7.3-7.45 (2H, m), 7.5-7.65 (3H, m), 12.0 (1H, brs), 14.41 (1H, brs) |
| 349 | 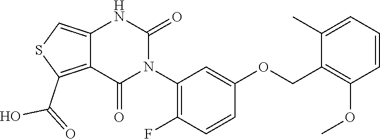 [SMILES: COc1cccc(C)c1COc1ccc(F)c(-n2c(=O)[nH]c3csc(C(=O)O)c3c2=O)c1] | (DMSO-d6) 2.33 (3H, s), 3.78 (3H, s), 5.04 (2H, s), 6.8-6.95 (2H, m), 7.1-7.3 (3H, m), 7.3-7.4 (2H, m), 12.01 (1H, s), 14.42 (1H, brs) |
| 350 | 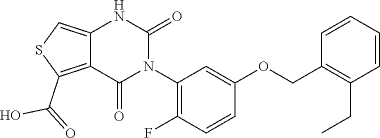 [SMILES: CCc1ccccc1COc1ccc(F)c(-n2c(=O)[nH]c3csc(C(=O)O)c3c2=O)c1] | (DMSO-d6) 1.19 (3H, t, J = 7.5 Hz), 2.68 (2H, q, J = 7.5 Hz), 5.07 (2H, s), 7.15-7.4 (7H, m), 7.4-7.5 (1H, m), 12.0 (1H, brs), 14.3-14.55 (1H, br) |
| 351 | 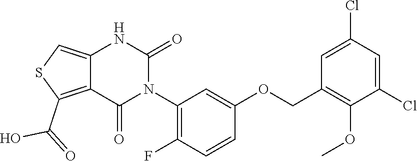 [SMILES: COc1c(Cl)cc(Cl)cc1COc1ccc(F)c(-n2c(=O)[nH]c3csc(C(=O)O)c3c2=O)c1] | (DMSO-d6) 3.84 (3H, s), 5.09 (2H, s), 7.2-7.25 (1H, m), 7.25-7.35 (1H, m), 7.35-7.45 (2H, m), 7.58 (1H, d, J = 2.7 Hz), 7.68 (1H, d, J = 2.7 Hz), 12.01 (1H, brs), 14.41 (1H, brs) |
| 352 | 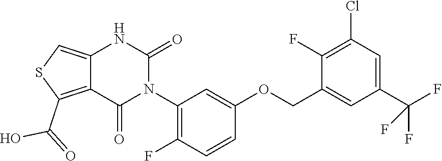 [SMILES: O=C(O)c1scc2[nH]c(=O)n(-c3cc(OCc4cc(C(F)(F)F)cc(Cl)c4F)ccc3F)c(=O)c12] | (DMSO-d6) 5.25 (2H, s), 7.2-7.35 (2H, m), 7.35-7.45 (2H, m), 7.95-8.05 (1H, m), 8.1-8.2 (1H, m), 12.01 (1H, brs), 14.4 (1H, brs) |
| 353 | 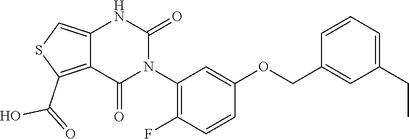 [SMILES: CCc1cccc(COc2ccc(F)c(-n3c(=O)[nH]c4csc(C(=O)O)c4c3=O)c2)c1] | (DMSO-d6) 1.19 (3H, t, J = 7.6 Hz), 2.62 (2H, q, J = 7.6 Hz), 5.05 (2H, s), 7.1-7.4 (8H, m), 12.01 (1H, brs), 14.43 (1H, brs) |
| 354 | 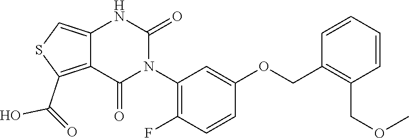 [SMILES: COCc1ccccc1COc1ccc(F)c(-n2c(=O)[nH]c3csc(C(=O)O)c3c2=O)c1] | (DMSO-d6) 3.3 (3H, s), 4.53 (2H, s), 5.13 (2H, s), 7.1-7.3 (2H, m), 7.3-7.45 (5H, m), 7.45-7.55 (1H, m), 12.01 (1H, brs), 14.42 (1H, brs) |
| 355 | 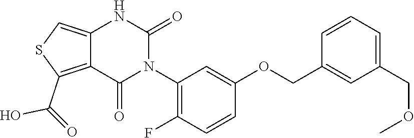 [SMILES: COCc1cccc(COc2ccc(F)c(-n3c(=O)[nH]c4csc(C(=O)O)c4c3=O)c2)c1] | (DMSO-d6) 3.29 (3H, s), 4.43 (2H, s), 5.09 (2H, s), 7.1-7.2 (1H, m), 7.2-7.45 (7H, m), 12.01 (1H, brs), 14.42 (1H, brs) |
| 356 | 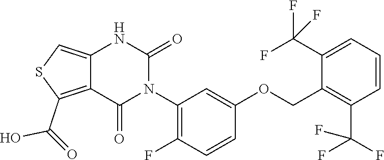 [SMILES: O=C(O)c1scc2[nH]c(=O)n(-c3cc(OCc4c(C(F)(F)F)cccc4C(F)(F)F)ccc3F)c(=O)c12] | (DMSO-d6) 5.19 (2H, s), 7.15-7.25 (1H, m), 7.25-7.3 (1H, m), 7.35-7.45 (2H, m), 7.92 (1H, t, J = 8.0 Hz), 8.21 (2H, d, J = 28.0 Hz), 12.01 (1H, s), 14.38 (1H, brs) |
| 357 | 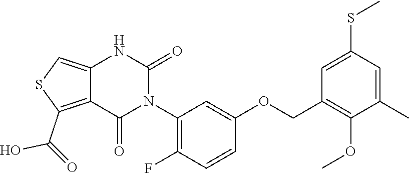 [SMILES: COc1c(C)cc(SC)cc1COc1ccc(F)c(-n2c(=O)[nH]c3csc(C(=O)O)c3c2=O)c1] | (DMSO-d6) 2.25 (3H, s), 2.44 (3H, s), 3.7 (3H, s), 5.02 (2H, s), 7.1-7.25 (3H, m), 7.25-7.3 (1H, m), 7.3-7.4 (2H, m), 12.01 (1H, brs), 14.3-14.55 (1H, br) |
| 358 | 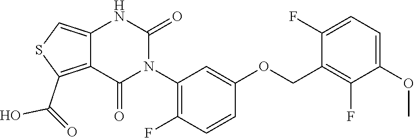 [SMILES: COc1ccc(F)c(COc2ccc(F)c(-n3c(=O)[nH]c4csc(C(=O)O)c4c3=O)c2)c1F] | (DMSO-d6) 3.85 (3H, s), 5.09 (2H, s), 7.05-7.35 (4H, m), 7.35-7.45 (2H, m), 12.02 (1H, brs), 14.39 (1H, brs) |
| 359 | 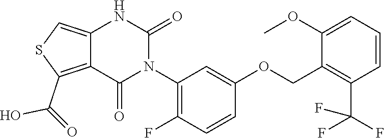 [SMILES: COc1cccc(C(F)(F)F)c1COc1ccc(F)c(-n2c(=O)[nH]c3csc(C(=O)O)c3c2=O)c1] | (DMSO-d6) 3.89 (3H, s), 5.07 (2H, s), 7.15-7.3 (2H, m), 7.3-7.5 (4H, m), 7.6- 7.7 (1H, m), 12.01 (1H, s), 14.4 (1H, brs) |
| 360 | 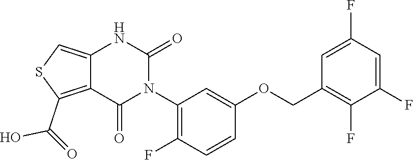 [SMILES: O=C(O)c1scc2[nH]c(=O)n(-c3cc(OCc4cc(F)cc(F)c4F)ccc3F)c(=O)c12] | (DMSO-d6) 5.18 (2H, s), 7.2-7.45 (5H, m), 7.5-7.65 (1H, m), 12.02 (1H, s), 14.4 (1H, brs) |
| 361 | 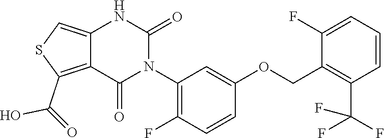 [SMILES: O=C(O)c1scc2[nH]c(=O)n(-c3cc(OCc4c(F)cccc4C(F)(F)F)ccc3F)c(=O)c12] | (DMSO-d6) 5.14 (2H, s), 7.15-7.3 (2H, m), 7.3-7.45 (2H, m), 7.6-7.8 (3H, m), 12.02 (1H, s), 14.39 (1H, s) |
| 362 | 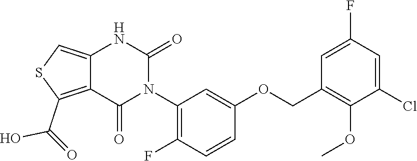 [SMILES: COc1c(Cl)cc(F)cc1COc1ccc(F)c(-n2c(=O)[nH]c3csc(C(=O)O)c3c2=O)c1] | (DMSO-d6) 3.82 (3H, s), 5.1(2H, s), 7.15-7.45 (5H, m), 7.45-7.55 (1H, m), 12.01 (1H s), 14.4 (1H, s) |
| 363 | 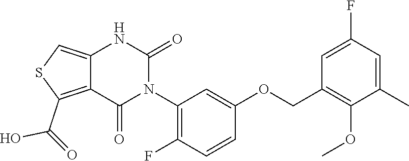 [SMILES: COc1c(C)cc(F)cc1COc1ccc(F)c(-n2c(=O)[nH]c3csc(C(=O)O)c3c2=O)c1] | (DMSO-d6) 2.27 (3H, s), 3.71 (3H, s), 5.05 (2H, s), 7.05-7.25 (3H, m), 7.25-7.3 (1H, m), 7.3-7.4 (2H, m), 12.02 (1H, s), 14.42 (1H, brs) |
| 364 | 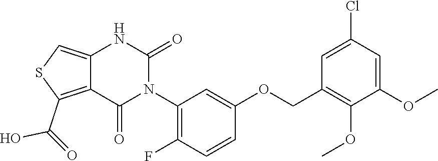 [SMILES: COc1cc(Cl)cc(COc2ccc(F)c(-n3c(=O)[nH]c4csc(C(=O)O)c4c3=O)c2)c1OC] | (DMSO-d6) 3.76 (3H, s), 3.85 (3H, s), 5.03 (2H, s), 7.05-7.3 (4H, m), 7.3-7.4 (2H, m), 12.02 (1H, brs), 14.42 (1H, brs) |
| 365 | 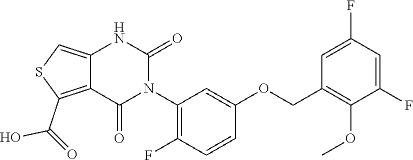 [SMILES: COc1c(F)cc(F)cc1COc1ccc(F)c(-n2c(=O)[nH]c3csc(C(=O)O)c3c2=O)c1] | (DMSO-d6) 3.8-3.9 (3H, m), 5.08 (2H, s), 7.15-7.25 (2H, m), 7.25-7.45 (4H, m), 12.02 (1H, brs), 14.41 (1H, brs) |
| 366 | 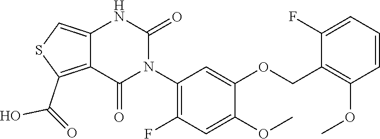 [SMILES: COc1cc(F)c(-n2c(=O)[nH]c3csc(C(=O)O)c3c2=O)cc1OCc1c(F)cccc1OC] | (DMSO-d6) 3.79 (3H, s), 3.83 (3H, s), 4.92 (2H, s), 6.8-7.0 (2H, m), 7.11 (1H, d, J = 11.2 Hz), 7.27 (1H, d, J = 27.6 Hz), 7.38 (1H, s), 7.4-7.5 (1H, m), 11.99 (1H, brs), 14.55 (1H, brs) |
| 367 | 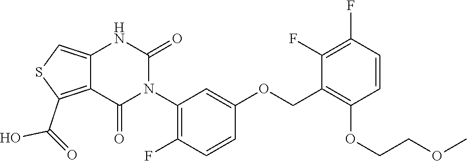 [SMILES: COCCOc1ccc(F)c(F)c1COc1ccc(F)c(-n2c(=O)[nH]c3csc(C(=O)O)c3c2=O)c1] | (DMSO-d6) 3.23 (3H, s), 3.55-3.65 (2H, m), 4.1-4.2 (2H, m), 5.09 (2H, s), 6.9-7.0 (1H, m), 7.15-7.3 (2H, m), 7.3-7.4 (2H, m), 7.4-7.5 (1H, m), 12.03 (1H, brs), 14.42 (1H, brs) |
| 368 | 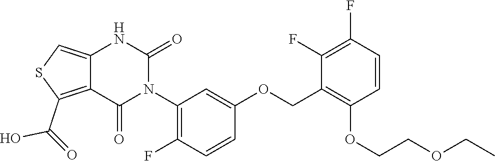 [SMILES: CCOCCOc1ccc(F)c(F)c1COc1ccc(F)c(-n2c(=O)[nH]c3csc(C(=O)O)c3c2=O)c1] | (DMSO-d6) 1.02 (3H, t, J = 6.9 Hz), 3.43 (2H, q, J = 6.9 Hz), 3.6-3.7 (2H, m), 4.1-4.2 (2H, m), 5.08 (2H, s), 6.9-7.0 (1H, m), 7.15-7.25 (2H, m), 7.3-7.4 (2H, m), 7.4-7.5 (1H, m), 12.02 (1H, s), 14.42 (1H, brs) |
| 369 | 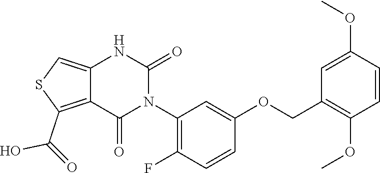 [SMILES: COc1ccc(OC)c(COc2ccc(F)c(-n3c(=O)[nH]c4csc(C(=O)O)c4c3=O)c2)c1] | (DMSO-d6) 3.71 (3H, s), 3.77 (3H, 5), 4.99 (2H, s), 6.85-6.95 (1H, m), 6.95- 7.05 (2H, m), 7.1-7.2 (1H, m), 7.2-7.3 (1H, m), 7.3-7.4 (2H, m), 12.01 (1H, s), 14.44 (1H, brs) |
| 370 | 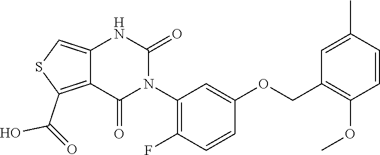 [SMILES: COc1ccc(C)cc1COc1ccc(F)c(-n2c(=O)[nH]c3csc(C(=O)O)c3c2=O)c1] | (DMSO-d6) 2.25 (3H, s), 3.78 (3H, s), 4.98 (2H, s), 6.94 (1H, d, J = 7.9 Hz), 7.1-7.2 (2H, m), 7.2-7.3 (2H, m), 7.3-7.4 (2H, m), 12.01 (1H, brs), 14.45 (1H, brs) |
| 371 | 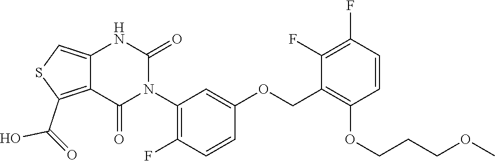 [SMILES: COCCCOc1ccc(F)c(F)c1COc1ccc(F)c(-n2c(=O)[nH]c3csc(C(=O)O)c3c2=O)c1] | (DMSO-d6) 1.85-1.95 (2H, m), 3.16 (3H, s), 3.39 (2H, t, J = 6.2 Hz), 4.08 (2H, t, J = 6.2 Hz), 5.06 (2H, s), 6.85-6.95 (1H, m), 7.15-7.3 (2H, m), 7.3-7.5 (3H, m), 12.03 (1H, s), 14.42 (1H, brs) |
| 372 | 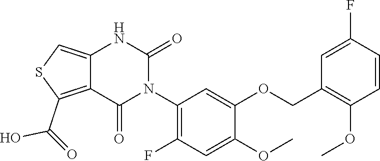 [SMILES: COc1ccc(F)cc1COc1cc(-n2c(=O)[nH]c3csc(C(=O)O)c3c2=O)c(F)cc1OC] | (DMSO-d6) 3.78 (3H, s), 3.85 (3H, s), 4.94 (2H, s), 7.0-7.1 (1H, m), 7.1-7.2 (2H, m), 7.2-7.35 (2H, m), 7.36 (1H, s), 11.96 (1H, brs), 14.56 (1H, brs) |
| 373 | 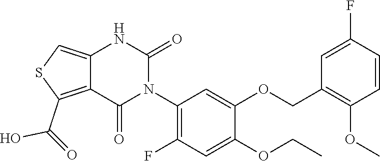 [SMILES: CCOc1cc(F)c(-n2c(=O)[nH]c3csc(C(=O)O)c3c2=O)cc1OCc1cc(F)ccc1OC] | (DMSO-d6) 1.36 (3H, t, J = 6.9 Hz), 3.78 (3H, s), 4.13 (2H, q, J = 6.9 Hz), 4.96 (2H, s), 7.0-7.1 (1H, m), 7.1-7.2 (2H, m), 7.2-7.35 (2H, m), 7.37 (1H, s), 11.97 (1H, brs), 14.56 (1H, brs) |
| 374 | 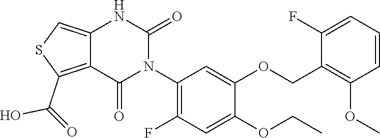 [SMILES: CCOc1cc(F)c(-n2c(=O)[nH]c3csc(C(=O)O)c3c2=O)cc1OCc1c(F)cccc1OC] | (DMSO-d6) 1.3 (3H, t, J = 7.0 Hz), 3.82 (3H, s), 4.08 (2H, q, J = 27.0 Hz), 4.94 (2H, s), 6.8-7.0 (2H, m), 7.1 (1H, d, J = 11.6 Hz), 7.26 (1H, d, J = 7.6 Hz), 7.35- 7.5 (2H, m), 11.99 (1H, s), 14.55 (1H, brs) |
| 375 | 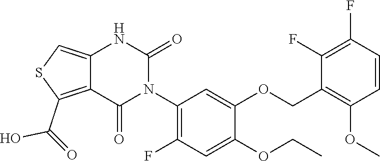 [SMILES: CCOc1cc(F)c(-n2c(=O)[nH]c3csc(C(=O)O)c3c2=O)cc1OCc1c(OC)ccc(F)c1F] | (DMSO-d6) 1.31 (3H, t, J = 7.0 Hz), 3.79 (3H, s), 4.09 (2H, q, J = 7.0 Hz), 4.95-5.05 (2H, m), 6.85-6.95 (1H, m), 7.12 (1H, d, J = 11.7 Hz), 7.23 (1H, d, J = 7.3 Hz), 7.38 (1H, s), 7.4-7.5 (1H, m), 11.99 (1H, s), 14.54 (1H, brs) |
| 376 | 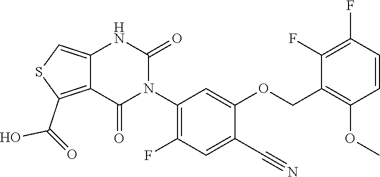 [SMILES: COc1ccc(F)c(F)c1COc1cc(-n2c(=O)[nH]c3csc(C(=O)O)c3c2=O)c(F)cc1C#N] | (DMSO-d6) 3.83 (3H, s), 5.18 (2H, s), 6.9-7.0 (1H, m), 7.38 (1H, s), 7.45-7.6 (1H, m), 7.7-7.85 (1H, m), 8.0-8.1 (1H, m), 12.08 (1H, s), 14.16 (1H, brs) |
| 377 | 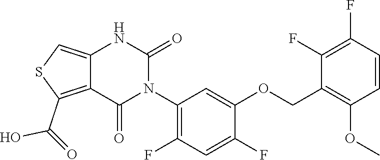 [SMILES: COc1ccc(F)c(F)c1COc1cc(-n2c(=O)[nH]c3csc(C(=O)O)c3c2=O)c(F)cc1F] | (DMSO-d6) 3.81 (3H, s), 5.07 (2H, s), 6.9-6.95 (1H, m), 7.39 (1H, s), 7.45- 7.55 (1H, m), 7.55-7.65 (2H, m), 12.06 (1H, s), 14.37 (1H, brs) |
| 378 | 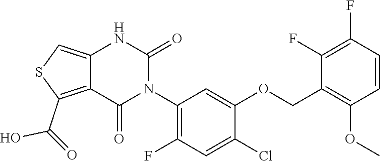 [SMILES: COc1ccc(F)c(F)c1COc1cc(-n2c(=O)[nH]c3csc(C(=O)O)c3c2=O)c(F)cc1Cl] | (DMSO-d6) 3.82 (3H, s), 5.08 (2H, s), 6.9-7.0 (1H, m), 7.39 (1H, s), 7.45-7.6 (1H, m), 7.61 (1H, d, J = 6.7 Hz), 7.73 (1H, d J = 9.1 Hz), 12.07 (1H, s), 14.33 (1H, brs) |
| 379 | 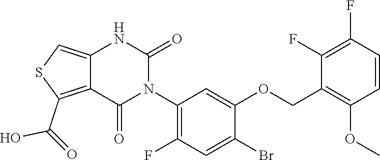 [SMILES: COc1ccc(F)c(F)c1COc1cc(-n2c(=O)[nH]c3csc(C(=O)O)c3c2=O)c(F)cc1Br] | (DMSO-d6) 3.82 (3H, s), 5.07 (2H, s), 6.9-7.0 (1H, m), 7.39 (1H, s), 7.45-7.55 (1H, m), 7.58 (1H, d, J = 6.5 Hz), 7.84 (1H, d, J = 8.8 Hz), 12.07 (1H, s), 14.33 (1H, s) |
| 380 | 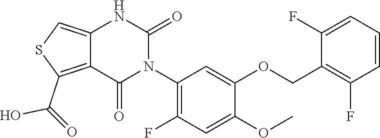 [SMILES: COc1cc(F)c(-n2c(=O)[nH]c3csc(C(=O)O)c3c2=O)cc1OCc1c(F)cccc1F] | (DMSO-d6) 3.81 (3H, s), 5.0 (2H, s), 7.1-7.25 (3H, m), 7.29 (1H, d, J = 7.4 Hz), 7.39 (1H, s), 7.45-7.6 (1H, m), 12.01 (1H, s), 14.54 (1H, s) |
| 381 | 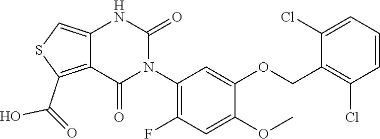 [SMILES: COc1cc(F)c(-n2c(=O)[nH]c3csc(C(=O)O)c3c2=O)cc1OCc1c(Cl)cccc1Cl] | (DMSO-d6) 3.82 (3H, s), 5.13 (2H, s), 7.15 (1H, d, J = 11.4 Hz), 7.33 (1H, d, J = 7.3 Hz), 7.39 (1H, s), 7.45-7.6 (3H, m), 12.01 (1H, s), 14.54 (1H, s) |
| 382 | 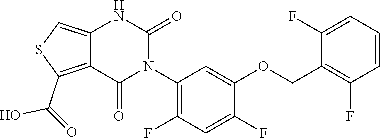 [SMILES: O=C(O)c1scc2[nH]c(=O)n(-c3cc(OCc4c(F)cccc4F)c(F)cc3F)c(=O)c12] | (DMSO-d6) 5.12 (2H, s), 7.15-7.25 (2H, m), 7.39 (1H, s), 7.5-7.7 (3H, m), 12.06 (1H, s), 14.36 (1H, brs) |
| 383 | 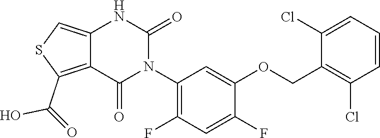 [SMILES: O=C(O)c1scc2[nH]c(=O)n(-c3cc(OCc4c(Cl)cccc4Cl)c(F)cc3F)c(=O)c12] | (DMSO-d6) 5.24 (2H, s), 7.39 (1H, s), 7.45-7.55 (1H, m), 7.55-7.75 (4H, m), 12.06 (1H, s), 14.36 (1H, brs) |
| 384 | 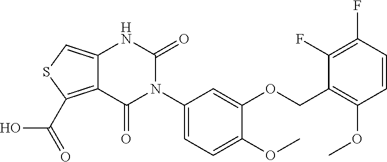 [SMILES: COc1ccc(-n2c(=O)[nH]c3csc(C(=O)O)c3c2=O)cc1OCc1c(OC)ccc(F)c1F] | (DMSO-d6) 3.78 (3H, s), 3.81 (3H, s), 4.96 (2H, s), 6.85-7.0 (2H, m), 7.07 (1H, d, J = 8.4 Hz), 7.18 (1H, d, J = 2.4 Hz), 7.37 (1H, s), 7.4-7.55 (1H, m), 11.87 (1H, s), 14.97 (1H, s) |
| 385 | 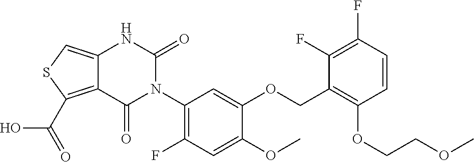 [SMILES: COCCOc1ccc(F)c(F)c1COc1cc(-n2c(=O)[nH]c3csc(C(=O)O)c3c2=O)c(F)cc1OC] | (DMSO-d6) 3.2 (3H, s), 3.5-3.6 (2H, m), 3.81 (3H, s), 4.05-4.15 (2H, m), 5.01 (2H, s); 6.85-6.95 (1H, m), 7.13 (1H, d, J = 11.2 Hz), 7.23 (1H, d, J = 7.0 Hz), 7.39 (1H, s), 7.4-7.5 (1H, m), 11.99 (1H, s), 14.52 (1H, s) |
| 386 | 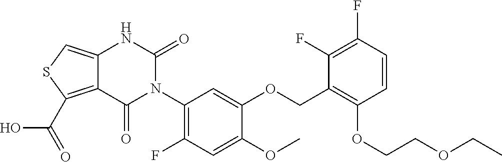 [SMILES: CCOCCOc1ccc(F)c(F)c1COc1cc(-n2c(=O)[nH]c3csc(C(=O)O)c3c2=O)c(F)cc1OC] | (DMSO-d6) 1.0 (3H, t, J = 7.0 Hz), 3.4 (2H, q, J = 7.0 Hz), 3.55-3.65 (2H, m), 3.81 (3H, s), 4.05-4.15 (2H, m), 5.0 (2H, s), 6.85-6.95 (1H, m), 7.13 (1H, d, J = 11.4 Hz), 7.23 (1H, d, J = 7.7 Hz), 7.38 (1H, s), 7.4-7.5 (1H, m), 11.99 (1H, s), 14.53 (1H, brs) |
| 387 | 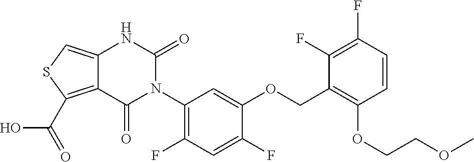 [SMILES: COCCOc1ccc(F)c(F)c1COc1cc(-n2c(=O)[nH]c3csc(C(=O)O)c3c2=O)c(F)cc1F] | (DMSO-d6) 3.19 (3H, s), 3.5-3.65 (2H, m), 4.05-4.2 (2H, m), 5.13 (2H, s), 6.9- 7.0 (1H, m), 7.38 (1H, s), 7.4-7.65 (3H, m), 12.03 (1H, s), 14.34 (1H, brs) |
| 388 | 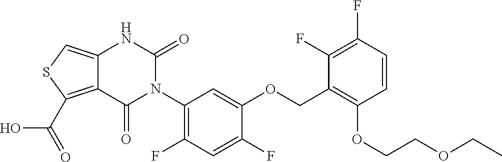 [SMILES: CCOCCOc1ccc(F)c(F)c1COc1cc(-n2c(=O)[nH]c3csc(C(=O)O)c3c2=O)c(F)cc1F] | (DMSO-d6) 0.99 (3H, t, J = 7.0 Hz), 3.4 (2H, q, J = 7.0 Hz), 3.55-3.7 (2H, m), 4.05-4.2 (2H, m), 5.11 (2H, s), 6.9-7.0 (1H, m), 7.38 (1H, s), 7.4-7.65 (3H, m), 12.03 (1H, s), 14.35 (1H, brs) |
| 389 | 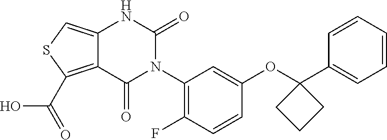 [SMILES: O=C(O)c1scc2[nH]c(=O)n(-c3cc(OC4(c5ccccc5)CCC4)ccc3F)c(=O)c12] | (DMSO-d6) 1.75-1.9 (1H, m), 1.9-2.05 (1H, m), 2.5-2.7 (4H, m), 6.55-6.6 (1H, m), 6.9-6.95 (1H, m), 7.1-7.2 (1H, m), 7.25-7.45 (4H, m), 7.45-7.55 (2H, m), 11.96 (1H, s), 14.41 (1H, s) |
| 390 | 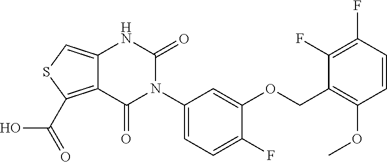 [SMILES: COc1ccc(F)c(F)c1COc1cc(-n2c(=O)[nH]c3csc(C(=O)O)c3c2=O)ccc1F] | (DMSO-d6) 3.82 (3H, s), 5.08 (2H, s), 6.9-7.0 (1H, m), 7.0-7.1 (1H, m), 7.3- 7.4 (2H, m), 7.4-7.55 (2H, m), 11.91 (1H, brs), 14.82 (1 H, brs) |
| 391 | 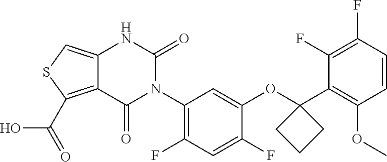 [SMILES: COc1ccc(F)c(F)c1C1(Oc2cc(-n3c(=O)[nH]c4csc(C(=O)O)c4c3=O)c(F)cc2F)CCC1] | (DMSO-d6) 1.8-1.95 (1H, m), 2.25-2.4 (1H, m), 2.6-2.8 (4H, m), 3.6 (3H, s), 6.65-6.75 (1H, m), 7.05-7.15 (1H, m), 7.25-7.35 (2H, m), 7.35-7.5 (1H, m), 11.7-12.2 (1H, br), 14.1-14.8 (1H, br) |
| 392 | 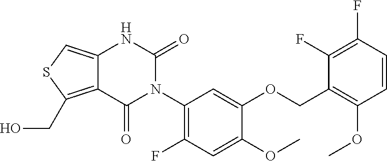 [SMILES: COc1cc(F)c(-n2c(=O)[nH]c3csc(CO)c3c2=O)cc1OCc1c(OC)ccc(F)c1F] | (DMSO-d6) 3.78 (3H, s), 3.81 (3H, s), 4.9-5.1 (4H, m), 5.95 (1H, t, J = 5.6 Hz), 6.71 (1H, s), 6.85-6.95 (1H, m), 7.05 (1H, d, J = 11.2 Hz), 7.17 (1H, d, J = 7.4 Hz), 7.4-7.55 (1H, m), 11.32 (1H, s) |
| 393 | 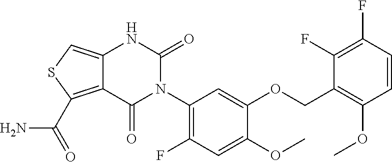 [SMILES: COc1cc(F)c(-n2c(=O)[nH]c3csc(C(N)=O)c3c2=O)cc1OCc1c(OC)ccc(F)c1F] | (DMSO-d6) 3.8 (3H, s), 3.81 (3H, s), 4.95 (2H, s), 6.85-6.95 (1H, m), 7.1 (1H, d, J = 11.5 Hz), 7.2-7.3 (2H, m), 7.4- 7.55 (1H, m), 8.05-8.15 (1H, m), 9.65- 9.75 (1H, m), 11.77 (1H, s) |
| 394 | 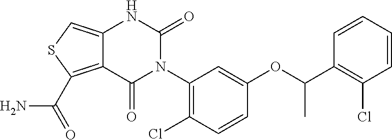 [SMILES: CC(Oc1ccc(Cl)c(-n2c(=O)[nH]c3csc(C(N)=O)c3c2=O)c1)c1ccccc1Cl] | (DMSO-d6) 1.58 (3H, d, J = 6.4 Hz), 5.65-5.75 (1H, m), 6.85-6.9 (1H, m), 7.2-7.25 (2H, m), 7.3-7.45 (2H, m), 7.45-7.55 (3H, m), 8.05-8.15 (1H, m), 9.6-9.7 (1H, m), 11.7-11.8 (1H, m) |
| 395 | 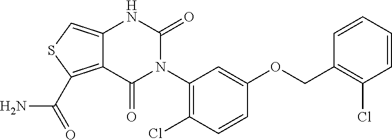 [SMILES: NC(=O)c1scc2[nH]c(=O)n(-c3cc(OCc4ccccc4Cl)ccc3Cl)c(=O)c12] | (DMSO-d6) 5.16 (2H, s), 7.19 (1H, dd, J = 8.9 Hz, 3.2 Hz), 7.24 (1H, s), 7.35- 7.45 (3H, m), 7.5-7.7 (3H, m), 8.11 (1H, d, J = 2.1 Hz), 9.65-9.7 (1H, m), 11.8 (1H, s) |
| 396 | 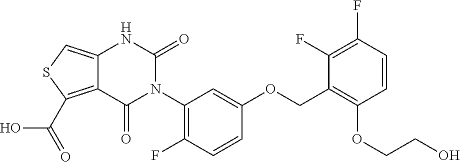 [SMILES: O=C(O)c1scc2[nH]c(=O)n(-c3cc(OCc4c(OCCO)ccc(F)c4F)ccc3F)c(=O)c12] | (DMSO-d6) 3.6-3.75 (2H, m), 4.04 (2H, t, J = 4.9 Hz), 4.8-4.95 (1H, m), 5.13 (2H, s), 6.9-6.95 (1H, m), 7.15-7.3 (2H, m), 7.3-7.4 (2H, m), 7.4-7.5 (1H, m), 12.01 (1H, s), 14.4 (1H, brs) |
| 397 | 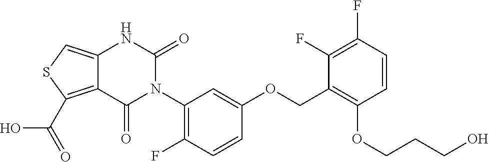 [SMILES: O=C(O)c1scc2[nH]c(=O)n(-c3cc(OCc4c(OCCCO)ccc(F)c4F)ccc3F)c(=O)c12] | (DMSO-d6) 1.75-1.9 (2H, m), 3.45-3.55 (2H, m), 4.1 (2H, t, J = 6.2 Hz), 4.9-5.0 (1H, m), 5.06 (2H, s), 6.85-6.95 (1H, m), 7.15-7.25 (2H, m), 7.3-7.4 (2H, m), 7.4- 7.5 (1H, m), 12.02 (1H, s), 14.4 (1H, brs) |
| 398 | 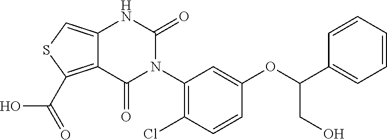 [SMILES: O=C(O)c1scc2[nH]c(=O)n(-c3cc(OC(CO)c4ccccc4)ccc3Cl)c(=O)c12] | (DMSO-d6) 3.55-3.65 (1H, m), 3.7-3.8 (1H, m), 5.15-5.25 (1H, m), 5.25-5.35 (1H, m), 7.0-7.05 (1H, m), 7.2-7.5 (8H, m), 11.95-12.05 (1H, m), 14.42 (1H, s) |
| 399 | 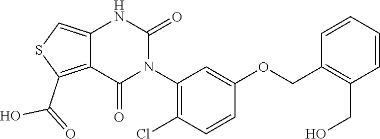 [SMILES: O=C(O)c1scc2[nH]c(=O)n(-c3cc(OCc4ccccc4CO)ccc3Cl)c(=O)c12] | (DMSO-d6) 4.61 (2H, s), 5.16 (2H, s), 5.21 (1H, brs), 7.15-7.5 (7H, m), 7.57 (1H, d, J = 8.6 Hz), 1.20 (1H, s) |
| 400 | 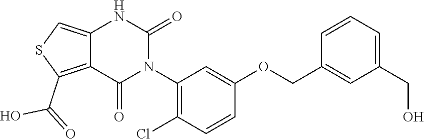 [SMILES: O=C(O)c1scc2[nH]c(=O)n(-c3cc(OCc4cccc(CO)c4)ccc3Cl)c(=O)c12] | (DMSO-d6) 4.51 (2H, d, J = 5.2 Hz), 5.1 (2H, s), 5.22 (1H, t, J = 5.2 Hz), 7.15-7.2 (1H, m), 7.25-7.45 (6H, m), 7.57 (1H, d, J = 8.6 Hz), 12.03 (1H, s), 14.44 (1H, s) |
| 401 | 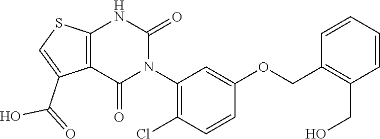 [SMILES: O=C(O)c1csc2[nH]c(=O)n(-c3cc(OCc4ccccc4CO)ccc3Cl)c(=O)c12] | (DMSO-d6) 4.61 (2H, s), 5.15 (2H, s), 7.15-7.5 (6H, m), 7.57 (1H, d, J = 9.2 Hz), 7.95 (1H, s), 13.95 (1H, s) |
| 402 | 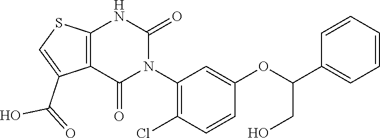 [SMILES: O=C(O)c1csc2[nH]c(=O)n(-c3cc(OC(CO)c4ccccc4)ccc3Cl)c(=O)c12] | (DMSO-d6) 3.55-3.65 (1H, m), 3.7-3.8 (1H, m), 5.0-5.4 (2H, m), 7.0-7.1 (1H, m), 7.15-7.5 (7H, m), 7.95 (1H, d, J = 5.9 Hz), 12.98 (1H, brs), 13.95 (1H, s) |
| 403 | 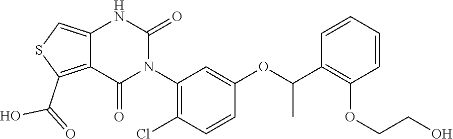 [SMILES: CC(Oc1ccc(Cl)c(-n2c(=O)[nH]c3csc(C(=O)O)c3c2=O)c1)c1ccccc1OCCO] | (DMSO-d6) 1.55 (3H, d, J = 6.3 Hz), 3.6-3.8 (2H, m), 3.95-4.15 (2H, m), 4.85-4.95 (1H, m), 5.75-5.9 (1H, m), 6.9-7.05 (3H, m), 7.15-7.5 (5H, m), 11.95-12.05 (1H, m), 14.43 (1H, s) |
| 404 | 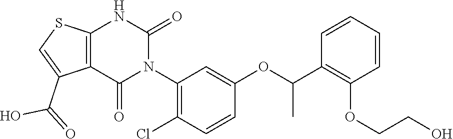 [SMILES: CC(Oc1ccc(Cl)c(-n2c(=O)[nH]c3scc(C(=O)O)c3c2=O)c1)c1ccccc1OCCO] | (DMSO-d6) 1.55 (3H, d, J = 6.3 Hz), 3.6-3.8 (2H, m), 3.95-4.15 (2H, m), 4.8- 5.0 (1H, br), 5.75-5.85 (1H, m), 6.9-7.05 (3H, m), 7.15-7.35 (3H, m), 7.43 (1H, d, J = 8.9 Hz), 7.9-8.0 (1H, m), 12.8-13.2 (1H, br), 13.99 (1H, s) |
| 405 | 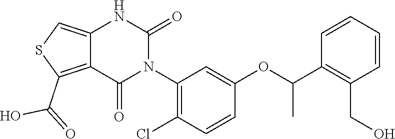 [SMILES: CC(Oc1ccc(Cl)c(-n2c(=O)[nH]c3csc(C(=O)O)c3c2=O)c1)c1ccccc1CO] | (DMSO-d6) 1.57 (3H, d, J = 6.2 Hz), 4.55-4.65 (1H, m), 4.65-4.75 (1H, m), 5.3-5.35 (1H, m), 5.7-5.8 (1H, m), 6.95- 7.05 (1H, m), 7.2-7.5 (7H, m), 11.95- 12.05 (1H m), 14.42 (1H, s) |
| 406 | 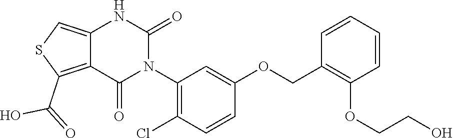 [SMILES: O=C(O)c1scc2[nH]c(=O)n(-c3cc(OCc4ccccc4OCCO)ccc3Cl)c(=O)c12] | (DMSO-d6) 3.65-3.75 (2H, m), 4.0-4.1 (2H, m), 4.87 (1H, brs), 5.12 (2H, s), 6.95-7.0 (1H, m), 7.05 (1H, d, J = 8.1 Hz), 7.19 (1H, dd, J = 9.0 Hz, 3.0 Hz), 7.25-7.45 (4H, m), 7.56 (1H, d, J = 9.0 Hz), 12.04 (1H, s), 14.46 (1H, s) |
| 407 | 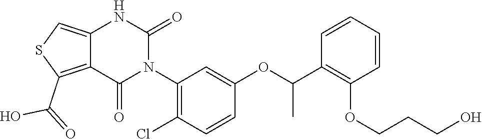 [SMILES: CC(Oc1ccc(Cl)c(-n2c(=O)[nH]c3csc(C(=O)O)c3c2=O)c1)c1ccccc1OCCCO] | (DMSO-d6) 1.54 (3H, d, J = 6.3 Hz), 1.85-1.95 (2H, m), 3.55-3.65 (2H, m), 4.05-4.2 (2H, m), 4.54 (1H, brs), 5.65- 5.75 (1H, m), 6.85-7.0 (2H m), 7.0-7.1 (1H, m), 7.15-7.2 (1H, m), 7.2-7.35 (2H, m), 7.4 (1H, d, J = 4.6 Hz), 7.47 (1H, d, J = 9.0 Hz), 11.95-12.05 (1H, m), 14.42 (1H, s) |
| 408 | 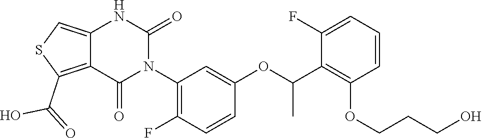 [SMILES: CC(Oc1ccc(F)c(-n2c(=O)[nH]c3csc(C(=O)O)c3c2=O)c1)c1c(F)cccc1OCCCO] | (DMSO-d6) 1.66 (3H, d, J = 6.4 Hz), 1.85-1.95 (2H, m), 3.5-3.6 (2H, m), 4.05-4.2 (2H, m), 5.75-5.85 (1H, m), 6.7-7.1 (4H, m), 7.2-7.4 (3H, 7), 11.95- 12.05 (1H, m), 14.35-14.45 (1H, m) |
| 409 | 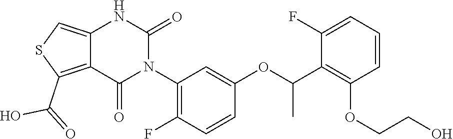 [SMILES: CC(Oc1ccc(F)c(-n2c(=O)[nH]c3csc(C(=O)O)c3c2=O)c1)c1c(F)cccc1OCCO] | (DMSO-d6) 1.66 (3H, d, J = 6.6 Hz), 3.55-3.8 (2H, m), 3.85-4.0 (1H, m), 4.05-4.2 (1H, m), 4.9-5.05 (1H, m), 5.9- 6.0 (1H, m), 6.7-6.9 (2H, m), 7.0-7.4 (5H, m), 11.95-12.05 (1H, m), 14.44 (1H, s) |
| 410 | 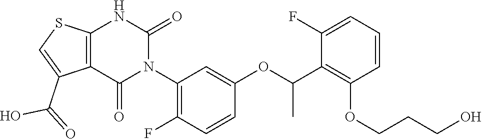 [SMILES: CC(Oc1ccc(F)c(-n2c(=O)[nH]c3scc(C(=O)O)c3c2=O)c1)c1c(F)cccc1OCCCO] | (DMSO-d6) 1.66 (3H, d, J = 6.4 Hz), 1.8-1.95 (2H, m), 3.5-3.6 (2H, m), 4.0- 4.2 (2H, m), 5.8 (1H, q, J = 6.4 Hz), 6.7- 6.8 (1H, m), 6.8-7.0 (2H, m), 7.0-7.05 (1H, m), 7.2-7.35 (2H, m), 7.9-7.95 (1H, m), 13.93 (1H, s) |
| 411 | 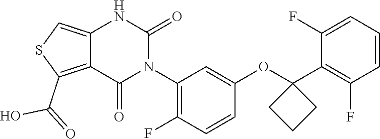 [SMILES: O=C(O)c1scc2[nH]c(=O)n(-c3cc(OC4(c5c(F)cccc5F)CCC4)ccc3F)c(=O)c12] | (DMSO-d6) 1.85-2.0 (1H, m), 2.2-2.35 (1H, m), 2.65-2.85 (4H, m), 6.75-6.85 (1H, m), 7.0-7.1 (3H, m), 7.24 (1H, t, J = 9.3 Hz), 7.3-7.45 (2H, m), 11.97 (1H, brs), 13.5-15.0 (1H, br) |
| 412 | 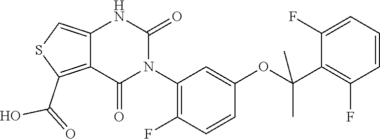 [SMILES: CC(C)(Oc1ccc(F)c(-n2c(=O)[nH]c3csc(C(=O)O)c3c2=O)c1)c1c(F)cccc1F] | (DMSO-d6) 1.820 (3H, s), 1.823 (3H, s), 6.75-6.8 (1H, m), 6.95-7.1 (3H, m), 7.15-7.25 (1H, m), 7.3-7.45 (2H, m), 11.96 (1H, brs), 14.43 (1H, brs) |
| 413 | 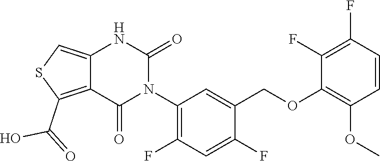 [SMILES: COc1ccc(F)c(F)c1OCc1cc(-n2c(=O)[nH]c3csc(C(=O)O)c3c2=O)c(F)cc1F] | (DMSO-d6) 3.79 (3H, s), 5.05-5.2 (2H, m), 6.8-6.9 (1H, m), 7.05-7.2 (1H, m), 7.37 (1H, s), 7.5-7.6 (1H, m), 7.65-7.75 (1H, m), 12.02 (1H, s), 14.35 (1H, s) |
| 414 | 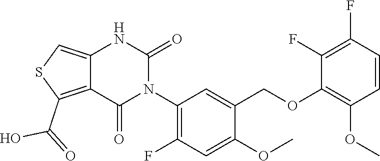 [SMILES: COc1cc(F)c(-n2c(=O)[nH]c3csc(C(=O)O)c3c2=O)cc1COc1c(OC)ccc(F)c1F] | (DMSO-d6) 3.79 (3H, s), 3.85 (3H, s), 5.0-5.1 (2H, m), 6.8-6.9 (1H, m), 7.05- 7.2 (2H, m), 7.38 (1H, s), 7.51 (1H, d, J = 8.5 Hz), 11.98 (1H s), 14.54 (1H, brs) |
| 415 | 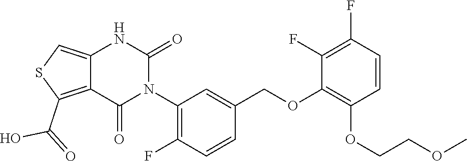 [SMILES: COCCOc1ccc(F)c(F)c1OCc1ccc(F)c(-n2c(=O)[nH]c3csc(C(=O)O)c3c2=O)c1] | (DMSO-d6) 3.27 (3H, s), 3.6-3.7 (2H, m), 4.1-4.2 (2H, m), 5.13 (2H, s), 6.85- 6.95 (1H, m), 7.05-7.2 (1H, m), 7.39 (1H, s), 7.4-7.5 (1H, m), 7.6-7.7 (2H, m), 12.05 (1H, s), 14.42 (1H, s) |
| 416 | 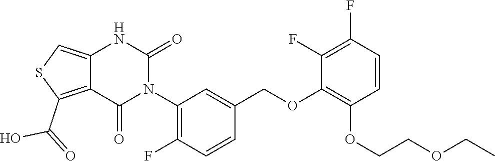 [SMILES: CCOCCOc1ccc(F)c(F)c1OCc1ccc(F)c(-n2c(=O)[nH]c3csc(C(=O)O)c3c2=O)c1] | (DMSO-d6) 1.05 (3H, t, J = 7.1 Hz), 3.46 (2H, q, J = 7.1 Hz), 3.65-3.75 (2H, m), 4.1-4.2 (2H, m), 5.14 (2H, s), 6.85- 6.95 (1H, m), 7.05-7.2 (1H, m), 7.38 (1H, s), 7.4-7.5 (1H, m), 7.55-7.7 (2H, m), 12.05 (1H, s), 14.41 (1H, s) |
| 417 | 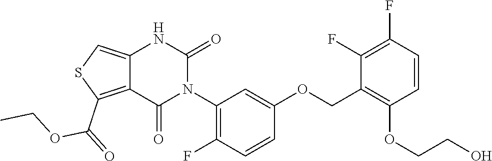 [SMILES: CCOC(=O)c1scc2[nH]c(=O)n(-c3cc(OCc4c(OCCO)ccc(F)c4F)ccc3F)c(=O)c12] | (DMSO-d6) 1.27 (3H, t, J = 7.1 Hz), 3.68 (2H, t, J = 4.8 Hz), 4.05 (2H, t, J = 4.8 Hz), 4.3 (2H, q, J = 7.1 Hz), 5.12 (2H, s), 6.85-6.95 (1H, m), 7.1-7.25 (3H, m), 7.25-7.35 (1H, m), 7.4-7.5 (1H, m), 11.63 (1H, s) |
| 418 | 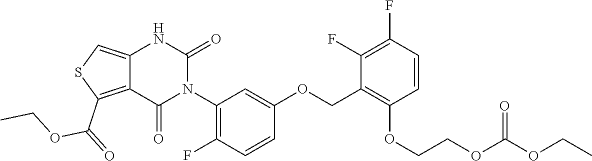 [SMILES: CCOC(=O)OCCOc1ccc(F)c(F)c1COc1ccc(F)c(-n2c(=O)[nH]c3csc(C(=O)OCC)c3c2=O)c1] | (DMSO-d6) 1.13 (3H, t, J = 7.0 Hz), 12.7 (3H, t, J = 7.1 Hz), 4.03 (2H, q, J = 7.0 Hz), 4.25-4.35 (4H, m), 4.35-4.45 (2H, m), 5.07 (2H, s), 6.9-7.0 (1H, m), 7.1-7.2 (3H, m), 7.25-7.35 (1H, m), 7.4- 7.55 (1H, m), 11.63 (1H, s) |
| 419 | 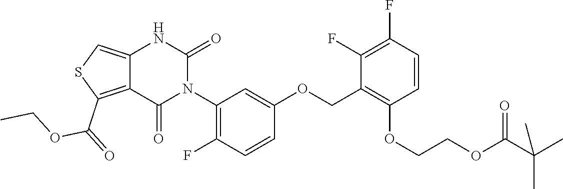 [SMILES: CCOC(=O)c1scc2[nH]c(=O)n(-c3cc(OCc4c(OCCOC(=O)C(C)(C)C)ccc(F)c4F)ccc3F)c(=O)c12] | (DMSO-d6) 1.04 (9H, s), 1.27 (3H, t, J = 7.1 Hz), 4.2-4.35 (6H, m), 5.05 (2H, s), 6.9-7.05 (1H, m), 7.05-7.2 (3H, m), 7.25-7.35 (1H, m), 7.4-7.55 (1H, m), 11.63 (1H, s) |
| 420 | 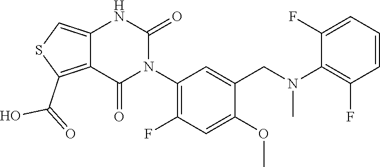 [SMILES: COc1cc(F)c(-n2c(=O)[nH]c3csc(C(=O)O)c3c2=O)cc1CN(C)c1c(F)cccc1F] | (DMSO-d6) 2.7-2.75 (3H, m), 3.71 (3H, s), 4.1-4.25 (2H, m), 6.95-7.15 (4H, m), 7.38 (1H, s), 7.41 (1H, d, J = 8.6 Hz), 11.95 (1H, s), 14.57 (1H, brs) |
| 421 | 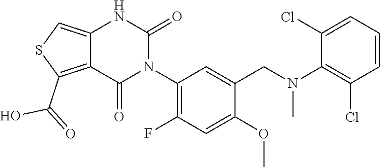 [SMILES: COc1cc(F)c(-n2c(=O)[nH]c3csc(C(=O)O)c3c2=O)cc1CN(C)c1c(Cl)cccc1Cl] | (DMSO-d6) 2.73 (3H, s), 3.74 (3H, s), 4.15-4.25 (2H, m), 7.08 (1H, d, J = 12.4 Hz), 7.15-7.2 (1H, m), 7.36 (1H, s), 7.4- 7.45 (2H, m), 7.56 (1H, d, J = 8.7 Hz), 11.96 (1H, brs), 14.61 (1H, brs) |
| 422 | 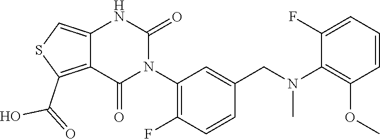 [SMILES: COc1cccc(F)c1N(C)Cc1ccc(F)c(-n2c(=O)[nH]c3csc(C(=O)O)c3c2=O)c1] | (DMSO-d6) 2.68 (3H, s), 3.81 (3H, s), 4.15 (2H, s), 6.7-6.85 (2H, m), 7.0-7.1 (1H, m), 7.3-7.4 (2H, m), 7.45-7.55 (2H, m), 11.98 (1H, s), 14.45 (1H, s) |
| 423 | 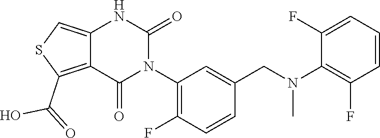 [SMILES: CN(Cc1ccc(F)c(-n2c(=O)[nH]c3csc(C(=O)O)c3c2=O)c1)c1c(F)cccc1F] | (DMSO-d6) 2.75 (3H, s), 4.22 (2H, s), 6.95-7.15 (3H, m), 7.35-7.45 (2H, m), 7.45-7.5 (2H, m), 12.02 (1H, s) |
| 424 | 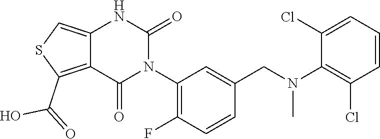 [SMILES: CN(Cc1ccc(F)c(-n2c(=O)[nH]c3csc(C(=O)O)c3c2=O)c1)c1c(Cl)cccc1Cl] | (DMSO-d6) 2.72 (3H, s), 4.3 (2H, s), 7.15-7.25 (1H, m), 7.35-7.5 (4H, m), 7.55-7.65 (2H, m), 12.03 (1H, s) |
| 425 | 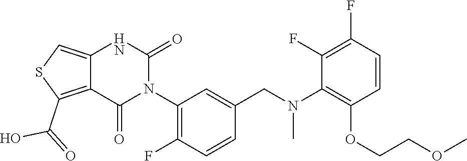 [SMILES: COCCOc1ccc(F)c(F)c1N(C)Cc1ccc(F)c(-n2c(=O)[nH]c3csc(C(=O)O)c3c2=O)c1] | (DMSO-d6) 2.7-2.75 (3H, m), 3.23 (3H, s), 3.6-3.7 (2H, m), 4.05-4.1 (2H, m), 4.22 (2H, s), 6.7-6.8 (1H, m), 7.0-7.1 (1H, m), 7.3-7.4 (2H, m), 7.45-7.55 (2H, m), 12.03 (1H, s), 14.46 (1H, brs) |
| 426 | 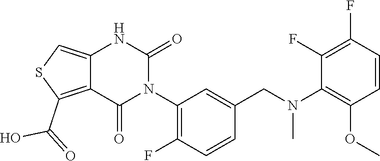 [SMILES: COc1ccc(F)c(F)c1N(C)Cc1ccc(F)c(-n2c(=O)[nH]c3csc(C(=O)O)c3c2=O)c1] | (DMSO-d6) 2.7-2.75 (3H, m), 3.79 (3H, s), 4.2 (2H, s), 6.7-6.8 (1H, m), 7.0-7.15 (1H, m), 7.3-7.4 (2H, m), 7.45-7.55 (2H, m), 12.02 (1H, s), 14.47 (1H, brs) |
| 427 | 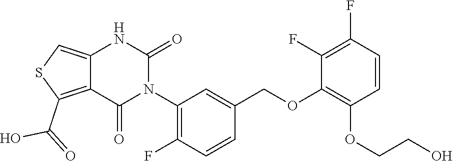 [SMILES: O=C(O)c1scc2[nH]c(=O)n(-c3cc(COc4c(OCCO)ccc(F)c4F)ccc3F)c(=O)c12] | (DMSO-d6) 3.7-3.8 (2H, m), 4.04 (2H, t, J = 4.6 Hz), 4.85-4.95 (1H, m), 5.17 (2H, s), 6.85-6.95 (1H, m), 7.05-7.15 (1H, m), 7.39 (1H, s), 7.4-7.5 (1H, m), 7.6-7.7 (2H, m), 12.05 (1H, s), 14.44 (1H, s) |
| 428 | 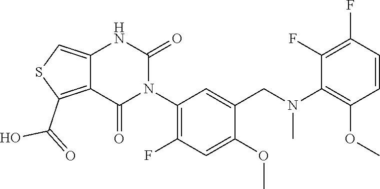 [SMILES: COc1cc(F)c(-n2c(=O)[nH]c3csc(C(=O)O)c3c2=O)cc1CN(C)c1c(OC)ccc(F)c1F] | (DMSO-d6) 2.7-2.75 (3H, m), 3.7-3.8 (6H, m), 4.1-4.2 (2H, m), 6.7-6.75 (1H, m), 6.95-7.1 (2H, m), 7.37 (1H, s), 7.41 (1H, d, J = 8.8 Hz), 11.95 (1H, s), 14.57 (1H, s) |

For example, the GnRH antagonist may be 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid, or a pharmaceutically acceptable salt thereof. The salt may be, for instance, the choline salt thereof, represented by formula (VIa), below.

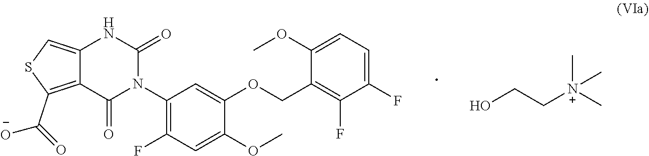

Compound (VI) and pharmaceutically acceptable salts thereof, such as the choline salt thereof (compound (VIa)), can be synthesized, for example, using the methodology described in WO 2014/042176, the disclosure of which is incorporated herein by reference in its entirety. An exemplary synthetic scheme that may be used for the preparation of compound (VI) and the choline salt thereof is shown in Scheme 1, below.

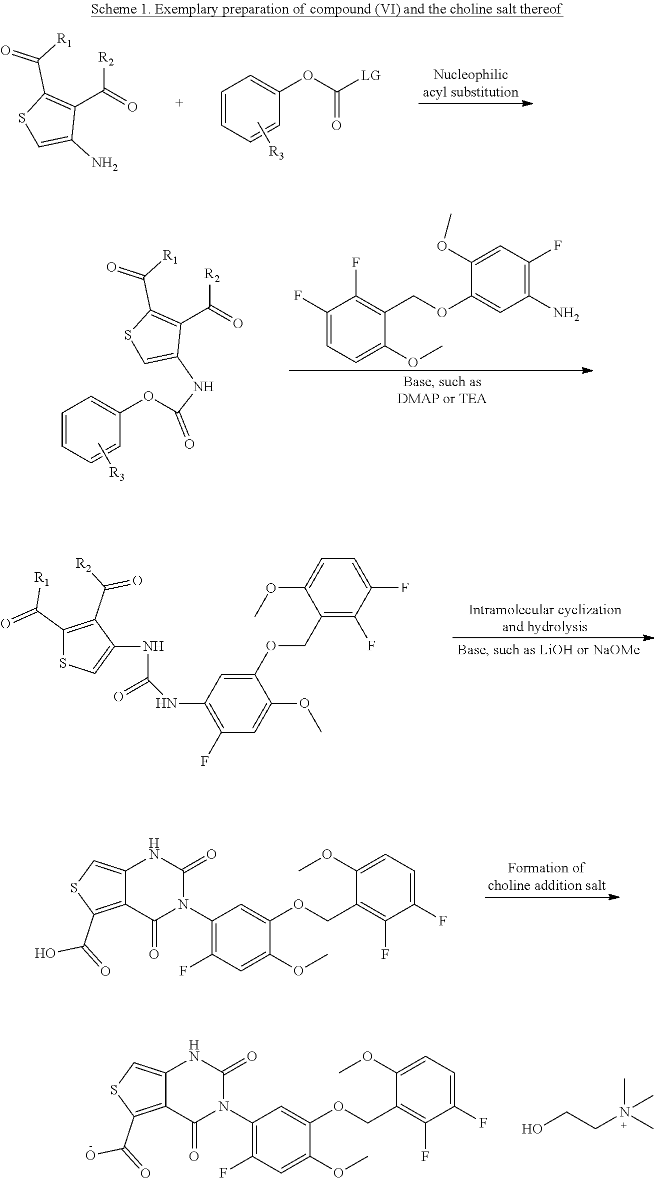

wherein R₁ and R₂ are independently C₁₋₆ alkoxy groups; LG is a nucleofugal leaving group, such as chlorine or bromine, among others; R₃ represents an optional substituent, such as halogen, acyl group, C₁₋₆ alkyl group, or a nitro substituent; DMAP denotes N-dimethylaminopyridine; and TEA denotes trimethylamine.

Crystalline compound (VIa) has been characterized spectroscopically, for instance, in U.S. Pat. No. 9,169,266, the disclosure of which is incorporated herein by reference in its entirety. The foregoing crystalline form has been shown to exhibit characteristic X-ray powder diffraction peaks at about 7.1° 2θ, about 11.5° 2θ, about 19.4° 2θ, about 21.5° 2θ, about 22.0° 2θ, about 22.6° 2θ, about 23.5° 2θ, and about 26.2° 2θ. Additionally, this crystalline form exhibits ¹³C solid-state nuclear magnetic resonance (NMR) peaks centered at about 55.5 ppm, about 57.1 ppm, about 58.7 ppm, about 69.8 ppm, about 98.1 ppm, about 110.3 ppm, about 111.6 ppm, about 113.7 ppm, about 118.0 ppm, about 145.3 ppm, about 149.8 ppm, and about 155.8 ppm. This crystalline form further exhibits ¹⁹F solid-state NMR peaks centered at about −151.8 ppm, −145.2 ppm, and −131.6 ppm.

Compound (VI), as well as pharmaceutically acceptable salts thereof, such as the choline salt thereof, exhibit a high affinity for human GnRH receptor (27.4 nM). Using the compositions and methods described herein, a patient that is presenting with or has been diagnosed as having an estrogen-dependent disease (e.g., uterine fibroids, endometriosis, such as rectovaginal endometriosis, and/or adenomyosis, among others described herein) may be administered a compound of formula (VI), or a pharmaceutically acceptable salt thereof, such as the choline salt thereof, to treat the disease or ameliorate one or more symptoms of the disease. Exemplary doses of compound (VI) and pharmaceutically acceptable salts thereof, such as the choline salt thereof, include doses of from 25 mg to 500 mg daily, such as doses of 100 mg per day and 200 mg per day. Additional dosing information is provided below. 3-Aminoalkyl pyrimidine-2,4(1H,3H)-diones

Additional GnRH antagonists that may be used in conjunction with the compositions and methods described herein include optionally substituted 3-aminoalkyl pyrimidine-2,4(1H,3H)-dione derivatives, such as compounds represented by formula (VII)

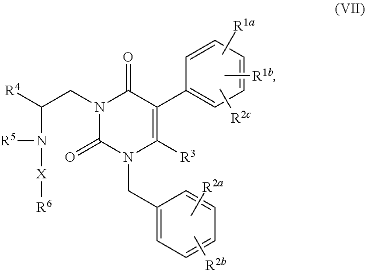

wherein R₁ₐ, R_{1b} and R_{1c} are the same or different and are each independently hydrogen, halogen, C₁₋₄alkyl, hydroxy or alkoxy, or R₁ₐ and R_{1b} taken together form —OCH₂O— or —OCH₂CH₂—;

R₂ₐ and R_{2b} are the same or different and are each independently hydrogen, halogen, trifluoromethyl, cyano or —SO₂CH₃;

R₃ is hydrogen or methyl;

R₄ is phenyl or C₃₋₇alkyl;

R₅ is hydrogen or C₁₋₄alkyl;

R₆ is —COOH or an acid isostere; and

X is C₁₋₆alkanediyl optionally substituted with from 1 to 3 C₁₋₆alkyl groups;

or a pharmaceutically acceptable salt thereof.

For example, the GnRH antagonist may be the conjugate acid of elagolix, which is represented by formula (VIII),

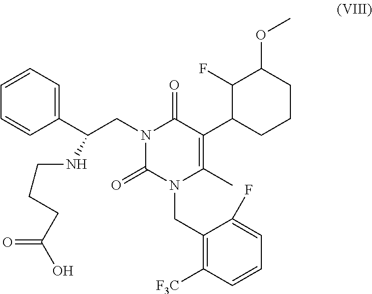 [SMILES: COC1CCCC(c2c(C)n(Cc3c(F)cccc3C(F)(F)F)c(=O)n(C[C@H](NCCCC(=O)O)c3ccccc3)c2=O)C1F]

or a pharmaceutically acceptable salt thereof. In some embodiments, the GnRH antagonist is the sodium salt of the compound represented by formula (VIII), which is represented by formula (IX), below.

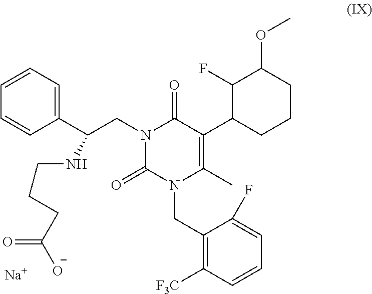 [SMILES: COC1CCCC(c2c(C)n(Cc3c(F)cccc3C(F)(F)F)c(=O)n(C[C@H](NCCCC(=O)[O-])c3ccccc3)c2=O)C1F ; [Na+]]

Compound (IX), also referred to as sodium 4-({(1R)-2-[5-(2-fluoro-3-methoxyphenyl)-3-{[2-fluoro-6-(trifluoromethyl)phenyl]methyl}-4-methyl-2,6-dioxo-3,6-dihydropyrimidin-1(2H)-yl]-1-phenylethyl}amino)butanoate, is known as elagolix. Other GnRH antagonists of this chemical class that may be used for the treatment of estrogen-dependent diseases in accordance with the compositions and methods of the disclosure include compounds described in U.S. Pat. No. 7,056,927, the contents of which are incorporated herein by reference. Exemplary GnRH antagonists of this chemical class that may be used for the treatment of estrogen-dependent diseases in accordance with the present disclosure include the compounds set forth in Table 2, below.

TABLE 2
Exemplary 3-Aminoalkyl pyrimidine-2,4(1H,3H)-dione GnRH Antagonists Useful for the
Treatment of Estrogen-Dependent Diseases

| No. | Compound | Reported spectral properties |
|---|---|---|
| 1 | 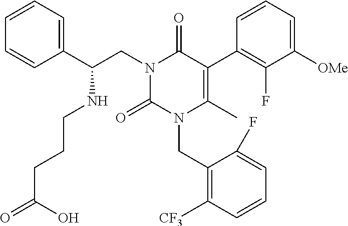 [SMILES: COc1cccc(-c2c(C)n(Cc3c(F)cccc3C(F)(F)F)c(=O)n(C[C@H](NCCCC(=O)O)c3ccccc3)c2=O)c1F] | HPLC-MS (Cl) m/z 632.2 (MH+), tR = 26.45 |
| 2 | 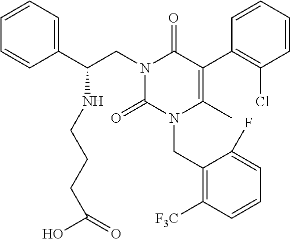 [SMILES: Cc1c(-c2ccccc2Cl)c(=O)n(C[C@H](NCCCC(=O)O)c2ccccc2)c(=O)n1Cc1c(F)cccc1C(F)(F)F] | MS (Cl) m/z 618.2 (MH+) tR = 1.005 |
| 3 | 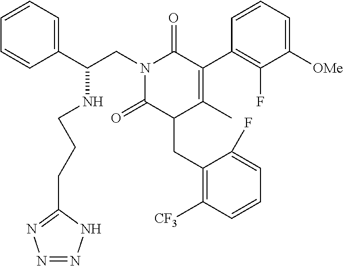 [SMILES: COc1cccc(C2=C(C)C(Cc3c(F)cccc3C(F)(F)F)C(=O)N(C[C@H](NCCCc3nnn[nH]3)c3ccccc3)C2=O)c1F] | HPLC-MS (Cl) m/z 656.2 (MH+), tR = 2.128 min |
| 4 | 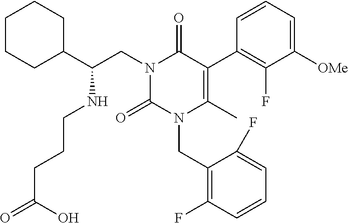 [SMILES: COc1cccc(-c2c(C)n(Cc3c(F)cccc3F)c(=O)n(C[C@H](NCCCC(=O)O)C3CCCCC3)c2=O)c1F] | MS (Cl) m/z 588.3 (MH+) |
| 5 | 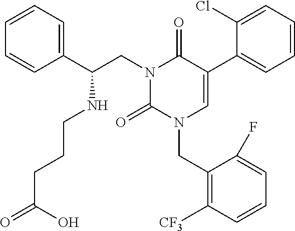 [SMILES: O=C(O)CCCN[C@@H](Cn1c(=O)c(-c2ccccc2Cl)cn(Cc2c(F)cccc2C(F)(F)F)c1=O)c1ccccc1] | HPLC-MS (Cl) m/z 604.1, 606.1 (MH+), tR = 2.511 |
| 6 | 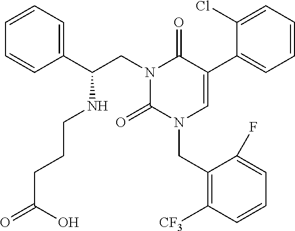 [SMILES: O=C(O)CCCN[C@@H](Cn1c(=O)c(-c2ccccc2Cl)cn(Cc2c(F)cccc2C(F)(F)F)c1=O)c1ccccc1] | HPLC-MS (Cl) m/z 604.1, 606.1 (MH+), tR = 26.98 |
| 7 | 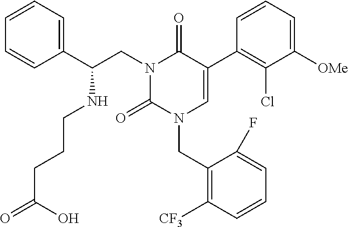 [SMILES: COc1cccc(-c2cn(Cc3c(F)cccc3C(F)(F)F)c(=O)n(C[C@H](NCCCC(=O)O)c3ccccc3)c2=O)c1Cl] | HPLC-MS (Cl) m/z 634.2, 636.2 (MH+), tR-24.925 |
| 8 | 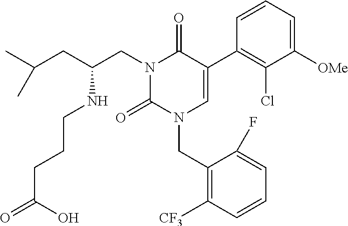 [SMILES: COc1cccc(-c2cn(Cc3c(F)cccc3C(F)(F)F)c(=O)n(C[C@@H](CC(C)C)NCCCC(=O)O)c2=O)c1Cl] | MS (Cl) m/z 614.1, 616.1 (MH+), tR = 6.550 min |
| 9 | 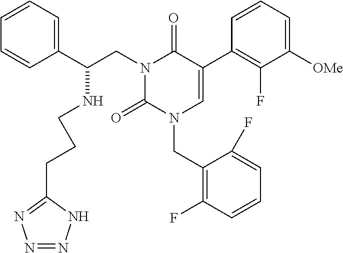 [SMILES: COc1cccc(-c2cn(Cc3c(F)cccc3F)c(=O)n(C[C@H](NCCCc3nnn[nH]3)c3ccccc3)c2=O)c1F] | HPLC-MS (Cl) m/z 592.3 (MH+), tR = 2.150 |
| 10 | 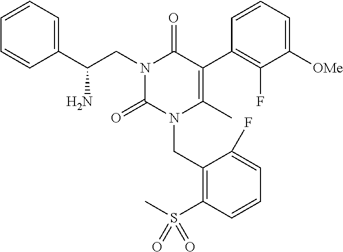 [SMILES: COc1cccc(-c2c(C)n(Cc3c(F)cccc3S(C)(=O)=O)c(=O)n(C[C@H](N)c3ccccc3)c2=O)c1F] | HPLC-MS (Cl) m/z = 556.2 (M + H+), tR = 2.354 |
| 11 | 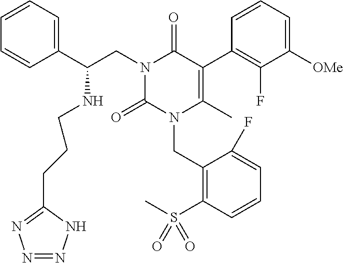 [SMILES: COc1cccc(-c2c(C)n(Cc3c(F)cccc3S(C)(=O)=O)c(=O)n(C[C@H](NCCCc3nnn[nH]3)c3ccccc3)c2=O)c1F] | |

### Thieno[2,3d]pyrimidines

Additional GnRH antagonists that may be used in conjunction with the compositions and methods described herein include optionally substituted thieno[2,3d]pyrimidine derivatives, such as compounds represented by formula (X)

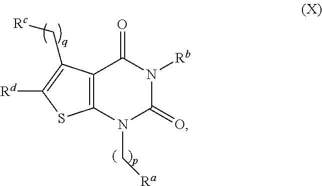

wherein R^{a} is a hydrogen atom, an optionally substituted aryl group (such as an aryl group that may have 1 to 5 substituents selected from halogen, nitro, cyano, amino, a carboxyl group that may be esterified or amidated, an alkylenedioxy, alkyl, alkoxy, alkylthio, alkylsulfinyl, and alkylsulfonyl), an optionally substituted cycloalkyl group, or an optionally substituted heterocyclic group;

R^{b} is an optionally substituted nitrogen-containing heterocyclic group;

R^{c} is an optionally substituted amino group;

R^{d} is an optionally substituted aryl group;

p is an integer from 0 to 3; and

q is an integer from 0 to 3;

or a pharmaceutically acceptable salt thereof.

In some embodiments, the GnRH antagonist is a thieno[2,3d]pyrimidine compound represented by formula (XI)

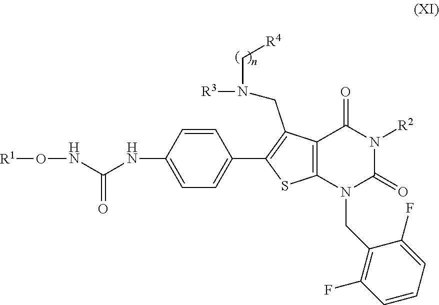

wherein R¹ is C₁₋₄alkyl;

R² is (1) a C₁₋₆alkyl which may have a substituent selected from the group consisting of (1′) a hydroxy group, (2′) a C₁₋₄alkoxy, (3′) a C₁₋₄alkoxy-carbonyl, (4′) a di-C₁₋₄alkyl-carbamoyl, (5′) a 5- to 7-membered nitrogen-containing heterocyclic group, (6′) a C₁₋₄alkyl-carbonyl and (7′) a halogen, (2) a C₃₋₈ cycloalkyl which may have (1′) a hydroxy group or (2′) a mono-C₁₋₄alkyl-carbonylamino, (3) a 5- to 7-membered nitrogen-containing heterocyclic group which may have a substituent selected from the group consisting of (1′) a halogen, (2′) a hydroxy group, (3′) a C₁₋₄alkyl and (4′) a C₁₋₄alkoxy, (4) a phenyl which may have a substituent selected from the group consisting of (1′) a halogen, (2′) a C₁₋₄alkoxy-C₁₋₄alkyl, (3′) a mono-C₁₋₄alkyl-carbamoyl-C₁₋₄alkyl, (4′) a C₁₋₄alkoxy and (5′) a mono-C₁₋₄alkylcarbamoyl-C₁₋₄alkoxy, or (5) a C₁₋₄alkoxy;

R³ is C₁₋₄alkyl;

R⁴ is (1) hydrogen, (2) C₁₋₄alkoxy, (3) C₆₋₁₀aryl, (4)N—C₁₋₄alkyl-N—C₁₋₄alkylsulfonylamino, (5) hydroxyl, or (6) a 5- to 7-membered nitrogen-containing heterocyclic group which may have a substituent selected from the group consisting of (1′) oxo, (2′) a C₁₋₄alkyl, (3′) a hydroxy-C₁₋₄alkyl, (4′) a C₁₋₄alkoxy-carbonyl, (5′) a mono-C₁₋₄alkyl-carbamoyl and (6′) a C₁₋₄alkylsulfonyl; and

n is an integer from 1 to 4;

optionally provided that when R² is a phenyl which may have a substituent, R⁴ is a 5- to 7-membered nitrogen-containing heterocyclic group which may have a substituent selected from the group consisting of (1) oxo, (2) hydroxy-C₁₋₄alkyl, (3) C₁₋₄alkoxy-carbonyl, (4) mono-C₁₋₄alkyl-carbamoyl and (5) C₁₋₄alkylsulfonyl;
or a pharmaceutically acceptable salt thereof. For example, the GnRH antagonist may be a compound represented by formula (XII), below.

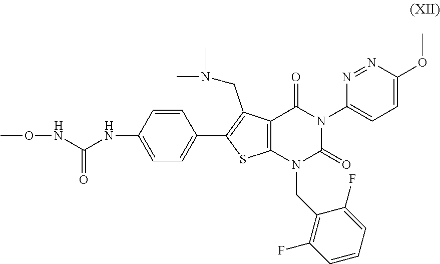

Compound (XII), also referred to as N-(4-(1-(2,6-difluorobenzyl)-5-((dimethylamino)methyl)-3-(6-methoxy-3-pyridazinyl)-2,4-dioxo-1,2,3,4-tetrahydrothieno[2,3-d]pyrimidin-6-yl)phenyl)-N′-methoxyurea, is known as relugolix. Other GnRH antagonists of this chemical class that may be used for the treatment of estrogen-dependent diseases in accordance with the compositions and methods of the disclosure include compounds described in U.S. Pat. No. 7,300,935, the contents of which are incorporated herein by reference. Exemplary GnRH antagonists of this chemical class that may be used for the treatment of estrogen-dependent diseases in accordance with the present disclosure include the compounds set forth in Table 3, below.

TABLE 3
Exemplary Thieno[2,3d]pyrimidine GnRH Antagonists Useful for the Treatment of Estrogen-Dependent Diseases

| No. | Compound | Reported spectral properties |
|---|---|---|
| 1 | 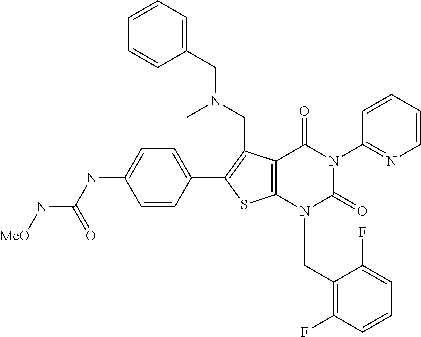 [SMILES: CONC(=O)Nc1ccc(-c2sc3c(c2CN(C)Cc2ccccc2)c(=O)n(-c2ccccn2)c(=O)n3Cc2c(F)cccc2F)cc1] | ¹H-NMR (CDCl₃) δ: 2.05 (3H, s), 3.56 (2H, s), 3.82 (3H, s), 3.89 (2H, s), 5.34 (2H, brs), 6.91 (2H, t, J = 8.0 Hz), 7.1- 7.45 (9H, m), 7.56 (2H, d, J = 8.8 Hz), 7.65 (1H, s), 7.75 (2H, d, J = 8.8 Hz), 7.91 (1H, dt, J = 2.0, 7.7 Hz), 8.7-8.75 (1H, m). |
| 2 | 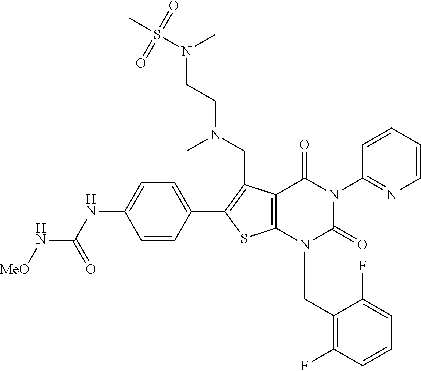 [SMILES: CONC(=O)Nc1ccc(-c2sc3c(c2CN(C)CCN(C)S(C)(=O)=O)c(=O)n(-c2ccccn2)c(=O)n3Cc2c(F)cccc2F)cc1] | ¹H-NMR (CDCl₃) δ: 2.11 (3H, s), 2.45-2.6 (2H, m), 2.70 (3H, s), 2.75 (3H, s), 3.1-3.25 (2H, m), 3.80 (2H, s), 3.83 (3H, s), 5.36 (2H, brs), 6.93 (2H, t, J = 8.2 Hz), 7.14 (1H, s), 7.2- 7.6 (7H, m), 7.65 (1H s), 7.85- 7.95 (1H, m), 8.65-8.75 (1H, m). |
| 3 | 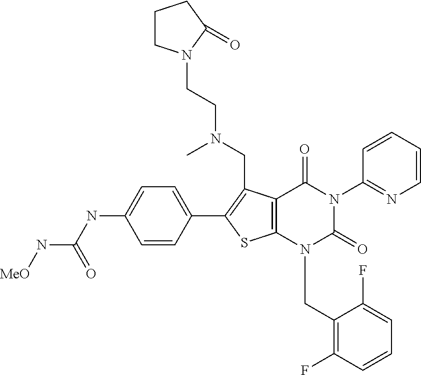 [SMILES: CONC(=O)Nc1ccc(-c2sc3c(c2CN(C)CCN2CCCC2=O)c(=O)n(-c2ccccn2)c(=O)n3Cc2c(F)cccc2F)cc1] | ¹H-NMR (CDCl₃) δ: 1.7-1.85 (2H, m), 2.17 (3H, s), 2.15-2.3 (2H, m), 2.5-2.6 (2H, m), 3.15 (2H, t, J = 7.0 Hz), 3.2-3.4 (2H, m), 3.76 (2H, s), 3.83 (3H, s), 5.36 (2H, brs), 6.93 (2H, t, J = 8.4 Hz), 7.16 (1H, s), 7.2- 7.7 (8H, m), 7.85-7.95 (1H, m), 8.65-8.75 (1H, m). |
| 4 | 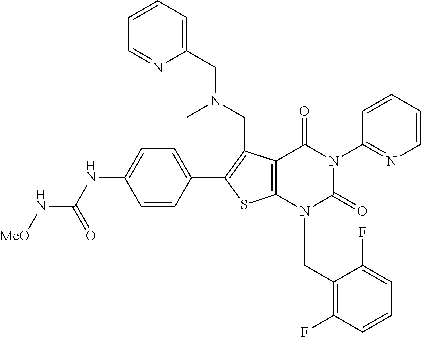 [SMILES: CONC(=O)Nc1ccc(-c2sc3c(c2CN(C)Cc2ccccn2)c(=O)n(-c2ccccn2)c(=O)n3Cc2c(F)cccc2F)cc1] | ¹H-NMR (CDCl₃) δ: 2.10 (3H, s), 3.70 (2H, s), 3.82 (3H, s), 3.96 (3H, s), 5.34 (2H, brs), 6.85-7.7 (14H, m), 7.85-7.95 (1H, m), 8.4-8.5 (1H, m), 8.65- 8.75 (1H, m). |
| 5 | 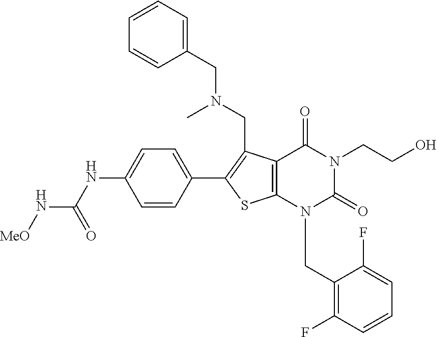 [SMILES: CONC(=O)Nc1ccc(-c2sc3c(c2CN(C)Cc2ccccc2)c(=O)n(CCO)c(=O)n3Cc2c(F)cccc2F)cc1] | ¹H-NMR (CDCl₃) δ: 2.04 (3H, s), 2.5-2.65 (1H, m), 3.58 (2H, s), 3.83 (3H, s), 3.91 (2H, s), 3.9-4.0 (2H, m), 4.37 (2H, t, J = 5.0 Hz), 5.34 (2H, s), 6.92 (2H, t, J = 8.2 Hz), 7.1-7.4 (7H, m), 7.54 (2H, d, J = 8.6 Hz), 7.66 (2H, d, J = 8.6 Hz), 7.6- 7.7(1H, m). |
| 6 | 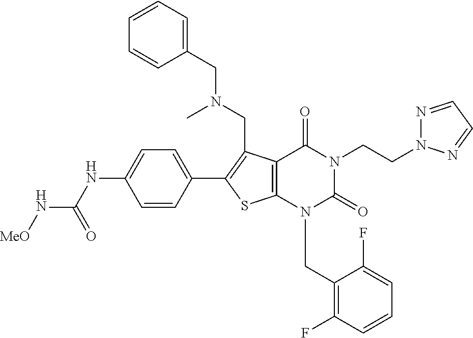 [SMILES: CONC(=O)Nc1ccc(-c2sc3c(c2CN(C)Cc2ccccc2)c(=O)n(CCn2nccn2)c(=O)n3Cc2c(F)cccc2F)cc1] | ¹H-NMR (CDCl₃) δ: 2.04 (3H, s), 3.51 (2H, s), 3.82 (3H, s), 3.86 (2H, s), 4.57 (2H, t, J = 6.2 Hz), 4.81 (2H, t, J = 6.2 Hz), 5.28 (2H, s), 6.91 (2H, t, J = 8.4 Hz), 7.15-7.35 (6H, m), 7.46 (2H, s), 7.53 (2H, d, J = 8.6 Hz), 7.62 (1H, s), 7.70 (2H, t, J = 8.6 Hz), 7.75 (1H, s). |
| 7 | 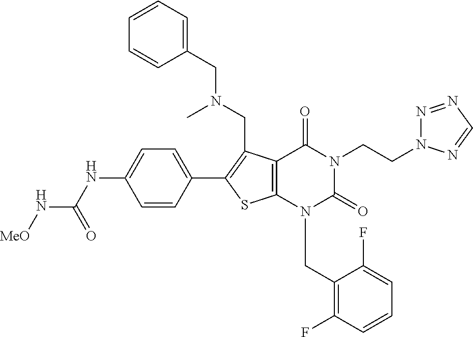 [SMILES: CONC(=O)Nc1ccc(-c2sc3c(c2CN(C)Cc2ccccc2)c(=O)n(CCn2ncnn2)c(=O)n3Cc2c(F)cccc2F)cc1] | 2-yl Form (1) ¹H-NMR (CDCl₃) δ: 2.02 (3H, s), 3.51 (2H, s), 3.83 (5H, s), 4.6-4.7 (2H, m), 5.0-5.1 (2H, m), 5.28 (2H, s), 6.92 (2H, t, J = 8.2 Hz), 7.12 (1H, s), 7.2- 7.75 (11H, m), 8.38 (1H, s). |
| | 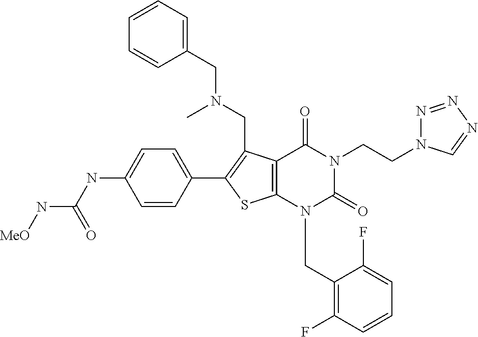 [SMILES: CONC(=O)Nc1ccc(-c2sc3c(c2CN(C)Cc2ccccc2)c(=O)n(CCn2cnnn2)c(=O)n3Cc2c(F)cccc2F)cc1] | 1-yl Form (2) ¹H-NMR (CDCl₃) δ: 2.01 (3H, s), 3.51 (2H, s), 3.83 (3H, s), 3.8-4.0 (2H, m), 4.6-4.7 (2H, m), 4.8-4.9 (2H, m), 5.30 (2H, s), 6.65-6.75 (1H, m), 6.85-7.0 (2H, m), 7.1-7.7 (11H, m), 8.68 (1H, s). |
| 8 | 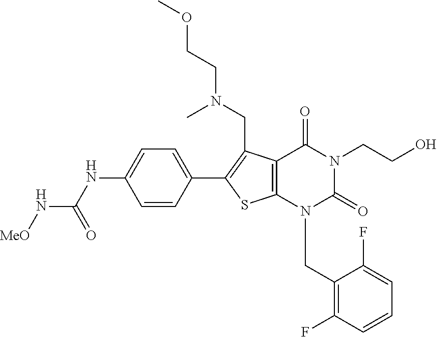 [SMILES: COCCN(C)Cc1c(-c2ccc(NC(=O)NOC)cc2)sc2c1c(=O)n(CCO)c(=O)n2Cc1c(F)cccc1F] | ¹H-NMR (CDCl₃) δ: 2.11 (3H, s), 2.65 (2H, t, J = 5.8 Hz), 3.30 (3H, s), 3.46 (2H, t, J = 5.8 Hz), 3.82 (5H, s), 3.9-4.0 (2H, m), 4.35 (2H, t, J = 5.2 Hz), 5.34 (2H, s), 6.92 (2H, t, J = 8.0 Hz), 7.14 (1H, s), 7.2-7.35 (1H, m), 7.5-7.65 (5H, m). |
| 9 | 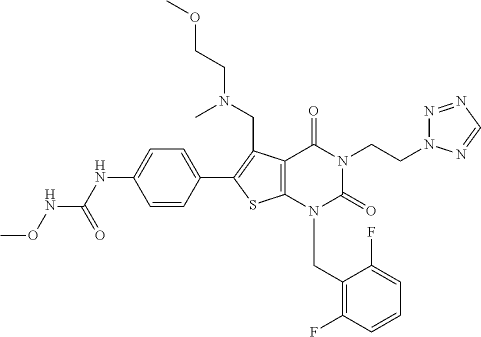 [SMILES: COCCN(C)Cc1c(-c2ccc(NC(=O)NOC)cc2)sc2c1c(=O)n(CCn1ncnn1)c(=O)n2Cc1c(F)cccc1F] | 2-yl Form (1) ¹H-NMR (CDCl₃) δ: 2.10 (3H, brs), 2.55-2.65 (2H, m), 3.31 (3H, s), 3.4-3.5 (2H, m), 3.76 (2H, s), 3.82 (3H, s), 4.62 (2H, t, J = 5.8 Hz), 5.02 (2H, t, J = 5.8 Hz), 5.27 (2H, s), 6.92 (2H, t, J = 8.2 Hz), 7.13 (1H, s), 7.25- 7.4 (1H, m), 7.5-7.65 (5H, m), 8.43 (1H, s). |
| | 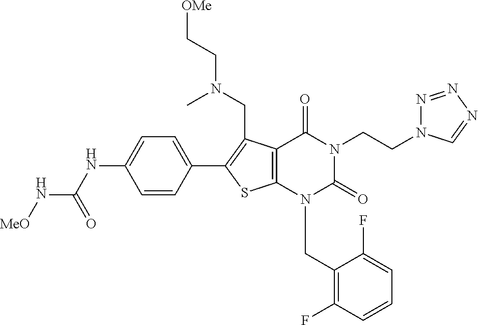 [SMILES: COCCN(C)Cc1c(-c2ccc(NC(=O)NOC)cc2)sc2c1c(=O)n(CCn1cnnn1)c(=O)n2Cc1c(F)cccc1F] | 1-yl Form (2) ¹H-NMR (CDCl₃) δ: 2.07 (3H, s), 2.55-2.65 (2H, m), 3.30 (3H, s), 3.4-3.5 (2H, m), 3.74 (2H, s), 3.82 (3H, s), 4.55-4.65 (2H, m), 4.8-4.9 (2H, m), 5.30 (2H, s), 6.93 (2H, t, J = 7.8 Hz), 7.10 (1H, s), 7.2-7.4 (1H, m), 7.5-7.65 (5H, m), 8.69 (1H, s). |
| 10 | 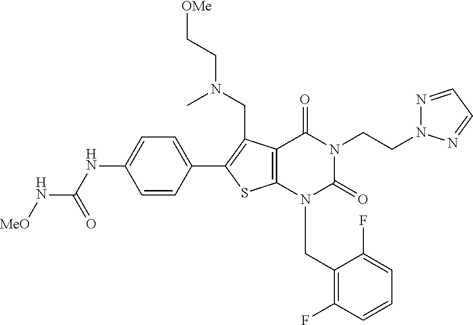 [SMILES: COCCN(C)Cc1c(-c2ccc(NC(=O)NOC)cc2)sc2c1c(=O)n(CCn1nccn1)c(=O)n2Cc1c(F)cccc1F] | ¹H-NMR (CDCl₃) δ: 2.11 (3H, s), 2.62 (2H, t, J = 5.8 Hz), 3.31 (3H, s), 3.44 (2H, t, J = 5.8 Hz), 3.78 (2H, s), 3.82 (3H, s), 4.55 (2H, t, J = 5.6 Hz), 4.79 (2H, t, J = 5.6 Hz), 5.27 (2H, s), 6.91 (2H, t, J = 8.2 Hz), 7.10 (1H, s), 7.2-7.4 (2H, m), 7.51 (2H, s), 7.45-7.65 (4H, m). |
| 11 | 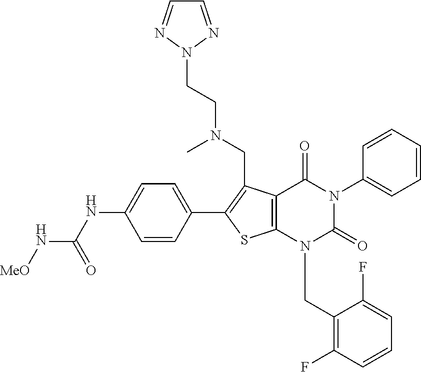 [SMILES: CONC(=O)Nc1ccc(-c2sc3c(c2CN(C)CCn2nccn2)c(=O)n(-c2ccccc2)c(=O)n3Cc2c(F)cccc2F)cc1] | 2-yl Form (1) ¹H-NMR (CDCl₃) δ: 2.23 (3H, s), 3.00 (2H, t, J = 6.3 Hz), 3.78 (2H, s), 3.82 (3H, s), 4.45 (2H, t, J = 6.3 Hz), 5.37 (2H, s), 6.92 (2H, t, J = 8.2 Hz), 6.85-6.95 (1H, m), 7.11 (1H, s), 7.2-7.6 (12H, m). |
| | 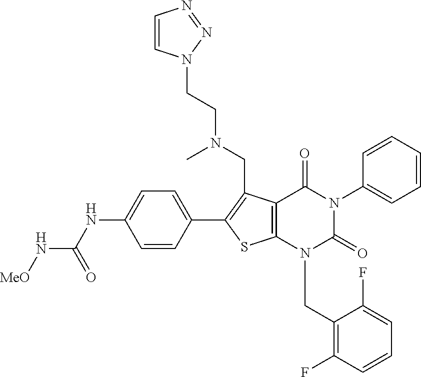 [SMILES: CONC(=O)Nc1ccc(-c2sc3c(c2CN(C)CCn2ccnn2)c(=O)n(-c2ccccc2)c(=O)n3Cc2c(F)cccc2F)cc1] | 1-yl Form (2) ¹H-NMR (CDCl₃) δ: 2.14 (3H, s), 2.82 (2H, t, J = 6.0 Hz), 3.80 (2H, s), 3.82 (3H, s), 4.39 (2H, t, J = 6.0 Hz), 5.37 (2H, s), 6.92 (2H, t, J = 8.2 Hz), 6.85-6.95 (1H, m), 7.14 (1H, s), 7.2-7.55 (11H, m), 7.63 (1H, s). |
| 12 | 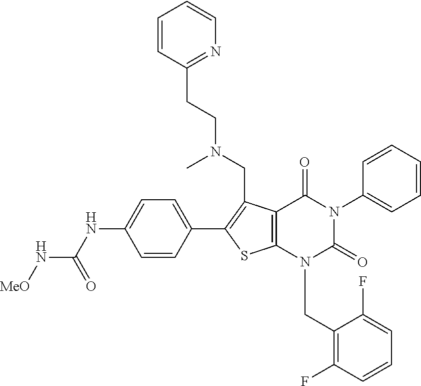 [SMILES: CONC(=O)Nc1ccc(-c2sc3c(c2CN(C)CCc2ccccn2)c(=O)n(-c2ccccc2)c(=O)n3Cc2c(F)cccc2F)cc1] | ¹H-NMR (CDCl₃) δ: 2.20 (3H, s), 2.85 (4H, s), 3.82 (5H, s), 5.37 (2H, s), 6.93 (2H, t, J = 8.2 Hz), 6.95-7.1 (2H, m), 7.14 (1H, s), 7.2-7.55 (11H, m), 7.60 (1H, s), 8.43 (1H, d, J = 4.0 Hz). |
| 13 | 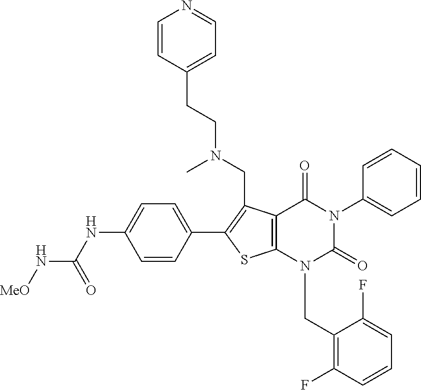 [SMILES: CONC(=O)Nc1ccc(-c2sc3c(c2CN(C)CCc2ccncc2)c(=O)n(-c2ccccc2)c(=O)n3Cc2c(F)cccc2F)cc1] | ¹H-NMR (CDCl₃) δ: 2.18 (3H, s), 2.68 (4H, s), 3.83 (5H, s), 5.37 (2H, s), 6.85-7.0 (5H, m), 7.16 (1H, s), 7.2-7.65 (10H, m), 8.35-8.4 (2H, m). |
| 14 | 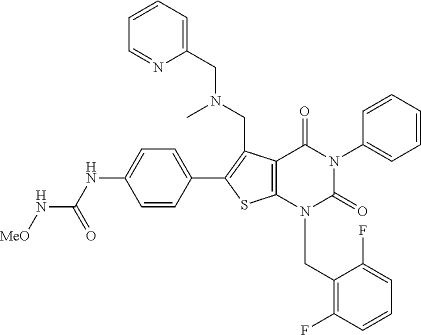 [SMILES: CONC(=O)Nc1ccc(-c2sc3c(c2CN(C)Cc2ccccn2)c(=O)n(-c2ccccc2)c(=O)n3Cc2c(F)cccc2F)cc1] | ¹H-NMR (CDCl₃) δ: 2.10 (3H, s), 3.71 (2H, s), 3.83 (3H, s), 3.99 (2H, s), 5.36 (2H, s), 6.92 (2H, t, J = 8.2 Hz), 7.0-7.1 (1H, m), 7.15 (1H, s), 7.2-7.35 (4H, m), 7.4-7.65 (9H, m), 8.4-8.5 (1H, m). |
| 15 | 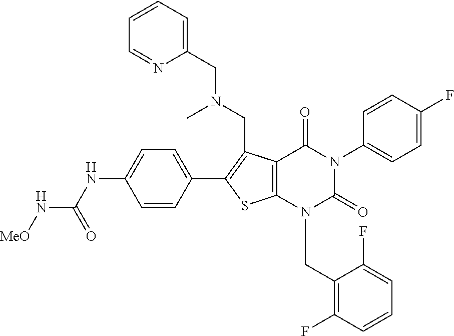 [SMILES: CONC(=O)Nc1ccc(-c2sc3c(c2CN(C)Cc2ccccn2)c(=O)n(-c2ccc(F)cc2)c(=O)n3Cc2c(F)cccc2F)cc1] | ¹H-NMR (CDCl₃) δ: 2.10 (3H, s), 3.71 (2H, s), 3.83 (3H, s), 3.97 (2H, s), 5.35 (2H, s), 6.93 (2H, t, J = 8.2 Hz), 7.0-7.7 (14H, m), 8.4-8.5 (1H, m). |
| 16 | 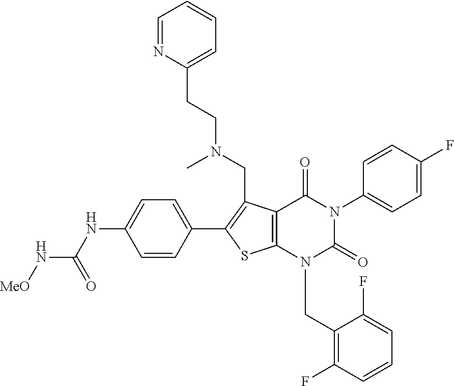 [SMILES: CONC(=O)Nc1ccc(-c2sc3c(c2CN(C)CCc2ccccn2)c(=O)n(-c2ccc(F)cc2)c(=O)n3Cc2c(F)cccc2F)cc1] | ¹H-NMR (CDCl₃) δ: 2.19 (3H, s), 2.85 (4H, s), 3.82 (5H, s), 5.36 (2H, s), 6.9 -7.55 (13H, m), 7.60 (1H s), 8.4-8.45 (1H m). |
| 17 | 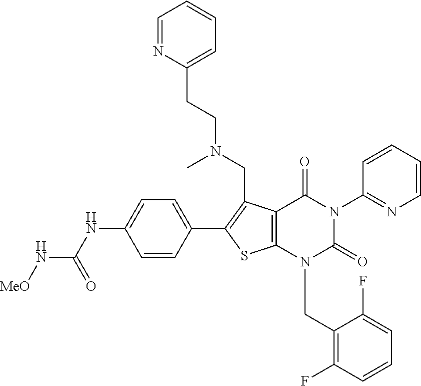 [SMILES: CONC(=O)Nc1ccc(-c2sc3c(c2CN(C)CCc2ccccn2)c(=O)n(-c2ccccn2)c(=O)n3Cc2c(F)cccc2F)cc1] | ¹H-NMR (CDCl₃) δ: 2.21 (3H, s), 2.8-2.9 (4H, m), 3.82 (5H, s), 5.34 (2H, brs), 6.85-7.75 (13H, m), 7.60 (1H, s), 7.85- 7.95 (1H, m), 8.4-8.5 (1H, m), 8.65-8.75 (1H, m). |
| 18 | 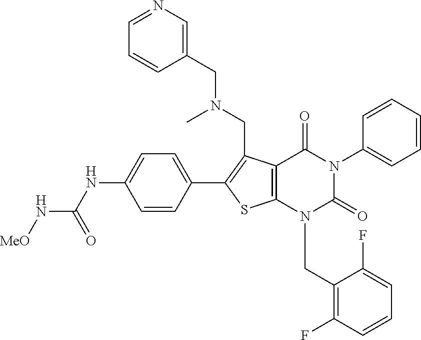 [SMILES: CONC(=O)Nc1ccc(-c2sc3c(c2CN(C)Cc2cccnc2)c(=O)n(-c2ccccc2)c(=O)n3Cc2c(F)cccc2F)cc1] | ¹H-NMR (CDCl₃) δ: 2.04 (3H, s), 3.58 (2H, s), 3.83 (3H, s), 3.92 (2H, s), 5.37 (2H, s), 6.92 (2H, t, J = 8.2 Hz), 7.05-7.7 (14H, m), 8.35-8.45 (2H, m). |
| 19 | 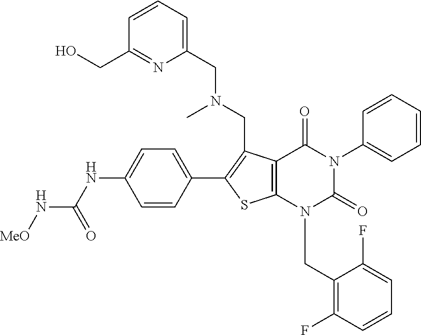 [SMILES: CONC(=O)Nc1ccc(-c2sc3c(c2CN(C)Cc2cccc(CO)n2)c(=O)n(-c2ccccc2)c(=O)n3Cc2c(F)cccc2F)cc1] | ¹H-NMR (CDCl₃) δ: 2.13 (3H, s), 3.72 (2H, s), 3.83 (3H, s), 3.97 (2H, s), 4.05-4.1 (1H, m), 4.65-4.75 (2H, m), 5.37 (2H, s), 6.93 (2H, t, J = 8.2 Hz), 6.9- 7.05 (1H, m), 7.1-7.2 (2H, m), 7.2-7.7 (12H, m). |
| 20 | 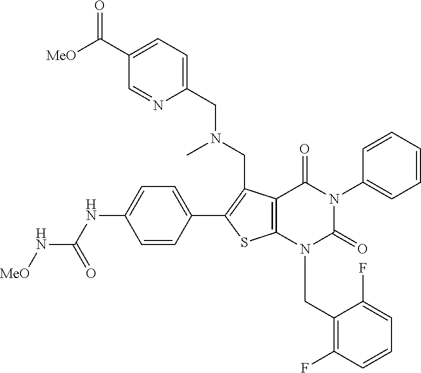 [SMILES: CONC(=O)Nc1ccc(-c2sc3c(c2CN(C)Cc2ccc(C(=O)OC)cn2)c(=O)n(-c2ccccc2)c(=O)n3Cc2c(F)cccc2F)cc1] | ¹H-NMR (CDCl₃) δ: 2.09 (3H, s), 3.76 (2H, s), 3.83 (3H, s), 3.93 (3H, s), 3.99 (2H, s), 5.35 (2H, s), 6.92 (2H, t, J = 8.2 Hz), 7.26 (1H, s), 7.2-7.6 (11H, m), 7.64 (1H, s), 8.05-8.15 (1H, m), 9.0-9.05 (1H, s). |
| 21 | 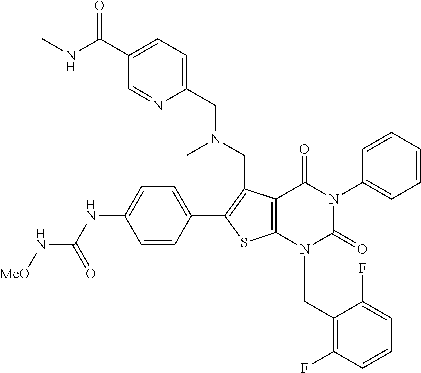 [SMILES: CNC(=O)c1ccc(CN(C)Cc2c(-c3ccc(NC(=O)NOC)cc3)sc3c2c(=O)n(-c2ccccc2)c(=O)n3Cc2c(F)cccc2F)nc1] | ¹H-NMR (CDCl₃) δ: 2.21(3H, s), 3.00 (3H, d, J = 4.6 Hz), 3.74 (2H, s), 3.84 (3H, s), 3.92 (2H, s), 5.36 (2H, s), 6.5-6.65 (1H, m), 6.92 (2H, t, J = 8.0 Hz), 7.15-7.6 (12H, m), 7.71 (1H, s), 7.9-8.0 (1H, m), 8.80-8.85 (1H, m). |
| 22 | 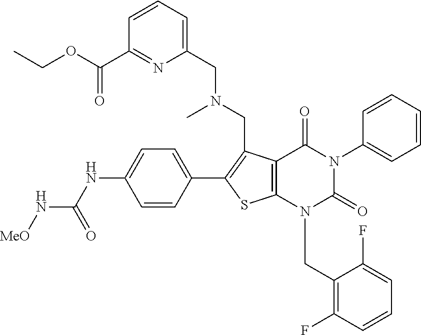 [SMILES: CCOC(=O)c1cccc(CN(C)Cc2c(-c3ccc(NC(=O)NOC)cc3)sc3c2c(=O)n(-c2ccccc2)c(=O)n3Cc2c(F)cccc2F)n1] | ¹H-NMR (CDCl₃) δ: 1.40 (3H, t, J = 7.2 Hz), 2.10 (3H, s), 3.79 (2H, s), 3.83 (3H, s), 4.01(2H, s), 4.43 (2H, q, J = 7.2 Hz), 5.35 (2H, s), 6.92 (2H, t, J = 8.2 Hz), 7.15 (1H, s), 7.2-7.7 (13H, m), 7.88 (1H, d, J = 7.4 Hz). |
| 23 | 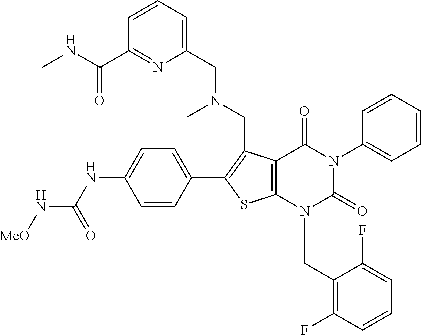 [SMILES: CNC(=O)c1cccc(CN(C)Cc2c(-c3ccc(NC(=O)NOC)cc3)sc3c2c(=O)n(-c2ccccc2)c(=O)n3Cc2c(F)cccc2F)n1] | ¹H-NMR (CDCl₃) δ: 2.13 (3H, s), 2.95 (3H, d, J = 5.2 Hz), 3.74 (2H, s), 3.84 (3H, s), 3.99 (2H, s), 5.36 (2H, s), 6.93 (2H, t, J = 8.2 Hz), 7.15-7.7 (13H, m), 7.9-8.1 (3H, m). |
| 24 | 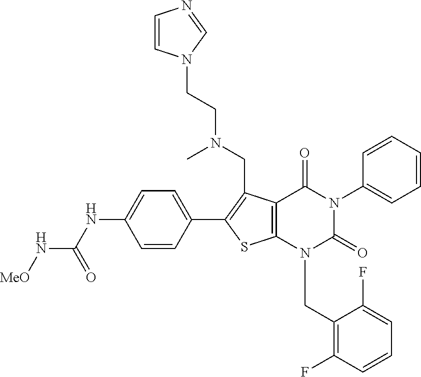 [SMILES: CONC(=O)Nc1ccc(-c2sc3c(c2CN(C)CCn2ccnc2)c(=O)n(-c2ccccc2)c(=O)n3Cc2c(F)cccc2F)cc1] | ¹H-NMR (CDCl₃) δ: 2.17 (3H, s), 2.6-2.8 (2H, m), 3.80 (2H, s), 3.83 (3H, s), 3.9-4.0 (2H, m), 5.38 (2H, s), 6.78 (1H, s), 6.85-7.0 (3H, m), 7.2-7.6 (12H, m), 7.71 (1H, s). |
| 25 | 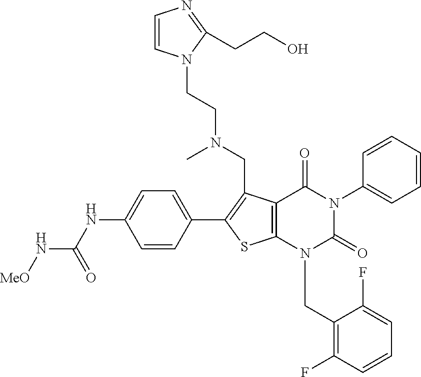 [SMILES: CONC(=O)Nc1ccc(-c2sc3c(c2CN(C)CCn2ccnc2CCO)c(=O)n(-c2ccccc2)c(=O)n3Cc2c(F)cccc2F)cc1] | ¹H-NMR (CDCl₃) δ: 1.9-2.1 (2H, m), 2.16 (3H, s), 2.6-2.8 (4H, m), 3.78 (2H, s), 3.83 (2H, s), 3.9-4.0 (2H, m), 5.38 (2H, s), 6.70 (1H, s), 6.80 (1H, s), 6.94 (2H, t, J = 8.0 Hz), 7.2- 7.6 (11H, m), 7.73 (1H, s). |
| 26 | 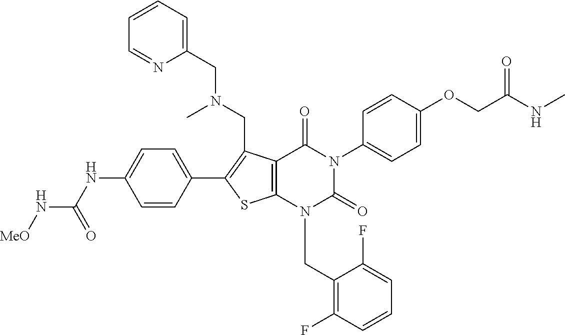 [SMILES: CNC(=O)COc1ccc(-n2c(=O)c3c(CN(C)Cc4ccccn4)c(-c4ccc(NC(=O)NOC)cc4)sc3n(Cc3c(F)cccc3F)c2=O)cc1] | ¹H-NMR (CDCl₃) δ: 2.09 (3H, s), 2.93 (3H, d, J = 4.8 Hz), 3.72 (2H, s), 3.83 (3H, s), 3.97 (2H, s), 4.55 (2H, s), 5.35 (2H, s), 6.55-6.65 (1H, m), 6.92 (2H, t, J = 8.0 Hz), 7.0-7.65 (14H, m), 8.44 (1H, d, J = 6.0 Hz). |
| 27 | 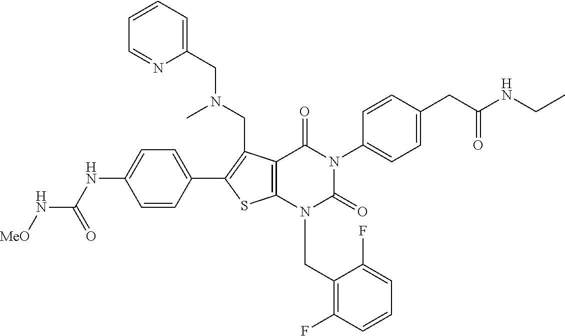 [SMILES: CCNC(=O)Cc1ccc(-n2c(=O)c3c(CN(C)Cc4ccccn4)c(-c4ccc(NC(=O)NOC)cc4)sc3n(Cc3c(F)cccc3F)c2=O)cc1] | ¹H-NMR (CDCl₃) δ: 1.10 (3H, t, J = 7.2 Hz), 2.10 (3H, s), 3.2- 3.4 (2H, m), 3.64 (2H, s), 3.72 (2H, s), 3.83 (3H, s), 3.98 (2H, s), 5.36 (2H, s), 5.45-5.55 (1H, m), 6.93 (2H, t, J = 8.0 Hz), 7.0- 7.1 (1H, m), 7.16 (1H, s), 7.25- 7.7 (12H, m), 8.44 (1H, d, J = 4.0 Hz). |
| 28 | 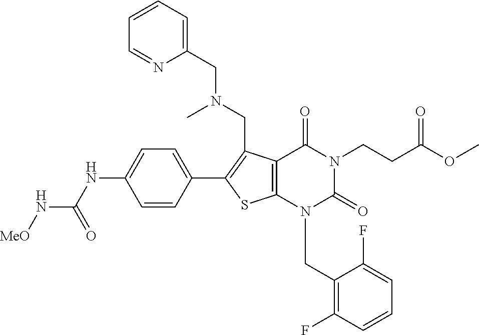 [SMILES: CONC(=O)Nc1ccc(-c2sc3c(c2CN(C)Cc2ccccn2)c(=O)n(CCC(=O)OC)c(=O)n3Cc2c(F)cccc2F)cc1] | ¹H-NMR (CDCl₃) δ: 2.10 (3H, s), 2.65-2.8 (2H, m), 3.68 (3H, s), 3.72 (2H, s), 3.83 (3H, s), 3.98 (2H, s), 4.3-4.5 (2H, m), 5.31 (2H, s), 6.91 (2H, t, J = 7.6 Hz), 7.0-7.4 (5H, m), 7.45-7.65 (5H, m), 8.4-8.5 (1H, m). |
| 29 | 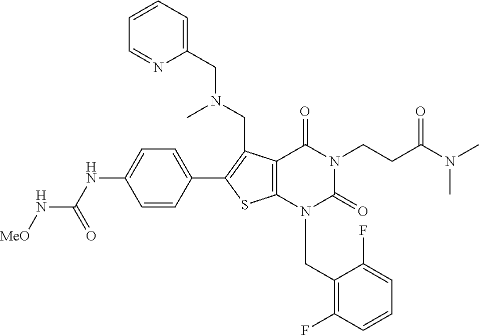 [SMILES: CONC(=O)Nc1ccc(-c2sc3c(c2CN(C)Cc2ccccn2)c(=O)n(CCC(=O)N(C)C)c(=O)n3Cc2c(F)cccc2F)cc1] | ¹H-NMR (CDCl₃) δ: 2.09 (3H, s), 2.75 (3H, d, J = 7.8 Hz), 2.94 (3H, s), 3.02 (3H, s), 3.73 (2H, s), 3.83 (3H, s), 4.07 (2H, s), 4.40 (2H, t, J = 7.8 Hz), 5.33 (2H, s), 6.91 (2H, t, J = 8.2 Hz), 7.0-7.35 (5H, m), 7.5-7.65 (5H m), 8.45 (1H, d, J = 4.0 Hz). |
| 30 | 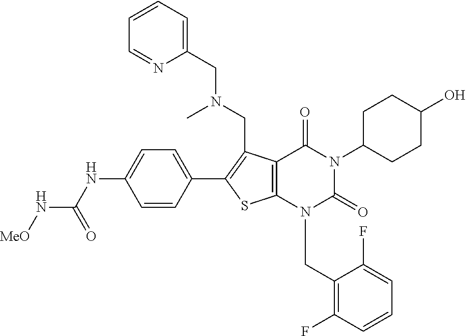 [SMILES: CONC(=O)Nc1ccc(-c2sc3c(c2CN(C)Cc2ccccn2)c(=O)n(C2CCC(O)CC2)c(=O)n3Cc2c(F)cccc2F)cc1] | ¹H-NMR (CDCl₃) δ: 1.4-1.8 (4H, m), 2.0-2.1 (2H, m), 2.12 (3H, s), 2.55-2,75 (2H, m), 3.73 (2H, s), 3.7-3.8 (1H, m), 3.82 (3H, s), 3.98 (2H, s), 4.9- 5.1 (1H, brm), 5.29 (2H, s), 6.91 (2H, t, J = 8.2 Hz), 7.0-7.4 (7H, m), 7.45-7.65 (5H, m), 8.45 (1H, d, J = 4.8 Hz). |
| 31 | 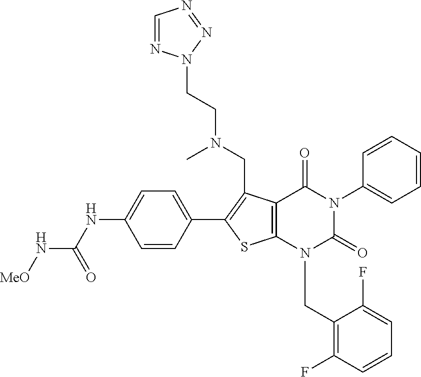 [SMILES: CONC(=O)Nc1ccc(-c2sc3c(c2CN(C)CCn2ncnn2)c(=O)n(-c2ccccc2)c(=O)n3Cc2c(F)cccc2F)cc1] | 2-yl Form (1) ¹H-NMR (CDCl₃) δ: 2.25 (3H, s), 3.04 (2H, t, J = 6.2 Hz), 3.78 (2H, s), 3.83 (3H, s), 4.66 (2H, t, J = 6.2 Hz), 5.38 (2H, s), 6.94 (2H, t, J = 8.0 Hz), 7.16 (1H, s), 7.2-7.6 (10H, m), 7.64 (1H, s), 8.30 (1H, s). |
| | 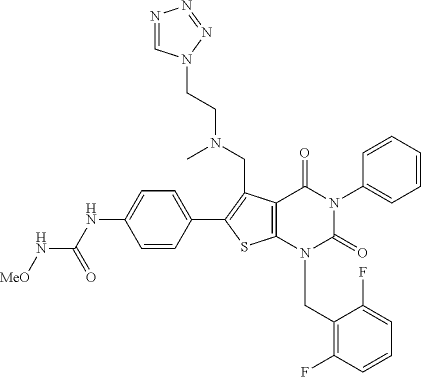 [SMILES: CONC(=O)Nc1ccc(-c2sc3c(c2CN(C)CCn2cnnn2)c(=O)n(-c2ccccc2)c(=O)n3Cc2c(F)cccc2F)cc1] | 1-yl Form (2) ¹H-NMR (CDCl₃) δ: 2.02 (3H, s), 2.7-2.8 (2H, m), 3.78 (2H, s), 3.83 (3H, s), 4.4-4.5 (2H, m), 5.38 (2H, s), 6.92 (2H, t, J = 8.0 Hz), 7.17 (1H, s), 7.25- 7.65 (10H, m), 7.66 (1H, s). |
| 32 | 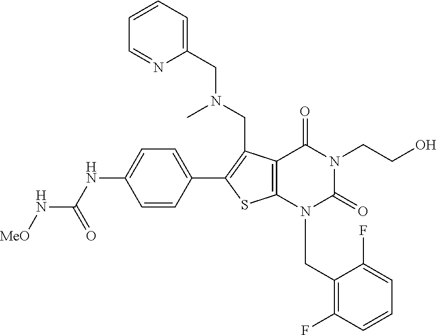 [SMILES: CONC(=O)Nc1ccc(-c2sc3c(c2CN(C)Cc2ccccn2)c(=O)n(CCO)c(=O)n3Cc2c(F)cccc2F)cc1] | ¹H-NMR (CDCl₃) δ: 2.22 (3H, s), 3.3-3.5 (1H, br), 3.78 (2H, s), 3.83 (3H, s), 3.9-4.05 (2H, m), 3.99 (2H, s), 4.35-4.4 (2H, m), 5.27 (2H, s), 6.91 (2H, t, J = 8.0 Hz), 6.9-7.1 (1H, m), 7.15 (1H, s), 7.2-7.65 (8H, m), 8.35-8.4 (1H, m). |
| 33 | 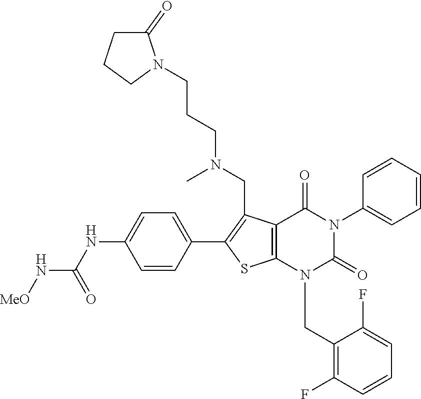 [SMILES: CONC(=O)Nc1ccc(-c2sc3c(c2CN(C)CCCN2CCCC2=O)c(=O)n(-c2ccccc2)c(=O)n3Cc2c(F)cccc2F)cc1] | ¹H-NMR (CDCl₃) δ: 1.5-1.7 (2H, m), 1.8-2.0 (2H, m), 2.05 (3H, s), 2.25-2.45 (4H, m), 3.15 (2H, t, J = 7.8 Hz), 3.23 (2H, t, J = 7.2 Hz), 3.76 (2H, s), 3.83 (3H, s), 5.37 (2H, s), 6.93 (2H, t, J = 8.0 Hz), 7.15 (1H, s), 7.2-7.6 (10H, m), 7.67 (1H, s). |
| 34 | 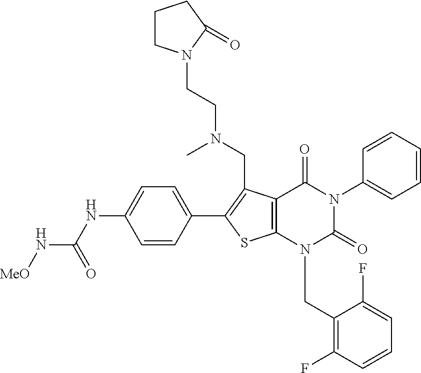 [SMILES: CONC(=O)Nc1ccc(-c2sc3c(c2CN(C)CCN2CCCC2=O)c(=O)n(-c2ccccc2)c(=O)n3Cc2c(F)cccc2F)cc1] | ¹H-NMR (CDCl₃) δ: 1.7-1.85 (2H, m), 2.14 (3H, s), 2.21 (2H, t, J = 8.1 Hz), 2.54 (2H, t, J = 6.2 Hz), 3.19 (2H, t, J = 7.0 Hz), 3.29 (2H, t, J = 6.2 Hz), 3.77 (2H, s), 3.83 (3H, s), 5.38 (2H, s), 6.93 (2H, t, J = 8.1 Hz), 7.18 (1H, s), 7.25-7.7 (11H, m). |
| 35 | 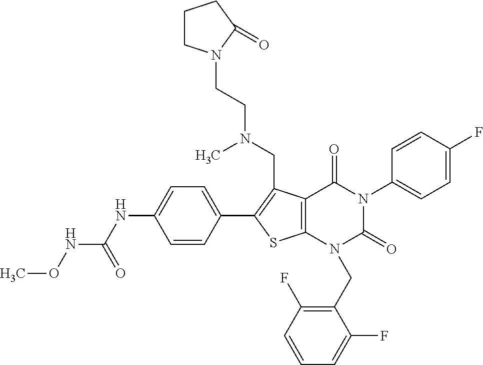 [SMILES: CONC(=O)Nc1ccc(-c2sc3c(c2CN(C)CCN2CCCC2=O)c(=O)n(-c2ccc(F)cc2)c(=O)n3Cc2c(F)cccc2F)cc1] | ¹H-NMR (CDCl₃) δ: 1.7-1.9 (2H, m), 2.15 (3H, s), 2.15-2.3 (2H, m), 2.52 (2H, t, J = 6.2 Hz), 3.20 (2H, t, J = 6.8 Hz), 3.29 (2H, t, J = 6.2 Hz), 3.77 (2H, s), 3.83 (3H, s), 5.37 (2H, s), 6.93 (2H, t, J = 8.2 Hz), 7.1-7.35 (6H, m), 7.5-7.65 (4H m), 7.64 (1H s). |
| 36 | 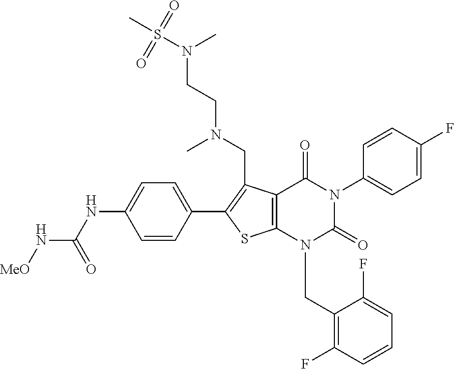 [SMILES: CONC(=O)Nc1ccc(-c2sc3c(c2CN(C)CCN(C)S(C)(=O)=O)c(=O)n(-c2ccc(F)cc2)c(=O)n3Cc2c(F)cccc2F)cc1] | ¹H-NMR (CDCl₃) δ: 2.13 (3H, s), 2.55 (2H, t, J = 6.4 Hz), 2.70 (3H, s), 2.71 (3H, s), 3.15 (2H, t, J = 6.4 Hz), 3.81 (2H, s), 3.83 (3H, s), 5.37 (2H, s), 6.93 (2H, t, J = 8.2 Hz), 7.1-7.7 (11H, m). |
| 37 | 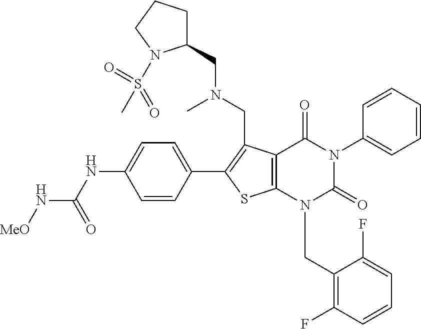 [SMILES: CONC(=O)Nc1ccc(-c2sc3c(c2CN(C)C[C@@H]2CCCN2S(C)(=O)=O)c(=O)n(-c2ccccc2)c(=O)n3Cc2c(F)cccc2F)cc1] | ¹H-NMR (CDCl₃) δ: 1.7-1.85 (4H, m), 2.08 (3H, s), 2.2-2.4 (1H, m), 2.5-2.65 (1H, m), 2.72 (3H, s), 3.15-3.3 (2H, m), 3.7- 3.9 (3H, m), 3.83 (3H, s), 5.37 (2H, s), 6.92 (2H, t, J = 8.2 Hz), 7.13 (1H, s), 7.2-7.7 (11H, m). |
| 38 | 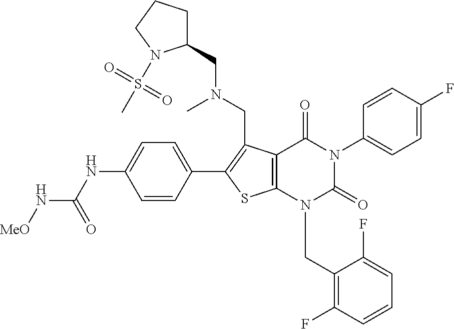 [SMILES: CONC(=O)Nc1ccc(-c2sc3c(c2CN(C)C[C@@H]2CCCN2S(C)(=O)=O)c(=O)n(-c2ccc(F)cc2)c(=O)n3Cc2c(F)cccc2F)cc1] | ¹H-NMR (CDCl₃) δ: 1.7-1.85 (4H, m), 2.09 (3H s), 2.25- 2.35 (1H, m), 2.55 -2.65 (1H, m), 2.71 (3H, s), 3.15-3.3 (2H, m), 3.65-3.7 (2H, m), 3.74 (1H, d, J = 12.0 Hz), 3.83 (3H, s), 3.87 (1H, d, J = 12.0 Hz), 5.36 (2H, s), 6.92 (2H, t, J = 8.2 Hz), 6.85-6.95 (1H, m), 7.1-7.35 (5H, m), 7.49 (2H, d, J = 8.4 Hz), 7.55 (2H, d, J = 8.4 Hz), 7.62 (1H, s). |
| 39 | 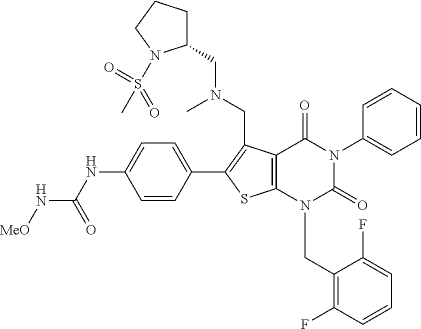 [SMILES: CONC(=O)Nc1ccc(-c2sc3c(c2CN(C)C[C@H]2CCCN2S(C)(=O)=O)c(=O)n(-c2ccccc2)c(=O)n3Cc2c(F)cccc2F)cc1] | ¹H-NMR (CDCl₃) δ: 1.7-1.85 (4H, m), 2.08 (3H, s), 2.2-2.4 (1H, m), 2.5-2.65 (1H, m), 2.72 (3H, s), 3.15-3.3 (2H, m), 3.7- 3.9 (3H, m), 3.83 (3H, s), 5.37 (2H, s), 6.92 (2H, t, J = 8.2 Hz), 7.13 (1H, s), 7.2-7.7 (11H, m). |
| 40 | 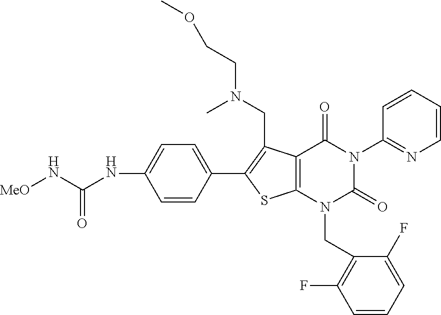 [SMILES: COCCN(C)Cc1c(-c2ccc(NC(=O)NOC)cc2)sc2c1c(=O)n(-c1ccccn1)c(=O)n2Cc1c(F)cccc1F] | ¹H-NMR (CDCl₃) δ: 2.15 (3H, s), 2.62 (2H, t, J = 5.9 Hz), 3.26 (3H, s), 3.41 (2H, t, J = 5.9 Hz), 3.80 (3H, s), 3.81 (2H, brs), 5.34 (2H, brs), 6.91 (2H, t, J = 8.1 Hz), 7.24-7.40 (4H, m), 7.53 (2H, d, J = 8.4 Hz), 7.62 (2H, d, J = 8.4 Hz), 7.65 (1H, s), 7.88 (1H, dt, J = 1.5 Hz, 7.8 Hz), 8.67-8.69 (1H, m). |
| 41 | 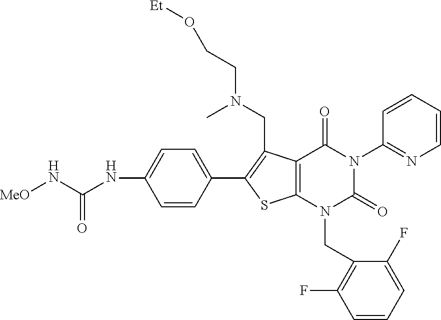 [SMILES: CCOCCN(C)Cc1c(-c2ccc(NC(=O)NOC)cc2)sc2c1c(=O)n(-c1ccccn1)c(=O)n2Cc1c(F)cccc1F] | ¹H-NMR (CDCl₃) δ: 1.13 (3H, t, J = 6.9 Hz), 2.15 (3H, s), 2.63 (2H, t, J = 6.2 Hz), 3.39 (2H, q, J = 6.9 Hz), 3.44 (2H, t, J = 6.2 Hz), 3.80 (2H, brs), 3.81 (3H, s), 5.34 (2H, brs), 6.91 (2H, t, J = 8.1 Hz), 7.19 (1H, s), 7.27- 7.32 (1H, m), 7.35-7.41 (2H, m), 7.53 (2H, d, J = 8.4 Hz), 7.63 (1H, s), 7.64 (2H, d, J = 8.4 Hz), 7.88 (1H, dt, J = 1.2 Hz, 7.5 Hz), 8.68 (1H, dt, J = 0.9 Hz, 4.8 Hz). |
| 42 | 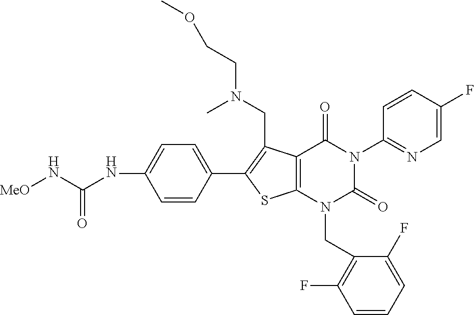 [SMILES: COCCN(C)Cc1c(-c2ccc(NC(=O)NOC)cc2)sc2c1c(=O)n(-c1ccc(F)cn1)c(=O)n2Cc1c(F)cccc1F] | ¹H-NMR (CDCl₃) δ: 2.13 (3H, s), 2.62 (2H, t, J = 5.9 Hz), 3.26 (3H, s), 3.41 (2H, t, J = 5.9 Hz), 3.80 (2H, brs), 3.82 (3H, s), 5.33 (2H, brs), 6.92 (2H, t, J = 8.3 Hz), 7.19 (1H, s), 7.28- 7.38 (2H, m), 7.52-7.63 (6H, m), 8.51 (1H, d, J = 3.0 Hz). |
| 43 | 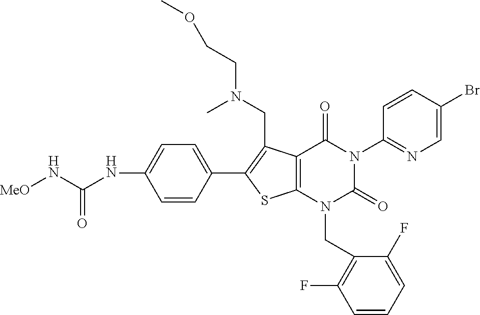 [SMILES: COCCN(C)Cc1c(-c2ccc(NC(=O)NOC)cc2)sc2c1c(=O)n(-c1ccc(Br)cn1)c(=O)n2Cc1c(F)cccc1F] | ¹H-NMR (CDCl₃) δ: 2.13 (3H, s), 2.62 (2H, t, J = 5.9 Hz), 3.26 (3H, s), 3.41 (2H, t, J = 5.9 Hz), 3.78 (2H, brs), 3.80 (3H, s), 5.32 (2H, brs), 6.92 (2H, t, J = 8.1 Hz), 7.27 (1H, d, J = 8.4 Hz), 7.27-7.33 (1H, m), 7.37 (1H, s), 7.54 (2H, d, J = 9.0 Hz), 7.60 (2H, d, J = 9.0 Hz), 7.64 (1H, s), 7.98 (1H, dd, J = 2.7 Hz, 8.4 Hz), 8.72 (1H, d, J = 2.7 Hz). |
| 44 | 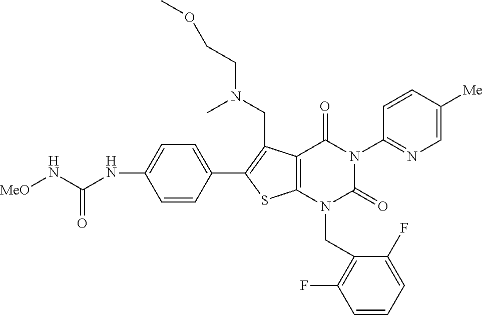 [SMILES: COCCN(C)Cc1c(-c2ccc(NC(=O)NOC)cc2)sc2c1c(=O)n(-c1ccc(C)cn1)c(=O)n2Cc1c(F)cccc1F] | ¹H-NMR (CDCl₃) δ: 2.14 (3H, s), 2.39 (3H, s), 2.62 (2H, t, J = 5.7 Hz), 3.26 (3H, s), 3.41 (2H, t, J = 5.7 Hz), 3.77 (2H, brs), 3.80 (3H, s), 5.26 (1H, brs), 5.38 (1H, brs), 6.91 (2H, t, J = 8.3 Hz), 7.23-7.34 (2H, m), 7.42 (1H, s), 7.53 (2H, d, J = 8.7 Hz), 7.62 (2H, d, J = 8.7 Hz), 7.66 (1H, s), 7.66-7.69 (1H, m), 8.48 (1H, d, J = 2.4 Hz). |
| 45 | 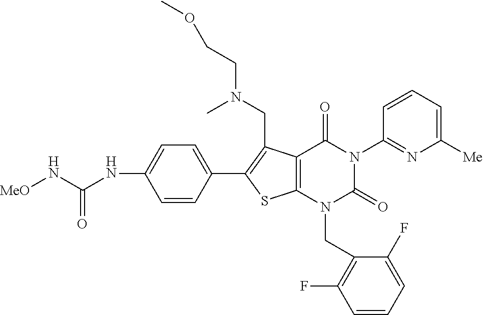 [SMILES: COCCN(C)Cc1c(-c2ccc(NC(=O)NOC)cc2)sc2c1c(=O)n(-c1cccc(C)n1)c(=O)n2Cc1c(F)cccc1F] | ¹H-NMR (CDCl₃) δ: 2.15 (3H, s), 2.60 (3H, s), 2.62 (2H, t, J = 5.8 Hz), 3.27 (3H, s), 3.41 (2H, t, J = 5.8 Hz), 3.66-3.94 (2H, m), 3.81 (3H, s), 5.15 (1H, d, J = 15.3 Hz), 5.48 (1H, d, J = 15.3 Hz), 6.91 (2H, t, J = 8.1 Hz), 7.16 (1H, d, J = 7.8 Hz), 7.21 (1H, d, J = 7.8 Hz), 7.25 (1H, s), 7.26-7.35 (1H, m), 7.53 (2H, d, J = 8.7 Hz), 7.63 (1H, s), 7.53 (2H, d, J = 8.7 Hz), 7.64 (2H, d, J = 8.7 Hz), 7.76 (1H, t, J = 7.8 Hz). |
| 46 | 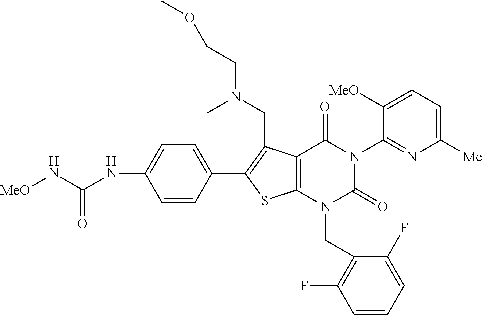 [SMILES: COCCN(C)Cc1c(-c2ccc(NC(=O)NOC)cc2)sc2c1c(=O)n(-c1nc(C)ccc1OC)c(=O)n2Cc1c(F)cccc1F] | ¹H-NMR (CDCl₃) δ: 2.13 (3H, s), 2.51 (3H, s), 2.62 (2H, t, J = 5.9 Hz), 3.26 (3H, s), 3.40 (2H, t, J = 5.9 Hz), 3.77 (1H, d, J = 12.3 Hz), 3.77 (3H, s), 3.79 (3H, s), 3.86 (1H, d, J = 12.3 Hz), 5.24 (1H, d, J = 15.6 Hz), 5.40 (1H, d, J = 15.6 Hz), 6.90 (2H, t, J = 8.1 Hz), 7.19 (1H, d, J = 8.4 Hz), 7.23-7.34 (1H, m), 7.27 (1H, d, J = 8.4 Hz), 7.51 (2H, d, J = 8.7 Hz), 7.58 (2H, d, J = 8.7 Hz), 7.65 (1H, s), 7.69 (1H, s). |
| 47 | 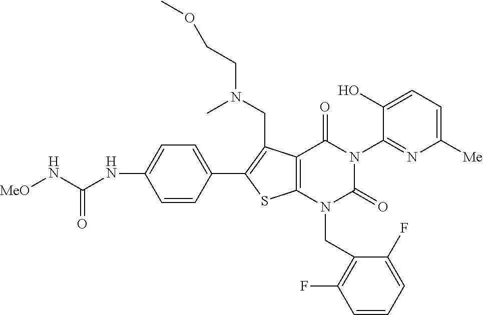 [SMILES: COCCN(C)Cc1c(-c2ccc(NC(=O)NOC)cc2)sc2c1c(=O)n(-c1nc(C)ccc1O)c(=O)n2Cc1c(F)cccc1F] | ¹H-NMR (CDCl₃) δ: 2.11 (3H, s), 2.48 (3H, s), 2.51-2.59 (2H, m), 3.20 (3H, s), 3.30-3.46 (4H, m), 3.60 (1H, d, J = 12.3 Hz), 3.79 (3H, s), 4.05 (1H, d, J = 12.3 Hz), 5.21 (1H, d, J = 15.6 Hz), 5.31 (1H, d, J = 15.6 Hz), 6.88 (2H, t, J = 8.1 Hz), 7.07 (1H, d, J = 8.1 Hz), 7.21-7.31 (2H, m), 7.43-7.51 (4H, m), 7.69 (1H, s). |
| 48 | 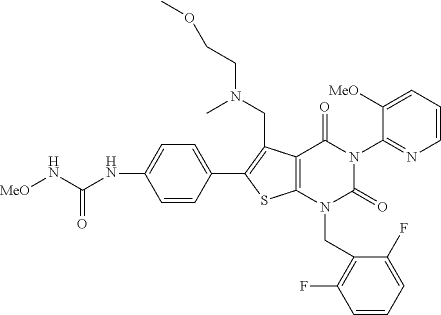 [SMILES: COCCN(C)Cc1c(-c2ccc(NC(=O)NOC)cc2)sc2c1c(=O)n(-c1ncccc1OC)c(=O)n2Cc1c(F)cccc1F] | ¹H-NMR (CDCl₃) δ: 2.20 (3H, s), 2.63 (2H, t, J = 6.2 Hz), 3.28 (3H, s), 3.41 (2H, t, J = 6.2 Hz), 3.78 (1H, d, J = 12.3 Hz), 3.82 (3H, s), 3.84 (3H, s), 3.88 (1H, d, J = 12.3 Hz), 5.35 (2H, s), 6.92 (2H, t, J = 8.1 Hz), 7.17 (1H, s), 7.23-7.39 (3H, m), 7.54 (2H, d, J = 8.8 Hz), 7.57 (1H, s), 7.69 (2H, d, J = 8.8 Hz), 8.23-8.27 (1H, m). |
| 49 | 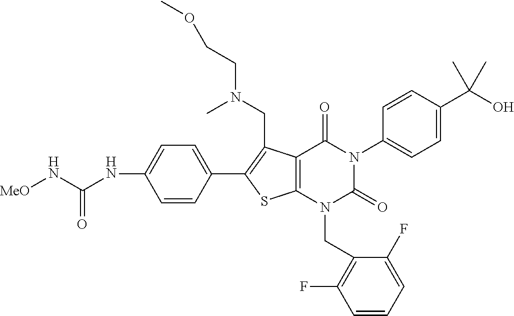 [SMILES: COCCN(C)Cc1c(-c2ccc(NC(=O)NOC)cc2)sc2c1c(=O)n(-c1ccc(C(C)(C)O)cc1)c(=O)n2Cc1c(F)cccc1F] | ¹H-NMR (CDCl₃) δ: 1.60 (6H, s), 1.79 (1H, s), 2.14 (3H, s), 2.63 (2H, t, J = 5.9 Hz), 3.27 (3H, s), 3.41 (2H, t, J = 5.9 Hz), 3.81 (3H, s), 3.82 (2H, s), 5.36 (2H, s), 6.92 (2H, t, J = 8.3 Hz), 7.20-7.34 (4H, m), 7.53 (2H, d, J = 8.7 Hz), 7.60-7.63 (5H, m). |
| 50 | 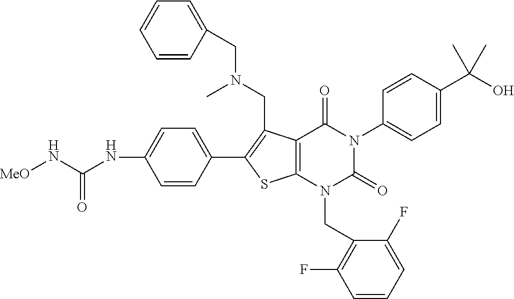 [SMILES: CONC(=O)Nc1ccc(-c2sc3c(c2CN(C)Cc2ccccc2)c(=O)n(-c2ccc(C(C)(C)O)cc2)c(=O)n3Cc2c(F)cccc2F)cc1] | ¹H-NMR (CDCl₃) δ: 1.62 (6H, s), 1.77 (1H, s), 2.05 (3H, s), 3.56 (2H, s), 3.82 (3H, s), 3.90 (2H, s), 5.36 (2H, s), 6.91 (2H, t, J = 8.1 Hz) 7.14-7.38 (9H, m), 7.55 (2H, d, J = 9.0 Hz), 7.62 (1H, s), 7.64 (2H, d, J = 8.7 Hz), 7.72 (2H, d, J = 8.4 Hz). |
| 51 | 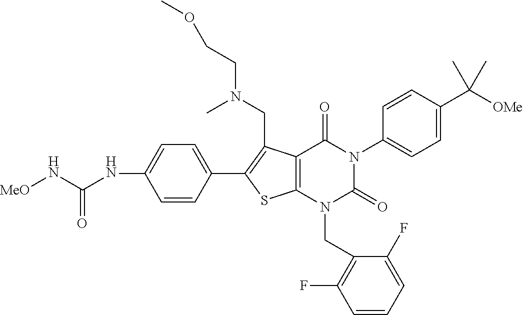 [SMILES: COCCN(C)Cc1c(-c2ccc(NC(=O)NOC)cc2)sc2c1c(=O)n(-c1ccc(C(C)(C)OC)cc1)c(=O)n2Cc1c(F)cccc1F] | ¹H-NMR (CDCl₃) δ: 1.55 (6H, s), 2.15 (3H, s), 2.64 (2H, t, J = 5.9 Hz), 3.11 (3H, s), 3.27 (3H, s), 3.41 (2H, t, J = 5.9 Hz), 3.82 (3H, s), 3.83 (2H, s), 5.36 (2H, s), 6.92 (2H, t, J = 8.3 Hz), 7.16 (1H, s), 7.24-7.36 (4H, m), 7.51-7.63 (6H, m). |
| 52 | 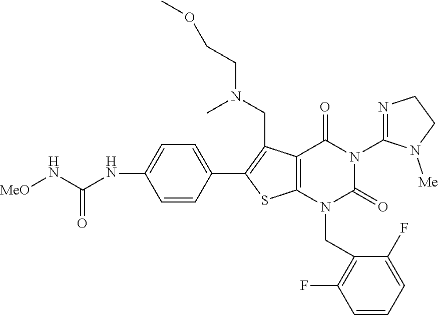 [SMILES: COCCN(C)Cc1c(-c2ccc(NC(=O)NOC)cc2)sc2c1c(=O)n(C1=NCCN1C)c(=O)n2Cc1c(F)cccc1F] | ¹H-NMR (CDCl₃) δ: 2.15 (3H, s), 2.61 (2H, dt, J = 1.8 Hz, 6.90 Hz), 3.27 (3H, s), 3.40 (2H, dt, J = 1.8 Hz, 6.0 Hz), 3.53 (3H, s), 3.75 (1H, d, J = 12.3 Hz), 3.80 (3H, s), 3.81 (1H, d, J = 12.3 Hz), 5.12 (1H, d, J = 15.9 Hz), 5.57 (1H, d, J = 15.9 Hz), 6.91 (2H, t, J = 8.1 Hz), 6.99 (1H, d, J = 1.5 Hz), 7.14 (1H, d, J = 1.5 Hz), 7.28 (1H, s), 7.25-7.34 (1H, m), 7.53 (2H, d, J = 9.0 Hz), 7.60 (2H, d, J = 9.0 Hz), 7.70 (1H, s). |
| 53 | 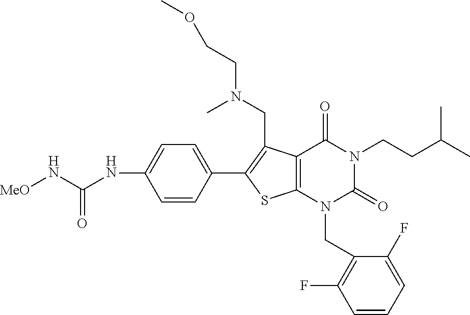 [SMILES: COCCN(C)Cc1c(-c2ccc(NC(=O)NOC)cc2)sc2c1c(=O)n(CCC(C)C)c(=O)n2Cc1c(F)cccc1F] | ¹H-NMR (CDCl₃) δ: 0.98 (6H, d, J = 6.3 Hz), 1.52-1.58 (2H, m), 1.64-1.71 (1H, m), 2.14 (3H, s), 2.66 (2H, t, J = 5.9 Hz), 3.30 (3H, s), 3.45 (2H, t, J = 5.9 Hz), 3.81 (3H, s), 3.85 (2H, s), 4.04-4.09 (2H, m), 5.33 (2H, s), 6.90 (2H, t, J = 8.3 Hz), 7.17 (1H, s), 7.24-7.35 (1H, m), 7.51 (2H, d, J = 8.7 Hz), 7.57 (2H, d, J = 8.7 Hz), 7.60 (1H, s). |
| 54 | 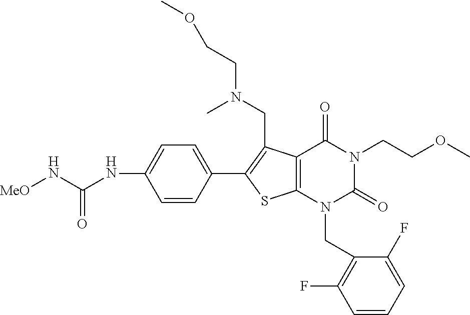 [SMILES: COCCN(C)Cc1c(-c2ccc(NC(=O)NOC)cc2)sc2c1c(=O)n(CCOC)c(=O)n2Cc1c(F)cccc1F] | ¹H-NMR (CDCl₃) δ: 2.14 (3H, s), 2.65 (2H, t, J = 5.9 Hz), 3.30 (3H, s), 3.36 (3H, s), 3.45 (2H, t, J = 5.9 Hz), 3.66 (2H, t, J = 5.9 Hz), 3.81 (3H, s), 3.84 (2H, s), 4.30 (2H, t, J = 5.9 Hz), 5.33 (2H, s), 6.90 (2H, t, J = 8.3 Hz), 7.15 (1H, s), 7.24-7.34 (1H, m), 7.51 (2H, d, J = 9.0 Hz), 7.56 (2H, d, J = 9.0 Hz), 7.60 (1H, m). |
| 55 | 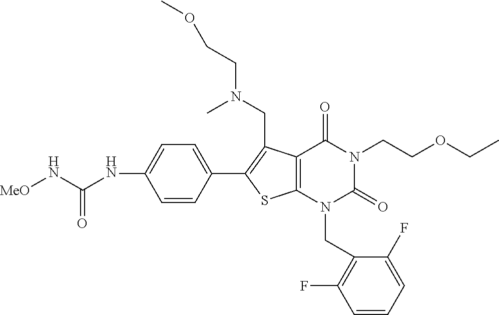 [SMILES: CCOCCn1c(=O)c2c(CN(C)CCOC)c(-c3ccc(NC(=O)NOC)cc3)sc2n(Cc2c(F)cccc2F)c1=O] | ¹H-NMR (CDCl₃) δ: 1.15 (3H, t, J = 6.9 Hz), 2.14 (3H, s), 2.66 (2H, t, J = 6.0 Hz), 3.30 (3H, s), 3.45 (2H, t, J = 6.0 Hz), 3.54 (2H, q, J = 6.9 Hz), 3.69 (2H, t, J = 6.0 Hz), 3.81 (3H, s), 3.84 (2H, s), 4.29 (2H, t, J = 6.0 Hz), 5.32 (2H, s), 6.89 (2H, t, J = 8.1 Hz), 7.17 (1H, s), 7.23-7.34 (1H, m), 7.52 (2H, d, J = 8.7 Hz), 7.57 (2H, d, J = 8.7 Hz), 7.60 (1H, m). |
| 56 | 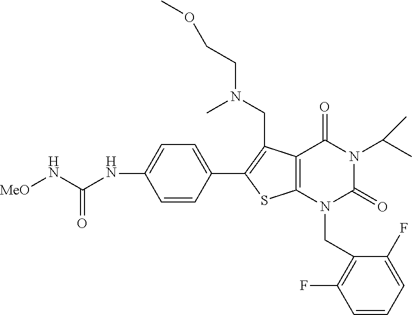 [SMILES: COCCN(C)Cc1c(-c2ccc(NC(=O)NOC)cc2)sc2c1c(=O)n(C(C)C)c(=O)n2Cc1c(F)cccc1F] | ¹H-NMR (CDCl₃) δ: 1.52 (6H, d, J = 6.9 Hz), 2.13 (3H, s), 2.66 (2H, t, J = 5.9 Hz), 3.31 (3H, s), 3.46 (2H, t, J = 5.9 Hz), 3.82 (3H, s), 3.84 (2H, s), 5.31 (2H, s), 5.34 (1H, m), 6.90 (2H, t, J = 8.1 Hz), 7.16 (1H, s), 7.24- 7.35 (1H, m), 7.52 (2H, d, J = 8.4 Hz), 7.55 (2H, d, J = 8.4 Hz), 7.60 (1H, m). |
| 57 | 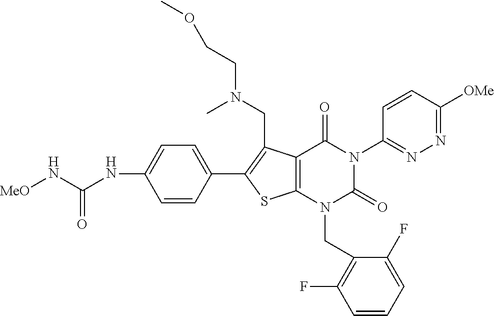 [SMILES: COCCN(C)Cc1c(-c2ccc(NC(=O)NOC)cc2)sc2c1c(=O)n(-c1ccc(OC)nn1)c(=O)n2Cc1c(F)cccc1F] | ¹H-NMR (CDCl₃) δ: 2.13 (3H, s), 2.62 (2H, t, J = 5.7 Hz), 3.26 (3H, s), 3.41 (2H, t, J = 5.7 Hz), 3.74 (2H, brs), 3.82 (3H, s), 4.18 (3H, s), 5.32 (2H, brs), 6.92 (2H, t, J = 8.3 Hz), 7.12 (1H, d, J = 9.3 Hz), 7.24 (1H, s), 7.29-7.35 (1H, m), 7.41 (2H, d, J = 9.3 Hz), 7.54 (2H, d, J = 9.0 Hz), 7.59 (2H, d, J = 8.7 Hz), 7.66 (1H, s). |
| 58 | 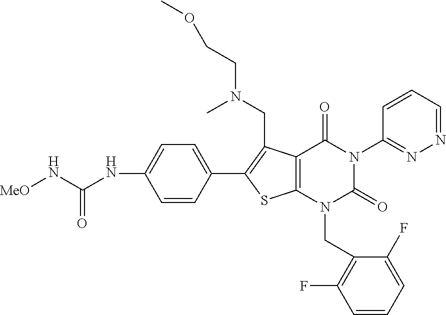 [SMILES: COCCN(C)Cc1c(-c2ccc(NC(=O)NOC)cc2)sc2c1c(=O)n(-c1cccnn1)c(=O)n2Cc1c(F)cccc1F] | ¹H-NMR (CDCl₃) δ: 2.12 (3H, s), 2.61 (2H, t, J = 5.7 Hz), 3.26 (3H, s), 3.39 (2H, t, J = 5.7 Hz), 3.78 (2H, brs), 3.82 (3H, s), 5.34 (2H, brs), 6.93 (2H, t, J = 8.1 Hz), 7.26 (1H, s), 7.29- 7.37 (1H, m), 7.53-7.61 (5H, m), 7.67 (1H, s), 7.69 (1H, dd, J = 4.8 Hz, 8.4 Hz), 9.28 (1H, dd, J = 1.8 Hz, 4.8 Hz). |
| 59 | 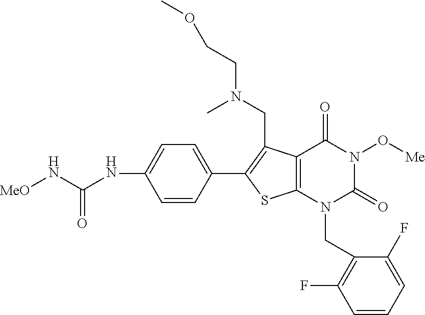 [SMILES: COCCN(C)Cc1c(-c2ccc(NC(=O)NOC)cc2)sc2c1c(=O)n(OC)c(=O)n2Cc1c(F)cccc1F] | ¹H-NMR (CDCl₃) δ: 2.14 (3H, s), 2.68 (2H, t, J = 6.0 Hz), 3.31 (3H, s), 3.47 (2H, t, J = 6.0 Hz), 3.82 (3H, s), 3.83 (2H, s), 4.06 (3H, s), 5.35 (2H, s), 6.92 (2H, t, J = 8.3 Hz), 7.20 (1H, s), 7.29-7.35 (1H, m), 7.55 (4H, s), 7.63 (1H, s). |
| 60 | 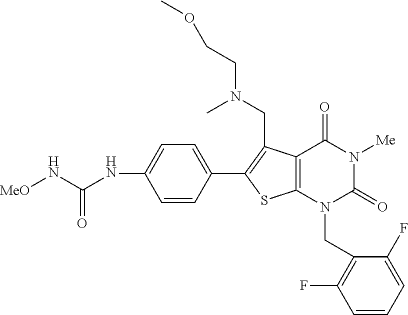 [SMILES: COCCN(C)Cc1c(-c2ccc(NC(=O)NOC)cc2)sc2c1c(=O)n(C)c(=O)n2Cc1c(F)cccc1F] | ¹H-NMR (CDCl₃) δ: 2.13 (3H, s), 2.66 (2H, t, J = 5.9 Hz), 3.31 (3H, s), 3.45 (3H, s), 3.48 (2H, t, J = 5.9 Hz), 3.82 (3H, s), 3.84 (2H, s), 5.33 (2H, s), 6.91 (2H, t, J = 8.3 Hz), 7.17 (1H, s), 7.25-7.35 (1H, m), 7.55 (4H, s), 7.62 (1H, s). |
| 61 | 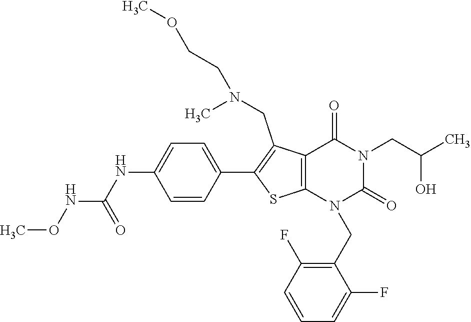 [SMILES: COCCN(C)Cc1c(-c2ccc(NC(=O)NOC)cc2)sc2c1c(=O)n(CC(C)O)c(=O)n2Cc1c(F)cccc1F] | ¹H-NMR (CDCl₃) δ: 1.27 (3H, d, J = 5.6 Hz), 2.12 (3H, s), 2.64 (2H, t, J = 5.8 Hz), 2.9-3.05 (1H, m), 3.30 (3H, s), 3.45 (2H, d, J = 5.8 Hz), 3.82 (5H, s), 4.05- 4.25 (1H, m), 4.18 (2H, s), 5.34 (2H, s), 6.91 (2H, t, J = 8.2 Hz), 7.2-7.4 (1H, m), 7.5-7.6 (3H, m), 7.63 (1H, s). |
| 62 | 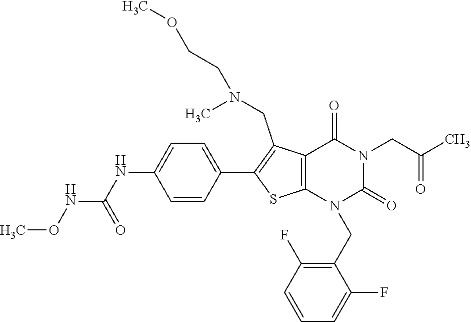 [SMILES: COCCN(C)Cc1c(-c2ccc(NC(=O)NOC)cc2)sc2c1c(=O)n(CC(C)=O)c(=O)n2Cc1c(F)cccc1F] | ¹H-NMR (CDCl₃) δ: 2.13 (3H, s), 2.27 (3H, s), 2.55-2.65 (2H, m), 3.29 (3H, s), 3.4-3.5 (2H, m), 3.82 (5H, s), 4.88 (2H, s), 5.33 (2H, s), 6.91 (2H, t, J = 8.0 Hz), 7.2-7.35 (1H, m), 7.5-7.65 (4H, m). |
| 63 | 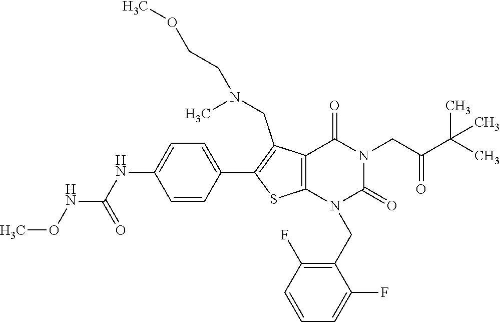 [SMILES: COCCN(C)Cc1c(-c2ccc(NC(=O)NOC)cc2)sc2c1c(=O)n(CC(=O)C(C)(C)C)c(=O)n2Cc1c(F)cccc1F] | ¹H-NMR (CDCl₃) δ: 1.30 (9H, s), 2.12 (3H, s), 2.62 (2H, t, J = 5.8 Hz), 3.29 (3H, s), 3.44 (2H, d, J = 5.8 Hz), 3.80 (2H, s), 3.82 (3H, s), 5.04 (2H, s), 5.33 (2H, s), 6.91 (2H, t, J = 8.2 Hz), 7.14 (1H, s), 7.2-7.3 (1H, m), 7.5-7.6 (4H, m), 7.61 (1H, s). |
| 64 | 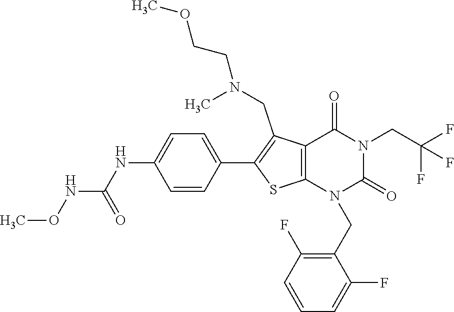 [SMILES: COCCN(C)Cc1c(-c2ccc(NC(=O)NOC)cc2)sc2c1c(=O)n(CC(F)(F)F)c(=O)n2Cc1c(F)cccc1F] | ¹H-NMR (CDCl₃) δ: 2.12 (3H, s), 2.65 (2H, t, J = 6.0 Hz), 3.31 (3H, s), 3.45 (2H, d, J = 6.0 Hz), 3.82 (5H, s), 4.75-4.85 (2H, m), 5.36 (2H, s), 6.92 (2H, t, J = 8.2 Hz), 7.13 (1H, s), 7.2- 7.35 (1H, m), 7.55-7.6 (4H, m), 7.62 (1H, s). |
| 65 | 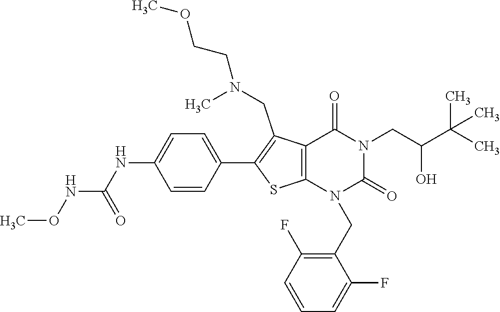 [SMILES: COCCN(C)Cc1c(-c2ccc(NC(=O)NOC)cc2)sc2c1c(=O)n(CC(O)C(C)(C)C)c(=O)n2Cc1c(F)cccc1F] | ¹H-NMR (CDCl₃) δ: 1.04 (9H, s), 2.12 (3H, s), 2.64 (2H, t, J = 6.0 Hz), 2.96 (1H, d, J = 6.0 Hz), 3.31 (3H, s), 3.46 (2H, d, J = 6.0 Hz), 3.5-3.6 (1H, m), 3.82 (3H, s), 3.75-3.9 (2H, m), 4.05-4.2 (1H, m), 4.3-4.45 (1H, m), 5.25-5.45 (2H, m), 6.91 (2H, t, J = 8.2 Hz), 7.14 (1H, s), 7.2-7.35 (1H, m), 7.5-7.6 (4H, m), 7.61 (1H, s). |
| 66 | 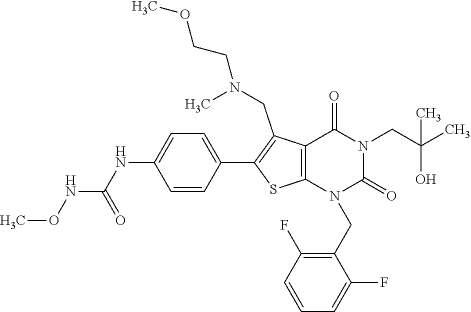 [SMILES: COCCN(C)Cc1c(-c2ccc(NC(=O)NOC)cc2)sc2c1c(=O)n(CC(C)(C)O)c(=O)n2Cc1c(F)cccc1F] | ¹H-NMR (CDCl₃) δ: 1.28 (6H, s), 2.3 (3H, s), 2.64 (2H, t, J = 5.8 Hz), 3.30 (3H, s), 3.45 (2H, t, J = 5.8 Hz), 3.82 (5H, s), 3.99 (1H, s), 4.25 (2H, s), 5.36 (2H, s), 6.91 (2H, t, J = 8.2 Hz), 7.12 (1H, s), 7.2-7.4 (1H, m), 7.5-7.6 (4H, m), 7.61 (1H, s). |
| 67 | 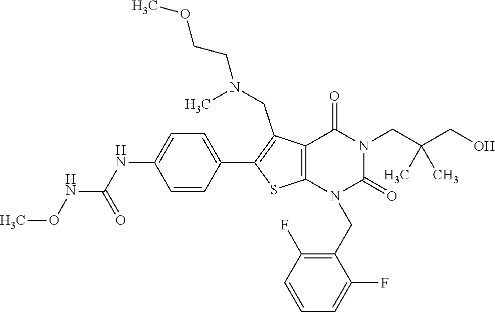 [SMILES: COCCN(C)Cc1c(-c2ccc(NC(=O)NOC)cc2)sc2c1c(=O)n(CC(C)(C)CO)c(=O)n2Cc1c(F)cccc1F] | ¹H-NMR (CDCl₃) δ: 0.96 (6H, s), 2.13 (3H, s), 2.64 (2H, t, J = 6.2 Hz), 3.13 (2H, s), 3.30 (3H, s), 3.45 (2H, t, J = 6.2 Hz), 3.82 (5H, s), 3.95-4.15 (2H, brm), 5.1-5.5 (2H, br), 6.91 (2H, t, J = 8.2 Hz), 7.14 (1H, s), 7.2-7.4 (1H, m), 7.5-7.6 (4H, m), 7.61 (1H, s). |
| 68 | 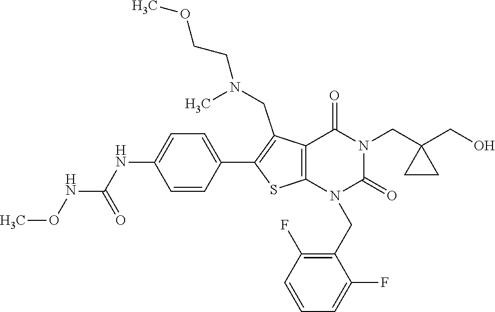 [SMILES: COCCN(C)Cc1c(-c2ccc(NC(=O)NOC)cc2)sc2c1c(=O)n(CC1(CO)CC1)c(=O)n2Cc1c(F)cccc1F] | ¹H-NMR (CDCl₃) δ: 0.46 (2H, t, J = 5.4 Hz), 0.85 (2H, t, J = 5.4 Hz), 2.11 (3H, s), 2.64 (2H, t, J = 6.0 Hz), 3.25 (2H, s), 3.31 (3H, s), 3.46 (2H, t, J = 6.0 Hz), 3.82 (5H, s), 3.95-4.15 (1H, br), 4.14 (2H, s), 5.37 (2H, s), 6.91 (2H, t, J = 8.0 Hz), 7.12 (1H, s), 7.2-7.4 (1H, m), 7.54 (4H, s), 7.61 (1H, s). |
| 69 | 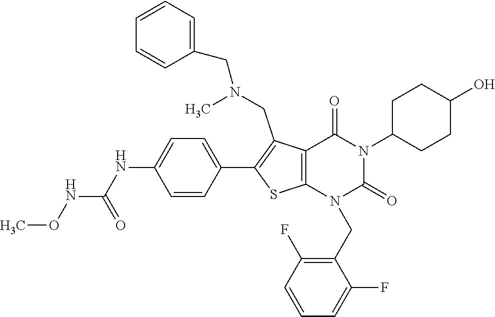 [SMILES: CONC(=O)Nc1ccc(-c2sc3c(c2CN(C)Cc2ccccc2)c(=O)n(C2CCC(O)CC2)c(=O)n3Cc2c(F)cccc2F)cc1] | ¹H-NMR (CDCl₃) δ: 1.35-1.65 (3H, m), 1.65-1.8 (2H, m), 2.07 (3H, s), 2.5-2.8 (2H, m), 3.58 (2H, s), 3.7-3.9 (1H, m), 3.82 (3H, s), 3.91(2H, s), 4.9-5.1 (1H, m), 5.29 (2H s), 6.90 (2H, t, J = 7.8 Hz), 7.13 (1H, s), 7.15-7.35 (6H, m), 7.53 (2H, d, J = 8.6 Hz), 7.61 (1H, s), 7.66 (2H, d, J = 8.6 Hz). |
| 70 | 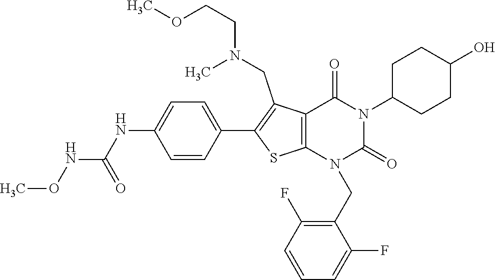 [SMILES: COCCN(C)Cc1c(-c2ccc(NC(=O)NOC)cc2)sc2c1c(=O)n(C1CCC(O)CC1)c(=O)n2Cc1c(F)cccc1F] | ¹H-NMR (CDCl₃) δ: 1.2-1.6 (3H, m), 1.6-1.8 (2H, m), 2.0- 2.1 (2H, m), 2.14 (3H, s), 2.5- 2.75 (4H, m), 3.31 (3H, s), 3.45 (2H, t, J = 5.8 Hz), 3.65- 3.85 (3H, m), 3.82 (3H, s), 4.9- 5.05 (1H, br), 5.30 (2H, s), 6.90 (2H, t, J = 8.0 Hz), 7.12 (1H, s), 7.25-7.4 (1H, m), 7.5- 7.6 (4H, m), 7.60 (1H, s). |
| 71 | 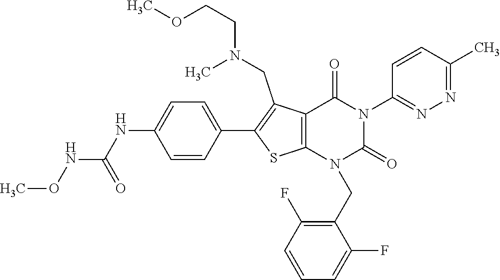 | ¹H-NMR (CDCl₃) δ: 2.13 (3H, s), 2.61 (2H, t, J = 5.8 Hz), 2.79 (3H, s), 3.26 (3H, s), 3.41 (2H, t, J = 5.8 Hz), 3.75-3.85 (2H, m), 3.82 (3H, s), 5.25-5.45 (2H, brm), 6.92 (2H, t, J = 8.2 Hz), 7.18 (1H, s), 7.2-7.7 (8H, m). |
| 72 | 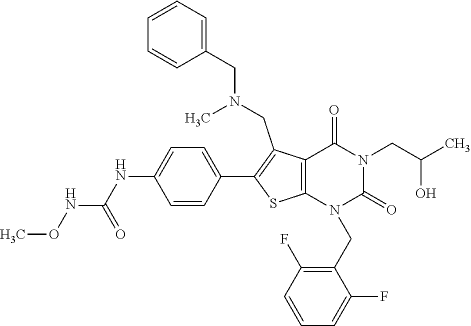 [SMILES: CONC(=O)Nc1ccc(-c2sc3c(c2CN(C)Cc2ccccc2)c(=O)n(CC(C)O)c(=O)n3Cc2c(F)cccc2F)cc1] | ¹H-NMR (CDCl₃) δ: 1.30 (3H, d, J = 6.0 Hz), 2.06 (3H, s), 2.90 (1H, d, J = 5.2 Hz), 3.57 (2H, s), 3.82 (3H, s), 3.91 (2H, s), 4.1- 4.25 (1H, m), 4.20 (2H, s), 5.34 (2H, s), 6.91 (2H, t, J = 8.0 Hz), 7.16 (1H, s), 7.2-7.4 (6H, m), 7.54 (2H, d, J = 8.8 Hz), 7.62 (1H, s), 7.67 (2H, d, J = 8.8 Hz). |
| 73 | 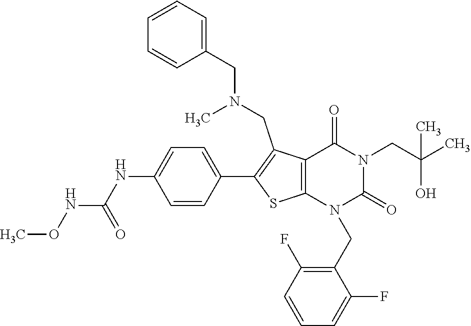 [SMILES: CONC(=O)Nc1ccc(-c2sc3c(c2CN(C)Cc2ccccc2)c(=O)n(CC(C)(C)O)c(=O)n3Cc2c(F)cccc2F)cc1] | ¹H-NMR (CDCl₃) δ: 1.29 (6H, s), 2.06 (3H, s), 3.56 (2H, s), 3.83 (3H, s), 3.91 (2H, s), 3.96 (1H, s), 4.28 (2H, s), 5.36 (2H, s), 6.91 (2H, t, J = 8.2 Hz), 7.13 (1H, s), 7.2-7.35 (6H, m), 7.54 (2H, d, J = 8.8 Hz), 7.62 (1H, s), 7.67 (2H, d, J = 8.8 Hz). |
| 74 | 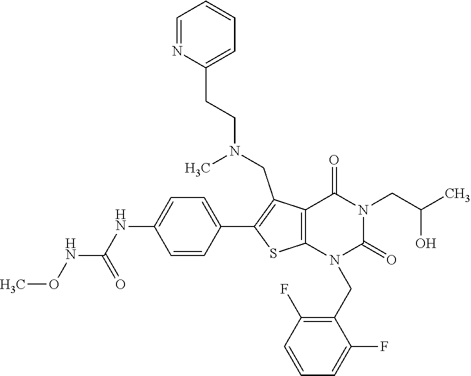 [SMILES: CONC(=O)Nc1ccc(-c2sc3c(c2CN(C)CCc2ccccn2)c(=O)n(CC(C)O)c(=O)n3Cc2c(F)cccc2F)cc1] | ¹H-NMR (CDCl₃) δ: 1.28 (3H, d, J = 5.8 Hz), 2.21 (3H, s), 2.75-2.95 (4H, m), 3.0-3.1 (1H, m), 3.82 (5H, s), 4.1-4.2 (1H, m), 4.17 (2H, s), 5.34 (2H, s), 6.91 (2H, t, J = 8.2 Hz), 6.95- 7.1 (2H, m), 7.14 (1H, s), 7.2- 7.4 (1H, m), 7.4-7.55 (5H, m), 7.59 (1H, s), 8.43 (1H, d, J = 5.0 Hz). |
| 75 | 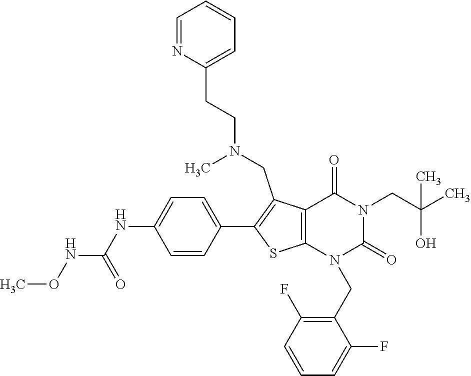 [SMILES: CONC(=O)Nc1ccc(-c2sc3c(c2CN(C)CCc2ccccn2)c(=O)n(CC(C)(C)O)c(=O)n3Cc2c(F)cccc2F)cc1] | ¹H-NMR (CDCl₃) δ: 1.26 (6H, s), 2.21 (3H, s), 2.75-2.95 (4H, m), 3.82 (5H, s), 3.99 (1H, s), 4.24 (2H, s), 5.36 (2H, s), 6.91 (2H, t, J = 8.2 Hz), 7.0-7.1 (2H m), 7.13 (1H, s), 7.2-7.35 (1H, m), 7.45-7.55 (5H, m), 7.59 (1H, s), 8.43 (1H, d, J = 4.0 Hz). |
| 76 | 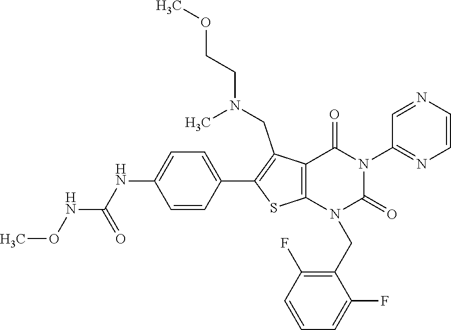 [SMILES: COCCN(C)Cc1c(-c2ccc(NC(=O)NOC)cc2)sc2c1c(=O)n(-c1cnccn1)c(=O)n2Cc1c(F)cccc1F] | ¹H-NMR (CDCl₃) δ: 2.13 (3H, s), 2.62 (2H, t, J = 6.0 Hz), 3.26 (3H, s), 3.41 (2H, t, J = 6.0 Hz), 3.79 (2H, s), 3.83 (1H, s), 5.36 (2H, s), 6.94 (2H, t, J = 8.0 Hz), 7.12 (1H, s), 7.2-7.4 (1H, m), 7.5-7.65 (5H, m), 8.65-8.7 (3H, m). |
| 77 | 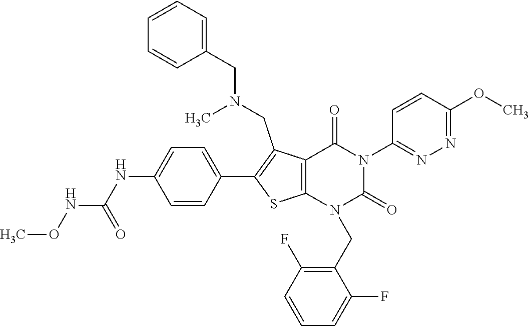 | ¹H-NMR (CDCl₃) δ: 2.06 (3H, s), 3.55 (2H, s), 3.83 (3H, s), 3.87 (2H, s), 4.19 (3H, s), 5.35 (2H, s), 6.92 (2H, t, J = 8.2 Hz), 7.1-7.45 (9H, m), 7.55 (2H, d, J = 8.4 Hz), 7.63 (1H, s), 7.72 (2H, d, J = 8.4 Hz). |
| 78 | 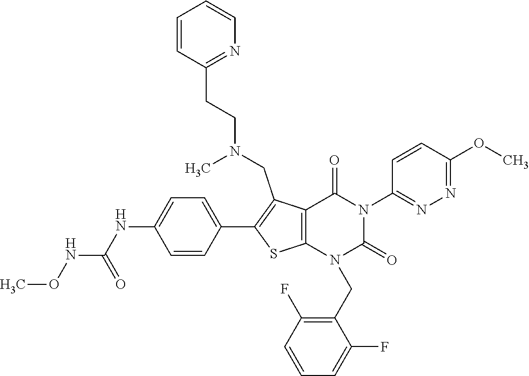 | ¹H-NMR (CDCl₃) δ: 2.20 (3H, s), 2.7-2.9 (4H, m), 3.78 (2H, s), 3.82 (3H, s), 4.19 (3H, s), 5.34 (2H, s), 6.85-7.2 (5H, m), 7.25-7.45 (2H, m), 7.45-7.7 (7H, m), 8.42 (1H, d, J = 4.0 Hz). |
| 79 | 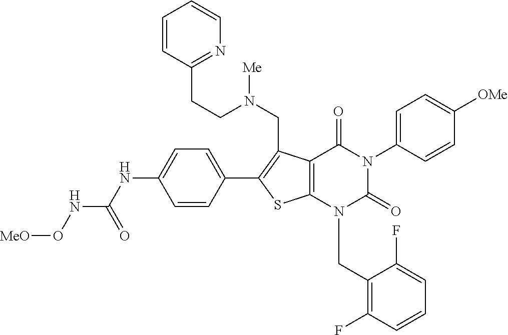 [SMILES: COONC(=O)Nc1ccc(-c2sc3c(c2CN(C)CCc2ccccn2)c(=O)n(-c2ccc(OC)cc2)c(=O)n3Cc2c(F)cccc2F)cc1] | ¹H NMR (CDCl₃) δ 2.20 (3H, s), 2.86 (4H, m), 3.82-3.84 (8H, m), 5.36 (2H, s), 6.92 (2H, d, J = 8.3 Hz), 7.00-7.06 (4H, m), 7.14-7.33 (4H, in), 7.46-7.51 (5H, m), 7.61 (1H, s), 8.42 (1H, d, J = 5.7 Hz). |
| 80 | 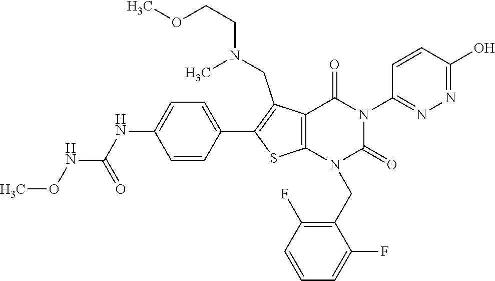 [SMILES: COCCN(C)Cc1c(-c2ccc(NC(=O)NOC)cc2)sc2c1c(=O)n(-c1ccc(O)nn1)c(=O)n2Cc1c(F)cccc1F] | ¹H-NMR (CDCl₃) δ: 2.12 (3H, s), 2.63 (2H, t, J = 5.8 Hz), 3.28 (3H, s), 3.43 (2H, t, J = 5.8 Hz), 3.79 (2H, s), 3.83 (3H, s), 5.35 (2H, s), 6.94 (2H, t, J = 8.2 Hz), 7.0-7.1 (1H, m), 7.2-7.4 (3H, m), 7.5-7.65 (4H, m), 7.63 (1H, s), 10.5-10.6 (1H, brs). |
| 81 | 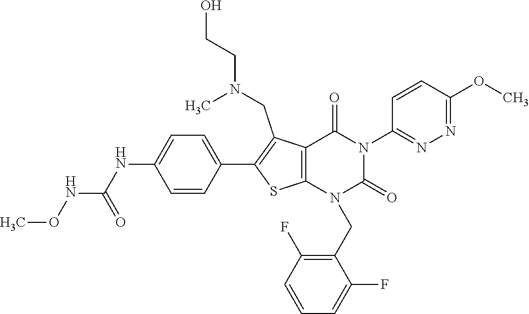 | ¹H-NMR (CDCl₃) δ: 1.98 (3H, s), 2.45-2.5 (2H, m), 2.9-3.2 (1H, m), 3.5-3.55 (2H, m), 3.65-3.85 (2H, brm), 3.82 (3H, s), 4.18 (3H, s), 5.34 (2H, s), 6.93 (2H, t, J = 8.0 Hz), 7.11 (1H, d, J = 9.0 Hz), 7.18 (1H, s), 7.25-7.35 (1H, m), 7.35-7.45 (3H, m), 7.57 (2H, d, J = 8.7 Hz), 7.66 (1H, s). |
| 82 | 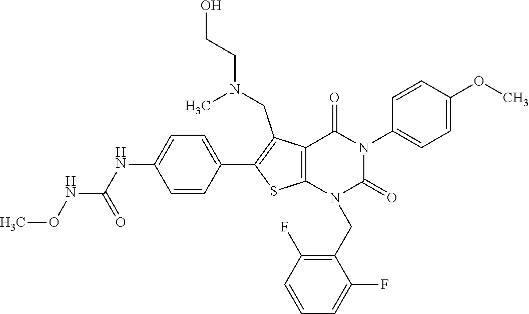 | ¹H-NMR (CDCl₃) δ: 1.99 (3H, s), 2.45-2.55 (2H, m), 3.5-3.6 (2H, m), 3.79 (2H, s), 3.82 (3H, s), 3.83 (3H, s), 5.36 (2H, s), 6.92 (2H, t, J = 8.0 Hz), 6.99 (2H, d, J = 8.8 Hz), 7.1-7.3 (4H, m), 7.39 (2H, d, J = 8.8 Hz), 7.56 (2H, d, J = 8.8 Hz), 7.64 (1H, s). |
| 83 | 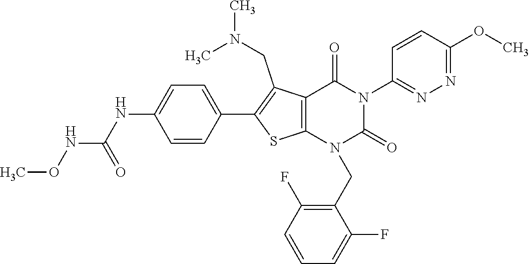 | ¹H-NMR (CDCl₃) δ: 2.15 (6H, s), 3.6-3.8 (2H, m), 3.82 (3H, s), 4.18 (3H, s), 5.35 (2H), 6.92 (2H, t, J = 8.2 Hz), 7.12 (1H, d, J = 8.8 Hz), 7.2-7.65 (7H, m), 7.69 (1H, s). |
| 84 | 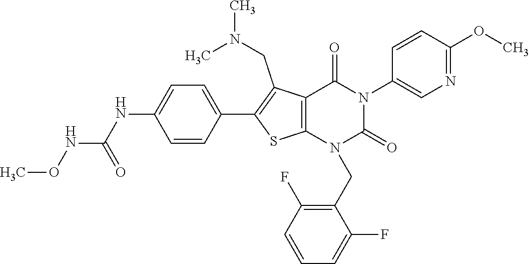 | ¹H-NMR (CDCl₃) δ: 2.13 (6H, s), 3.68 (2H, s), 3.83 (3H, s), 3.96 (3H, s), 5.36 (2H, s), 6.8- 7.0 (3H, m), 7.13 (1H, s), 7.2- 7.4 (1H, m), 7.45-7.65 (6H, m), 8.10 (1H, d, J = 2.6 Hz). |

### Propane-1,3-diones

Additional GnRH antagonists that may be used in conjunction with the compositions and methods described herein include optionally substituted propane-1,3-dione derivatives, such as (2R)—N-{5-[3-(2,5-difluorophenyl)-2-(1,3-dihydro-2H-benzimidazol-2-ylidene)-3-oxopropanoyl]-2-fluorobenzene-1-sulfonyl}-2-hydroxypropanimidamide, also referred to as opigolix or ASP-1707. Other GnRH antagonists of this chemical class that may be used for the treatment of estrogen-dependent diseases in accordance with the compositions and methods of the disclosure include compounds described in U.S. Pat. No. 6,960,591, the contents of which are incorporated herein by reference.

### Add-Back Therapy

Among the potential side-effects of GnRH antagonist therapy is a reduction in bone mineral density due to excessive depletion of estrogen (Newhall-Perry et al., American Journal of Obstetrics and Gynecology 173:824-829 (1995)). To combat this potential side effect, a patient undergoing GnRH antagonist therapy using the compositions and methods described herein can be administered add-back therapy. Add-back therapy may contain an estrogen (such as β17-estradiol, ethinyl estradiol, or a conjugated estrogen, such as a conjugated equine estrogen) optionally in combination with a progestin (such as norethindrone or an ester thereof, e.g., norethindrone acetate, or another agent such as progesterone, norgestimate, medroxyprogesterone, or drospirenone).

Endogenous estrogens are largely responsible for the development and maintenance of the female reproductive system and secondary sexual characteristics. Although circulating estrogens exist in a dynamic equilibrium of metabolic interconversions, estradiol is the principal intracellular human estrogen and is substantially more potent than its metabolites, estrone and estriol, at the receptor level. The primary source of estrogen in normally cycling adult women is the ovarian follicle, which secretes 70 to 500 μg of estradiol daily, depending on the phase of the menstrual cycle. After menopause, most endogenous estrogen is produced by conversion of androstenedione, secreted by the adrenal cortex, to estrone by peripheral tissues. Thus, estrone and the sulfate conjugated form, estrone sulfate, are the most abundant circulating estrogens in postmenopausal women. Circulating estrogens modulate the pituitary secretion of the gonadotropins, LH and FSH, through a negative feedback mechanism. Estrogens act to reduce the elevated levels of these hormones seen in postmenopausal women.

Progestin compounds, such as norethindrone and esters thereof (e.g., norethindrone acetate), as well as progesterone, norgestimate, medroxyprogesterone, and drospirenone, enhance cellular differentiation and generally oppose the actions of estrogens by decreasing estrogen receptor levels, increasing local metabolism of estrogens to less active metabolites, or inducing gene products that blunt cellular responses to estrogen. Progestins exert their effects in target cells by binding to specific progesterone receptors that interact with progesterone response elements in target genes. Progesterone receptors have been identified in the female reproductive tract, breast, pituitary, hypothalamus, and central nervous system. Progestins produce similar endometrial changes to those of the naturally occurring hormone progesterone. Progestins may be included in combination with estrogen in add-back therapy. For instance, according to the methods described herein, one can administer estrogen (e.g., E2) in conjunction with a progestin (e.g., norethindrone or an ester thereof, such as norethindrone acetate) to a patient undergoing GnRH antagonist therapy as to counteract the hypoestrogenemia that may be induced by the antagonist. In this way, add-back therapy can be used to mitigate or prevent potentially deleterious side effects, such as a reduction in bone mineral density.

Add-back therapy may be formulated for oral administration. For instance, add-back therapy administered in conjunction with the compositions and methods described herein may be formulated as a tablet, capsule, gel cap, powder, liquid solution, or liquid suspension. In some embodiments, the add-back therapy includes both an estrogen, such as β17-estradiol, and a progestin, such as norethindrone or norethindrone acetate. The estrogen and progestin may be administered separately or admixed in a single composition, such as a single tablet, capsule, gel cap, powder, liquid solution, or liquid suspension.

For example, add-back therapy may feature a co-formulation containing estrogen (e.g., in the form of E2) and an additional agent such as a progestin (e.g., norethindrone or a compound that is metabolized in vivo to produce norethindrone, such as an ester of norethindrone that is de-esterified in vivo to produce norethindrone, for instance, norethindrone acetate). In some embodiments, add-back therapy is administered to a patient in the form of a single tablet, capsule, gel cap, powder, liquid solution, or liquid suspension that contains both estrogen (e.g., in the form of E2) and a progestin (e.g., norethindrone or a compound that is metabolized in vivo to produce norethindrone, such as an ester of norethindrone that is de-esterified in vivo to produce norethindrone, for instance, norethindrone acetate). In some embodiments, add-back therapy is administered as a fixed dose combination containing a GnRH antagonist, estrogen, and one or more additional agents, such as a progestin, in a single pharmaceutical composition. For instance, add-back therapy may be administered as a fixed dose combination of a GnRH antagonist, estrogen (e.g., in the form of E2) and a progestin (e.g., norethindrone or a compound that is metabolized in vivo to produce norethindrone, such as an ester of norethindrone that is de-esterified in vivo to produce norethindrone, for instance, norethindrone acetate) in the form of a single pharmaceutical composition, such as a single tablet, capsule, gel cap, powder, liquid solution, or liquid suspension.

### Methods of Treating Estrogen-Dependent Diseases

Using the compositions and methods described herein, a patient having an estrogen-dependent diseases, such as uterine fibroids, endometriosis (e.g., rectovaginal endometriosis), and/or adenomyosis, among others, may be administered a GnRH antagonist as described herein. Additional examples of estrogen-dependent diseases that may be treated using the compositions and methods of the disclosure include, without limitation, benign prostatic hypertrophy, precocious puberty, premenstrual syndrome, polycystic ovary syndrome, hirsutism, short stature, a sleep disorder, acne, baldness, and irritable bowel syndrome.

A variety of methods known in the art and described herein can be used to determine whether a patient is responding favorably to GnRH antagonist treatment. For instance, beneficial clinical results in response to GnRH antagonist therapy include, without limitation, alleviation of symptoms of the endometrial growth disorder. Indications of successful treatment of a uterine fibroids patient include, for example, (i) a reduction in serum concentration of FSH, LH, and/or E2 following administration of the GnRH antagonist to the patient, (ii) a reduction in uterine blood loss following administration of the GnRH antagonist to the patient, (iii) elimination of heavy menstrual bleeding following administration of the GnRH antagonist to the patient, and (iv) induction of amenorrhea in the patient following administration of the GnRH antagonist to the patient.

Similarly, clinical indicators of successful treatment of an endometriosis patient (e.g., a rectovaginal endometriosis patient) that is administered a GnRH antagonist include, without limitation, (i) a reduction in serum concentration of FSH, LH, and/or E2 following administration of the GnRH antagonist to the patient, (ii) a reduction in the volume of one or more endometriosis nodes (e.g., rectovaginal endometriosis nodes) following administration of the GnRH antagonist to the patient, (iii) a reduction in bowel involvement of one or more type III endometriosis nodes following administration of the GnRH antagonist to the patient, (iv) a reduction in pelvic pain following administration of the GnRH antagonist to the patient; (v) a reduction in dysmenorrhea following administration of the GnRH antagonist to the patient; (vi) a reduction in dyspareunia following administration of the GnRH antagonist to the patient; (vii) a reduction in dyschezia following administration of the GnRH antagonist to the patient; (viii) a reduction in uterine bleeding following administration of the GnRH antagonist to the patient; (ix) achievement of amenorrhea following administration of the GnRH antagonist to the patient; and (x) an improvement in the patient's overall well-being as determined by an improvement in the patient's Endometriosis Health Profile questionnaire (EHP-30)score following administration of the GnRH antagonist to the patient and/or by way of a positive Patient Global Impression of Change (PGIC) score following administration of the GnRH antagonist to the patient.

Exemplary indicia of successful treatment of an adenomyosis patient that is administered a gonadotropin-releasing hormone (GnRH) antagonist include, without limitation, (i) a reduction in serum concentration of FSH, LH, and/or E2 following administration of the GnRH antagonist to the patient, (ii) a reduction in uterine volume following administration of the GnRH antagonist to the patient, (iii) a reduction in the thickness of the anterior and/or posterior region of the uterine myometrium following administration of the GnRH antagonist to the patient, (iv) a reduction in pelvic pain following administration of the GnRH antagonist to the patient; (v) a reduction in dysmenorrhea following administration of the GnRH antagonist to the patient; (vi) a reduction in dyspareunia following administration of the GnRH antagonist to the patient; (vii) a reduction in dyschezia following administration of the GnRH antagonist to the patient; (viii) a reduction in uterine tenderness following administration of the GnRH antagonist to the patient; (ix) a reduction in uterine bleeding following administration of the GnRH antagonist to the patient; (x) achievement of amenorrhea following administration of the GnRH antagonist to the patient; (xi) a reduction in the diameter of a junctional zone of adenomyosis following administration of the GnRH antagonist to the patient; and (xii) an improvement in the patient's overall well-being as determined by an improvement in the patient's EHP-30 score following administration of the GnRH antagonist to the patient and/or by way of a positive PGIC score following administration of the GnRH antagonist to the patient.

### Modified Biberoglu and Behrman Symptom Severity Scale

Exemplary methods for assessing a patient's response to GnRH antagonist therapy for the treatment of estrogen-dependent diseases, such as uterine fibroids, endometriosis (e.g., rectovaginal endometriosis) and/or adenomyosis, among others described herein, include administration of a modified Biberoglu and Behrman questionnaire, as described herein. An exemplary mB&B questionnaire for use in conjunction with the compositions and methods described herein is shown in Table 4, below.

### Endometriosis Health Profile Questionnaire

Additional methods for assessing patient respond to GnRH antagonist therapy for the treatment of estrogen-dependent diseases, such as uterine fibroids, endometriosis (e.g., rectovaginal endometriosis) and/or adenomyosis, among others described herein, include analyzing the patient's score on an Endometriosis Health Profile questionnaire. An exemplary Endometriosis Health Profile questionnaire for use in conjunction with the compositions and methods described herein is the EHP-30 questionnaire shown in Table 5, below.

### Patient Global Impression of Change Score

Additional methods for assessing patient response to GnRH antagonist therapy for the treatment of an estrogen-dependent disease described herein, such as uterine fibroids, endometriosis (e.g., rectovaginal endometriosis, and/or adenomyosis, among others) include analyzing the patient's score on a Patient Global Impression of Change (PGIC) scale. An exemplary PGIC questionnaire for use in conjunction with the compositions and methods described herein is shown in Table 6, below.

### Quantitation of Uterine Blood Loss by the Alkaline Hematin Method

Techniques for quantifying uterine blood loss are known in the art and include, for instance, the alkaline hematin method, as described, for instance, in Hallberg et al., Scand. J. Clin. Lab. Invest. 16:244-248 (1964), the disclosure of which is incorporated herein by reference as it pertains to techniques for assessing the volume of blood lost by a patient. In the alkaline hematin approach, uterine blood soaked into, for example, a sanitary napkin, vaginal tampon, or cotton pad, is reconstituted in a basic aqueous solution, such as a solution of 5% (w/v) sodium hydroxide. This incubation enables (i) extraction of the iron-containing porphyrin of hemoglobin and (ii) oxidation of the ferrous ion to a hydroxy-coordinated ferric ion in each chelate, thus forming hematin. Hematin is a detectable chromophore, absorbing light at between 550 and 546 nm. By comparing the concentration of hematin obtained from incubation of a soaked menstrual blood sample with aqueous sodium hydroxide to the concentration of hematin obtained from incubation of a sample of venous blood with aqueous sodium hydroxide, one can stoichiometrically determine the volume of menstrual blood lost by a patient, such as a patient having an estrogen-dependent disease. Improvements to the original alkaline hematin method are known in the art and are described, for example, in Newton et al., Contraception 16:269-282 (1977), and in van Eijkeren et al., Eur. J. Obstet. Gynecol. Reprod. Biol. 22:345-351 (1986), the disclosures of each of which are incorporated herein by reference as they pertain to methods of determining the volume of blood lost by a patient.

### Routes of Administration and Dosing of GnRH Antagonists

The GnRH antagonists described herein may be administered to a patient in need thereof (e.g., a patient suffering from an estrogen-dependent disease, such as uterine fibroids, endometriosis (e.g., rectovaginal endometriosis) and/or adenomyosis, among others described herein) by a variety of routes of administration. For instance, the GnRH antagonists described herein may be formulated for oral administration, among other routes. Exemplary non-oral routes of administration of the GnRH antagonists described herein include, without limitation, intravenous administration, parenteral administration, subcutaneous administration, intramuscular administration, and intradermal administration, among others.

In some embodiments, the GnRH antagonist is a compound of any one of formulas (I)-(VIa), above, and is administered to the patient in an amount of from about 25 mg to about 500 mg per dose, and may be administered in one or more doses per day, for example, in accordance with a dosing schedule described above. For instance, the GnRH antagonist may a compound of any one of formulas (I)-(VIa), above, and may be administered to the patient in an amount of from 25 mg to 500 mg, 30 mg to 495 mg, 35 mg to 490 mg, 40 mg to 485 mg, 45 mg to 480 mg, 50 mg to 475 mg, 55 mg to 470 mg, 60 mg to 465 mg, 65 mg to 460 mg, 70 mg to 455 mg, 75 mg to 450 mg, 80 mg to 445 mg, 85 mg to 440 mg, 90 mg to 435 mg, 95 mg to 430 mg, 100 mg to 425 mg, 105 mg to 420 mg, 110 mg to 415 mg, 115 mg to 410 mg, 120 mg to 405 mg, 125 mg to 400 mg, 130 mg to 395 mg, 135 mg to 390 mg, 140 mg to 385 mg, 145 mg to 380 mg, or 150 mg to 375 mg, per dose, and may be administered in one or more doses per day (e.g., in a single daily dose). In some embodiments, the GnRH antagonist is a compound of any one of formulas (I)-(VIa), above, and is administered to the patient once daily in an amount of from 100 mg to 300 mg, per dose, such as from 105 mg to 295 mg, 110 mg to 290 mg, 115 mg to 285 mg, 120 mg to 280 mg, 125 mg to 275 mg, 130 mg to 270 mg, 135 mg to 265 mg, 140 mg to 260 mg, 145 mg to 255 mg, 150 mg to 250 mg, 155 mg to 245 mg, 160 mg to 240 mg, 165 mg to 235 mg, 170 mg to 230 mg, 175 mg to 225 mg, 180 mg to 220 mg, 185 mg to 215 mg, 190 mg to 210 mg, or 195 mg to 205 mg, per dose. In some embodiments, the GnRH antagonist is a compound of any one of formulas (I)-(VIa), above, and is administered to the patient once daily in an amount of about 100 mg per dose or 200 mg per dose.

The GnRH antagonists described herein may be administered to a patient a plurality of times over the course of a treatment period. For instance, the GnRH antagonists described herein may be administered to a patient periodically over a treatment period of at least two weeks (e.g., a treatment period of from about two weeks to about six months, about three weeks to about five months, about four weeks to about four months, or about one month to about three months). The GnRH antagonist may be administered to the patient, for example, over a treatment period of from about four weeks to about six months (e.g., from about 28 days to about 180 days, about 30 days to about 175 days, about 35 days to about 170 days, about 40 days to about 165 days, about 45 days to about 160 days, about 50 days to about 155 days, about 55 days to about 150 days, about 60 days to about 145 days, about 65 days to about 140 days, about 70 days to about 135 days, about 75 days, to about 130 days, about 80 days to about 125 days, about 85 days, to about 120 days, or about 90 days to about 115 days). In some embodiments, the GnRH antagonist is periodically administered to the patient over the course of from about eight weeks to about sixteen weeks (e.g., from about 60 days to about 110 days, about 65 days to about 105 days, about 70 days to about 100 days, about 75 days to about 95 days, or about 80 days, to about 90 days). In some embodiments, the GnRH antagonist is periodically administered to the patient over a treatment period of about 12 weeks. In some embodiments, the GnRH antagonist is periodically administered to the patient over a treatment period of about 24 weeks.

Additional dosing schedules for the treatment of estrogen-dependent diseases, such as uterine fibroids, endometriosis (e.g., rectovaginal endometriosis) and/or adenomyosis, among others described herein, using other GnRH antagonists disclosed herein are described in detail above.

### Pharmaceutical Compositions

GnRH antagonists suitable for use with the compositions and methods described herein can be formulated into a pharmaceutical composition for administration to a patient, such as a female human patient, in a biologically compatible form suitable for administration in vivo. A pharmaceutical composition containing a GnRH antagonist, such as a compound described herein (e.g., 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylic acid or a pharmaceutically acceptable salt thereof, such as the choline salt thereof), may additionally contain a suitable diluent, carrier, or excipient. GnRH antagonists can be administered to a patient, for example, orally or by intravenous injection. Under ordinary conditions of storage and use, a pharmaceutical composition may contain a preservative, e.g., to prevent the growth of microorganisms.

Conventional procedures and ingredients for the selection and preparation of suitable formulations are described, for example, in Remington: The Science and Practice of Pharmacy (2012, 22^{nd} ed.) and in The United States Pharmacopeia: The National Formulary (2015, USP 38 NF 33).

Pharmaceutical compositions may include sterile aqueous solutions, dispersions, or powders, e.g., for the extemporaneous preparation of sterile solutions or dispersions. In all cases the form may be sterilized using techniques known in the art and may be fluidized to the extent that may be easily administered to a patient in need of treatment.

A pharmaceutical composition may be administered to a patient, e.g., a human patient, alone or in combination with one or more pharmaceutically acceptable carriers, e.g., as described herein, the proportion of which may be determined by the solubility and/or chemical nature of the compound, chosen route of administration, and standard pharmaceutical practice.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regards as their invention.

### Example 1. Concurrent Onset of GnRH Antagonist Therapy and Add-Back Therapy Depletes Endogenous Estradiol and Reduces Uterine Blood Loss to a Greater Extent than GnRH Antagonist Pre-Treatment

This example describes a series of experiments conducted in order to investigate the estrogen-modulating properties of GnRH antagonists, such as those of formulas (I)-(VIa) described herein, as well as the effect of GnRH antagonists on menstrual blood loss. To conduct these experiments, a set of pre-menopausal female human subjects were periodically administered a GnRH antagonist represented by formula (VI). Particularly, subjects were treated with the choline salt of compound (VI) in combination with add-back therapy. Treatment with this compound, among other GnRH antagonists described herein, can alleviate endometriosis-associated pain as well as uterine fibroids-associated heavy menstrual bleeding. In pharmacodynamic studies, compounds of formulas (I)-(VIa), such as compounds of formula (VI) (e.g., the choline salt of compound (VI)) have been shown to effectuate dose-dependent reductions in β17-estradiol (E2) levels in female human subjects, which, in turn, reduced uterine bleeding and increased the incidence of amenorrhea.

Significant reductions in E2 are associated with treatment-limiting bone mineral density (BMD) loss and hot flushes. To prevent BMD reduction, a GnRH antagonist, such as a compound of formulas (I) -(VIa) described herein, among others, can be co-administered with hormonal add-back therapy to restore appropriate endogenous levels of E2.

However, previous studies have shown that the inclusion of add-back therapy results in fewer women exhibiting amenorrhea. It has been observed that four weeks of daily administration of a GnRH antagonist, such as a GnRH antagonist represented by formula (VI) (e.g., delivered in the form of the choline salt of compound (VI)), to pre-menopausal female human subjects results in endogenous E2 levels and an endometrium status that are comparable to those of postmenopausal women.

Postmenopausal women treated with hormonal add-back therapy have amenorrhea rates exceeding those of pre-menopausal women treated with a GnRH antagonist and add-back therapy. Therefore, the experiments described in this example were conducted to assess whether co-administration of add-back therapy and GnRH antagonist therapy following four weeks of pre-treatment with a GnRH antagonist alone would result in reduced E2 levels and improved uterine bleeding patterns compared to dosing regimens in which subjects receive a simultaneous onset of GnRH antagonist and add-back therapy administration.

### Methods

The experiments described in this example were conducted as part of a single-center, open-label, randomized, parallel-group clinical trial in 32 pre-menopausal women. Following screening assessments, and during the second half of the menstrual cycle, women received norethindrone (5 mg, three-times daily for 10 days) to synchronize menstrual cycles. After menstrual cycle synchronization, subjects were randomized with a 1:1 ratio to one of two treatment arms: (i) a cohort receiving once-daily administration of compound (VI) (200 mg per day, administered in the form of a choline salt) with concurrent onset of add-back therapy administration for 10 weeks (“Concurrent-ABT”), or (ii) a cohort receiving pre-treatment with compound (VI) (200 mg per day, administered in the form of a choline salt) for 4 weeks followed by combined daily administration of compound (VI) and add-back therapy (“Delayed-ABT”). The add-back therapy used in these experiments contained 1.0 mg of E2 and 0.5 mg of norethindrone acetate, and was administered to subjects once daily.

The main treatment outcomes and endpoints measured during these experiments included (i) a qualitative assessment of uterine blood loss, which was recorded on a daily basis throughout the study by characterizing each subject's bleeding pattern using one of the following four categories: “no bleeding,” “spotting” (light staining, dark blood, no sanitary protection needed or only panty liner), “bleeding” (1-4 completely soiled maxi sanitary towels or 1-8 soiled tampons or equivalent combination of the two), or “heavy bleeding” (>4 completely soiled maxi sanitary towels or >8 soiled tampons or equivalent combination of the two); (ii) a bi-weekly assessment of each subject's circulating E2 concentration; (iii) a bi-weekly assessment of each subject's circulating progesterone concentration (measured using a validated commercial chemiluminescence immunoassay method (Elecsys Progesterone III, Roche, Burgess Hill, UK)); (iv) the pharmacokinetic profile of compound (VI) and the add-back therapy; and (v) adverse events.

### Results

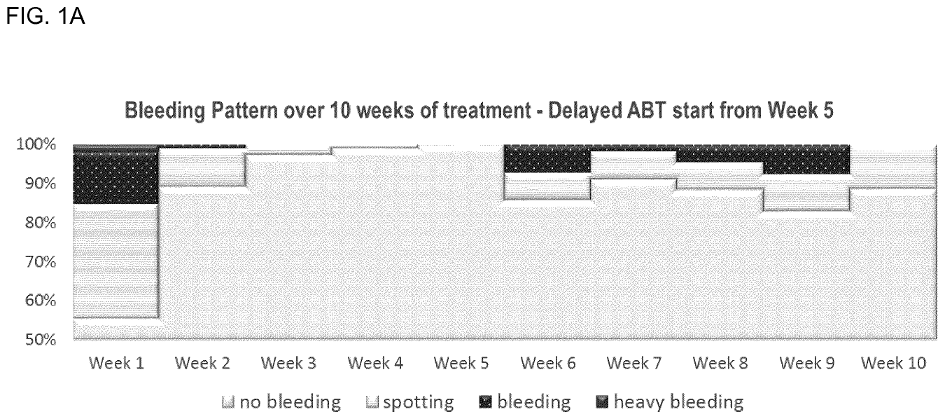

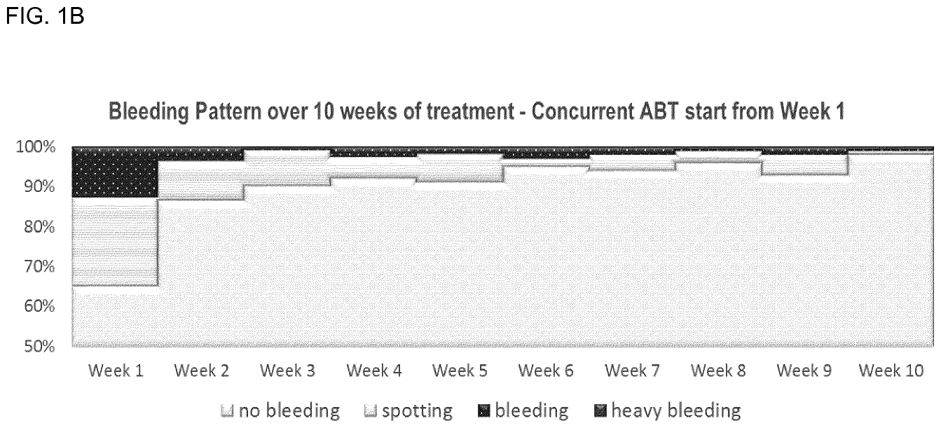

Compound (VI) alone promptly reduced uterine bleeding, leading to amenorrhea in all subjects by week 5 (FIG. 1A, “Delayed-ABT”). When add-back therapy was initiated in this cohort in week 5 of GnRH antagonist administration, spotting (≤11%) and bleeding (≤8%) occurred. The uterine bleeding in this cohort was markedly more frequent than after the simultaneous initiation of add-back therapy and GnRH antagonist therapy in the “Concurrent-ABT” group (≤4%) (FIG. 1B). In the “Concurrent-ABT” group, some spotting (≤10%) and occasional bleeding (≤4%) occurred during the first half of treatment, with a tendency to further decrease during the second half of the treatment period, during which spotting occurred with a frequency of about 1-5%, bleeding occurred with a frequency of less than 3%, and amenorrhea was induced with a frequency of up to 98%.

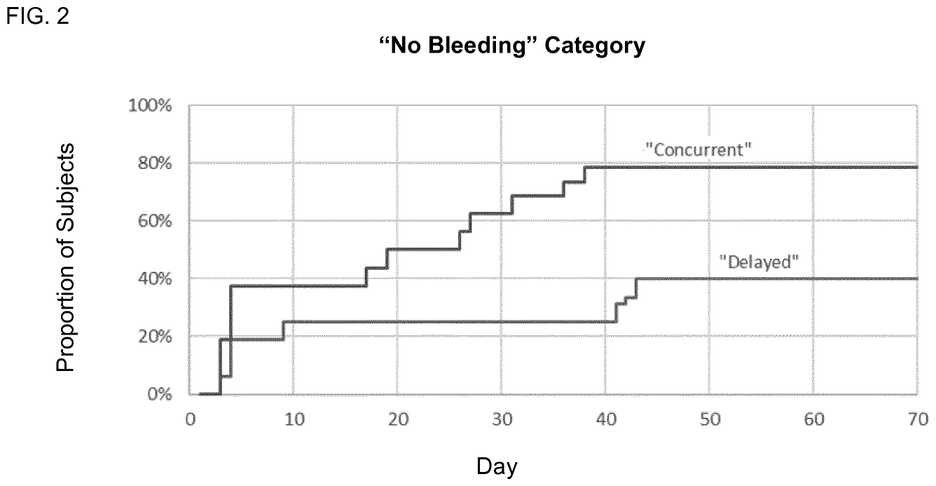

The occurrence of amenorrhea, which, in this study, was defined as either “no bleeding” or the combination of “no bleeding” and “spotting” during the last 4 weeks of treatment, was investigated over the duration of GnRH antagonist treatment. In the “Concurrent-ABT” group, close to 80% of women were in complete amenorrhea (“no bleeding”) during the last 4 weeks of treatment, approximately half of which (˜40%) had reached amenorrhea within 5 days of treatment. In the “Delayed-ABT” group, significantly less women (40%) were in amenorrhea (p=0.03) during the last 4 weeks of treatment, and only ˜20% of women were in amenorrhea within 5 days of treatment. The proportions of patients in each cohort falling into the “no bleeding” category over the duration of the GnRH antagonist are shown in FIG. 2.

The uterine bleeding patterns observed in each of the “Concurrent-ABT” and “Delayed-ABT” cohorts are summarized in Table 7, below.

TABLE 7
Comparison of uterine bleeding patterns observed in the “Concurrent-
ABT” and “Delayed-ABT” cohorts

| Treatment Arm | Time of Study | Uterine Bleeding Pattern |
|---|---|---|
| “Concurrent-ABT” | Weeks 2-4 | Spotting: ≤8% |
| | | Bleeding: ≤4% |
| | | Spotting and bleeding generally decreased over time |
| | Weeks 7-10 | Spotting: 1-5% |
| | | Bleeding: ≤2% |
| | | Amenorrhea: Up to 98% |
| “Delayed-ABT” | Weeks 2-4 | Occasional spotting and bleeding, decreasing to |
| | | almost 100% amenorrhea (achieved in week 5) |
| | Weeks 6-10 | Spotting and bleeding began after 1 week of add- |
| | | back therapy (i.e., week 6); |
| | | Bleeding more frequent than during initial weeks of |
| | | “Concurrent-ABT” treatment |

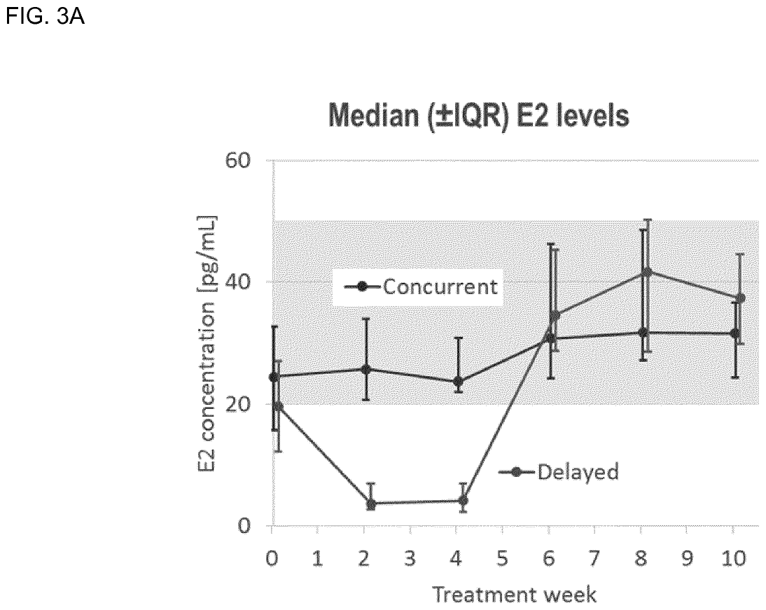

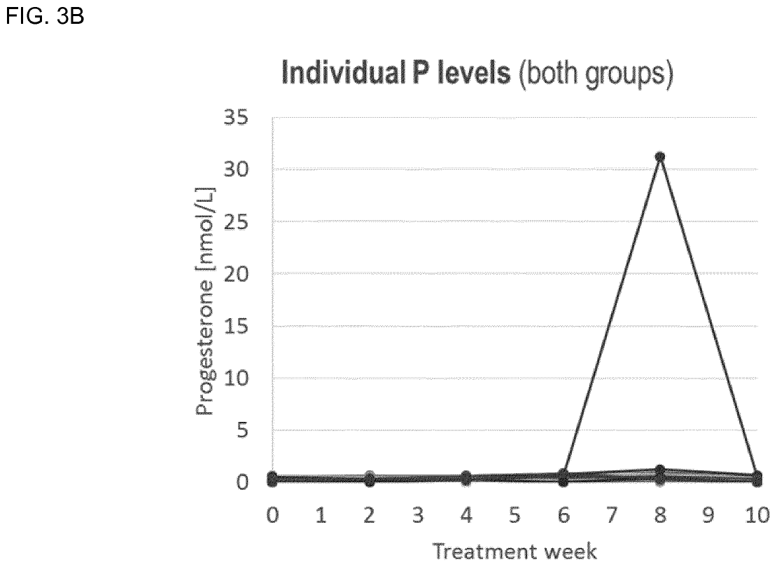

In addition to characterizing the bleeding patterns of subjects in each of the “Delayed-ABT” and “Concurrent-ABT” cohorts described above, subjects were assessed to determine the extent to which each dosing regimen suppressed endogenous E2 levels and modulated serum progesterone levels. Both cohorts resulted in a sustained reduction in circulating progesterone (FIG. 3B), with the exception of a single outlier subject. Pre-treatment of subjects in the “Delayed-ABT” cohort with Compound (VI) alone rapidly reduced E2 to reach a median level of 3.7 μg/ml by week 2 and 4.1 μg/mL by week 4 (FIG. 3A). In contrast, the median E2 levels observed in subjects in the “Concurrent-ABT” cohort never deviated from the healthy window of 20-50 μg/ml, indicating a steady, sustained reduction in E2 to levels low enough to reduce uterine blood loss, yet sufficiently high enough to prevent undesirable side effects associated with excessive estrogen depletion, such as bone mineral density loss. Strikingly, subjects in the “Concurrent-ABT” cohort exhibited more substantial reductions in E2 levels by the conclusion of the study relative to subjects in the “Delayed-ABT” cohort. Median E2 levels at the end of this experiment ranged from 24 pg/ml to 32 μg/mL for the “Concurrent-ABT” group. In contrast, final E2 levels for the “Delayed-ABT” cohort ranged from 35 μg/ml to 42 μg/mL.

The more marked reduction in E2 levels effectuated by simultaneous onset of GnRH antagonist therapy and add-back therapy was manifest not only as reduced uterine blood loss among subjects in the “Concurrent-ABT” cohort relative to the “Delayed-ABT” cohort, but also as a lower incidence of adverse events associated with excessive estrogen depletion. Compound (VI) was well-tolerated, with the most frequently reported adverse events being headache (38/166) and hot flushes (19/166). Notably, hot flushes exclusively occurred in the “Delayed-ABT” group.

A detailed summary of the E2 concentrations, uterine bleeding patterns, and incidences of hot flushes in patients within the “Concurrent-ABT” cohort (labelled as treatment group “A”) and “Delayed-ABT” (labelled as treatment group “B”) cohort over the duration of GnRH antagonist administration is shown in Table 8, below.

TABLE 8
Comparison of serum E2 levels, bleeding patterns, and incidences of hot flushes in “Concurrent-
ABT” and “Delayed-ABT” cohorts over the duration of GnRH antagonist administration

| | | | | Plasma Estradiol Level (pg/mL) | | | | | |
|---|---|---|---|---|---|---|---|---|---|
| Time Point | Subject Characteristic | Treatment | N | <20 | | 20-50 | | >50 | |
| Days 1-14 | Hot Flush | A | 16 | 0 | (0.0%) | 0 | (0.0%) | 0 | (0.0%) |
| | | B | 16 | 2 | (12.5%) | 0 | (0.0%) | 0 | (0.0%) |
| | | Overall | 32 | 2 | (6.3%) | 0 | (0.0%) | 0 | (0.0%) |
| | No Bleeding | A | 16 | 0 | (0.0%) | 0 | (0.0%) | 1 | (6.3%) |
| | | B | 16 | 1 | (6.3%) | 0 | (0.0%) | 0 | (0.0%) |
| | | Overall | 32 | 1 | (3.1%) | 0 | (0.0%) | 1 | (3.1%) |
| | No Bleeding or Spotting | A | 16 | 0 | (0.0%) | 3 | (18.8%) | 1 | (6.3%) |
| | | B | 16 | 5 | (31.3%) | 0 | (0.0%) | 0 | (0.0%) |
| | | Overall | 32 | 5 | (15.6%) | 3 | (9.4%) | 1 | (3.1%) |
| Days 15-28 | Hot Flush | A | 16 | 0 | (0.0%) | 0 | (0.0%) | 0 | (0.0%) |
| | | B | 16 | 4 | (25.0%) | 0 | (0.0%) | 0 | (0.0%) |
| | | Overall | 32 | 4 | (12.5%) | 0 | (0.0%) | 0 | (0.0%) |
| | No Bleeding | A | 16 | 2 | (12.5%) | 6 | (37.5%) | 1 | (6.3%) |
| | | B | 16 | 14 | (87.5%) | 1 | (6.3%) | 0 | (0.0%) |
| | | Overall | 32 | 16 | (50.0%) | 7 | (21.9%) | 1 | (3.1%) |
| | No Bleeding or Spotting | A | 16 | 2 | (12.5%) | 10 | (62.5%) | 1 | (6.3%) |
| | | B | 16 | 15 | (93.8%) | 1 | (6.3%) | 0 | (0.0%) |
| | | Overall | 32 | 17 | (53.1%) | 11 | (34.4%) | 1 | (3.1%) |
| Days 29-42 | Hot Flush | A | 14 | 0 | (0.0%) | 0 | (0.0%) | 0 | (0.0%) |
| | | B | 15 | 2 | (13.3%) | 3 | (20.0%) | 0 | (0.0%) |
| | | Overall | 29 | 2 | (6.9%) | 3 | (10.3%) | 0 | (0.0%) |
| | No Bleeding | A | 14 | 1 | (7.1%) | 7 | (50.0%) | 3 | (21.4%) |
| | | B | 15 | 3 | (20.0%) | 5 | (33.3%) | 2 | (13.3%) |
| | | Overall | 29 | 4 | (13.8%) | 12 | (41.4%) | 5 | (17.2%) |
| | No Bleeding or Spotting | A | 14 | 1 | (7.1%) | 9 | (64.3%) | 3 | (21.4%) |
| | | B | 15 | 3 | (20.0%) | 7 | (46.7%) | 3 | (20.0%) |
| | | Overall | 29 | 4 | (13.8%) | 16 | (55.2%) | 6 | (20.7%) |
| Days 43-56 | Hot Flush | A | 14 | 0 | (0.0%) | 0 | (0.0%) | 0 | (0.0%) |
| | | B | 15 | 1 | (6.7%) | 0 | (0.0%) | 0 | (0.0%) |
| | | Overall | 29 | 1 | (3.4%) | 0 | (0.0%) | 0 | (0.0%) |
| | No Bleeding | A | 14 | 1 | (7.1%) | 7 | (50.0%) | 3 | (21.4%) |
| | | B | 15 | 2 | (13.3%) | 4 | (26.7%) | 1 | (6.7%) |
| | | Overall | 29 | 3 | (10.3%) | 11 | (37.9%) | 4 | (13.8%) |
| | No Bleeding or Spotting | A | 14 | 1 | (7.1%) | 8 | (57.1%) | 3 | (21.4%) |
| | | B | 15 | 7 | (20.0%) | 7 | (46.7%) | 2 | (13.3%) |
| | | Overall | 29 | 4 | (13.8%) | 15 | (51.7%) | 5 | (17.2%) |
| Days 57-70 | Hot Flush | A | 14 | 0 | (0.0%) | 0 | (0.0%) | 0 | (0.0%) |
| | | B | 15 | 0 | (0.0%) | 0 | (0.0%) | 0 | (0.0%) |
| | | Overall | 29 | 0 | (0.0%) | 0 | (0.0%) | 0 | (0.0%) |
| | No Bleeding | A | 14 | 3 | (21.4%) | 6 | (42.9%) | 3 | (21.4%) |
| | | B | 15 | 3 | (20.0%) | 6 | (40.0%) | 0 | (0.0%) |
| | | Overall | 29 | 6 | (20.7%) | 12 | (41.4%) | 3 | (10.3%) |
| | No Bleeding or Spotting | A | 14 | 3 | (21.4%) | 7 | (50.0%) | 3 | (21.4%) |
| | | B | 15 | 3 | (20.0%) | 7 | (46.7%) | 1 | (6.7%) |
| | | Overall | 29 | 6 | (20.7%) | 14 | (48.3%) | 4 | (13.8%) |

### CONCLUSIONS

Taken together, the results of these experiments demonstrate that concurrent onset of GnRH antagonist therapy and add-back therapy results in a more efficacious and controlled reduction in endogenous E2 levels, and indicate that dosing regimens employing a simultaneous onset of administration of these agents provide the benefit of effectively treating estrogen-dependent diseases without reducing estrogen to levels so low as to trigger undesirable side effects. That a concurrent onset of administration of GnRH antagonist therapy and add-back therapy engenders superior E2 reduction and more markedly diminished uterine blood loss relative to instances in which a subject is pre-treated with a GnRH antagonist is particularly unexpected. Since post-menopausal subjects, which exhibit endogenous E2 levels similar to pre-menopausal subjects that are treated with GnRH antagonist therapy alone, maintain low uterine bleeding patterns upon administration of add-back therapy, one might have concluded that pre-treatment with a GnRH antagonist prior to commencement of add-back therapy would result in more substantial reductions in E2, and thus, higher rates of amenorrhea. The results of the experiments described herein demonstrate that, surprisingly, the reverse is true.

In view of these findings, the compositions and methods of the present disclosure may be used to treat a variety of estrogen-dependent disorders. Examples 2-4, below, describe how a GnRH antagonist, such as a GnRH antagonist disclosed herein, may be used in combination with add-back therapy for the treatment of a few of these diseases.

### Example 2. Use of a GnRH Antagonist for the Treatment of a Patient Having Uterine Fibroids

Using the compositions and methods described herein, a patient may be administered a GnRH antagonist so as to treat, and/or ameliorate the symptoms of, uterine fibroids. The GnRH antagonist (e.g., a compound of formula (I), above, such as compound (VI) or the choline salt thereof) may be administered to the patient in an amount sufficient to reduce the serum concentration of LH, FSH, and/or E2 in circulation. The GnRH antagonist may be administered, for example, in an amount of from about 25 mg to about 500 mg per dose. Exemplary doses of the GnRH antagonist include, without limitation, an amount of from 25 mg to 500 mg, 30 mg to 495 mg, 35 mg to 490 mg, 40 mg to 485 mg, 45 mg to 480 mg, 50 mg to 475 mg, 55 mg to 470 mg, 60 mg to 465 mg, 65 mg to 460 mg, 70 mg to 455 mg, 75 mg to 450 mg, 80 mg to 445 mg, 85 mg to 440 mg, 90 mg to 435 mg, 95 mg to 430 mg, 100 mg to 425 mg, 105 mg to 420 mg, 110 mg to 415 mg, 115 mg to 410 mg, 120 mg to 405 mg, 125 mg to 400 mg, 130 mg to 395 mg, 135 mg to 390 mg, 140 mg to 385 mg, 145 mg to 380 mg, or 150 mg to 375 mg, per dose. For example, when the GnRH antagonist is compound (VI) or the choline salt thereof, the compound may be administered at a dose of 200 mg.

The GnRH antagonist may be administered to the patient periodically, for instance, over a treatment period of at least two weeks. The GnRH antagonist may be provided to the patient in combination with add-back therapy, such that administration of the GnRH antagonist therapy and the add-back therapy commence at substantially the same time (e.g., within from about 1 to about 48 hours of one another, such as on the same day). To determine the responsiveness of the patient to the GnRH antagonist, a physician may monitor the time required for the patient to achieve a reduction in uterine blood loss, such as the time required to achieve amenorrhea. For example, the physician may observe one or more of the following responses of the patient as an indicator of successful treatment: (i) a reduction in menstrual blood loss to less than 80 ml per menstrual cycle, such that the patient no longer exhibits heavy menstrual blood loss, the volume of one or more rectovaginal endometriosis nodes following administration of the GnRH antagonist to the patient, and/or (ii) an improvement in the patient's overall well-being as determined by an improvement in the patient's Endometriosis Health Profile questionnaire (EHP-30) score following administration of the GnRH antagonist to the patient and/or by way of a positive Patient Global Impression of Change (PGIC) score following administration of the GnRH antagonist to the patient.

### Example 3. Use of a GnRH Antagonist for the Treatment of a Patient Having Endometriosis

Using the compositions and methods described herein, a patient may be administered a GnRH antagonist so as to treat, and/or ameliorate the symptoms of, endometriosis (e.g., rectovaginal endometriosis). The GnRH antagonist (e.g., a compound of formula (I), above, such as compound (VI) or the choline salt thereof) may be administered to the patient in an amount sufficient to reduce the serum concentration of LH, FHS, and/or E2 in circulation. The GnRH antagonist may be administered, for example, in an amount of from about 25 mg to about 500 mg per dose. Exemplary doses of the GnRH antagonist include, without limitation, an amount of from 25 mg to 500 mg, 30 mg to 495 mg, 35 mg to 490 mg, 40 mg to 485 mg, 45 mg to 480 mg, 50 mg to 475 mg, 55 mg to 470 mg, 60 mg to 465 mg, 65 mg to 460 mg, 70 mg to 455 mg, 75 mg to 450 mg, 80 mg to 445 mg, 85 mg to 440 mg, 90 mg to 435 mg, 95 mg to 430 mg, 100 mg to 425 mg, 105 mg to 420 mg, 110 mg to 415 mg, 115 mg to 410 mg, 120 mg to 405 mg, 125 mg to 400 mg, 130 mg to 395 mg, 135 mg to 390 mg, 140 mg to 385 mg, 145 mg to 380 mg, or 150 mg to 375 mg, per dose. For example, when the GnRH antagonist is compound (VI) or the choline salt thereof, the compound may be administered at a dose of 200 mg.

The GnRH antagonist may be administered to the patient periodically, for instance, over a treatment period of at least two weeks. The GnRH antagonist may be provided to the patient in combination with add-back therapy, such that administration of the GnRH antagonist therapy and the add-back therapy commence at substantially the same time (e.g., within from about 1 to about 48 hours of one another, such as on the same day). To determine the responsiveness of the patient to the GnRH antagonist, a physician may monitor the length of one or more endometriosis lesions (e.g., rectovaginal endometriosis lesions) in the patient, for example, by way of magnetic resonance imaging (MRI) and/or transvaginal ultrasound (TVUS). For example, the physician may observe one or more of the following responses of the patient as an indicator of successful treatment: (i) a reduction in the volume of one or more rectovaginal endometriosis nodes following administration of the GnRH antagonist to the patient, (ii) a reduction in bowel involvement of one or more type III endometriosis nodes following administration of the GnRH antagonist to the patient, (iii) a reduction in pelvic pain following administration of the GnRH antagonist to the patient; (iv) a reduction in dysmenorrhea following administration of the GnRH antagonist to the patient; (v) a reduction in dyspareunia following administration of the GnRH antagonist to the patient; (vi) a reduction in dyschezia following administration of the GnRH antagonist to the patient; (vii) a reduction in uterine bleeding following administration of the GnRH antagonist to the patient; (viii) achievement of amenorrhea following administration of the GnRH antagonist to the patient; and (xi) an improvement in the patient's overall well-being as determined by an improvement in the patient's EHP-30 score following administration of the GnRH antagonist to the patient and/or by way of a positive PGIC score following administration of the GnRH antagonist to the patient.

### Example 4. Use of a GnRH Antagonist for the Treatment of a Patient Having Adenomyosis

Using the compositions and methods described herein, a patient may be administered a GnRH antagonist so as to treat, and/or ameliorate the symptoms of, adenomyosis. The GnRH antagonist (e.g., a compound of formula (I), above, such as compound (VI) or the choline salt thereof) may be administered to the patient in an amount sufficient to reduce the serum concentration of luteinizing hormone (LH), follicle-stimulating hormone (FHS), and/or β17-estradiol (E2) in circulation. The GnRH antagonist may be administered, for example, in an amount of from about 25 mg to about 500 mg per dose. Exemplary doses of the GnRH antagonist include, without limitation, an amount of from 25 mg to 500 mg, 30 mg to 495 mg, 35 mg to 490 mg, 40 mg to 485 mg, 45 mg to 480 mg, 50 mg to 475 mg, 55 mg to 470 mg, 60 mg to 465 mg, 65 mg to 460 mg, 70 mg to 455 mg, 75 mg to 450 mg, 80 mg to 445 mg, 85 mg to 440 mg, 90 mg to 435 mg, 95 mg to 430 mg, 100 mg to 425 mg, 105 mg to 420 mg, 110 mg to 415 mg, 115 mg to 410 mg, 120 mg to 405 mg, 125 mg to 400 mg, 130 mg to 395 mg, 135 mg to 390 mg, 140 mg to 385 mg, 145 mg to 380 mg, or 150 mg to 375 mg, per dose. For example, when the GnRH antagonist is compound (VI) or the choline salt thereof, the compound may be administered at a dose of 200 mg.

The GnRH antagonist may be administered to the patient periodically, for instance, over a treatment period of at least two weeks. The GnRH antagonist may be provided to the patient in combination with add-back therapy, such that administration of the GnRH antagonist therapy and the add-back therapy commence at substantially the same time (e.g., within from about 1 to about 48 hours of one another, such as on the same day). To determine the responsiveness of the patient to the GnRH antagonist, a physician may monitor the patient's uterine volume, as well as the level of pain experienced by the patient. For example, the physician may observe one or more of the following responses of the patient as an indicator of successful treatment: (i) a reduction in uterine volume following administration of the GnRH antagonist to the patient, (ii) a reduction in the thickness of the anterior and/or posterior region of the uterine myometrium following administration of the GnRH antagonist to the patient, (iii) a reduction in pelvic pain following administration of the GnRH antagonist to the patient; (iv) a reduction in dysmenorrhea following administration of the GnRH antagonist to the patient; (v) a reduction in dyspareunia following administration of the GnRH antagonist to the patient; (vi) a reduction in dyschezia following administration of the GnRH antagonist to the patient; (vii) a reduction in uterine tenderness following administration of the GnRH antagonist to the patient; (viii) a reduction in uterine bleeding following administration of the GnRH antagonist to the patient; (ix) achievement of amenorrhea following administration of the GnRH antagonist to the patient; (x) a reduction in the diameter of a junctional zone of adenomyosis following administration of the GnRH antagonist to the patient; and (xi) an improvement in the patient's overall well-being as determined by an improvement in the patient's EHP-30 score following administration of the GnRH antagonist to the patient and/or by way of a positive PGIC score following administration of the GnRH antagonist to the patient.

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A method of treating an estrogen-dependent disease in a human patient in need thereof, the method comprising periodically administering to the patient therapeutically effective amounts of a gonadotropin-releasing hormone (GnRH) antagonist and add-back therapy, wherein administration of the GnRH antagonist and administration of the add-back therapy begin on the same day.

2. The method of claim 1, wherein the estrogen-dependent disease is uterine fibroids.

3. A method of reducing the volume of menstrual blood loss a human patient diagnosed as having uterine fibroids, the method comprising periodically administering to the patient therapeutically effective amounts of a GnRH antagonist and add-back therapy, wherein administration of the GnRH antagonist and administration of the add-back therapy begin on the same day.

4. A method of inducing amenorrhea in a human patient diagnosed as having uterine fibroids, the method comprising periodically administering to the patient therapeutically effective amounts of a GnRH antagonist and add-back therapy, wherein administration of the GnRH antagonist and administration of the add-back therapy begin on the same day.

5. A method of reducing the volume of one or more uterine fibroids in a human patient diagnosed as having uterine fibroids, the method comprising periodically administering to the patient therapeutically effective amounts of a GnRH antagonist and add-back therapy, wherein administration of the GnRH antagonist and administration of the add-back therapy begin on the same day.

6. The method of claim 1, wherein the estrogen-dependent disease is endometriosis.

7. A method of reducing the volume of one or more endometriosis nodes in a human patient diagnosed as having endometriosis, the method comprising periodically administering to the patient therapeutically effective amounts of a GnRH antagonist and add-back therapy, wherein administration of the GnRH antagonist and administration of the add-back therapy begin on the same day.

8. A method of reducing pelvic pain in a human patient diagnosed as having endometriosis, the method comprising periodically administering to the patient therapeutically effective amounts of a GnRH antagonist and add-back therapy, wherein administration of the GnRH antagonist and administration of the add-back therapy begin on the same day.

9. A method of reducing dysmenorrhea in a human patient diagnosed as having endometriosis, the method comprising periodically administering to the patient therapeutically effective amounts of a GnRH antagonist and add-back therapy, wherein administration of the GnRH antagonist and administration of the add-back therapy begin on the same day.

10. A method of reducing dyspareunia in a human patient diagnosed as having endometriosis, the method comprising periodically administering to the patient therapeutically effective amounts of a GnRH antagonist and add-back therapy, wherein administration of the GnRH antagonist and administration of the add-back therapy begin on the same day.

11. A method of reducing dyschezia in a human patient diagnosed as having endometriosis, the method comprising periodically administering to the patient therapeutically effective amounts of a GnRH antagonist and add-back therapy, wherein administration of the GnRH antagonist and administration of the add-back therapy begin on the same day.

12. A method of reducing uterine bleeding in a human patient diagnosed as having endometriosis, the method comprising periodically administering to the patient therapeutically effective amounts of a GnRH antagonist and add-back therapy, wherein administration of the GnRH antagonist and administration of the add-back therapy begin on the same day.

13. A method of inducing amenorrhea in a human patient diagnosed as having endometriosis, the method comprising periodically administering to the patient therapeutically effective amounts of a GnRH antagonist and add-back therapy, wherein administration of the GnRH antagonist and administration of the add-back therapy begin on the same day.

14. The method of claim 1, wherein the estrogen-dependent disease is adenomyosis.

15. A method of reducing uterine volume in a human patient diagnosed as having adenomyosis, the method comprising periodically administering to the patient therapeutically effective amounts of a GnRH antagonist and add-back therapy, wherein administration of the GnRH antagonist and administration of the add-back therapy begin on the same day.

16. A method of reducing the thickness of the anterior and/or posterior region of the uterine myometrium in a human patient diagnosed as having adenomyosis, the method comprising periodically administering to the patient therapeutically effective amounts of a GnRH antagonist and add-back therapy, wherein administration of the GnRH antagonist and administration of the add-back therapy begin on the same day.

17. A method of reducing pelvic pain in a human patient diagnosed as having adenomyosis, the method comprising periodically administering to the patient therapeutically effective amounts of a GnRH antagonist and add-back therapy, wherein administration of the GnRH antagonist and administration of the add-back therapy begin on the same day.

18. A method of reducing dysmenorrhea in a human patient diagnosed as having adenomyosis, the method comprising periodically administering to the patient therapeutically effective amounts of a GnRH antagonist and add-back therapy, wherein administration of the GnRH antagonist and administration of the add-back therapy begin on the same day.

19. A method of reducing dyspareunia in a human patient diagnosed as having adenomyosis, the method comprising periodically administering to the patient therapeutically effective amounts of a GnRH antagonist and add-back therapy, wherein administration of the GnRH antagonist and administration of the add-back therapy begin on the same day.

20. A method of reducing dyschezia in a human patient diagnosed as having adenomyosis, the method comprising periodically administering to the patient therapeutically effective amounts of a GnRH antagonist and add-back therapy, wherein administration of the GnRH antagonist and administration of the add-back therapy begin on the same day.

21. A method of reducing uterine tenderness in a human patient diagnosed as having adenomyosis, the method comprising periodically administering to the patient therapeutically effective amounts of a GnRH antagonist and add-back therapy, wherein administration of the GnRH antagonist and administration of the add-back therapy begin on the same day.

22. A method of reducing uterine bleeding in a human patient diagnosed as having adenomyosis, the method comprising periodically administering to the patient therapeutically effective amounts of a GnRH antagonist and add-back therapy, wherein administration of the GnRH antagonist and administration of the add-back therapy begin on the same day.

23. A method of inducing amenorrhea in a human patient diagnosed as having adenomyosis, the method comprising periodically administering to the patient therapeutically effective amounts of a GnRH antagonist and add-back therapy, wherein administration of the GnRH antagonist and administration of the add-back therapy begin on the same day.

24. The method of claim 1, wherein the estrogen-dependent disease is rectovaginal endometriosis.

25. A method of reducing the volume of one or more rectovaginal endometriosis nodes in a human patient diagnosed as having rectovaginal endometriosis, the method comprising periodically administering to the patient therapeutically effective amounts of a GnRH antagonist and add-back therapy, wherein administration of the GnRH antagonist and administration of the add-back therapy begin on the same day.

26. A method of reducing the volume of one or more type III rectovaginal endometriosis nodes in a human patient diagnosed as having rectovaginal endometriosis, the method comprising periodically administering to the patient therapeutically effective amounts of a GnRH antagonist and add-back therapy, wherein administration of the GnRH antagonist and administration of the add-back therapy begin on the same day.

27. A method of reducing pelvic pain in a human patient diagnosed as having rectovaginal endometriosis, the method comprising periodically administering to the patient therapeutically effective amounts of a GnRH antagonist and add-back therapy, wherein administration of the GnRH antagonist and administration of the add-back therapy begin on the same day.

28. A method of reducing dysmenorrhea in a human patient diagnosed as having rectovaginal endometriosis, the method comprising periodically administering to the patient therapeutically effective amounts of a GnRH antagonist and add-back therapy, wherein administration of the GnRH antagonist and administration of the add-back therapy begin on the same day.

29. A method of reducing dyspareunia in a human patient diagnosed as having rectovaginal endometriosis, the method comprising periodically administering to the patient therapeutically effective amounts of a GnRH antagonist and add-back therapy, wherein administration of the GnRH antagonist and administration of the add-back therapy begin on the same day.

30. A method of reducing dyschezia in a human patient diagnosed as having rectovaginal endometriosis, the method comprising periodically administering to the patient therapeutically effective amounts of a GnRH antagonist and add-back therapy, wherein administration of the GnRH antagonist and administration of the add-back therapy begin on the same day.

31. A method of reducing uterine bleeding in a human patient diagnosed as having rectovaginal endometriosis, the method comprising periodically administering to the patient therapeutically effective amounts of a GnRH antagonist and add-back therapy, wherein administration of the GnRH antagonist and administration of the add-back therapy begin on the same day.

32. A method of inducing amenorrhea in a human patient diagnosed as having rectovaginal endometriosis, the method comprising periodically administering to the patient therapeutically effective amounts of a GnRH antagonist and add-back therapy, wherein administration of the GnRH antagonist and administration of the add-back therapy begin on the same day.

33. The method of any one of claims 1-32, wherein the GnRH antagonist is a compound represented by formula (I) 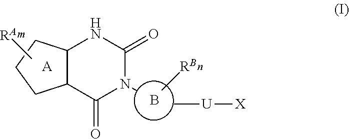
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof.

34. The method of claim 33, wherein the ring A is a thiophene ring represented by formula (IIa)

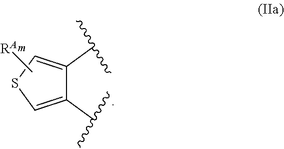

35. The method of claim 33 or 34, wherein m is 1.

36. The method of claim 35, wherein the ring A is an optionally substituted thiophene ring represented by formula (IIb)

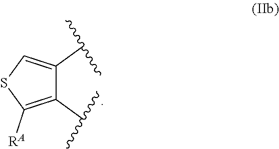

37. The method of any one of claims 33-36, wherein each R^{A} is independently a halogen atom, an optionally substituted lower alkyl group, COOW¹, or CONW²W³, wherein W¹ to W³ independently are a hydrogen atom or an optionally substituted lower alkyl group, or W² and W³ may bind together with the neighboring nitrogen atom to form an optionally substituted cyclic amino group.

38. The method of claim 37, wherein each R^{A} is COOH or pharmaceutically acceptable salt thereof.

39. The method of any one of claims 33-38, wherein the ring B is an optionally substituted benzene ring, pyridine ring, or thiophene ring.

40. The method of claim 39, wherein the ring B is represented by a formula selected from the group consisting of:

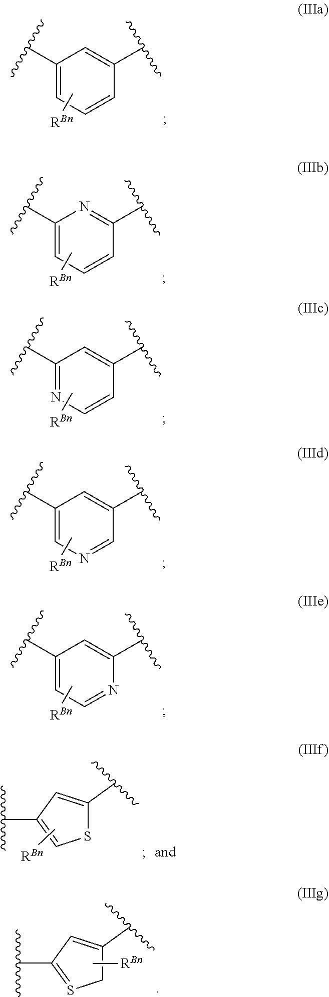

41. The method of any one of claims 33-40, wherein n is 2.

42. The method of claim 41, wherein the ring B is represented by a formula selected from the group consisting of:

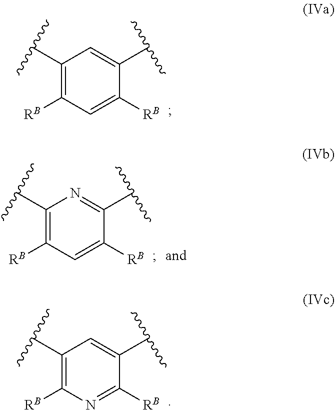

43. The method of any one of claims 33-42, wherein each R^{B} is independently a halogen atom, an optionally substituted lower alkyl group, or OW⁴, wherein each W⁴ is independently a hydrogen atom or an optionally substituted lower alkyl group.

44. The method of claim 43, wherein each R^{B} is independently a fluorine atom, chlorine atom, bromine atom, methyl group, or methoxy group.

45. The method of any one of claims 33-44, wherein U is a single bond.

46. The method of any one of claims 33-45, wherein X is a group represented by —O-L-Y.

47. The method of any one of claims 33-46, wherein L is a methylene group.

48. The method of any one of claims 33-47, wherein Y is an optionally substituted benzene ring represented by formula (V) 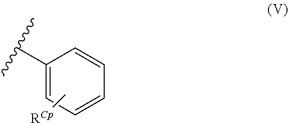
wherein each R^{C} is independently a halogen atom, an optionally substituted lower alkyl group, or OW⁹, wherein each W⁹ is independently a hydrogen atom or an optionally substituted lower alkyl group; and
p is an integer from 0 to 3.

49. The method of claim 48, wherein Y is a substituted benzene ring represented by formula (Va)

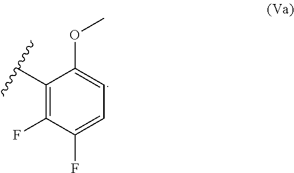 [SMILES: *c1c(OC)ccc(F)c1F]

50. The method of claim 33, wherein the compound is represented by formula (Ia) 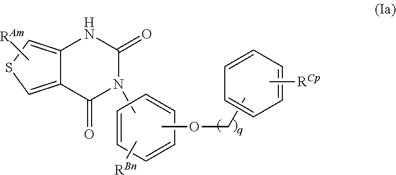
wherein each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W³ to W³ independently are a hydrogen atom or an optionally substituted lower alkyl group, or W² and W³ may bind together with the neighboring nitrogen atom to form an optionally substituted cyclic amino group;
m is an integer from 0 to 3;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
q is an integer from 0 to 3;
each R^{C} is independently a halogen atom, an optionally substituted lower alkyl group, or OW⁹, wherein each W⁹ is independently a hydrogen atom or an optionally substituted lower alkyl group; and
p is an integer from 0 to 3;
or a pharmaceutically acceptable salt thereof.

51. The method of claim 50, wherein the compound is represented by formula (Ib)

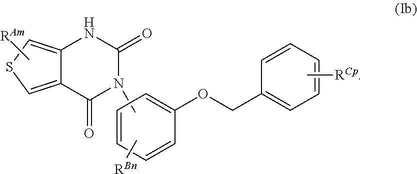

52. The method of claim 51, wherein the compound is represented by formula (Ic) 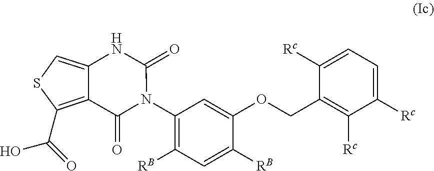
or a pharmaceutically acceptable salt thereof.

53. The method of claim any one of claims 33-52, wherein the compound is represented by formula (VI) 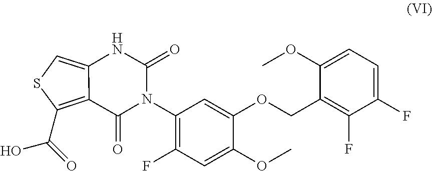 [SMILES: COc1cc(F)c(-n2c(=O)[nH]c3csc(C(=O)O)c3c2=O)cc1OCc1c(OC)ccc(F)c1F]
or a pharmaceutically acceptable salt thereof.

54. The method of claim 53, wherein the compound is administered to the patient in the form of the choline salt of (VI), choline 3-[2-fluoro-5-(2,3-difluoro-6-methoxybenzyloxy)4-methoxyphenyl]-2,4-dioxo-1,2,3,4-tetrahydrothieno [3,4d]pyrimidine-5-carboxylate.

55. The method of claim 54, wherein the compound is in a crystalline state.

56. The method of claim 55, wherein the compound exhibits characteristic X-ray powder diffraction (XRPD) peaks at about 7.1° 2θ, about 11.5° 2θ, about 19.4° 2θ, about 21.5° 2θ, about 22.0° 2θ, about 22.6° 2θ, about 23.5° 2θ, and about 26.2θ.

57. The method of claim 55 or 56, wherein the compound exhibits ¹³C solid-state nuclear magnetic resonance (NMR) peaks centered at about 55.5 ppm, about 57.1 ppm, about 58.7 ppm, about 69.8 ppm, about 98.1 ppm, about 110.3 ppm, about 111.6 ppm, about 113.7 ppm, about 118.0 ppm, about 145.3 ppm, about 149.8 ppm, and about 155.8 ppm.

58. The method of any one of claims 55-57, wherein the compound exhibits ¹⁹F solid-state NMR peaks centered at about −151.8 ppm, −145.2 ppm, and −131.6 ppm.

59. The method of any one of claims 33-58, wherein the compound is orally administered to the patient.

60. The method of any one of claims 33-59, wherein the compound is administered to the patient one or more times per day, week, or month.

61. The method of claim 60, wherein the compound is administered to the patient one or more times daily.

62. The method of claim 61, wherein the compound is administered to the patient once daily.

63. The method of any one of claims 60-62, wherein the compound is administered to the patient in an amount of from about 25 mg per day to about 400 mg per day.

64. The method of claim 63, wherein the compound is administered to the patient in an amount of from about 35 mg to about 65 mg per day.

65. The method of claim 64, wherein the compound is administered to the patient in an amount of about 50 mg per day.

66. The method of claim 63, wherein the compound is administered to the patient in an amount of from about 60 mg per day to about 90 mg per day.

67. The method of claim 66, wherein the compound is administered to the patient in an amount of about 75 mg per day.

68. The method of claim 63, wherein the compound is administered to the patient in an amount of from about 85 mg to about 115 mg per day.

69. The method of claim 68, wherein the compound is administered to the patient in an amount of about 100 mg per day.

70. The method of claim 63, wherein the compound is administered to the patient in an amount of from about 185 mg to about 215 mg per day.

71. The method of claim 70, wherein the compound is administered to the patient in an amount of about 200 mg per day.

72. The method of any one of claims 60-71, wherein the compound and add-back therapy are administered to the patient over a treatment period of at least four weeks.

73. The method of claim 72, wherein the compound and add-back therapy are administered to the patient over a treatment period of at least eight weeks.

74. The method of claim 73, wherein the compound and add-back therapy are administered to the patient over a treatment period of at least 10 weeks.

75. The method of claim 74, wherein the compound and add-back therapy are administered to the patient over a treatment period of at least 12 weeks.

76. The method of claim 75, wherein the compound and add-back therapy are administered to the patient over a treatment period of at least 24 weeks.

77. The method of any one of claims 60-71, wherein the compound and add-back therapy are administered to the patient over a treatment period of from about four weeks to about 48 months.

78. The method of claim 77, wherein the compound and add-back therapy are administered to the patient over a treatment period of from about five weeks to about 36 months.

79. The method of claim 78, wherein the compound and add-back therapy are administered to the patient over a treatment period of from about six weeks to about 24 months.

80. The method of claim 79, wherein the compound and add-back therapy are administered to the patient over a treatment period of from about seven weeks to about 12 months.

81. The method of claim 80, wherein the compound and add-back therapy are administered to the patient over a treatment period of from about eight weeks to about six months.

82. The method of any one of claims 60-71, wherein the compound and add-back therapy are administered to the patient over a treatment period of at least from about four weeks to about 12 months.

83. The method of claim 82, wherein the compound and add-back therapy are administered to the patient over a treatment period of at least from about five weeks to about 24 weeks.

84. The method of claim 83, wherein the compound and add-back therapy are administered to the patient over a treatment period of at least from about six weeks to about 18 wees.

85. The method of claim 84, wherein the compound and add-back therapy are administered to the patient over a treatment period of at least from about seven weeks to about 14 weeks.

86. The method of claim 85, wherein the compound and add-back therapy are administered to the patient over a treatment period of at least from about eight weeks to about 12 weeks.

87. The method of any one of claims 1-32, wherein the GnRH antagonist is elagolix, relugolix, opigolix (ASP1707), BAY-784, or SK-2706.

88. The method of claim 87, wherein the GnRH antagonist is elagolix.

89. The method of claim 88, wherein the GnRH antagonist is orally administered to the patient.

90. The method of claim 88 or 89, wherein the GnRH antagonist is administered to the patient one or more times per day, week, or month.

91. The method of claim 90, wherein the GnRH antagonist is administered to the patient one or more times daily.

92. The method of claim 91, wherein the GnRH antagonist is administered to the patient once daily.

93. The method of any one of claims 88-92, wherein the GnRH antagonist is administered to the patient in an amount of from about 100 mg to about 600 mg per day.

94. The method of claim 93, wherein the GnRH antagonist is administered to the patient in an amount of about 150 mg per day.

95. The method of claim 93, wherein the GnRH antagonist is administered to the patient in an amount of about 300 mg per day.

96. The method of claim 93, wherein the GnRH antagonist is administered to the patient in an amount of about 400 mg per day, optionally wherein the GnRH antagonist is administered to the patient in two daily doses of 200 mg per dose.

97. The method of claim 93, wherein the GnRH antagonist is administered to the patient in an amount of about 600 mg per day, optionally wherein the GnRH antagonist is administered to the patient in two daily doses of 300 mg per dose.

98. The method of claim 87, wherein the GnRH antagonist is relugolix.

99. The method of claim 98, wherein the GnRH antagonist is orally administered to the patient.

100. The method of claim 98 or 99, wherein the GnRH antagonist is administered to the patient one or more times per day, week, or month.

101. The method of claim 100, wherein the GnRH antagonist is administered to the patient one or more times daily.

102. The method of claim 101, wherein the GnRH antagonist is administered to the patient once daily.

103. The method of any one of claims 98-102, wherein the GnRH antagonist is administered to the patient in an amount of from about 10 mg to about 60 mg per day.

104. The method of claim 103, wherein the GnRH antagonist is administered to the patient in an amount of from about 20 mg to about 50 mg per day.

105. The method of claim 104, wherein the GnRH antagonist is administered to the patient in an amount of about 40 mg per day.

106. The method of any one of claims 1-105, wherein the add-back therapy is administered to the patient one or more times daily.

107. The method of claim 106, wherein the add-back therapy is administered to the patient once daily, concurrently with the GnRH antagonist.

108. The method of claim 106, wherein the add-back therapy is administered to the patient once daily, prior to administration of the GnRH antagonist.

109. The method of claim 106, wherein the add-back therapy is administered to the patient once daily, following administration of the GnRH antagonist.

110. The method of claim 107, wherein the add-back therapy is administered to the patient in the form of a pharmaceutical composition comprising the GnRH antagonist.

111. The method of any one of claims 1-110, wherein the add-back therapy comprises an estrogen.

112. The method of claim 111, wherein the estrogen is selected from the group consisting of β17-estradiol, ethinyl estradiol, and conjugated estrogens.

113. The method of claim 112, wherein the estrogen is β17-estradiol.

114. The method of claim 113, wherein the β17-estradiol is administered to the patient in an amount of about 1.0 mg/day.

115. The method of claim 113, wherein the β17-estradiol is administered to the patient in an amount of about 0.5 mg/day.

116. The method of claim 112, wherein the estrogen is ethinyl estradiol.

117. The method of claim 116, wherein the ethinyl estradiol is administered to the patient in an amount of about 5.0 μg/day.

118. The method of claim 116, wherein the ethinyl estradiol is administered to the patient in an amount of about 2.5 μg/day.

119. The method of claim 112, wherein the estrogen is a conjugated estrogen.

120. The method of claim 119, wherein the conjugated estrogen is administered to the patient in an amount of about 0.625 mg/day.

121. The method of claim 119, wherein the conjugated estrogen is administered to the patient in an amount of about 0.45 mg/day.

122. The method of claim 119, wherein the conjugated estrogen is administered to the patient in an amount of about 0.3 mg/day

123. The method of any one of claims 1-122, wherein the add-back therapy comprises a progestin.

124. The method of claim 123, wherein the progestin is selected from the group consisting of norethindrone or an ester thereof, progesterone, norgestimate, medroxyprogesterone, and drospirenone.

125. The method of claim 124, wherein the progestin is norethindrone or norethindrone acetate.

126. The method of claim 125, wherein the norethindrone or norethindrone acetate is administered to the patient in an amount of about 1.0 mg/day.

127. The method of claim 125, wherein the norethindrone or norethindrone acetate is administered to the patient in an amount of about 0.5 mg/day.

128. The method of claim 125, wherein the norethindrone or norethindrone acetate is administered to the patient in an amount of about 0.1 mg/day.

129. The method of claim 124, wherein the progestin is progesterone.

130. The method of claim 129, wherein the progesterone is administered to the patient in an amount of about 200 mg/day.

131. The method of claim 129, wherein the progesterone is administered to the patient in an amount of about 100 mg/day.

132. The method of claim 124, wherein the progestin is norgestimate.

133. The method of claim 132, wherein the norgestimate is administered to the patient in an amount of about 0.09 mg/day.

134. The method of claim 124, wherein the progestin is medroxyprogesterone.

135. The method of claim 134, wherein the medroxyprogesterone is administered to the patient in an amount of about 5 mg/day.

136. The method of claim 134, wherein the medroxyprogesterone is administered to the patient in an amount of about 2.5 mg/day.

137. The method of claim 134, wherein the medroxyprogesterone is administered to the patient in an amount of about 1.5 mg/day.

138. The method of claim 124, wherein the progestin is drospirenone.

139. The method of claim 138, wherein the drospirenone is administered to the patient in an amount of about 0.5 mg/day.

140. The method of claim 138, wherein the drospirenone is administered to the patient in an amount of about 0.25 mg/day.

141. The method of any one of claims 1-140, wherein the add-back therapy comprises about 1.0 mg of β17-estradiol and about 0.5 mg of norethindrone acetate.

142. The method of any one of claims 1-140, wherein the add-back therapy comprises about 0.5 mg of β17-estradiol and about 0.1 mg of norethindrone acetate.

143. The method of any one of claims 1-142, wherein the patient is a pre-menopausal female of from about 18 to about 48 years of age.

144. The method of any one of claims 1-143, wherein the patient exhibits a serum concentration of follicle-stimulating hormone (FSH) of about 20 IU/L or less prior to administration of the GnRH antagonist to the patient.

145. The method of any one of claims 1-144, wherein the patient exhibits a rectal (type II) and/or vaginal (type III) endometriosis node of at least 2 cm prior to administration of the GnRH antagonist to the patient.

146. The method of claim 145, wherein the length of the type II and/or type III endometriosis node is assessed by way of magnetic resonance imaging (MRI).

147. The method of any one of claims 1-146, wherein the patient exhibits a junctional-zone width of about 12 mm or more prior to administration of the GnRH antagonist to the patient.

148. The method of claim 147, wherein the junctional-zone width is assessed by way of MRI.

149. The method of any one of claims 1-148, wherein the patient exhibits a reduction in serum concentration of luteinizing hormone (LH), follicle-stimulating hormone (FSH), and/or β17-estradiol (E2) following administration of the GnRH antagonist to the patient.

150. The method of claim 149, wherein the patient exhibits the reduction in serum concentration of LH, FSH, and/or E2 within from about one day to about 36 weeks of the first administration of the GnRH antagonist to the patient, optionally wherein the patient exhibits the reduction within from about 12 weeks to about 24 weeks of the first administration of the GnRH antagonist to the patient.

151. The method of any one of claims 1, 2, 5-11, 14-21, 24-30, and 33-150, wherein the patient exhibits a reduction in uterine bleeding following administration of the GnRH antagonist to the patient.

152. The method of claim 151, wherein the patient exhibits the reduction in uterine bleeding within from about one day to about 36 weeks of the first administration of the GnRH antagonist to the patient, optionally wherein the patient exhibits the reduction within from about 12 weeks to about 24 weeks of the first administration of the GnRH antagonist to the patient.

153. The method of any one of claims 3, 4, 12, 13, 22, 23, 31, 32, 151, and 152, wherein the reduction in uterine bleeding is assessed by way of an alkaline hematin method.

154. The method of any one of claims 1-3, 5-12, 14-22, 24-31, and 33-153, wherein the patient exhibits amenorrhea following administration of the GnRH antagonist to the patient.

155. The method of claim 154, wherein the patient exhibits the amenorrhea within from about one day to about 36 weeks of the first administration of the GnRH antagonist to the patient, optionally wherein the patient exhibits the amenorrhea within from about 12 weeks to about 24 weeks of the first administration of the GnRH antagonist to the patient.

156. The method of any one of claims 1-24 and 27-155, wherein the patient exhibits a reduction in the volume of one or more rectovaginal endometiosis nodes following administration of the GnRH antagonist to the patient.

157. The method of claim 156, wherein the patient exhibits the reduction in volume of the one or more rectovaginal endometriosis nodes within from about one day to about 36 weeks of the first administration of the GnRH antagonist to the patient, optionally wherein the patient exhibits the reduction within from about 12 weeks to about 24 weeks of the first administration of the GnRH antagonist to the patient.

158. The method of any one of claims 26, 156, and 157, wherein the reduction in volume of the one or more rectovaginal endometriosis nodes is assessed by way of MRI or TVUS.

159. The method of any one of claims 1-158, wherein the patient exhibits a reduction in bowel involvement of one or more type III endometriosis nodes following administration of the GnRH antagonist to the patient.

160. The method of claim 159, wherein the patient exhibits the reduction in bowel involvement of the one or more type III endometriosis nodes within from about one day to about 36 weeks of the first administration of the GnRH antagonist to the patient, optionally wherein the patient exhibits the reduction within from about 12 weeks to about 24 weeks of the first administration of the GnRH antagonist to the patient.

161. The method of any one of claims 1-7, 9-16, 18-26, and 28-160, wherein the patient exhibits a reduction in pelvic pain following administration of the GnRH antagonist to the patient.

162. The method of claim 161, wherein the patient exhibits the reduction in pelvic pain within from about one day to about 36 weeks of the first administration of the GnRH antagonist to the patient, optionally wherein the patient exhibits the reduction within from about 12 weeks to about 24 weeks of the first administration of the GnRH antagonist to the patient.

163. The method of any one of claims 8, 17, 27, 161, and 162, wherein the reduction in pelvic pain is assessed by way of a modified Biberoglu & Behrman (mB&B) score, Numerical Rating Scale (NRS) score, or Verbal Rating Scale (VRS) score.

164. The method of any one of claims 1-8, 10-17, 19-27, and 29-163, wherein the patient exhibits a reduction in dysmenorrhea following administration of the GnRH antagonist to the patient.

165. The method of claim 164, wherein the patient exhibits the reduction in dysmenorrhea within from about one day to about 36 weeks of the first administration of the GnRH antagonist to the patient, optionally wherein the patient exhibits the reduction within from about 12 weeks to about 24 weeks of the first administration of the GnRH antagonist to the patient.

166. The method of any one of claims 9, 18, 28, 164, and 165, wherein the reduction in dysmenorrhea is assessed by way of an mB&B score, NRS score, or VRS score.

167. The method of any one of claims 1-9, 11-18, 20-28, and 30-166, wherein the patient exhibits a reduction in dyspareunia following administration of the GnRH antagonist to the patient.

168. The method of claim 167, wherein the patient exhibits the reduction in dyspareunia within from about one day to about 36 weeks of the first administration of the GnRH antagonist to the patient, optionally wherein the patient exhibits the reduction within from about 12 weeks to about 24 weeks of the first administration of the GnRH antagonist to the patient.

169. The method of any one of claims 10, 19, 29, 167, and 168, wherein the reduction in dyspareunia is assessed by way of an mB&B score, NRS score, or VRS score.

170. The method of any one of claims 1-10, 12-19, 21-29, and 31-169, wherein the patient exhibits a reduction in dyschezia following administration of the GnRH antagonist to the patient.

171. The method of claim 170, wherein the patient exhibits the reduction in dyschezia within from about one day to about 36 weeks of the first administration of the GnRH antagonist to the patient, optionally wherein the patient exhibits the reduction within from about 12 weeks to about 24 weeks of the first administration of the GnRH antagonist to the patient.

172. The method of any one of claims 11, 20, 30, 170, and 171, wherein the reduction in dyschezia is assessed by way of an mB&B score, NRS score, or VRS score.

173. The method of any one of claims 1-14 and 16-172, wherein the patient exhibits a reduction in uterine volume following administration of the GnRH antagonist to the patient.

174. The method of claim 173, wherein the patient exhibits the reduction in uterine volume within from about one day to about 36 weeks of the first administration of the GnRH antagonist to the patient, optionally wherein the patient exhibits the reduction within from about 12 weeks to about 24 weeks of the first administration of the GnRH antagonist to the patient.

175. The method of any one of claims 15, 173, and 174, wherein the reduction in uterine volume is assessed by way of MRI or transvaginal ultrasound (TVUS).

176. The method of any one of claims 1-15 and 17-175, wherein the patient exhibits a reduction in the thickness of the anterior and/or posterior region of the uterine myometrium following administration of the GnRH antagonist to the patient.

177. The method of claim 176, wherein the patient exhibits the reduction in thickness of the anterior and/or posterior region of the uterine myometrium within from about one day to about 36 weeks of the first administration of the GnRH antagonist to the patient, optionally wherein the patient exhibits the reduction within from about 12 weeks to about 24 weeks of the first administration of the GnRH antagonist to the patient.

178. The method of any one of claims 1-20 and 22-177, wherein the patient exhibits a reduction in uterine tenderness following administration of the GnRH antagonist to the patient.

179. The method of claim 178, wherein the patient exhibits the reduction in uterine tenderness within from about one day to about 36 weeks of the first administration of the GnRH antagonist to the patient, optionally wherein the patient exhibits the reduction within from about 12 weeks to about 24 weeks of the first administration of the GnRH antagonist to the patient.

180. The method of any one of claims 1-179, wherein the patient exhibits a reduction in the diameter of a junctional zone of adenomyosis following administration of the GnRH antagonist to the patient.

181. The method of claim 180, wherein the patient exhibits the reduction in the diameter of the junctional zone of adenomyosis within from about one day to about 36 weeks of the first administration of the GnRH antagonist to the patient, optionally wherein the patient exhibits the reduction within from about 12 weeks to about 24 weeks of the first administration of the GnRH antagonist to the patient.

182. The method of anyone of claims 1-181, wherein the patient exhibits an improvement in Endometriosis Health Profile questionnaire (EHP-30) score following administration of the GnRH antagonist to the patient.

183. The method of claim 182, wherein the patient exhibits the improvement in the EHP-30 score within from about one day to about 36 weeks of the first administration of the GnRH antagonist to the patient, optionally wherein the patient exhibits the improvement within from about 12 weeks to about 24 weeks of the first administration of the GnRH antagonist to the patient.

184. The method of any one of claims 1-183, wherein the patient exhibits a positive Patient Global Impression of Change (PGIC) score core following administration of the GnRH antagonist to the patient.

185. The method of claim 184, wherein the patient exhibits the positive PGIC score within from about one day to about 36 weeks of the first administration of the GnRH antagonist to the patient, optionally wherein the patient exhibits the positive PGIC score within from about 12 weeks to about 24 weeks of the first administration of the GnRH antagonist to the patient.

186. The method of any one of claims 1-185, wherein the patient does not exhibit a reduction in bone mineral density (BMD) of greater than 5% following administration of the GnRH antagonist to the patient.

187. The method of claim 186, wherein the patient does not exhibit a reduction in BMD of greater than 3% following administration of the GnRH antagonist to the patient.

188. The method of claim 187, wherein the patient does not exhibit a reduction in BMD of greater than 2% following administration of the GnRH antagonist to the patient.

189. The method of claim 188, wherein the patient does not exhibit a reduction in BMD of greater than 1% following administration of the GnRH antagonist to the patient.

190. The method of any one of claims 186-189, wherein the BMD is assessed by dual energy X-ray absorptiometry.

191. The method of claim 190, wherein the BMD is assessed in the spine or femur of the patient.

192. The method of any one of claims 186-189, wherein the BMD is assessed by comparing the concentration of bone specific alkaline phosphatase (BAP) in a sample isolated from the patient following the administration to the concentration of BAP in a sample isolated from the patient prior to the administration.

193. The method of any one of claims 186-189, wherein the BMD is assessed by comparing the concentration of deoxypyridinoline (DPD) in a sample isolated from the patient following the administration to the concentration of DPD in a sample isolated from the patient prior to the administration.

194. The method of any one of claims 186-189, wherein the BMD is assessed by comparing the concentration of type I collagen C-terminal telopeptide (CTX) in a sample isolated from the patient following the administration to the concentration of CTX in a sample isolated from the patient prior to the administration.

195. The method of any one of claims 186-189, wherein the BMD is assessed by comparing the concentration of procollagen 1 N-terminal peptide (P1NP) in a sample isolated from the patient following the administration to the concentration of P1NP in a sample isolated from the patient prior to the administration.

196. A kit comprising a GnRH antagonist and a package insert instructing a user of the kit to administer the GnRH antagonist to a human patient in accordance with the method of any one of claims 1-195.

197. The kit of claim 196, wherein the GnRH antagonist is a compound represented by formula (I) 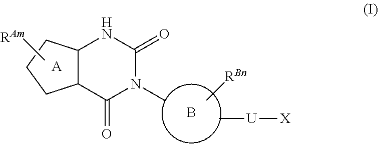
wherein ring A is a thiophene ring;
each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
ring B is an aryl group or a monocyclic heteroaryl group;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and Wa may bind together with the neighboring nitrogen atom to form an optionally substituted cyclic amino group;
n is an integer from 0 to 2;
U is a single bond;
X is a group represented by —S-L-Y, —O-L-Y, —CO-L-Y, or —SO₂-L-Y, wherein L is an optionally substituted lower alkylene group;
Y is a group represented by Z or —NW⁷W⁸, wherein W⁷ and W⁸ independently are a hydrogen atom, an optionally substituted lower alkyl group, or Z with the proviso that W⁷ and W⁸ are not simultaneously hydrogen atoms, or W⁷ and W⁸ may bind together with the neighboring nitrogen atom to form an optionally substituted cyclic amino group; and
Z is an optionally fused and optionally substituted cycloalkyl group, an optionally fused and optionally substituted heterocycloalkyl group, an optionally fused and optionally substituted aryl group, or an optionally fused and optionally substituted heteroaryl group;
or a pharmaceutically acceptable salt thereof.

198. The kit of claim 197, wherein the ring A is a thiophene ring represented by formula (IIa)

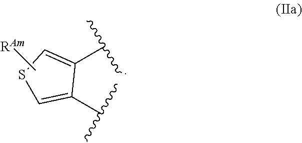

199. The kit of claim 197 or 198, wherein m is 1.

200. The kit of claim 199, wherein the ring A is an optionally substituted thiophene ring represented by formula (IIb)

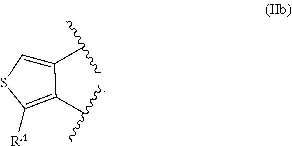

201. The kit of any one of claims 197-200, wherein each R^{A} is independently a halogen atom, an optionally substituted lower alkyl group, COOW¹, or CONW²W³, wherein W¹ to W³ independently are a hydrogen atom or an optionally substituted lower alkyl group, or W² and W³ may bind together with the neighboring nitrogen atom to form an optionally substituted cyclic amino group.

202. The kit of claim 201, wherein each R^{A} is COOH or pharmaceutically acceptable salt thereof.

203. The kit of any one of claims 197-202, wherein the ring B is an optionally substituted benzene ring, pyridine ring, or thiophene ring.

204. The kit of claim 203, wherein the ring B is represented by a formula selected from the group consisting of:

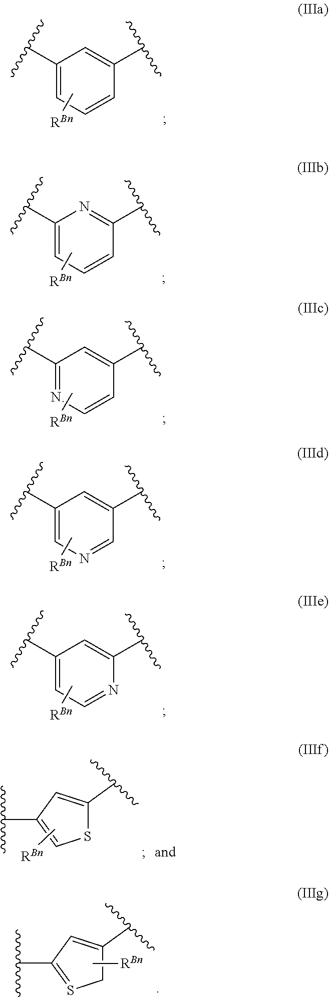

205. The kit of any one of claims 197-204, wherein n is 2.

206. The kit of claim 205, wherein ring B is represented by a formula selected from the group consisting of:

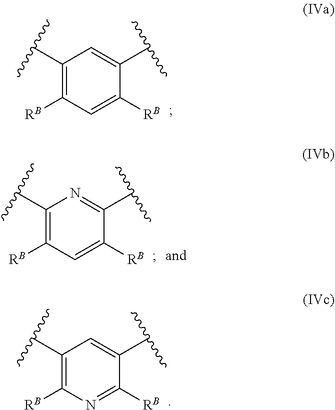

207. The kit of any one of claims 197-206, wherein each R^{B} is independently a halogen atom, an optionally substituted lower alkyl group, or OW⁴, wherein each W⁴ is independently a hydrogen atom or an optionally substituted lower alkyl group.

208. The kit of claim 207, wherein each R^{B} is independently a fluorine atom, chlorine atom, bromine atom, methyl group, or methoxy group.

209. The kit of any one of claims 197-208, wherein U is a single bond.

210. The kit of any one of claims 197-209, wherein X is a group represented by —O-L-Y.

211. The kit of any one of claims 197-210, wherein L is a methylene group.

212. The kit of any one of claims 197-211, wherein Y is an optionally substituted benzene ring represented by formula (V) 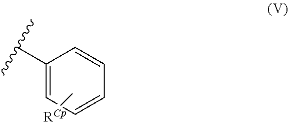
wherein each R^{C} is independently a halogen atom, an optionally substituted lower alkyl group, or OW⁹, wherein each W⁹ is independently a hydrogen atom or an optionally substituted lower alkyl group; and
p is an integer from 0 to 3.

213. The kit of claim 212, wherein Y is a substituted benzene ring represented by formula (Va)

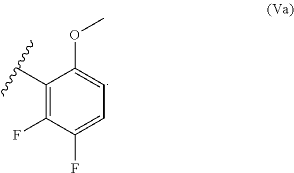 [SMILES: *c1c(OC)ccc(F)c1F]

214. The kit of claim 196, wherein the compound is represented by formula (Ia) 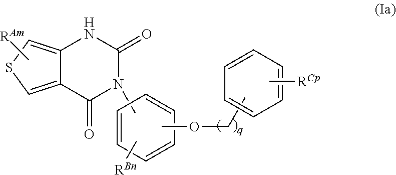
wherein each R^{A} is independently a halogen atom, a cyano group, a nitro group, an optionally substituted lower alkyl group, an optionally substituted lower alkenyl group, an optionally substituted lower alkynyl group, a hydroxyiminomethyl group, an optionally substituted sulfonyl group, an optionally substituted sulfinyl group, a tetrazolyl group, OW¹, SW¹, COW¹, COOW¹, NHCOW¹, NHCONW²W³, NW²W³, CONW²W³, or SO₂NW²W³, wherein W¹ to W³ independently are a hydrogen atom or an optionally substituted lower alkyl group, or W² and W³ may bind together with the neighboring nitrogen atom to form an optionally substituted cyclic amino group;
m is an integer from 0 to 3;
each R^{B} is independently a halogen atom, a cyano group, an optionally substituted lower alkyl group, OW⁴, COW⁴, COOW⁴, or CONW⁵W⁶, wherein W⁴ to W⁶ independently are a hydrogen atom or an optionally substituted lower alkyl group, or W⁵ and W⁶ may bind together with the neighboring nitrogen atom to form an optionally substituted cyclic amino group;
n is an integer from 0 to 2;
q is an integer from 0 to 3;
each R^{C} is independently a halogen atom, an optionally substituted lower alkyl group, or OW⁹, wherein each W⁹ is independently a hydrogen atom or an optionally substituted lower alkyl group; and
p is an integer from 0 to 3;
or a pharmaceutically acceptable salt thereof.

215. The kit of claim 214, wherein the compound is represented by formula (Ib)

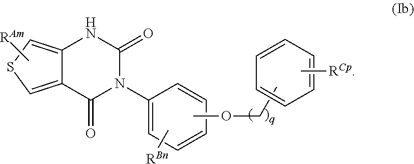

216. The kit of claim 215, wherein the compound is represented by formula (Ic) 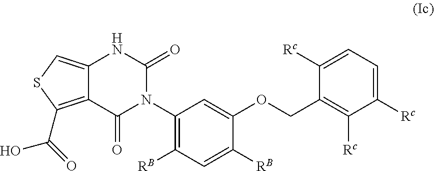
or a pharmaceutically acceptable salt thereof.

217. The kit of any one of claims 197-216, wherein the compound is represented by formula (VI) 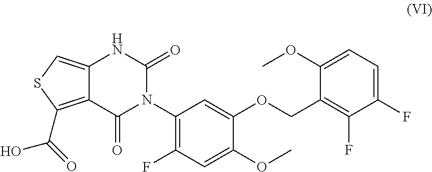 [SMILES: COc1cc(F)c(-n2c(=O)[nH]c3csc(C(=O)O)c3c2=O)cc1OCc1c(OC)ccc(F)c1F]
or a pharmaceutically acceptable salt thereof.

218. The kit of claim 217, wherein the compound is the choline salt of the compound represented by formula (VI).

219. The kit of claim 196, wherein the GnRH antagonist is elagolix, relugolix, opigolix (ASP1707), BAY-784, or SK-2706.
